(19) United States
(12) Patent Application Publication
(10) Pub. No.: US 2024/0299297 A1
(43) Pub. Date: Sep. 12, 2024

(54) STABLE EMULSIONS AND METHODS

(71) Applicant: Hemp Hunter Labs Inc., Geneva, NY (US)

(72) Inventors: John Nicholas Jackowetz, Geneva, NY (US); Soheil Hajirahimkhan, Smiths Falls (CA)

(21) Appl. No.: 18/273,732

(22) PCT Filed: Jan. 27, 2022
(86) PCT No.: PCT/US22/14060

§ 371 Date: Jul. 21, 2023

Related U.S. Application Data
(60) Provisional application No. 63/143,167, filed on Jan. 29, 2021.

(51) Int. Cl. A61K 9/107; A61K 9/50; A61K 31/00; A61K 47/44

(52) U.S. Cl. CPC A61K 9/1075; A61K 9/50; A61K 31/658; A61K 47/44

(57) ABSTRACT

In one aspect, the disclosure provides a stable emulsion that comprises a hydrophobic phase and a hydrophilic phase. In another aspect, the disclosure provides a stable emulsion, comprising a hydrophobic phase and a hydrophilic phase, for electrostatic spray drying. In still another aspect, the disclosure provides a stable, water-dispersible particulate. In yet another aspect, the disclosure provides a method for making a stable emulsion. In a further aspect, the disclosure provides a method for making a stable, water-dispersible particulate. In a still-further aspect, the disclosure provides for compositions comprising an emulsion of this disclosure and particulate.

### CROSS-REFERENCE TO RELATED APPLICATIONS

This application is the U.S. National Phase application of PCT application number PCT/US22/14060 filed on Jan. 27, 2022 which in turn claims the benefit of U.S. provisional application No. 63/143,167 filed on Jan. 29, 2021, the disclosures of which are hereby incorporated by reference in their entirety.

### BACKGROUND

### 1. Field of the Disclosure

The present disclosure relates to stable emulsions. In particular, the disclosure relates to stable cannabinoid emulsions. The disclosure also includes methods of making the emulsions and products comprising the emulsions.

### 2. Description of Related Art

Plants, and portions of plants, are sought out by consumers for use in a variety of products. Oil-based portions or components of plants may be used as an active biological ingredient. An active biological ingredient may exhibit properties and characteristics that consumers seek in different consumer products. For example, essential oils provide not only aromas for products such as perfumes and scented products but also flavors for foodstuffs. An active biological ingredient also may have a salutary effect on health and energy. Many other uses of such components are contemplated.

The hydrophobic nature of oil-based components makes it difficult to incorporate such components into hydrophilic material in amounts that provide the desired effect. Further, the active biological ingredient may be a solid or a viscous material. These properties and characteristics complicate mixing of the active biological ingredient with hydrophilic materials. The dissimilarity of the two fractions also tends to yield an unstable composition.

The disparate properties and characteristics of ingredients of an emulsion make it difficult to make a stable composition including hydrophobic components having consistent consumer acceptance.

There is a need in the art for a stable composition comprising an active biological ingredient, and for a method for making the stable composition.

### SUMMARY OF THE DISCLOSURE

In one aspect, the disclosure provides a stable emulsion that comprises a hydrophobic phase and a hydrophilic phase. The hydrophobic phase comprises an active biological ingredient and a carrier oil. The hydrophilic phase comprises a continuous phase and an emulsifier. The median particle size of the hydrophobic phase in the emulsion may be less than or equal to about 100 nm. The disclosure also provides for products comprising the emulsion.

In another aspect, the disclosure provides a stable emulsion for electrostatic spray drying. In one embodiment, the hydrophobic phase comprises an active biological ingredient and a carrier oil. In one embodiment, the hydrophilic phase comprises a continuous phase, an emulsifier, and an encapsulant. In one embodiment, the median particle size of the hydrophobic phase in the emulsion is less than or equal to about 1,000 nm. In many cases, these aspects of the disclosed stable emulsions may make the emulsion particularly suitable for electrostatic spray drying to form a stable, water-dispersible particle.

In still another aspect, the disclosure provides a stable, water-dispersible particulate. In some embodiments, the particulate comprises an active biological ingredient and carrier oil in an encapsulating shell. In some embodiments, the encapsulant forms a shell or wall around the active biological ingredient and carrier oil to form the particulate. In some embodiments, the stable, water-dispersible particulate may have a median particle size of less than or equal to about 100 μm. In some embodiments, the particulate is suitable for use in both solid and liquid products. The disclosure also provides for products comprising the particulate.

In yet another aspect, the disclosure provides a method for making a stable emulsion. In accordance with the method, a hydrophobic phase is formed by blending hydrophobic components. In some embodiments, the hydrophobic components comprise an active biological ingredient and a carrier oil. In some embodiments, separately, a hydrophilic phase is formed by blending hydrophilic components. In some embodiments, the hydrophilic components comprise a continuous phase and an emulsifier. In some embodiments, the hydrophobic phase and the hydrophilic phase are emulsified in a homogenizer to yield the stable emulsion. In some embodiments, the median particle size of the hydrophobic phase in the emulsion is less than or equal to about 100 nm.

In still another aspect, the disclosure provides a method for making a stable emulsion for electrostatic spray drying. In some embodiments, the method comprises forming a hydrophobic phase by blending hydrophobic components and separately forming a hydrophilic phase. In one embodiment, the hydrophobic components comprise an active biological ingredient and a carrier oil. In one embodiment, the hydrophilic components comprise a continuous phase, an emulsifier, and an encapsulant that forms a shell or a wall around the hydrophobic components when the emulsion is electrostatically spray dried. In some embodiments, the hydrophobic phase and the hydrophilic phase are homogenized in a homogenizer to form the stable emulsion. In some embodiments, the median particle size of the hydrophobic phase in the emulsion is less than or equal to about 1,000 nm. In some embodiments, this stable emulsion is used to make a stable, water-dispersible particulate.

In a further aspect, the disclosure provides a method for making a stable, water-dispersible particulate. In some embodiments, the particulate is made by electrostatically spray drying an emulsion having a hydrophobic phase and a hydrophilic phase. In some embodiments, the hydrophobic phase comprises an active biological ingredient and carrier oil. In some embodiments, the hydrophilic phase comprises a continuous phase, an emulsifier, and an encapsulant. In some embodiments, the encapsulant encapsulates or forms a wall around the active biological ingredient and carrier oil in the stable, water-dispersible particulate. In some embodiments, the median particle size of the hydrophobic phase in the emulsion is less than or equal to about 1,000 nm, and the median particle size of the stable, water-dispersible particulate is less than or equal to about 100 μm.

In a still-further aspect, the disclosure provides for compositions comprising an emulsion of this disclosure and particulate.

Other systems, methods, features, and advantages of the disclosure will be, or will become, apparent to one of ordinary skill in the art upon examination of the following figures and detailed description. It is intended that all such additional systems, methods, features, and advantages be included within this description and this summary, be within the scope of the disclosure, and be protected by the following claims.

### BRIEF DESCRIPTION OF THE DRAWINGS

The disclosure can be better understood with reference to the following drawings and description. The components in the figures are not necessarily to scale, emphasis instead being placed upon illustrating the principles of the disclosure. Moreover, in the figures, like reference numerals designate corresponding parts throughout the different views.

FIG. 1 illustrates a schematic flow diagram for an embodiment of a method of the disclosure;

FIG. 2 illustrates a schematic flow diagram for another embodiment of a method of the disclosure;

FIG. 3 illustrates spray drying an embodiment of a particulate of the disclosure;

FIG. 4A depicts a known spray dried particulate;

FIG. 4B depicts an embodiment of particulate of the disclosure; and

FIG. 5 illustrates properties and characteristics of an embodiment of the disclosure and of an emulsion having larger median particle size.

### DETAILED DESCRIPTION

Oil-based plant components have many uses. Consumer products and other products containing these components are sought by consumers. Consumer products includes edible foodstuff products intended for oral consumption by a mammal. In some embodiments, consumer products include cosmetics, perfumes, and other products. Some embodiments of consumer products have nutritional value. In some embodiments, a consumer product does not have nutritional value. Some embodiments of the disclosure have caloric value; other embodiments are calorie-free.

In some embodiments, foodstuff products include foods, dietary supplements, and beverages. In some embodiments, oil-based plant components provide aroma or flavor to these products. In some embodiments, such components also may supply a therapeutic benefit or another salutary effect to a user. The oil-based plant components may have other uses.

The oil-based components also may be called active biological ingredients. As used herein, an active biological ingredient is a composition that produces an intended effect when ingested, inhaled, applied to, or otherwise introduced to a body of a mammal. In some embodiments, active biological ingredients are natural or synthetic products known in the art. Some embodiments of active biological ingredients are natural ingredients. In other embodiments, an active biological ingredient is a synthetic composition, a semi-synthetic ingredient, or an otherwise-modified composition. In some embodiments, such modification may be made by fractionation, fermentation, biological engineering, or other processes. In some embodiments, an active biological ingredient is a composition that is converted by the body to a biologically active composition.

Generally, oil-based components are hydrophobic and thus often not easily combined with hydrophilic products. The incompatibility between the hydrophobic components and hydrophilic products makes it difficult to combine a quantity of the hydrophobic plant component sufficient to achieve the desired result with a hydrophilic product.

In accordance with the disclosure, bioavailability of the active biological ingredient is superior to bioavailability achieved by known delivery techniques. In embodiments of the disclosure, bioavailability of the active biological ingredient is increased over known delivery techniques by selection of carrier oil and control of median particle size.

In some embodiments, the compositions that form the emulsion or particulate have purity sufficient to be characterized as food grade. In some embodiments, the compositions are listed in or meet the properties and characteristics of such components listed in the United States Pharmacopeia, the European Pharmacopoeia, or similar authoritative standards publications.

### Nanoparticle Liquid Emulsion

The disclosure is directed to emulsions and particulates that enable introduction of an oil-based plant component into a hydrophilic composition. The disclosure is directed to a stable emulsion that comprises a hydrophobic phase and a hydrophilic phase. In some embodiments, the hydrophobic phase comprises an active biological ingredient and a carrier oil. In some embodiments, the hydrophilic phase comprises a continuous phase and an emulsifier. In embodiments, the median particle size of the hydrophobic phase in the emulsion is less than or equal to about 500 nm, and in some embodiments is between about 1 nm and about 500 nm.

In various embodiments, the median particle size of the hydrophobic phase in the emulsion may be between 2 nm and 450 nm, or between 3 nm and 400 nm, or between 4 nm and 350 nm, or between 5 nm and 300 nm, or between 6 nm and 250 nm, or between 7 nm and 200 nm, or between 8 nm and 150 nm, or between 9 nm and 125 nm, or between 10 nm and 100 nm.

In still other embodiments, the median particle size of the hydrophobic phase is between about 25 nm and about 400 nm, and in yet other embodiments is between about 30 nm and about 300 nm. In some embodiments, the median particle size of the hydrophobic phase in the emulsion is between about 35 nm and about 250 nm. In some embodiments, the median particle size of the hydrophobic phase in the emulsion is between about 40 nm and about 200 nm. In some embodiments, the median particle size of the hydrophobic phase in the emulsion is between about 45 nm and about 200 nm. In some embodiments, the median particle size of the hydrophobic phase in the emulsion is between about 50 nm and about 200 nm. In some embodiments, the median particle size of the hydrophobic phase in the emulsion is between about 50 nm and about 190 nm. In some embodiments, the median particle size of the hydrophobic phase in the emulsion is between about 50 nm and about 180 nm. In some embodiments, the median particle size of the hydrophobic phase in the emulsion is between about 50 nm and about 170 nm. In some embodiments, the median particle size of the hydrophobic phase in the emulsion is between about 50 nm and about 160 nm. In some embodiments, the median particle size of the hydrophobic phase in the emulsion is between about 50 nm and about 150 nm. In some embodiments, the median particle size of the hydrophobic phase in the emulsion is between about 50 nm and about 140 nm. In some embodiments, the median particle size of the hydrophobic phase in the emulsion is between about 50 nm and about 130 nm. In some embodiments, the median particle size of the hydrophobic phase in the emulsion is between about 50 nm and about 120 nm. In some embodiments, the median particle size of the hydrophobic phase in the emulsion is between about 50 nm and about 110 nm. In some embodiments, the median particle size of the hydrophobic phase in the emulsion is between about 50 nm and about 100 nm.

The skilled practitioner recognizes that the medium into which the emulsion is dissolved may affect the particle size in the emulsion. In embodiments, this phenomenon is related to the interaction of emulsifiers with other constituents in the liquid. For example, lecithin is an ionic surfactant. Therefore, other ions in solution, such as salts, can disrupt the ability of lecithin to emulsify. This disruption leads to larger particles and overall instability. As used herein, ‘particle size’ identifies particle size in the emulsion, unless the context makes it clear that the particle size is measured after the emulsion is dissolved in another medium, such as a consumer product.

As used herein, the word ‘particle’ means a minute portion, quantity, or portion of tiny size of a composition. Further, ‘particulate’ means an agglomeration or a combination of particles, typically held together in association with each other, with or without a binder.

Stable emulsions that are an embodiment of the disclosure contain a combination of emulsifiers and carrier oils. In some embodiments, the active biological ingredient, emulsifiers, carrier oils, and any additives, are food grade ingredients. In embodiments of the disclosure, the median particle size is controlled to provide improved bioavailability of the active biological ingredient and stability of the stable emulsion. Some embodiments of the disclosure also are clear in appearance and do not adversely affect the clarity of clear products.

In embodiments of the disclosure, active biological ingredients are incorporated into a stable emulsion. A stable emulsion is a water-soluble liquid emulsion comprising an active biological ingredient or a combination of active biological ingredients. In some embodiments, the total weight percent of active biological ingredients in the stable emulsion is between about 0.001 weight percent and about 30 weight percent, based on the total weight of the stable emulsion. In embodiments, the total weight percent of active biological ingredients in a stable emulsion is between about 0.5 weight percent and about 25 weight percent. In some embodiments, the total weight percent of active biological ingredients in a stable emulsion is between about 0.5 weight percent and about 22.5 weight percent, based on the total weight of the stable emulsion. In some embodiments, the total weight percent of active biological ingredients in a stable emulsion is between about 0.5 weight percent and about 20 weight percent, based on the total weight of the stable emulsion. In some embodiments, the total weight percent of active biological ingredients in a stable emulsion is between about 0.5 weight percent and about 15 weight percent. In other embodiments, the total weight percent of active biological ingredients is between about 0.5 weight percent and about 10 weight percent, based on the total weight of the stable emulsion. Finally, in some embodiments the total weight percent of active biological ingredients may be about 22.5%.

In accordance with the disclosure, the hydrophobic phase comprises at least one active biological ingredient and at least one carrier oil. In one embodiment of this disclosure, one or more active biological ingredients are selected from oil-based plant components, for example, extracts of plants. In one embodiment, the active biological ingredient is an oil-soluble extract of one or more plants including teas, curcumin, melatonin, grape stilbene, capsicum, capsaicin, carnosic acid, a-pinene, coenzyme Q10, menthol, omega-3, Vitamin D, Vitamin K, Vitamin E, oopept, khomitoxin, menthol, St. John's wort, neem, cardamom, cinnamon, palmitic acid, lineolic acid, galantamine, rosmarinic acid, 1,8-cineole, sage, hyperazine A, passion flower, Camellia sinensis, C. ternatea extracts, C. asiatica extracts, Acorus calamus extracts, C. paniculatus extracts, Salvia mitorhizza extracts, Crocus sativu extracts, Evodia rutaecarpa extracts, coriander, linden, lemon verbena, Brahmi, apigenin, sinomenine, decursinol, jujube, soya glycine, hop, Tulsi, rosehip extract, Coenzyme Q10, tocopherol, tocotrienol cannabinoids, and blends thereof.

In some embodiments, the active biological ingredient is a cannabinoid. As used herein, a cannabinoid is a member of a group of related compounds that include CBD and the active constituents of cannabis. Approximately 150 cannabinoids have been identified to date, and more may be identified in the future. In some embodiments, cannabinoid includes Cannabigerovarin (CBGV), Cannabigerovarinic acid (CBGVA), Cannabigerol (CBG), Cannabigerolic acid (CBGA), Cannabivarichromene (CBCV), Cannabichromene (CBC), Cannabidivarin (CBDV), Cannabidiol (CBD), Cannabidiolic acid (CBDA), trans-delta-8-Tetrahydrocannabinol (delta-8-THC), delta-9-trans-Tetrahydrocannabivarin (THCV), delta-9-trans-Tetrahydrocannabinol (delta-9-THC), Cannabicitran (citrilidene-cannabis) (CBT), Cannabielsoin (CBE), Cannabicyclol (CBL), Cannabivarin (CBV), Cannabinol (CBN), Tetrahydrocannabiphorol (THCP), Cannabidiphorol (CBDP), Cannabimovone (CBM). O-Methlycannabigerol, Cannabigerolic acid methylether, Cannabinerolic acid, Cannabinerol, Carmagerol, rac-6′-Epoxycannabigerol, rac-6′-Epoxycannabigerolic acid, rac-6′-Epoxycannabinerol, rac-6′-Epoxycannabinerolic acid, gamma-Eudesmyl cannabigerolate, gamma-Cadinyl cannabigerolate, Sesquicannabigerol, Deprenyl O-methyl cannabigerolic acid (Amorfrutin 2), 5-Acetyl-4-hydroxycannabigerol, Acetylcannabigeroquinol, Cannabigeroquinone, Abnormal cannabigerol, Acetyl abnormal hydrocannabigeroquinol, Abnormal cannabigeroquinol, 2′-Hydroxy-1′,2′-dihydrocannabichromene (Cyclo-CBG), Cannabiorcichromene, Cannabiorcichromenic acid, Cannabichromevarinic acid, Cannabichromenic acid, 4-Acetoxycannabichromene, Anthopogochromenic acid, Confluentin, Daurichromenic acid, 8′-Hydroxyisocannabichromene, 4-Acetoxycannabichromene, Cannabidiorcol, Cannabidivarinic acid, nor-Cannabidiol, O-Methlycannabidiol, O-Propylcannabidiol, O-Pentylcannabidiol, CBDA-THC ester, Ferruginene C, Cannabioxepane, Cannabinodivarin, Cannabinodiol, Cannabifuran, Dehydrocannabifuran, trans-delta-8-Tetrahydrocannabinolic acid, 10α-Hydroxy trans-delta-8-tetrahydrocannabinol, 10β-Hydroxy trans-delta-8-tetrahydrocannabinol, 11-Acetoxy-delta-8-tetrahydrocannabinoic acid, 10-Hydroxy-9-oxo-delta-8-tetrahydrocannabinol, delta-9-trans-Tetrahydrocannabiorcol, delta-9-trans-Tetrahydrocannabiorcolic acid, delta-9-trans-Tetrahydrocannabivarinic acid, delta-9-trans-nor-Tetrahydrocannabinol, delta-9-trans-nor-Tetrahydrocannabinolic acid, delta-9-trans-Tetrahydrocannabinolic acid A, delta-9-trans-Tetrahydrocannabinolic acid B, 8α-Hydroxy-delta-9-trans-tetrahydrocannabinol, 8β-Hydroxy-delta-9-trans-tetrahydrocannabinol, 8-Oxo-delta-9-trans-tetrahydrocannabinol, O-Propyl-delta-9-trans-tetrahydrocannabinol, O-Pentyl-delta-9-trans-tetrahydrocannabinol, 2-Formyl-delta-9-trans-tetrahydrocannabinol, β-Fenchyl delta-9-trans-Tetrahydrocannabinolate, α-Fenchyl delta-9-trans-Tetrahydrocannabinolate, Bornyl delta-9-trans-Tetrahydrocannabinolate, epi-Bornyl delta-9-trans-Tetrahydrocannabinolate, α-Terpinyl delta-9-trans-Tetrahydrocannabinolate, 4-Terpinyl delta-9-trans-Tetrahydrocannabinolate, gamma-Eudesmyl delta-9-trans-Tetrahydrocannabinolate, α-Cadinyl delta-9-trans-Tetrahydrocannabinolate, Hexahydrocannabinol, Hydroxy delta-9,11-hexahydrocannabinol, Methylen-bis delta-9-trans-Tetrahydrocannabinol (Cannabisol), Tetrahydrocannabinol epoxide, delta-9-trans-Tetrahydrocannabinol glycol (cannabiripsol), 6a,7,10a-Trihydroxy-delta-9-tetrahydrocannabinol, delta-9-cis-Tetrahydrocannabivarin, delta-9-cis-Tetrahydrocannabinol, Cannabiorcicitran, Bis-nor cannabitriol, Bis-nor-Cannabitriol isomer, 10-O-Ethyl bis-nor cannabitriol, Isocannabitriol, Cannabitriol, Cannabitriol isomer, 10-O-Ethyl cannabitriol isomer, 10-Oxo-delta-6a(10a)-tetrahydrocannabinol, 9,10-Anhydrocannabitirol, Cannabiglendol, 7,8-Dehydro-10-O-ethylcannabitriol, delta-7-cis-Isotetrahydrocannabivarin, delta-7-trans-Isotetrahydrocannabivarin, delta-7-trans-isotetrahydrocannabinol, Bis-nor-cannabielsoin, Bis-nor-Cannabielsoic acid B, Cannabielsoic acid A, Cannabielsoic acid B, Ferruginene A, Ferruginene B, Cannabiorcicyclol, Cannabiorcicyclolic acid, Cannabicyclovarin, Cannabicyclolic acid, Anthopogocyclolic acid, Rhododaurichromanic acid A, Cannabiorcol, nor-Cannabivarin, nor-Cannabinol, Cannabinolic acid, O-Methylcannabinol, O-Propylcannabinol, O-Penthylcannabinol, 7-Hydroxcannabinol, 8-Hydroxycannabinol, 8-Hydroxcannabinolic acid, 7,8-Dihydrocannabinol, 4-Terpenyl cannabinolate, Cannabicoumaronone, Cannabicourmarononic acid, Bisnor-Cannabichromanone, Cannabichromanone, (6aR)-Cannabichromanone B, (6aR)-Cannabichromanone C, Cannabichromanone, Cannabimovone, Anhydrocannabimovone, Demethyldecarboxyamorfrutin A, and mixtures thereof.

In some embodiments, the active biological ingredient may be a cannabinoid selected from the group including Cannabigerovarin (CBGV), Cannabigerovarinic acid (CBGVA), Cannabigerol (CBG), Cannabigerolic acid (CBGA), Cannabivarichromene (CBCV), Cannabichromene (CBC), Cannabidivarin (CBDV), Cannabidiol (CBD), Cannabidiolic acid (CBDA), trans-delta-8-Tetrahydrocannabinol (delta-8-THC), delta-9-trans-Tetrahydrocannabivarin (THCV), delta-9-trans-Tetrahydrocannabinol (delta-9-THC), Cannabicitran (citrilidene-cannabis) (CBT), Cannabielsoin (CBE), Cannabicyclol (CBL), Cannabivarin (CBV), Cannabinol (CBN), Tetrahydrocannabiphorol (THCP), Cannabidiphorol (CBDP), Cannabimovone (CBM), and mixtures thereof.

In other embodiments, the active biological ingredient may be selected from the group including full spectrum cannabidiol (CBD) distillate containing delta-9-tetrahydrocannabinol (“THC”), broad spectrum cannabidiol essentially devoid of THC, cannabidiol isolate, cannabigerol (CBG) isolate, CBG distillate, cannabinol (CBN) isolate, cannabichromene (CBC) isolate, CBC distillate, cannabidivarin (CBDV) isolate, cannabidiolic acid (CBDA) isolate, cannabigerolic acid (CBGA) isolate, cannabichromenic acid (CBCA) isolate, THC distillate, and blends thereof. The isolate compositions in embodiments may have a purity of at least about 95% by weight, or at least about 99% by weight, or at least about 99.5% by weight.

In various embodiments, the active biological ingredient may be a full spectrum cannabidiol distillate that includes one or more cannabinoids, and also contains less than about 0.3 weight percent delta-9-tetrahydrocannabinol (“THC”); or the active biological ingredient may be a broad spectrum cannabidiol essentially devoid of THC (i.e., having a THC weight percent below limits of analytical detection by standard tests); or the active biological ingredient may be one or more of cannabidiol isolate, cannabigerol (CBG) isolate, cannabinol (CBN) isolate, and blends thereof. In some embodiments, the active biological ingredient may be a full spectrum cannabidiol distillate containing tetrahydrocannabinol (“THC”), a broad spectrum cannabidiol essentially devoid of THC, cannabidiol isolate, and blends thereof.

In some embodiments, the active biological ingredient used in formulating the emulsion may be added in the form of a viscous fluid or a solid. In such embodiments, a carrier oil may be used to facilitate formation of the emulsion. In some embodiments, the carrier oil is selected to increase bioavailability of the active biological ingredient. For example, the bioavailability of THC and CBD in pure CBD isolate crystals is between about 3 percent and about 8 percent. However, in some embodiments, selected carrier oils may increase this bioavailability.

In embodiments of the disclosure, the carrier oil is selected from one or more triglycerides. In one embodiment, that triglyceride comprises a medium chain fatty acid. In one embodiment, that triglyceride comprises a long chain fatty acid. In one embodiment, the carrier oil comprises both a medium chain fatty acid and a long chain fatty acid. Within the context of this disclosure, the fatty acids have between about 14 and about 18 carbon atoms. In some embodiments, these carrier oils have a melting point less than about 80° C. The fatty acid in some embodiments may be saturated, monounsaturated (one double bond), or polyunsaturated (two or more double bonds). These carrier oils may be absorbed in a human consumer of the product through the lymphatic system. These carrier oils thus may avoid first pass metabolism and, in so doing, increase bioavailability of the active biological ingredient.

In embodiments of the disclosure, the carrier oil may be selected from the group including sesame seed oil, soybean oil, olive oil, canola oil, grape seed oil, safflower oil, sunflower seed oil, pumpkin seed oil, peanut oil, corn oil, cottonseed oil, butternut squash seed oil, flaxseed oil, palm oil, hemp seed oil, Maisine CC® (glyceryl monolinoleate, a triglyceride of fatty acids having 18 carbon atoms and two double bonds), Peceol® (glyceryl monooleate, a triglyceride of monounsaturated fatty acids having 18 carbon atoms), and blends thereof. In embodiments, the carrier oil may be selected from the group including sesame seed oil, soybean oil, olive oil, canola oil, and blends thereof. In some embodiments, blends of carrier oils in some embodiments may be used to take advantage of the various properties and characteristics of each oil. For example, a first carrier oil in some embodiments may be particularly effective at solubilizing the active biological ingredient. In some embodiments, both a first and second carrier oil may be included in the disclosed compositions. Including a second carrier oil, in some embodiments, may be particularly effective at stabilizing the emulsion.

Short and medium chain fatty acids, such as MCT oils, are linear saturated triglycerides having fatty acid chains having up to about 12 carbon atoms. Use of these short and medium chain fatty acids and oils as the carrier oil may encourage absorption of the active biological ingredient through the portal vein and into the liver. Such first pass metabolism is not ideal, as the active biological ingredient will be metabolized by the liver. Metabolism by the liver alters the structure of the active biological ingredient before the active biological ingredient reaches systemic circulation. However, in some embodiments, a minor weight percent of MCT oil is used in combination with a carrier oil. In embodiments, the weight percent of MCT oil is between about 0.5 weight percent and about 40.0 weight percent, based on the weight of the carrier oils. In other embodiments, the weight percent of MCT oil is between about 0.5 weight percent and about 30.0 weight percent, based on the total weight of the carrier oils. In some embodiments, MCT oil is present at between about 0.5 weight percent and about 25.0 weight percent, based on the weight of the carrier oils. In other embodiments, the weight percent of MCT oil is between about 1.0 weight percent and about 25.0 weight percent, based on the weight of the carrier oils. MCT oil may help dissolve CBDs into other carrier oils. If MCT oil is used, it may be appropriate to increase the weight percent of active biological ingredient to compensate for the lower bioavailability.

In embodiments of the disclosure, the weight percent of the carrier oil is between about 0.5 weight percent and about 40.0 weight percent, based on the total weight of the stable emulsion. In other embodiments, the weight percent of carrier oil is between about 0.5 weight percent and about 30.0 weight percent, based on the total weight of the stable emulsion. In some embodiments, carrier oil is present at between about 0.5 weight percent and about 25.0 weight percent, based on the total weight of the stable emulsion. In other embodiments, the weight percent of carrier oil is between about 1.0 weight percent and about 25.0 weight percent, based on the total weight of the stable emulsion.

In accordance with the disclosure, the stable emulsion also contains a hydrophilic phase. This hydrophilic phase has a continuous phase including a first (primary) emulsifier. In some embodiments, the hydrophilic phase may include a second (secondary) emulsifier. In some embodiments, the hydrophilic phase may include a third (tertiary) emulsifier. In some embodiments, the hydrophilic phase may include a co-solvent. Further, a water-soluble active biological ingredient may be included in the hydrophilic phase in some embodiments of the disclosure.

In embodiments of the disclosure, the continuous phase comprises greater than 30% of the hydrophilic phase. In some embodiments, the continuous phase comprises between about 30 weight percent and about 98.5 weight percent of the stable emulsion, based on the total weight of the stable emulsion. In some embodiments, the continuous phase forms between about 50 weight percent and about 97 weight percent, based on the total weight of the stable emulsion. In some embodiments, the continuous phase carries a primary emulsifier to enable emulsification of the hydrophobic phase. In some embodiments, the continuous phase includes a primary emulsifier and secondary emulsifier. In some embodiments, the continuous phase includes a primary emulsifier, a secondary emulsifier, and a tertiary emulsifier. In some examples, including a secondary and/or a tertiary emulsifier may modify a property of the emulsion such as stability and turbidity in dilution. In some embodiments, including a secondary and/or a tertiary emulsifier may allow for greater compatibility in various finish goods or products. Such improvements in compatibility may be apparent upon examining one or more properties such as turbidity in dilution and stability parameters. A co-solvent in some embodiments may be present to increase the solubility of both hydrophilic and hydrophobic components in the emulsion. In some embodiments when co-solvent is present, the continuous phase is between about 50 weight percent and about 90 weight percent, and in some embodiments is between about 60 weight percent and about 85 weight percent, based on the total weight of the stable emulsion.

Water in some embodiments may be used as a continuous phase despite general recognition in the art that an aqueous continuous phase introduces increased risk of microbial growth and spoilage. In embodiments, water is used as a continuous phase in the form of aqueous solutions with reduced water activity. In some embodiments, sugar solutions have sufficiently reduced water activity. In some embodiments, the water activity is reduced to about 0.90 to suppress bacterial growth and to about 0.80 to suppress mold growth. The rate of bacterial and mold growth can be measured by methods known in the art including agar plating. In other embodiments, the water activity is reduced to between about 0.05 and about 0.9 to provide particular properties and characteristics for the emulsion or for a consumer good. In various other embodiments, the water activity may be reduced to between 0.01 and 0.8, or between 0.01 and 0.7, or between 0.01 and 0.6, or between 0.01 and 0.5, or between 0.001 and 0.04, or between 0.01 and 0.03.

Water activity is defined in the art as the ratio between the vapor pressure of the food itself, when in a completely undisturbed balance with the surrounding air media, and the vapor pressure of distilled water under identical conditions. Water activity can be measured by methods known in the art including using a water activity meter.

In some embodiments, the non-aqueous continuous phase may be selected from the group including glycerin, propylene glycol, 1,3-propanediol, sugar alcohol solution, sugar syrup, and blends thereof. In embodiments, sugar alcohol solutions include solutions of sorbitol, maltitol, erythritol, sucralose xylitol, and blends thereof. In embodiments, the non-aqueous continuous phase may be selected from the group including glycerin, propylene glycol, sugar syrup, and blends thereof.

In accordance with the disclosure, an emulsifier is used to enable formation of an emulsion of a hydrophobic phase and a hydrophilic phase. In some embodiments of this disclosure, an emulsifier may be selected based on its ability to effectively coat and solubilize the hydrophobic payload. In some embodiments, effective emulsifiers create stable microemulsions, or nanoparticle emulsions (<100 nm median particle size), that are compatible with the finished good (e.g. beverage). In embodiments of the disclosure, the total weight percent of emulsifier in the stable emulsion may be between about 0.001 weight percent and about 30 weight percent, based on the total weight of the stable emulsion. In some embodiments, the total weight percent of emulsifier in the stable emulsion is between about 0.01 weight percent and about 30 weight percent, based on the total weight of the stable emulsion. In some embodiments, the total weight percent of emulsifier in the stable emulsion is between about 0.1 weight percent and about 30 weight percent, based on the total weight of the stable emulsion. In some embodiments, the total weight percent of emulsifier in the stable emulsion is between about 0.5 weight percent and about 30 weight percent, based on the total weight of the stable emulsion. In some embodiments, the total weight percent of emulsifier in the stable emulsion is between about 0.5 weight percent and about 27.5 weight percent, based on the total weight of the stable emulsion. In some embodiments, the total weight percent of emulsifier in the stable emulsion is between about 0.5 weight percent and about 25.0 weight percent, based on the total weight of the stable emulsion. In some embodiments, the total weight percent of emulsifier in the stable emulsion is between about 0.5 weight percent and about 22.5 weight percent, based on the total weight of the stable emulsion. In some embodiments, the total weight percent of emulsifier in the stable emulsion is between about 0.5 weight percent and about 20.0 weight percent, based on the total weight of the stable emulsion. In some embodiments, the total weight percent of emulsifier in the stable emulsion is between about 0.5 weight percent and about 19.0 weight percent, based on the total weight of the stable emulsion. In some embodiments, the total weight percent of emulsifier in the stable emulsion is between about 0.5 weight percent and about 18.0 weight percent, based on the total weight of the stable emulsion. In some embodiments, the total weight percent of emulsifier in the stable emulsion is between about 0.5 weight percent and about 17.0 weight percent, based on the total weight of the stable emulsion. In some embodiments, the total weight percent of emulsifier in the stable emulsion is between about 0.5 weight percent and about 16.0 weight percent, based on the total weight of the stable emulsion. In some embodiments, the total weight percent of emulsifier in the stable emulsion is between about 0.5 weight percent and about 15.0 weight percent, based on the total weight of the stable emulsion.

The emulsifier in some embodiments may be selected from lecithins, saponins, gum Arabic and modified gum Arabic, sucrose esters, mono- and di-glycerides, ethoxylated mono- and di-glycerides, rhamnolipids having between about 10 percent and about 90 percent rhamnolipids, sophorolipids having between about 10 percent and about 90 percent sophorolipids, and blends thereof. Lecithins include soy lecithin, sunflower lecithin, hydroxylated lecithin, acetylated lecithin, hydrolyzed lecithin, enzyme modified lecithin, and lyso-lecithin and other high phosphatidylcholine compositions. The percentage of phosphatidylcholine in soy lecithin in some embodiments may be between about 10 percent and 100 percent of the total weight of the soy lecithin, and is between about 25 weight percent and about 90 weight percent, based on the total weight of the soy lecithin, in other embodiments. In embodiments, hydrogenated soy lecithin has at least about 70 percent phosphatidylcholine. In some embodiments, hydrogenated soy lecithin has between about 70 weight percent and about 99 weight percent phosphatidylcholine, based on the total weight of the hydrogenated soy lecithin. In other embodiments, hydrogenated soy lecithin contains between about 70 weight percent and 80 weight percent phosphatidylcholine, based on the total weight of the hydrogenated soy lecithin. In other embodiments, hydrogenated sunflower lecithin has up to about 90 percent phosphatidylcholine. In embodiments, hydrogenated sunflower lecithin contains between about 10 weight percent and about 60 weight percent phosphatidylcholine, based on the total weight of the hydrogenated sunflower lecithin. Still other embodiments contain between about 30 weight percent and about 60 percent phosphatidylcholine, based on the total weight of the hydrogenated sunflower lecithin. In some embodiments, sunflower lecithin contains between about 1 weight percent and about 50 weight percent phosphatidylcholine, based on the total weight of the sunflower lecithin. Saponins include Quillaja extract, commercially available as Q-naturale®. Sucrose esters include sucrose monopalmitate, sucrose monolauriate, and sucrose acetate isobutyrate. Mono- and di-glycerides include glycerol monooleate, linoleoyl polyoxyl-6 glycerides, caprylocaproyl-8 glycerides, and oleoyl polyoxyl-6 glycerides. Pectins include pectin extracts from apples, citrus fruit, and beets.

Exemplary emulsifiers used to make oil in water emulsions are branded as Span® and Tween®. These are synthetically made, non-ionic surfactants composed of sorbitan esters and polyethoxylated sorbitan esters, respectively. While these emulsifiers are effective, their use is limited in common foodstuffs due to governmental regulations and they are not considered “clean label”. The skilled practitioner recognizes that ‘clean label’ includes the concept of making sure that ingredients in the product are items than consumers recognize and regard as wholesome. Moreover, the use of synthetic ingredients in combination with natural botanicals is incongruent with current food marketing and consumer trends. In some edible embodiments of the disclosure, such emulsifiers are not used. Such emulsifiers are be used in some embodiments of other products.

In embodiments, the emulsifier may be selected from the group including lecithins, saponins, gum Arabic, sucrose esters, mono- and di-glycerides, rhamnolipids having between about 10 percent and about 90 percent rhamnolipids, sophorolipids having between about 10 percent and about 90 percent sophorolipids, pectins, and blends thereof. In embodiments of the disclosure, the emulsifier may be selected from the group including lecithins, sucrose esters, rhamnolipids, saponins, and blends thereof.

In embodiments of the disclosure, a secondary emulsifier may be present to help strengthen the emulsion and acts to complement the primary emulsifier. For example, lecithin in some embodiments is blended with a sucrose ester to benefit from the non-ionic nature of lecithin and help increase the overall stability of emulsions in highly charged solutions (e.g. acidic beverages).

In embodiments of the disclosure, the secondary emulsifier may be selected from the groups including lecithins, saponins, gum Arabic and modified gum Arabic, sucrose esters, mono- and di-glycerides, ethoxylated mono- and di-glycerides, rhamnolipids having between about 10 percent and about 90 percent rhamnolipids, sophorolipids having between about 10 percent and about 90 percent sophorolipids, pectin, and blends thereof. Lecithins include soy lecithin, sunflower lecithin, hydroxylated lecithin, acetylated lecithin, hydrolyzed lecithin, enzyme modified lecithin, lyso-lecithin, and other high phosphatidylcholine compositions. The properties and characteristics of the lecithin-containing components are the same as the properties and characteristics of these components described herein. Saponins include Quillaja extract, commercially available as Q-naturale®. Sucrose esters include sucrose monopalmitate, sucrose monolauriate, and sucrose acetate isobutyrate. Mono- and di-glycerides include glycerol monooleate, linoleoyl polyoxyl-6 glycerides, caprylocaproyl-8 glycerides, and oleoyl polyoxyl-6 glycerides. Pectins in some embodiments are from apple, beet, and citrus sources.

In accordance with some embodiments of the disclosure, the weight percent of secondary emulsifier in the stable emulsion, if present, may be between about 0.001 weight percent and about 25.0 weight percent, based on the total weight of the stable emulsion. In some embodiments, the weight percent of secondary emulsifier in a stable emulsion is between about 0.01 weight percent and about 25.0 weight percent, based on the total weight of the stable emulsion. In some embodiments, the weight percent of secondary emulsifier in a stable emulsion is between about 0.1 weight percent and about 25.0 weight percent, based on the total weight of the stable emulsion. In some embodiments, the weight percent of secondary emulsifier in a stable emulsion is between about 0.1 weight percent and about 20.0 weight percent, based on the total weight of the stable emulsion. In some embodiments, the weight percent of secondary emulsifier in a stable emulsion is between about 0.1 weight percent and about 19.0 weight percent, based on the total weight of the stable emulsion. In some embodiments, the weight percent of secondary emulsifier in a stable emulsion is between about 0.1 weight percent and about 18.0 weight percent, based on the total weight of the stable emulsion. In some embodiments, the weight percent of secondary emulsifier in a stable emulsion is between about 0.1 weight percent and about 17.0 weight percent, based on the total weight of the stable emulsion. In some embodiments, the weight percent of secondary emulsifier in a stable emulsion is between about 0.1 weight percent and about 16.0 weight percent, based on the total weight of the stable emulsion. In some embodiments, the weight percent of secondary emulsifier in a stable emulsion is between about 0.1 weight percent and about 15.0 weight percent, based on the total weight of the stable emulsion. In some embodiments, the weight percent of secondary emulsifier in a stable emulsion is between about 0.2 weight percent and about 15.0 weight percent, based on the total weight of the stable emulsion. In some embodiments, the weight percent of secondary emulsifier in a stable emulsion is between about 0.3 weight percent and about 15.0 weight percent, based on the total weight of the stable emulsion. In some embodiments, the weight percent of secondary emulsifier in a stable emulsion is between about 0.4 weight percent and about 15.0 weight percent, based on the total weight of the stable emulsion. In some embodiments, the weight percent of secondary emulsifier in a stable emulsion is between about 0.5 weight percent and about 15.0 weight percent, based on the total weight of the stable emulsion.

In embodiments of the disclosure, a tertiary emulsifier may be present to help strengthen the emulsion and acts to complement the primary emulsifier and the secondary emulsifier.

In embodiments of the disclosure, the tertiary emulsifier may be selected from the group including lecithins, saponins, gum Arabic and modified gum Arabic, sucrose esters, mono- and di-glycerides, ethoxylated mono- and di-glycerides, rhamnolipids having between about 10 percent and about 90 percent rhamnolipids, sophorolipids having between about 10 percent and about 90 percent sophorolipids, pectin, and blends thereof. Lecithins include soy lecithin, sunflower lecithin, hydroxylated lecithin, acetylated lecithin, hydrolyzed lecithin, enzyme modified lecithin, lyso-lecithin, and other high phosphatidylcholine compositions. The properties and characteristics of the lecithin-containing components are the same as the properties and characteristics of these components described herein. Saponins include Quillaja extract, commercially available as Q-naturale®. Sucrose esters include sucrose monopalmitate, sucrose monolauriate, and sucrose acetate isobutyrate. Mono- and di-glycerides include glycerol monooleate, linoleoyl polyoxyl-6 glycerides, caprylocaproyl-8 glycerides, and oleoyl polyoxyl-6 glycerides. Pectins in some embodiments are from apple, beet, and citrus sources.

In accordance with some embodiments of the disclosure, the weight percent of tertiary emulsifier in the stable emulsion, if present, may be between about 0.001 weight percent and about 10 weight percent, based on the total weight of the stable emulsion. In some embodiments, the weight percent of tertiary emulsifier in a stable emulsion is between about 0.01 weight percent and about 5.0 weight percent, based on the total weight of the stable emulsion. In some embodiments, the weight percent of tertiary emulsifier in a stable emulsion is between about 0.1 weight percent and about 5.0 weight percent, based on the total weight of the stable emulsion. In some embodiments, the weight percent of tertiary emulsifier in a stable emulsion is between about 0.1 weight percent and about 4.5 weight percent, based on the total weight of the stable emulsion. In some embodiments, the weight percent of tertiary emulsifier in a stable emulsion is between about 0.1 weight percent and about 4.0 weight percent, based on the total weight of the stable emulsion. In some embodiments, the weight percent of tertiary emulsifier in a stable emulsion is between about 0.1 weight percent and about 3.5 weight percent, based on the total weight of the stable emulsion. In some embodiments, the weight percent of tertiary emulsifier in a stable emulsion is between about 0.1 weight percent and about 3.0 weight percent, based on the total weight of the stable emulsion.

In some embodiments, if present, the co-solvent may have a weight percent of between about 0.001 weight percent and about 50.0 weight percent, based on the total weight of the stable emulsion. In embodiments, the weight percent of co-solvent in a stable emulsion is between about 0.1 weight percent and about 50.0 weight percent, and in some embodiments is between about 0.5 weight percent and about 50.0 weight percent, based on the total weight of the stable emulsion. In other embodiments, the co-solvent may have a weight percent of between 0.5 and 30 weight percent.

In one embodiment, a co-solvent may be selected from the group including ethanol, water, glycerol, propylene glycol, 1,3-propanediol, and mixtures thereof. In embodiments, a co-solvent may be selected from the group including ethanol, water, and blends thereof. If water is used as a co-solvent, the weight percent of water may be limited to ensure that the water activity does not exceed 0.8.

In some embodiments, a water-soluble active biological ingredient is included in the hydrophilic phase. In embodiments, the water-soluble active biological ingredient complements the pharmacological effect of the active biological ingredient in the hydrophobic phase. In such embodiments, a water-soluble active biological ingredient is used to provide a separate effect. A water-soluble active biological ingredient may contribute flavor, color, or another property or characteristic to the aqueous phase.

In some embodiments, a water-soluble active biological ingredient may have botanical or fungal origin. For example, Ashwagandha extract from a natural source in some embodiments is added to the hydrophilic phase. In embodiments, at least one water soluble active biological ingredient is present in the stable emulsion at between about 0.001 weight percent and about 30.0 weight percent, based on the total weight of the stable emulsion. In some embodiments, the water soluble active biological ingredient is present at between about 0.05 weight percent and about 30.0 weight percent, based on the total weight of the stable emulsion. In some embodiments, the water soluble active biological ingredient is present at between about 0.1 weight percent and about 30.0 weight percent, based on the total weight of the stable emulsion. In some embodiments, the water soluble active biological ingredient is present at between about 1.0 weight percent and about 30.0 weight percent, based on the total weight of the stable emulsion.

In embodiments of the disclosure, the emulsion may be made by mixing the hydrophobic phase with the hydrophilic phase under conditions that yield an emulsion having small particles of hydrophobic material in a continuous hydrophilic phase. In embodiments, homogenization is carried out under conditions sufficient to yield particles having a median particle size of less than or equal to 100 nm. In embodiments, the median particle size of the hydrophobic phase in the emulsion is less than or equal to about 500 nm, and in some embodiments is between about 10 nm and about 500 nm. In still other embodiments, the median particle size of the hydrophobic phase is between about 25 nm and about 400 nm, and in yet other embodiments is between about 30 nm and about 300 nm. In some embodiments, the median particle size of the hydrophobic phase in the emulsion is between about 35 nm and about 250 nm. In some embodiments, the median particle size of the hydrophobic phase in the emulsion is between about 40 nm and about 200 nm. In some embodiments, the median particle size of the hydrophobic phase in the emulsion is between about 45 nm and about 200 nm. In some embodiments, the median particle size of the hydrophobic phase in the emulsion is between about 50 nm and about 200 nm. In some embodiments, the median particle size of the hydrophobic phase in the emulsion is between about 50 nm and about 190 nm. In some embodiments, the median particle size of the hydrophobic phase in the emulsion is between about 50 nm and about 180 nm. In some embodiments, the median particle size of the hydrophobic phase in the emulsion is between about 50 nm and about 170 nm. In some embodiments, the median particle size of the hydrophobic phase in the emulsion is between about 50 nm and about 160 nm. In some embodiments, the median particle size of the hydrophobic phase in the emulsion is between about 50 nm and about 150 nm. In some embodiments, the median particle size of the hydrophobic phase in the emulsion is between about 50 nm and about 140 nm. In some embodiments, the median particle size of the hydrophobic phase in the emulsion is between about 50 nm and about 130 nm. In some embodiments, the median particle size of the hydrophobic phase in the emulsion is between about 50 nm and about 120 nm. In some embodiments, the median particle size of the hydrophobic phase in the emulsion is between about 50 nm and about 110 nm. In some embodiments, the median particle size of the hydrophobic phase in the emulsion is between about 50 nm and about 100 nm.

In some embodiments, the pressure may be related to the choice of emulsifier. In some embodiments, the pressure required to obtain a given median particle size in a system having lecithin as an emulsifier is lower than the pressure required if modified starch were used as the emulsifier. The skilled practitioner recognizes that some embodiments may yield smaller median particle size at a given energy input than others. Physical conditions that affect homogenization include pressure, temperature, and the size of a screen, extruder, hammermill, or colloid mill. In embodiments of the disclosure, a two-step homogenization is used. Increasing the number of passes through the homogenizer generally decreases the breadth of distribution of the particles and shifts the median particle size lower. Suitable homogenizers are available from a number of sources.

In some embodiments, the sable emulsion discussed above may have a low separation rate. The separation rate may be measured by methods known in the art including measuring the rate of the movement of the particle in a liquid using Stoke's law to determine the percentage of the total amount of the particle that separates from the liquid per minute, day, and year. In particular embodiments, the stable emulsion has a separation rate of between 0.001 percent per year and 100 percent per year. In other embodiments, the stable emulsion may have a separation rate of between 0.01 percent and 75 percent per year. In yet other embodiments, the stable emulsion may have a separation rate of between 0.1 and 10 percent per year, or between 0.1 and 5 percent per year. In other embodiments, the stable emulsion may have a separation rate of less than 20 percent per year, or less than 10 percent per year, or less than 5 percent per year, or less than 3 percent per year, or less than 1 percent per year.

As another way of measuring the stability of the emulsion beyond the separation rate, the emulation may also have the active biological ingredient be present at a concentration of between 90 percent and 100 percent of a theoretical concentration of the active biological ingredient in the stable emulsion for greater than 100 weeks. The theoretical concentration of the active biological ingredient in product is known in the art to be calculated by multiplying the purity of the active biological ingredient by the usage rate of the active biological ingredient in product. The skilled practitioner recognizes that a concentration of active biological ingredient between 90 percent and 100 percent the theoretical concentration of the active biological ingredient in product is considered stable. The concentration of the active biological ingredient in product may be measured using methods known in the art including liquid chromatography assays. In various embodiments, the active biological ingredient may be present at a concentration between 90 percent and 100 percent of a theoretical concentration of the active biological ingredient in the stable emulsion for greater than 90 weeks, or for greater than 80 weeks, or for greater than 70 weeks, or for greater than 60 weeks, or for greater than 50 weeks, or for greater than 40 weeks, or for greater than 30 weeks, or for greater than 20 weeks.

### Emulsion for Spray Drying

In another aspect, the disclosure provides a stable emulsion for electrostatic spray drying. The hydrophobic phase comprises an active biological ingredient and a carrier oil. The hydrophilic phase comprises a composition that forms the continuous phase, a first or primary emulsifier, and a second or secondary emulsifier. The median particle size of the hydrophobic phase in embodiments of the stable emulsion may be less than or equal to about 1,000 nm, and may be greater than 5 nm. In other embodiments, the median particle size of the hydrophobic phase is between about 10 nm and about 750 nm, and in still other embodiments, is between about 11 nm and about 500 nm. In embodiments of this disclosure, the median particle size of the hydrophobic phase is between about 15 nm and about 400 nm. In some embodiments, the median particle size of the hydrophobic phase is between about 20 nm and about 300 nm. In some embodiments, the median particle size of the hydrophobic phase may be between 12 nm and 250 nm. In some embodiments, the median particle size of the hydrophobic phase is between about 25 nm and about 200 nm. In some embodiments, the median particle size of the hydrophobic phase is between about 15 nm and about 100 nm, or between 25 nm and 100 nm. In some embodiments, the median particle size of the hydrophobic phase is between about 30 nm and about 100 nm. In embodiments of this disclosure, the median particle size of the hydrophobic phase of the stable emulsion is controlled via modifications to the processing parameters. In embodiments of this disclosure, modifications to the processing parameters include modification of the homogenization pressure. In these embodiments of the disclosure, the emulsion may be different from the emulsion having a median particle size less than or equal to 100 nm. Rather, this stable emulsion is suitable for electrostatic spray drying to form a stable, water-dispersible particle.

Additionally, this disclosure also provides a method for modifying the mean particle size of the hydrophobic phase in the stable emulsion discussed herein where the method includes adjusting the homogenization pressure. Applying high pressure (for example, 40,000 psi) during homogenization may result in smaller particle sizes than achievable with low pressure (for example, 1,000 psi). In some embodiments, the homogenization pressure may be adjusted to between 1,000 psi and 40,000 psi. In other embodiments, the homogenization pressure may be adjusted to between 1,500 psi and 35,000 psi. In yet other embodiments, the homogenization pressure may be adjusted to between 3,000 psi and 33,000 psi.

In embodiments, the water-dispersible emulsions for spray drying may contain a greater weight percent of active biological ingredients than the nanoparticle emulsions disclosed herein. In some embodiments, the emulsion contains between about 5.0 weight percent and about 60.0 weight percent active biological ingredient, based on the total weight of the emulsion. This level may be advantageous because it greatly increases the loadings of active biological ingredient available upon ingestion and allows for use in both fluid and non-fluid products, such as dried drink or sports beverage compositions and cosmetics.

In embodiments of the disclosure, the active biological ingredient may be incorporated into a stable emulsion. A stable emulsion is a water-dispersible liquid emulsion comprising an active biological ingredient. In some embodiments, the weight percent of active biological ingredient in the stable emulsion is between about 0.001 weight percent and about 60.0 weight percent, based on the total weight of the stable emulsion. In some embodiments, the weight percent of active biological ingredient in the stable emulsion is between about 0.01 weight percent and about 60.0 weight percent, based on the total weight of the stable emulsion. In some embodiments, the weight percent of active biological ingredient in the stable emulsion is between about 0.1 weight percent and about 60.0 weight percent, based on the total weight of the stable emulsion. In some embodiments, the weight percent of active biological ingredient in the stable emulsion is between about 0.5 weight percent and about 60.0 weight percent, based on the total weight of the stable emulsion. In some embodiments, the weight percent of active biological ingredient in the stable emulsion is between about 1.0 weight percent and about 60.0 weight percent, based on the total weight of the stable emulsion. In some embodiments, the weight percent of active biological ingredient in the stable emulsion is between about 2.0 weight percent and about 60.0 weight percent, based on the total weight of the stable emulsion. In some embodiments, the weight percent of active biological ingredient in the stable emulsion is between about 3.0 weight percent and about 60.0 weight percent, based on the total weight of the stable emulsion. In some embodiments, the weight percent of active biological ingredient in the stable emulsion is between about 4.0 weight percent and about 60.0 weight percent, based on the total weight of the stable emulsion. In some embodiments, the weight percent of active biological ingredient in the stable emulsion is between about 5.0 weight percent and about 60.0 weight percent, based on the total weight of the stable emulsion. In some embodiments, the weight percent of active biological ingredient in the stable emulsion is between about 5.0 weight percent and about 55.0 weight percent, based on the total weight of the stable emulsion. In some embodiments, the weight percent of active biological ingredient in the stable emulsion is between about 5.0 weight percent and about 50.0 weight percent, based on the total weight of the stable emulsion. In some embodiments, the weight percent of active biological ingredient in the stable emulsion is between about 5.0 weight percent and about 45.0 weight percent, based on the total weight of the stable emulsion. In some embodiments, the weight percent of active biological ingredient in the stable emulsion is between about 5.0 weight percent and about 40.0 weight percent, based on the total weight of the stable emulsion. In some embodiments, the weight percent of active biological ingredient in the stable emulsion is between about 5.0 weight percent and about 35.0 weight percent, based on the total weight of the stable emulsion. In some embodiments, the weight percent of active biological ingredient in the stable emulsion is between about 5.0 weight percent and about 30.0 weight percent, based on the total weight of the stable emulsion.

In accordance with the disclosure, the hydrophobic phase comprises the active biological ingredient and the carrier oil. Active biological ingredients for embodiments of the disclosure may be selected from oil-based plant components. In particular, extracts of plants may be useful. Active biological ingredient in some embodiments may be cannabinoids and blends thereof.

In embodiments, the active biological ingredient is a cannabinoid as described above. Any of the cannabinoids identified above for use in a nanoparticle emulsion are suitable for use in embodiments of an emulsion for spray drying.

In some embodiments, the active biological ingredient is a viscous fluid or a solid. In such embodiments, a carrier oil is used to facilitate formation of the emulsion. In some embodiments, carrier oil is selected to increase bioavailability of the active biological ingredient. In some embodiments, mixtures of carrier oils are selected to increase bioavailability of the active biological ingredient. Carrier oils, and the reasons for their use, are set forth in detail above.

In embodiments of the disclosure, the weight percent of carrier oil in the stable emulsion may be between about 0.001 weight percent and about 60.0 weight percent, based on the total weight of the stable emulsion. In some embodiments, the weight percent of carrier oil in the stable emulsion is between about 0.01 weight percent and about 60.0 weight percent, based on the total weight of the stable emulsion. In some embodiments, the weight percent of carrier oil in the stable emulsion is between about 0.1 weight percent and about 60.0 weight percent, based on the total weight of the stable emulsion. In some embodiments, the weight percent of carrier oil in the stable emulsion is between about 0.5 weight percent and about 60.0 weight percent, based on the total weight of the stable emulsion. In some embodiments, the weight percent of carrier oil in the stable emulsion is between about 1.0 weight percent and about 60.0 weight percent, based on the total weight of the stable emulsion. In some embodiments, the weight percent of carrier oil in the stable emulsion is between about 2.0 weight percent and about 60.0 weight percent, based on the total weight of the stable emulsion. In some embodiments, the weight percent of carrier oil in the stable emulsion is between about 3.0 weight percent and about 60.0 weight percent, based on the total weight of the stable emulsion. In some embodiments, the weight percent of carrier oil in the stable emulsion is between about 4.0 weight percent and about 60.0 weight percent, based on the total weight of the stable emulsion. In some embodiments, the weight percent of carrier oil in the stable emulsion is between about 5.0 weight percent and about 60.0 weight percent, based on the total weight of the stable emulsion. In some embodiments, the weight percent of carrier oil in the stable emulsion is between about 5.0 weight percent and about 55.0 weight percent, based on the total weight of the stable emulsion. In some embodiments, the weight percent of carrier oil in the stable emulsion is between about 5.0 weight percent and about 50.0 weight percent, based on the total weight of the stable emulsion. In some embodiments, the weight percent of carrier oil in the stable emulsion is between about 5.0 weight percent and about 45.0 weight percent, based on the total weight of the stable emulsion. In some embodiments, the weight percent of carrier oil in the stable emulsion is between about 5.0 weight percent and about 40.0 weight percent, based on the total weight of the stable emulsion. In some embodiments, the weight percent of carrier oil in the stable emulsion is between about 5.0 weight percent and about 35.0 weight percent, based on the total weight of the stable emulsion. In some embodiments, the weight percent of carrier oil in the stable emulsion is between about 5.0 weight percent and about 30.0 weight percent, based on the total weight of the stable emulsion.

In accordance with the disclosure, the stable emulsion also contains a hydrophilic phase. This hydrophilic phase may have a continuous phase including primary emulsifier and an encapsulant. The continuous phase may be between 30 weight percent and 80 weight percent of the stable emulsion, based on the total weight of the stable emulsion. In embodiments, additional emulsifiers may be included. In some embodiments, the hydrophilic phase includes a secondary emulsifier to help strengthen the emulsion. In other embodiments, the hydrophilic phase includes a tertiary emulsifier. In still other embodiments, the hydrophilic phase includes a quaternary emulsifier. Emulsifiers are selected to provide a property or characteristic that improves the stable emulsion. For example, multiple emulsifiers may be selected in order to improve the particle size, particle size distribution, stability period, and other properties and characteristics.

The quantity of the continuous phase may be adjusted to form an emulsion having a solid weight percent between about 20 weight percent and about 70 weight percent, based on the total weight of the emulsion. Generally, the solids weight percent is the sum of the weight percents of all the emulsion components, except for the continuous phase. In embodiments, the solids weight percent of embodiments of the disclosure is between about 21 weight percent and 70 weight percent, based on the weight of the emulsion. In embodiments, the solids weight percent may be between about 22 weight percent and 70 weight percent, based on the weight of the emulsion. In embodiments, the solids weight percent may be between about 23 weight percent and 70 weight percent, based on the weight of the emulsion. In embodiments, the solids weight percent may be between about 24 weight percent and 70 weight percent, based on the weight of the emulsion. In embodiments, the solids weight percent of may be between about 25 weight percent and 70 weight percent, based on the weight of the emulsion. The quantity of the continuous phase also may be adjusted to ensure that the emulsion can be electrostatically spray dried.

In contrast with the disclosure directed to a nanoparticulate emulsion, in some embodiments water may comprise a continuous phase. In embodiments, the increased risk of degradation by the presence of microbial contaminants is minimal because the emulsion is not stored for extended periods and because the water does not form a part of the encapsulated particulate, as set forth below. Thus, in embodiments of the disclosure, a continuous phase comprises a composition that is selected from the group including water, ethanol, and blends thereof.

In accordance with the disclosure, the primary emulsifier is used to enable formation of an emulsion of the hydrophobic phase and the hydrophilic phase. In embodiments of the disclosure, the weight percent of primary emulsifier in the stable emulsion is between about 0.1 weight percent and about 90.0 weight percent, based on the total weight of the stable emulsion. In embodiments, the weight percent of primary emulsifier in a stable emulsion is between about 0.5 weight percent and about 80.0 weight percent, based on the total weight of the stable emulsion. In embodiments, the weight percent of primary emulsifier in a stable emulsion is between about 1.0 weight percent and about 75.0 weight percent, based on the total weight of the stable emulsion. In embodiments, the weight percent of primary emulsifier in a stable emulsion is between about 2.0 weight percent and about 75.0 weight percent, based on the total weight of the stable emulsion. In embodiments, the weight percent of primary emulsifier in a stable emulsion is between about 2.5 weight percent and about 75.0 weight percent, based on the total weight of the stable emulsion. In embodiments, the weight percent of primary emulsifier in a stable emulsion is between about 2.5 weight percent and about 70.0 weight percent, based on the total weight of the stable emulsion. In embodiments, the weight percent of primary emulsifier in a stable emulsion is between about 2.5 weight percent and about 65.0 weight percent, based on the total weight of the stable emulsion. In embodiments, the weight percent of primary emulsifier in a stable emulsion is between about 2.5 weight percent and about 60.0 weight percent, based on the total weight of the stable emulsion.

In embodiments of the disclosure, the primary emulsifier may be selected from the emulsifiers described above. Additional emulsifiers, such as secondary and tertiary emulsifiers, may also be selected from the emulsifiers described herein, and, in embodiments, are selected on the same bases described above.

In accordance with the disclosure, the weight percent of secondary emulsifier in the stable emulsion may be between about 0.1 weight percent and about 90.0 weight percent, based on the total weight of the stable emulsion. In embodiments of the disclosure, the weight percent of secondary emulsifier in the stable emulsion is between about 0.1 weight percent and about 90.0 weight percent, based on the total weight of the stable emulsion. In embodiments, the weight percent of secondary emulsifier in a stable emulsion is between about 0.5 weight percent and about 80.0 weight percent, based on the total weight of the stable emulsion. In embodiments, the weight percent of secondary emulsifier in a stable emulsion is between about 1.0 weight percent and about 75.0 weight percent, based on the total weight of the stable emulsion. In embodiments, the weight percent of secondary emulsifier in a stable emulsion is between about 2.0 weight percent and about 75.0 weight percent, based on the total weight of the stable emulsion. In embodiments, the weight percent of secondary emulsifier in a stable emulsion is between about 2.5 weight percent and about 75.0 weight percent, based on the total weight of the stable emulsion. In embodiments, the weight percent of secondary emulsifier in a stable emulsion is between about 2 . . . 5 weight percent and about 70.0 weight percent, based on the total weight of the stable emulsion. In embodiments, the weight percent of secondary emulsifier in a stable emulsion is between about 2.5 weight percent and about 65.0 weight percent, based on the total weight of the stable emulsion. In embodiments, the weight percent of secondary emulsifier in a stable emulsion is between about 2.5 weight percent and about 60.0 weight percent, based on the total weight of the stable emulsion.

In accordance with the disclosure, the weight percent of tertiary emulsifier in the stable emulsion may be between about 0.1 weight percent and about 90.0 weight percent, based on the total weight of the stable emulsion. In embodiments of the disclosure, the weight percent of tertiary emulsifier in the stable emulsion is between about 0.1 weight percent and about 90.0 weight percent, based on the total weight of the stable emulsion. In embodiments, the weight percent of tertiary emulsifier in a stable emulsion is between about 0.5 weight percent and about 80.0 weight percent, based on the total weight of the stable emulsion. In embodiments, the weight percent of tertiary emulsifier in a stable emulsion is between about 1.0 weight percent and about 75.0 weight percent, based on the total weight of the stable emulsion. In embodiments, the weight percent of tertiary emulsifier in a stable emulsion is between about 2.0 weight percent and about 75.0 weight percent, based on the total weight of the stable emulsion. In embodiments, the weight percent of tertiary emulsifier in a stable emulsion is between about 2.5 weight percent and about 75.0 weight percent, based on the total weight of the stable emulsion. In embodiments, the weight percent of tertiary emulsifier in a stable emulsion is between about 2 . . . 5 weight percent and about 70.0 weight percent, based on the total weight of the stable emulsion. In embodiments, the weight percent of tertiary emulsifier in a stable emulsion is between about 2.5 weight percent and about 65.0 weight percent, based on the total weight of the stable emulsion. In embodiments, the weight percent of tertiary emulsifier in a stable emulsion is between about 2.5 weight percent and about 60.0 weight percent, based on the total weight of the stable emulsion.

In embodiments directed to a spray-dryable emulsion, the hydrophilic phase may also contain an encapsulant or wall material. Encapsulant may be present in a quantity between about 0.01 weight percent and about 40.0 weight percent, based on the weight of the stable emulsion. In embodiments, the weight percent of encapsulant in a stable emulsion is between about 0.5 weight percent and about 80.0 weight percent, based on the weight of the stable emulsion. In some embodiments, the weight percent of encapsulant in a stable emulsion is between about 1.0 weight percent and about 75.0 weight percent, based on the weight of the stable emulsion. In some embodiments, the weight percent of encapsulant in a stable emulsion is between about 1.5 weight percent and about 70.0 weight percent, based on the weight of the stable emulsion. In some embodiments, the weight percent of encapsulant in a stable emulsion is between about 2.0 weight percent and about 65.0 weight percent, based on the weight of the stable emulsion. In some embodiments, the weight percent of encapsulant in a stable emulsion is between about 2.5 weight percent and about 60.0 weight percent, based on the weight of the stable emulsion. In some embodiments, the weight percent of encapsulant in a stable emulsion is between about 3.0 weight percent and about 55.0 weight percent, based on the weight of the stable emulsion. In some embodiments, the weight percent of encapsulant in a stable emulsion is between about 3.5 weight percent and about 50.0 weight percent, based on the weight of the stable emulsion. In some embodiments, the weight percent of encapsulant in a stable emulsion is between about 3.5 weight percent and about 45.0 weight percent, based on the weight of the stable emulsion.

In various embodiments, an encapsulant may be selected from the group including modified corn starch derived from corn starch, tapioca starch, or maltodextrin, Fibersol® brand modified corn starch, maltodextrin, arabinoxylan hemicellulose, fructans, inulin, alginic acids, agar, arabinogalactan, carrageenan, raffinose, polydextrose, and blends thereof. In embodiments, encapsulant is selected from the group including modified corn starch, Fibersol® brand modified corn starch, agar, arabinogalactan, and blends thereof.

In embodiments of the disclosure, the emulsion is made by mixing the hydrophobic phase with the hydrophilic phase under conditions that yield an emulsion having small particles of hydrophobic material in a continuous hydrophilic phase. Operating conditions of the homogenizer in some embodiments may be adjusted as required to achieve the desired particle size.

### Particulate

In still another aspect, the disclosure provides a stable, water-dispersible particulate. The particulate comprises an encapsulated active biological ingredient and carrier oil. The composition of particulate embodiments of the disclosure depends on the composition of the emulsion. Particulate embodiments comprise active biological ingredient and carrier oil in an encapsulant. The stable, water-dispersible particulate may have a median particle size of between about 1 μm and about 500 μm. Or, the stable, water-dispersible particulate may have a median particle size of between 2 μm and 300 μm, or between 3 μm and 200 μm, or between 4 μm and 100 μm, or between 5 μm and 50 μm, or between 5 μm and 30 μm.

In other embodiments the stable, water-dispersible particulate may have a median particle size of between about 5 μm and about 350 μm. In some embodiments, the particulate has a median particle size between about 5 μm and about 300 μm. In some embodiments, the particulate has a median particle size between about 5 μm and about 250 μm. In some embodiments, the particulate has a median particle size between about 5 μm and about 200 μm. In some embodiments, the particulate has a median particle size between about 5 μm and about 190 μm. In some embodiments, the particulate has a median particle size between about 5 μm and about 180 μm. In some embodiments, the particulate has a median particle size between about 5 μm and about 170 μm. In some embodiments, the particulate has a median particle size between about 5 μm and about 160 μm. In some embodiments, the particulate has a median particle size between about 5 μm and about 150 μm. In some embodiments, the particulate has a median particle size between about 5 μm and about 140 μm. In some embodiments, the particulate has a median particle size between about 5 μm and about 130 μm. In some embodiments, the particulate has a median particle size between about 5 μm and about 120 μm. In some embodiments, the particulate has a median particle size between about 5 μm and about 110 μm. In some embodiments, the particulate has a median particle size between about 5 μm and about 100 μm. In some embodiments, the particulate has a median particle size between about 5 μm and about 90 μm. In some embodiments, the particulate has a median particle size between about 5 μm and about 80 μm. In some embodiments, the particulate has a median particle size between about 5 μm and about 70 μm. In some embodiments, the particulate has a median particle size between about 5 μm and about 60 μm. In some embodiments, the particulate has a median particle size between about 5 μm and about 50 μm. In some embodiments, the particulate has a median particle size between about 5 μm and about 45 μm. In some embodiments, the particulate has a median particle size between about 5 μm and about 40 μm. In some embodiments, the particulate has a median particle size between about 5 μm and about 35 μm. In some embodiments, the particulate has a median particle size between about 5 μm and about 30 μm. The particulate may be suitable for use in both solid and liquid products.

In embodiments, the water activity of the particulate may be reduced to between about 0.01 and about 0.6. Generally, water activity is the ratio between the vapor pressure of the particulate in an undisturbed balance with the surrounding air media, and the vapor pressure of distilled water under identical conditions. Reducing water activity increases the purity of ingredients in the particulate, thus reducing the amount of ‘filler’ a purchaser pays for. Reducing the water activity also eliminates chances of microbial growth during storage. In some embodiments, the water activity of the particulate is between about 0.1 and about 0.5. In other embodiments, the water activity of the particulate is between about 0.2 and about 0.4. In other embodiments, the water activity of the particulate may be between 0.01 and 0.05, or between 0.01 and 0.4. In some embodiments, modifying the water activity to particular ranges affects the amount of moisture, thereby limiting chemical reactions, such as oxidation, that are facilitated by water.

Embodiments of the disclosure may yield encapsulated particles without structural deformities such as broken shells or folded structures. The presence or absence of any such structural deformities may be assessed by scanning electron microscope (SEM) imagery. Broken shells are known in the art as particulates with damaged outer layer, exposing the inside of the particulate. Folded structures as known in the art as non-spherical structures representing a folded plate-like structure. Such efficient encapsulation is desirable as it may affect the stability of the particulates, as well as the homogeneity of the particulates from a structural standpoint.

### Additional Components

In embodiments, the composition may include one or more additional components such as an inclusion or addition. These components may be present in either phase of the emulsions, or within the active biological ingredient and carrier oil encapsulated after spray drying. In some embodiments, the additional component may be present in an amount up to about 10 weight percent of the emulsion, or between 0.001 weight percent and 10.0 weight percent. Such components may serve as flavorants, antioxidants/preservatives, and agents that mask bitter flavor, especially bitter flavor introduced by the active biological ingredient. In some embodiments, an additional component includes essential oil, natural flavor, artificial flavor, and blends thereof. In other embodiments, an additional component includes essential oil, natural flavor, and blends thereof.

In one embodiment, the compositions disclosed herein may include an antioxidant. In one embodiment, the antioxidant may be selected from the group including chelators, alpha-tocopherols, rosemary extract, ascorbyl palmitate, BHT, BHA, TBHQ, stearyl citrate, sorbates, benzoates, and combinations thereof. Chelators include disodium EDTA, calcium disodium EDTA, tetrasodium EDTA, citric acid, sodium citrate, calcium citrate, phosphoric acid, glycine, and blends thereof. In embodiments, chelators serve as an antioxidant by sequestering transition metals. Trace amounts may play an important role in the propagation of oxidative reactions.

### Method for Making Emulsion

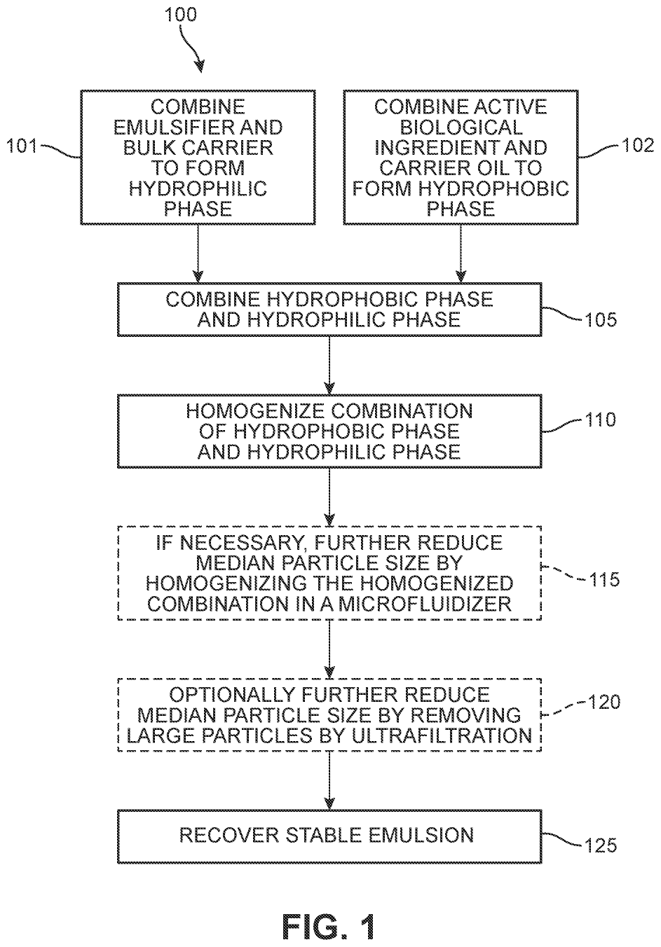

FIG. 1 is a schematic block diagram illustrating a method 100 for making a stable emulsion. In accordance with the method, a hydrophilic phase is formed at block 101 by blending a bulk carrier, or a continuous phase, and an emulsifier. The hydrophobic phase is formed by separately blending active biological ingredient and carrier oil at block 102. The fractions in some embodiments are heated to a temperature between about 50° C. and about 100° C. to ensure complete mixing.

At block 105, the hydrophobic phase and hydrophilic phase are fully combined, and then the combined phases are emulsified in a homogenizer at block 110. In some embodiments, the homogenized combination is subjected to microfluidization at block 115 to yield a stable emulsion. Block 120 illustrates an optional step of further reducing median particle size by removing large particles by ultrafiltration, if necessary. The median particle size of the hydrophobic phase in the emulsion is less than or equal to about 100 nm. The final stable emulsion in then recovered at block 125.

### Method for Making Emulsion for Electrostatic Spray Drying

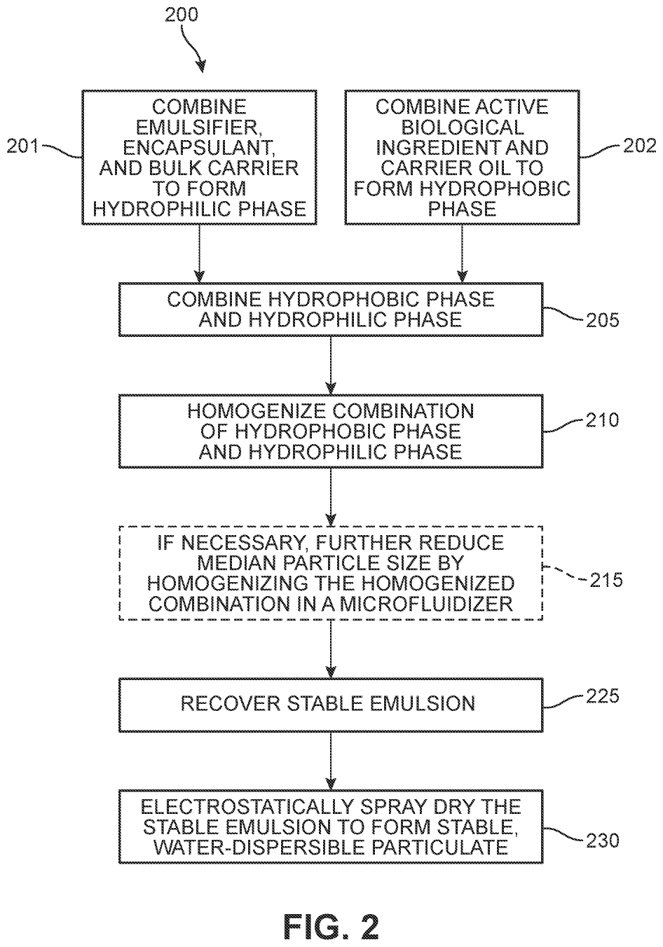

Turning to FIG. 2, schematic block diagram 200 summarizes a method for making a stable emulsion for electrostatic spray drying. Block 201 illustrates forming a hydrophilic phase by combining bulk carrier, or solvent, with emulsifier and encapsulant. Block 202 illustrates separately forming a hydrophobic phase by blending an active biological ingredient and a carrier oil. The hydrophobic phase and the hydrophilic phase are combined at block 205 and homogenized in a homogenizer at block 210. Block 215 indicates that, if it is necessary to further reduce median particle size, further homogenizination of the mixed combination of block 210 forms the stable emulsion. Stable emulsion is recovered at block 225. The median particle size of the hydrophobic phase in the emulsion is less than or equal to about 1000 nm. This stable emulsion in some embodiments used to make a stable, water-dispersible particulate by electrostatic spray drying.

### Electrostatic Spray Drying to Form Particulate

Block 230 of FIG. 2 illustrates formation of encapsulated particulate by electrostatically spray drying the emulsion of block 225. The median particle size of the stable, water-dispersible particulate may be greater than or equal to about 125 μm.

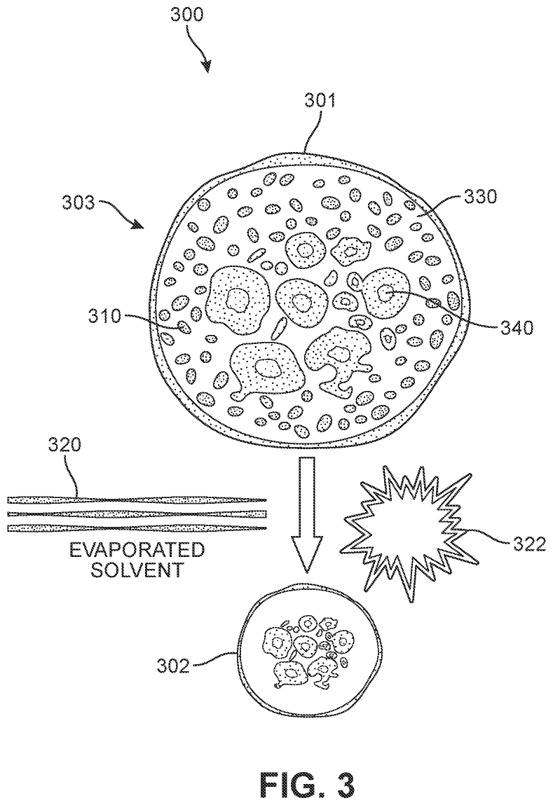

FIG. 3 is a schematic illustration of formation of stable particulate 300. Formation begins with structure 301, which is collection of particles 340 of active biological ingredient with carrier oil 330 in solvent 310 and surrounded by encapsulant 301. As solvent is evaporated at 320, structure 303 shrinks because solvent has been removed and forms encapsulated active biological ingredient and carrier oil 302.

In electrostatic spray drying, a voltage potential is applied across the particle as it is sprayed into a drying chamber. The voltage potential causes the non-polar active biological ingredient and carrier oil to gather at the center of a structure. The voltage potential also causes the encapsulant to migrate to the outside of the structure and encapsulate the active biological ingredient and carrier oil. This method affords the opportunity to use low temperatures to evaporate the solvent. Electrostatic spray driers are available from many manufacturers. Conditions of operation depend upon the material to be spray dried and are within the knowledge of the operator.

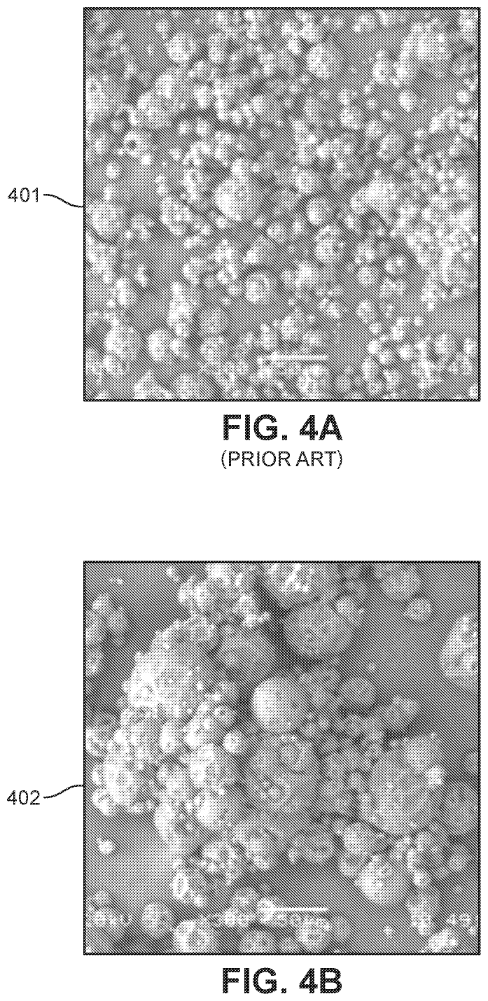

Electrostatic spray drying provides superior product with less energy use than typical spray drying. FIG. 4A and FIG. 4B are a comparison of the product 401 in FIG. 4A of conventional spray drying at a rate and at 190° C. Product of an embodiment of the disclosure is illustrated at 402 in FIG. 4B, which depicts the product of electrostatic spray drying the same emulsion as dried in the conventional spray dryer. The electrostatic spray drying was carried out at the same rate as the conventional process, but at 90° C.

Electrostatic spray drying produces a superior product with significant energy savings. As depicted in 402, the particulate forms aciniform product, whereas the conventional product depicted in 401 is more finely divided. Thus, the embodiment of the disclosure provides a superior product. The aciniform product is more easily combined with a solid product, dissolves more easily in a liquid product, and has a reduced dusting tendency than conventionally spray dried product.

In a still-further aspect, the disclosure provides for products comprising these emulsions and particulate. Products include beverages, dry beverage compositions, and cosmetics.

Emulsion embodiments of the disclosure are extraordinarily stable. Stokes' Law defines the movement of a particle in a liquid and thus can be used to evaluate emulsion stability. In accordance with Stokes' Law, stability is a function of density, interfacial properties, and viscosity, and is a function of the square of particle size. Thus, it can be seen that minimizing particle size has a significant effect on emulsion stability.

Particle size and other properties and characteristics of emulsions can be determined by many commercially available devices. For example, particle size in some embodiments determined by dynamic light scattering, a technique known in the art. Dynamic light scattering utilizes a spectrophotometric method that can identify both average particle size and spread of the size distribution. Dynamic light scattering is suitable for determination of particle size of polydisperse systems such as embodiments of the disclosure. The harmonic mean particle size also is useful. The harmonic mean is one of many types of averages. Generally, harmonic mean particle size is useful for calculating the average of a dataset that may have a few outliers, which can skew the data. Some particle size characterization devices may provide the harmonic mean particle size. In embodiments of the disclosure, the particle size can vary by many orders of magnitude. Therefore, a few large particles can skew median particle size. Particle size span, based on volume-based size distribution, provides an indication of how far apart the 10 percent and 90 percent points are, normalized by the 50 percent point. The Span value is another measure that aids understanding the spread of particle size with reduced skew by outliers (big and small).

Other devices and calculations based on particle size are used to identify particle and droplet velocity distributions, for example. Methods and techniques of determining creaming and sedimentation rate, particle size distribution, viscosity, density, and interfacial forces, for example, are known to the skilled practitioner. These and other properties and characteristics are measured and used to characterize emulsions, particulates, and products of embodiments of the disclosure. The creaming/phase separation, sedimentation/phase separation, and flocculation are gravity-driven unstable phenomena that are useful. Coagulation, Ostwald ripening, coalescence, and phase inversion are diffusion-driven unstable phenomena that can be identified. All of these phenomena or properties and characteristics are identified and quantified by the way light passes through the sample in the analyzer.

For example, stability index, which is a measure of relative separation, is useful for evaluating the stability of an emulsion in an accelerated manner. An emulsion having a stability index of zero is fully homogeneous, and a stability index of 1 is fully separated. Separation as a function of time is useful for determining shelf life. As the skilled practitioner recognizes, a separation rate of 3 percent/year indicates that 3 percent of the emulsion has separated by creaming or sedimentation. As the skilled practitioner recognizes, it may be possible to re-suspend the separated particles by mixing. However, the greater and faster the separation, the less likely re-suspension is possible.

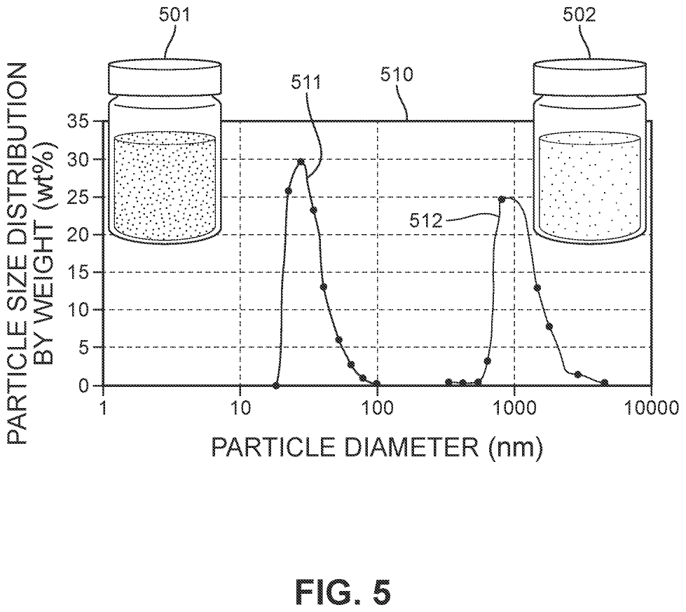

In accordance with the disclosure, particle size thus affects properties and characteristics of the emulsion embodiments of the disclosure significantly. FIG. 5 illustrates the visual appearance of two emulsions. As can be seen on graph 510, particle size distribution, by weight, is plotted as a function of particle diameter (nm). Line 511 illustrates the particle size distribution for emulsion product 501. As can be seen, emulsion product 501 is clear, or optically translucent. The fine particle size, a median particle size of about 40 nm, is thermodynamically stable and provides greater bioavailability than larger particles, as set forth above. Emulsion 502 is a product that has a median particle size of more than about 900 nm, as illustrated by line 512. As can be seen, emulsion product 502 is opaque. It also is less stable than emulsion product 501.

Properties and characteristics of emulsion of the disclosure are determined and compared to standards commonly used to characterize emulsions. Physical testing includes a shelf-life prediction; creaming rate, mm/s; and stability, rated on a stability index ranging from 0 to 1. Stability index of 1 means no emulsification was obtained. Stability index of 0 means that full homogenization was obtained. Particle size determinations include median size, nm; mode size, nm; Sauter diameter, nm; standard deviation, nm; particle size span, from 10 percent to 90 percent; volume diameter, nm (diameter of a sphere having the same volume of measured particles); volume specific surface area, nm³/nm; and particle size quantiles. The active biological ingredient content also is determined.

These parameters also are determined for products to which emulsion embodiments of the disclosure has been added. Turbidity (NTUs) is determined before and after infusion with the emulsion. Turbidity is a good measure of clarity or appearance. The skilled practitioner recognizes that a turbidity of up to about 10 NTUs is considered clear.

Stability of tested product is determined at manufacture. Then, the product is stored at 70° F. and retested at different time intervals for the concentration of the active biological ingredient in the product. The theoretical concentration of the active biological ingredient in product is known in the art to be calculated by multiplying the purity of the active biological ingredient and the usage rate of the active biological ingredient in product. The skilled practitioner recognizes that a concentration of active biological ingredient between 90 percent and 100 percent the theoretical concentration of the active biological ingredient in product is considered stable. The concentration of the active biological ingredient in product may be measured using methods known in the art including liquid chromatography assays.

Testing in vitro has been carried out to determine solubility over time. Product was placed for a time (here, 3 hours) in a stirred bath at pH about 2 to simulate stomach content to determine solubility.

Particulate also may be characterized by the “wettability”. This characterization measures the time required for a known amount of the particulate to completely penetrate the surface of and submerge in water when 0.1 g particulate is added to a 250 mL vessel containing 100 ml distilled water at 25±1° C. Following the method as described in A/S Niro Atomizer (1978a), the particulate is placed inside a glass funnel held on a ring stand, while the height from the bottom of the funnel and the water surface is approximately 10 cm. A test tube is placed inside the funnel to block the lower opening. Once the test tube is lifted, the stopwatch starts recording the time it takes for complete penetration of the water surface by the particulate. The skilled practitioner recognizes that the solubility of a particulate in an aqueous media is better for particulates with lower wettability. In one embodiment, wettability of the particulate is between 0.1 seconds and 350 seconds. In various other embodiments, the wettability of the particulate may be between 0.1 seconds and 200 seconds, or between 0.1 seconds and 100 seconds, or between 0.1 seconds and 50 seconds, or between 0.1 seconds and 40 seconds, or between 0.1 seconds and 35 seconds, or between 0.1 seconds and 30 seconds. In another embodiment, the wettability may be between 0.2 seconds and 200 seconds. In one embodiment, wettability of the particulate is between 0.3 seconds and 100 seconds. In yet another embodiment, wettability of the particulate is between 0.4 seconds and 50 seconds; and in still another embodiment, between 0.5 seconds and 40 seconds. In one embodiment, wettability of the particulate is between 0.6 seconds and 35 seconds. In one embodiment, wettability of the particulate is between 0.7 seconds and 30 seconds. In one embodiment, wettability of the particulate is between 0.8 seconds and 25 seconds. In one embodiment, wettability of the particulate is between 0.9 seconds and 23 seconds. In yet another embodiment, wettability of the particulate is between 1 seconds and 21 seconds. In embodiments of the disclosure, wettability is decreased using pulsed voltage when spray drying. The pulsed voltage can be utilized as a process parameter for spray drying where 1 second pulses of 25 kv and 2.5 kv are generated by the spray drying equipment.

In another aspect, this disclosure provides a method of modifying the wettability of the stable water-dispersible particulate. The method includes using pulsed voltage while spray drying, in accordance with the spray drying disclosures above and below in the examples. Pulsed voltage is a method capable of agglomerating the particulates by controlling the voltage applied to the feed formulation of the spray dryer, on an intermittent basis. Agglomeration is the process of creating a group of particulates where the particulate shells are attached to each other. As particulates are formed, some form an outer shell more readily than others which form their shell gradually. By controlling the voltage applied to the particulate during spray drying, the two aforementioned types of particulates are bonded together, forming agglomerated particles. The agglomeration of particulates provides particulates with superior solubility. Agglomeration of particulates is typically carried out in methods using secondary agglomeration processes. The method as described in this disclosure is capable of agglomerating particulates in a single process concurrent with spray drying without the need for secondary processing.

### EXAMPLES

### Examples 1-5

Emulsions were prepared in accordance with a method embodiment of the disclosure. The following tables identify the components and the quantities thereof in each example. The weight percent of each component is based on the total weight of the emulsion.

Hydrophobic phase was prepared by combining carrier oil of Table A1 with active biological ingredient of Table A2 in a glass flask/beaker/double boiler. This mixture was heated at 75° C. for about 10 minutes, until both hydrophobic components were melted together.

TABLE A1

| Example | Carrier Oil | Wt % Oil |
|---|---|---|
| 1 | Fractionated coconut oil (MCT) | 1 |
| 2 | Fractionated coconut oil (MCT) | 1 |
| 3 | Fractionated coconut oil (MCT) | 1 |
| 4 | Refined olive oil | 3 |
| 5 | Peceol ® glycerol monooleate | 2 |

TABLE A2

| | Active Biological Ingredient | | |
|---|---|---|---|
| Example | (ABI) | CBD, mg/g | Wt % ABI |
| 1 | Full Spectrum CBD Distillate | 8.24 | 1 |
| 2 | Full Spectrum CBD Distillate | 8.24 | 1 |
| 3 | Full Spectrum CBD Distillate | 8.24 | 1 |
| 4 | Broad Spectrum CBD Distillate | 27.33 | 3 |
| 5 | CBD Isolate | 9.8 | 1 |

Separately, a hydrophilic phase was prepared by combining emulsifier of Table A3 and continuous phase of Table A4. The combination was blended at 13,500 rpm using a rotor stator mixer at room temperature until the mixture was homogeneous, for about 2 minutes.

TABLE A3

| Example | Emulsifier | Wt % Emulsifier |
|---|---|---|
| 1 | Lyso-lecithin with >80% lyso-lecithin | 1 |
| | content | |
| 2 | Rhamnolipid with 25-90% rhamnolipid | 1 |
| | content | |
| 3 | Sunflower lecithin with 10-90% | 3 |
| | phosphotidylcholine content | |
| 4 | Sucrose monopalmitate, >90% purity | 1.5 |
| 5 | Quillaia extract, 5-50% saponin content | 3 |

TABLE A4

| Example | Continuous Phase | Wt % Continuous Phase |
|---|---|---|
| 1 | Glycerin USP | 97 |
| 2 | Glycerin USP | 97 |
| 3 | Glycerin USP | 95 |
| 4 | Glycerin USP | 92.5 |
| 5 | Glycerin USP | 96 |

The hydrophobic phase was added to the hydrophilic phase and the combination was blended for 2 minutes at 13,500 rpm. The combined mixture then was homogenized in a high pressure homogenizer at 30,000 psi to obtain an emulsion. The emulsion had the properties and characteristics set forth in Table A5A and Table A5B when added to distilled water to provide 8 mg CBD. In Table 5, ‘Stability’ means the stability index. The turbidity was determined in products comprising tap water and the emulsion.

TABLE A5A

| | Median, | Separation | | |
|---|---|---|---|---|
| Example | nm | Rate (%/day) | Stability | Turbidity |
| 1 | 32.83 | 0.008 | 0.05 | 6.6 |
| 2 | 32.65 | 0.027 | 0.08 | 27 |
| 3 | 41.8 | 0.052 | 0.3 | 7.2 |
| 4 | 23.97 | 0.019 | 0.17 | 7.5 |
| 5 | 24.27 | 0.026 | 0.08 | 43.8 |

TABLE A5B

| Exam- | Particle size characterizations, nm | | | | | | |
|---|---|---|---|---|---|---|---|
| ple | Median | Span | 10%≤ | 16%≤ | 50%≤ | 84%≤ | 90%≤ |
| 1 | 32.83 | 0.97 | 17.18 | 18.13 | 32.83 | 48.85 | 48.92 |
| 2 | 32.65 | 6.20 | 29.7 | 29.77 | 32.65 | 178.9 | 232.1 |
| 3 | 41.8 | 1.56 | 25.03 | 30.4 | 41.8 | 74.79 | 90.32 |
| 4 | 23.97 | 0.78 | 17.9 | 19.13 | 23.97 | 32.28 | 36.64 |
| 5 | 24.27 | 0.99 | 13.15 | 17.96 | 24.27 | 32.66 | 37.24 |

The emulsion had the properties and characteristics set forth in Table A6 when added to iced tea to obtain 25 mg CBD/250 mL.

TABLE A6A

| Example | Median, nm | Span | Stability |
|---|---|---|---|
| 1 | 262.7 | 4.8 | 0.52 |
| 2 | 139.6 | 14.2 | 0.03 |
| 3 | 98.16 | 3.25 | 0.14 |
| 4 | 212.9 | 5.7 | 0.25 |
| 5 | 101.6 | 11.9 | 0.08 |

TABLE A6B

| Exam- | Particle size characterizations, nm | | | | | | |
|---|---|---|---|---|---|---|---|
| ple | Median | Span | 10%≤ | 16%≤ | 50%≤ | 84%≤ | 90%≤ |
| 1 | 262.7 | 4.8 | 44.72 | 69.95 | 262.7 | 1000 | 1306 |
| 2 | 139.6 | 14.2 | 63.57 | 74.78 | 139.6 | 1171 | 2047 |
| 3 | 98.16 | 3.25 | 43.54 | 51.34 | 98.16 | 232.8 | 362.3 |
| 4 | 212.9 | 5.7 | 53.95 | 65.62 | 212.9 | 976.5 | 1270 |
| 5 | 101.6 | 11.9 | 40.86 | 41.34 | 101.6 | 564.9 | 1250 |

The emulsion had the properties and characteristics set forth in Table A7 when added to coconut water to obtain 25 mg CBD/250 mL.

TABLE A7A

| Example | Median, nm | Span | Stability |
|---|---|---|---|
| 1 | 1168 | 2.35 | 0.80 |
| 2 | 80.6 | 1.49 | 0.77 |
| 3 | 50.3 | 1.75 | 0.003 |
| 4 | 725 | 2.83 | 0.85 |
| 5 | 23.2 | 1.12 | 0.35 |

TABLE A7B

| | Particle size characterizations, nm | | | | | | |
|---|---|---|---|---|---|---|---|
| Example | Median | Span | 10%≤ | 16%≤ | 50%≤ | 84%≤ | 90%≤ |
| 1 | 1168 | 2.35 | 30.75 | 40.45 | 1,168 | 2,374 | 2,774 |
| 2 | 80.6 | 1.49 | 40.77 | 47.63 | 80.6 | 136.3 | 161.1 |
| 3 | 50.3 | 1.75 | 33.64 | 38.31 | 50.3 | 109.98 | 121.62 |
| 4 | 725 | 2.83 | 265.1 | 327.3 | 724.7 | 1,755 | 2,314 |
| 5 | 23.2 | 1.12 | 13.53 | 15.1 | 23.23 | 34.31 | 39.35 |

The coconut water example and the iced tea example illustrate how the properties and characteristics, particularly the median particle size, is affected by the composition of the finished product in the form of a coconut water beverage. Coconut water is high in electrolytes (salts). As can be seen, in embodiments, the median particle size is higher than for less ionic products, such as distilled water or iced tea.

### Examples 6-11

An emulsion suitable for spray drying is prepared in accordance with a method embodiment of the disclosure. A hydrophobic phase is prepared by combining carrier oil of Table B1 with active biological ingredient of Table B2 in a beaker. This mixture is heated at 75° C. until both hydrophobic phases are melted together, for about 10 minutes.

TABLE B1

| Example # | Carrier Oil | Wt % Oil |
|---|---|---|
| 6 | Fractionated coconut oil (MCT) | 30 |
| 7 | Refined olive oil | 24 |
| 8 | Canola oil | 22.2 |
| 9 | Refined sesame oil | 22.2 |
| 10 | Peceol ® glycerol monooleate | 20 |
| 11 | Fractionated coconut oil (MCT) | 20 |

TABLE B2

| Example | Active Biological Ingredient | CBD, mg/g | Wt % ABI |
|---|---|---|---|
| 6 | Full Spectrum CBD Distillate | 8.24 | 30 |
| 7 | Full Spectrum CBD Distillate | 8.24 | 24 |
| 8 | Broad Spectrum CBD Distillate | 27.33 | 22.2 |
| 9 | Full Spectrum CBD Distillate | 8.24 | 22.2 |
| 10 | CBD Isolate | 9.8 | 20 |
| 11 | Broad Spectrum CBD Distillate | 27.33 | 20 |

Separately, a hydrophilic phase is prepared by combining wall material of Table B3, emulsifier of Table B4, and sufficient distilled water sufficient to form an emulsion when the phases are combined having the percentage of solids set forth in Table B4.

TABLE B3

| Example | Wall Material | Wt % Wall Material |
|---|---|---|
| 6 | Fibersol ® Corn Maltodextrin | 25 |
| 7 | Fibersol ® Corn Maltodextrin | 40 |
| 8 | Maltodextrin DE 15 | 11.2 |
| 9 | Fibersol ® Corn Maltodextrin | 45.6 |
| 10 | None | 0 |
| 11 | Arabinoxylan (hemicellulose) | 50 |

TABLE B4

| | | Wt % |
|---|---|---|
| Example | Emulsifier | Emulsifier |
| 6 | Q-Naturale ® quillala extract, saponin | 15 |
| | between 5-50% | |
| 7 | Purity Gum Ultra ® modified corn starch | 12 |
| 8 | Capsul ® modified tapioca starch | 44.4 |
| 9 | Lyso-lecithin [?l-phosphatidylcholine?] >80 | 10 |
| 10 | Hi-Cap ® modified corn starch | 60 |
| 11 | Rhamnolipid with 25-90% rhamnolipid content | 10 |

This mixture is blended at 13,500 rpm using a rotor stator mixer at room temperature until the mixture is homogeneous, for about 2 minutes.

The hydrophobic phase is added to the hydrophilic phase and the combination is blended for 2 minutes at 13,500 rpm. The combined mixture is then homogenized in a high pressure homogenizer at 30,000 psi to obtain emulsions having the properties and characteristics of Table B5.

TABLE B5

| Parameter | Result | |
|---|---|---|
| Powder Density | >0.35 | g/cm3 |
| Powder Particle Size | >50% above 125 microns | |
| Powder Mesh Size | >50% above 120 mesh | |
| Powder Moisture Content | <5% | |
| Encapsulation Efficiency | >80% oil encapsulated | |
| Median particle size when dissolved in water | <1000 | nm |

| | |
|---|---|
| Time to dissolve 1 gram in 1000 ml of distilled | <30 seconds at 500 rpm |
| water | |

### Examples 12-15

Table S001 below shows the ability to control particle size by changing the pressure in stable emulsions for electrostatic spray drying. Namely, using a higher pressure results in smaller particle size. In each of examples 12-15: the oil carrier was MCT, the API was full spectrum distillate, the API % w/w was 20, the wall material was maltodextrin 1052001, the wall material % w/w was 10, the emulsifier was Modified Food Starch from Tapioca-Ingredion Inc., and emulsifier % w/w was 10, the solvent was distilled water, the solvent % w/w was 40.0, the weight percent solids was 60.0%, the powder was 28.3, the material/API ratio was 0.50, the emulsifier/API ratio was 0.50, the nm³/hr was 25, heat was at 140° C., pressure was at 200 kPa, the generator was set at PWM with 25 kv for 1 s and 2.5 kv for 1 s, the heat was then at 100° C., vacuum was at 0.1 kPA, the pump was 3.2 mm was set at 17.

TABLE S001

| | | Homogenization | |
|---|---|---|---|
| Example | Internal Trial # | Pressure (psi) | Average particle size |
| 12 | 3A7G1A | 3,200 | 75.24 |
| 13 | 3A7G1B | 10,000 | 38.98 |
| 14 | 3A7G1C | 20,000 | 37.75 |
| 15 | 3A7G1D | 30,000 | 38.18 |

### Examples 16-24

Tables S003A and S003B below show different formulations for a stable emulsion, namely the hydrophobic phase used in the emulation is shown in Table S003A and the hydrophilic phase used in the emulation is shown in Table S003B.

The formulations shown in these tables relate to the disclosures herein at least at paragraphs [0009], [0010], and [0011] above.

In any of the tables below, a blank cells coveys that none of that particular component was included in the example.

TABLE S003A
Hydrophobic Phase

| | | Active | Usage | Car- | Usage |
|---|---|---|---|---|---|
| Exam- | Prototype | Biological | Rate | rier | Rate |
| ple | ID | Ingredient | (% w/w) | Oil | (% w/w) |
| 16 | 56H | CBD Isolate | 7.5 | MCT | 7.5 |
| 17 | 25G5-CBG | CBG Distillate | 1.0 | MCT | 1.5 |
| | Distillate | | | | |
| 18 | 64A3 | CBD Isolate | 3.3 | MCT | 15.0 |
| | | CBN Isolate | 1.7 | | |
| 19 | 64D2 | Broad Spectrum | 2.5 | MCT | 20.0 |
| | | Distillate | | | |
| | | CBD Isolate | 2.5 | | |
| 20 | 12I | Broad Spectrum | 1.0 | MCT | 1.0 |
| | | Distillate | | | |
| 21 | 9T | Broad Spectrum | 1.0 | MCT | 1.0 |
| | | Distillate | | | |
| 22 | 64C5 | Broad Spectrum | 5.0 | MCT | 20.0 |
| | | Distillate | | | |
| 23 | 18A | Full Spectrum | 1.0 | MCT | 1.0 |
| | | Distillate | | | |
| 24 | 10F11B | Full Spectrum | 5.0 | MCT | 10.0 |
| | | Distillate | | | |

TABLE S003B
Hydrophilic Phase

| | | | Usage | | Usage | | Usage | | Usage |
|---|---|---|---|---|---|---|---|---|---|
| | | | Rate | | Rate | | Rate | | Rate |
| Ex | Prototype | | (% | | (% | Continuous | (% | Continuous | (% |
| # | ID | Emulsifier | w/w) | Emulsifier | w/w) | Phase | w/w) | Phase | w/w) |
| 16 | 56H | Quillaja Saponaria | 2.5 | Sunflower | 0.5 | Glycerin | 82.0 | | |
| | | Extract - Naturex | | Lecithin, ≥99% - | | | | | |
| | | | | Compass | | | | | |
| | | | | Foods Pte | | | | | |
| | | | | Ltd | | | | | |
| 17 | 25G5- | Sunflower Lecithin, ≥99% - | 2.0 | Sucrose | 1.0 | Glycerin | 94.5 | | |
| | CBG | Compass | | Monoester | | | | | |
| | Distillate | Foods Pte Ltd | | of Palmitic | | | | | |
| | | | | Acid - | | | | | |
| | | | | Compass | | | | | |
| | | | | Foods Pte | | | | | |
| | | | | Ltd | | | | | |
| 18 | 64A3 | Maltodextrin, | 5.0 | Modified | 5.0 | Distilled | 15.0 | Glycerin | 55.0 |
| | | 10520011 | | Food | | Water | | | |
| | | | | Starch | | | | | |
| | | | | from | | | | | |
| | | | | Tapioca - | | | | | |
| | | | | Ingredion | | | | | |
| | | | | Inc. | | | | | |
| 19 | 64D2 | Modified Food | 10.0 | | | Distilled | 15.0 | Glycerin | 50.0 |
| | | Starch from Waxy | | | | Water | | | |
| | | Maize - Ingredion | | | | | | | |
| | | Inc. | | | | | | | |
| 20 | 121 | Phosphatidylcholine | 6.0 | | | Glycerin | 92.0 | | |
| | | from soybean, D,L- | | | | | | | |
| | | α-tocopherol (n.m.t. | | | | | | | |
| | | 0.3%) - Lipoid | | | | | | | |
| 21 | 9T | Quillaja Saponaria | 30.0 | | | Glycerin | 68.0 | | |
| | | Extract - Ingredion | | | | | | | |
| | | Inc. | | | | | | | |
| 22 | 64C5 | Flavor-free | 10.0 | Modified | 10.0 | Distilled | 15.0 | Glycerin | 40.0 |
| | | Maltodextrin - | | Food | | Water | | | |
| | | Ingredion Inc. | | Starch | | | | | |
| | | | | from | | | | | |
| | | | | Tapioca - | | | | | |
| | | | | Ingredion | | | | | |
| | | | | Inc. | | | | | |
| 23 | 18A | Acetylated Lecithins - | 4.0 | | | Glycerin | 94.0 | | |
| | | Cargill Inc. | | | | | | | |
| 24 | 10F11B | Quillaja Saponaria | 2.5 | Sunflower | 0.5 | Glycerin | 82.0 | | |
| | | Extract - Ingredion | | Lecithin, ≥99% - | | | | | |
| | | Inc. | | Compass | | | | | |
| | | | | Foods Pte | | | | | |
| | | | | Ltd | | | | | |

### Examples 25-44

Tables S003C and S003D below show different formulations for a stable emulsion for electrostatic spray drying, namely the hydrophobic phase used in the emulation for electrostatic spray drying is shown in Table S003C and the hydrophilic phase used in the emulation for electrostatic spray drying is shown in Table S003D.

The formulations shown in these tables relate to the disclosures herein at least at paragraphs [0009], [0010], and [0011] above.

TABLE S003C

| | | Hydrophobic Phase | | | |
|---|---|---|---|---|---|
| | | Active | Usage | Car- | Usage |
| Exam- | Prototype | Biological | Rate | rier | Rate |
| ple | ID | Ingredient | (% w/w) | Oil | (% w/w) |
| 25 | P210412-1A | CBD Isolate | 8.0 | MCT | 8.0 |
| 26 | P210330-2B | CBD Isolate | 5.0 | MCT | 5.0 |
| 27 | P210412-3 | CBD Isolate | 5.0 | MCT | 5.0 |
| 28 | 3D | CBD Isolate | 5.0 | MCT | 5.0 |
| 29 | 3G | CBD Isolate | 5.0 | MCT | 5.0 |
| 30 | P210405-2C | CBD Isolate | 5.0 | MCT | 5.0 |
| 31 | 3A | CBD Isolate | 5.0 | MCT | 5.0 |
| 32 | 3E | CBD Isolate | 5.0 | MCT | 5.0 |
| 33 | 3B | CBD Isolate | 5.0 | MCT | 5.0 |
| 34 | 3C | CBD Isolate | 5.0 | MCT | 5.0 |
| 35 | 3F | CBD Isolate | 5.0 | MCT | 5.0 |
| 36 | 3A7D7D1 | Broad Spectrum | 20.0 | MCT | 20.0 |
| | | Distillate | | | |
| 37 | 3A7D7E1 | Broad Spectrum | 20.0 | MCT | 20.0 |
| | | Distillate | | | |
| 38 | 3A16A | Broad Spectrum | 12.5 | MCT | 12.5 |
| | | Distillate | | | |
| 39 | 3A17A1 | Broad Spectrum | 12.5 | MCT | 12.5 |
| | | Distillate | | | |
| 40 | 3A7D2A1 | Broad Spectrum | 22.5 | MCT | 22.5 |
| | | Distillate | | | |
| 41 | 3A7F2 | Full Spectrum | 12.5 | MCT | 12.5 |
| | | Distillate | | | |
| 42 | 3A7G1 | Full Spectrum | 20.0 | MCT | 20.0 |
| | | Distillate | | | |
| 43 | 1H1 | Full Spectrum | 7.5 | MCT | 15.0 |
| | | Distillate | | | |
| | | CBN Isolate | 7.5 | | |
| 44 | 3Z2 | Full Spectrum | 2.5 | MCT | 5.0 |
| | | Distillate | | | |
| | | CBG Distillate | 2.5 | | |

TABLE S003D
Hydrophilic Phase

| | | | Usage | | Usage | | Usage |
|---|---|---|---|---|---|---|---|
| | | | Rate | | Rate | | Rate |
| | Prototype | | (% | | (% | Continuous | (% |
| Example | ID | Encapsulant | w/w) | Emulsifier | w/w) | Phase | w/w) |
| 25 | P210412-1A | | | Modified Food | 24.0 | Distilled | 60.0 |
| | | | | Starch from | | Water | |
| | | | | Waxy Maize - | | | |
| | | | | Ingredion Inc. | | | |
| 26 | P210330-2B | Maltodextrin, | 25.0 | Modified Corn | 10.0 | Distilled | 55.0 |
| | | Tapioca | | Starch - | | Water | |
| | | Based - | | Ingredion Inc. | | | |
| | | Ingredion Inc. | | | | | |
| 27 | P210412-3 | Maltodextrin, | 25.0 | Modified Food | 10.0 | Distilled | 55.0 |
| | | 90% Dietary | | Starch from | | Water | |
| | | Fiber - | | Tapioca - | | | |
| | | Fibersol | | Ingredion Inc. | | | |
| 28 | 3D | Flavor-free | 25.0 | Modified Food | 10.0 | Distilled | 55.0 |
| | | Maltodextrin - | | Starch from | | Water | |
| | | Ingredion Inc. | | Tapioca - | | | |
| | | | | Ingredion Inc. | | | |
| 29 | 3G | Flavor-free | 25.0 | Modified Food | 10.0 | Distilled | 55.0 |
| | | Maltodextrin - | | Starch from | | Water | |
| | | Ingredion Inc. | | Tapioca - | | | |
| | | | | Ingredion Inc. | | | |
| 30 | P210405-2C | Maltodextrin, | 25.0 | Modified Corn | 10.0 | Distilled | 55.0 |
| | | Tapioca | | Starch - | | Water | |
| | | Based - | | Ingredion Inc. | | | |
| | | Ingredion Inc. | | | | | |
| 31 | 3A | Flavor-free | 25.0 | Modified Food | 10.0 | Distilled | 55.0 |
| | | Maltodextrin - | | Starch from | | Water | |
| | | Ingredion Inc. | | Tapioca - | | | |
| | | | | Ingredion Inc. | | | |
| 32 | 3E | Flavor-free | 25.0 | Modified Food | 10.0 | Distilled | 55.0 |
| | | Maltodextrin - | | Starch from | | Water | |
| | | Ingredion Inc. | | Tapioca - | | | |
| | | | | Ingredion Inc. | | | |
| 33 | 3B | Flavor-free | 25.0 | Modified Food | 10.0 | Distilled | 55.0 |
| | | Maltodextrin - | | Starch from | | Water | |
| | | Ingredion Inc. | | Tapioca - | | | |
| | | | | Ingredion Inc. | | | |
| 34 | 3C | Flavor-free | 25.0 | Modified Food | 10.0 | Distilled | 55.0 |
| | | Maltodextrin - | | Starch from | | Water | |
| | | Ingredion Inc. | | Tapioca - | | | |
| | | | | Ingredion Inc. | | | |
| 35 | 3F | Flavor-free | 25.0 | Modified Food | 10.0 | Distilled | 55.0 |
| | | Maltodextrin - | | Starch from | | Water | |
| | | Ingredion Inc. | | Tapioca - | | | |
| | | | | Ingredion Inc. | | | |
| 36 | 3A7D7D1 | Emulsifying | 10.0 | Modified Food | 10.0 | Distilled | 40.0 |
| | | Starch - | | Starch from | | Water | |
| | | Cargill Inc. | | Tapioca - | | | |
| | | | | Ingredion Inc. | | | |
| 37 | 3A7D7E1 | Inulin, water | 10.0 | Modified Food | 10.0 | Distilled | 40.0 |
| | | soluble fiber - | | Starch from | | Water | |
| | | It's Just | | Tapioca - | | | |
| | | | | Ingredion Inc. | | | |
| 38 | 3A16A | Flavor-free | 10.0 | Organic Gum | 10.0 | Distilled | 55.0 |
| | | Maltodextrin - | | Acacia - | | Water | |
| | | Ingredion Inc. | | Ingredion Inc. | | | |
| 39 | 3A17A1 | Flavor-free | 10.0 | Modified Gum | 10.0 | Distilled | 55.0 |
| | | Maltodextrin - | | Acacia - | | Water | |
| | | Ingredion Inc. | | Ingredion Inc. | | | |
| 40 | 3A7D2A1 | Flavor-free | 7.5 | Modified Food | 7.5 | Distilled | 40.0 |
| | | Maltodextrin - | | Starch from | | Water | |
| | | Ingredion Inc. | | Tapioca - | | | |
| | | | | Ingredion Inc. | | | |
| 41 | 3A7F2 | Flavor-free | 10.0 | Modified Food | 10.0 | Distilled | 55.0 |
| | | Maltodextrin - | | Starch from | | Water | |
| | | Ingredion Inc. | | Tapioca - | | | |
| | | | | Ingredion Inc. | | | |
| 42 | 3A7G1 | Flavor-free | 10.0 | Modified Food | 10.0 | Distilled | 40.0 |
| | | Maltodextrin - | | Starch from | | Water | |
| | | Ingredion Inc. | | Tapioca - | | | |
| | | | | Ingredion Inc. | | | |
| 43 | 1H1 | Flavor-free | 5.0 | Modified Food | 10.0 | Distilled | 55.0 |
| | | Maltodextrin - | | Starch from | | Water | |
| | | Ingredion Inc. | | Waxy Maize - | | | |
| | | | | Ingredion Inc. | | | |
| 44 | 3Z2 | Maltodextrin, | 25.0 | Modified Food | 10.0 | Distilled | 55.0 |
| | | 90% Dietary | | Starch from | | Water | |
| | | Fiber - | | Tapioca - | | | |
| | | Fibersol | | Ingredion Inc. | | | |

Tables S003E and S003F below show the compositions of various water-dispersible particulates prepared through spray drying of the stable emulsions for electrostatic spray drying per Table S003C and Table S003D above. Table S003E shows the hydrophobic phase of each example water-dispersible particulate, and Table S003F shows the hydrophilic phase of each example water-dispersible particulate.

The formulations shown in these tables relate to the disclosures herein at least at paragraphs [0009], [0010], and [0011] above.

TABLE S003E

| | | Hydrophobic Phase | | | |
|---|---|---|---|---|---|
| | | Active | Usage | Car- | Usage |
| Exam- | Prototype | Biological | Rate | rier | Rate |
| ple | ID | Ingredient | (% w/w) | Oil | (% w/w) |
| 25 | P210412- | CBD Isolate | 20.0 | MCT | 20.0 |
| | 1A | | | | |
| 26 | P210330- | CBD Isolate | 11.1 | MCT | 11.1 |
| | 2B | | | | |
| 27 | P210412- | CBD Isolate | 11.1 | MCT | 11.1 |
| | 3 | | | | |
| 28 | 3D (sta- | CBD Isolate | 11.1 | ssMCT | 11.1 |
| | bility | | | | |
| | sample) | | | | |
| 29 | 3G | CBD Isolate | 11.1 | MCT | 11.1 |
| 30 | P210405- | CBD Isolate | 11.1 | MCT | 11.1 |
| | 2C | | | | |
| 31 | 3A | CBD Isolate | 11.1 | MCT | 11.1 |
| 32 | 3E | CBD Isolate | 11.1 | MCT | 11.1 |
| 33 | 3B | CBD Isolate | 11.1 | MCT | 11.1 |
| 34 | 3C | CBD Isolate | 11.1 | MCT | 11.1 |
| 35 | 3F | CBD Isolate | 11.1 | MCT | 11.1 |
| 36 | 3A7D7D1 | Broad Spectrum | 33.3 | MCT | 33.3 |
| | | Distillate | | | |
| 37 | 3A7D7E1 | Broad Spectrum | 33.3 | MCT | 33.3 |
| | | Distillate | | | |
| 38 | 3A16A | Broad Spectrum | 27.8 | MCT | 27.8 |
| | | Distillate | | | |
| 39 | 3A17A1 | Broad Spectrum | 27.8 | MCT | 27.8 |
| | | Distillate | | | |
| 40 | 3A7D2A1 | Broad Spectrum | 37.5 | MCT | 37.5 |
| | | Distillate | | | |
| 41 | 3A7F2 | Full Spectrum | 27.8 | MCT | 27.8 |
| | | Distillate | | | |
| 42 | 3A7G1 | Full Spectrum | 33.3 | MCT | 33.3 |
| | | Distillate | | | |
| 43 | 1H1 | Full Spectrum | 16.7 | MCT | 33.3 |
| | | Distillate | | | |
| | | CBN Isolate | 16.7 | | |
| 44 | 3Z2 | Full Spectrum | 5.6 | MCT | 11.1 |
| | | Distillate | | | |
| | | CBG Distillate | 5.6 | | |

TABLE S003F
Hydrophilic Phase

| | | | Usage | | Usage |
|---|---|---|---|---|---|
| | Prototype | | Rate | | Rate |
| Example | ID | Encapsulant | (% w/w) | Emulsifier | (% w/w) |
| 25 | P210412-1A | | | Modified Food Starch from Waxy | 60.0 |
| | | | | Maize - Ingredion Inc. | |
| 26 | P210330-2B | Maltodextrin, Tapioca Based - | 55.6 | Modified Corn Starch - Ingredion Inc. | 22.2 |
| | | Ingredion Inc. | | | |
| 27 | P210412-3 | Maltodextrin, 90% Dietary | 55.6 | Modified Food Starch from Tapioca - | 22.2 |
| | | Fiber - Fibersol | | Ingredion Inc. | |
| 28 | 3D (stability | Flavor-free Maltodextrin - | 55.6 | Modified Food Starch from Tapioca - | 22.2 |
| | sample) | Ingredion Inc. | | Ingredion Inc. | |
| 29 | 3G | Flavor-free Maltodextrin - | 55.6 | Modified Food Starch from Tapioca - | 22.2 |
| | | Ingredion Inc. | | Ingredion Inc. | |
| 30 | P210405-2C | Maltodextrin, Tapioca Based - | 55.6 | Modified Corn Starch - Ingredion Inc. | 22.2 |
| | | Ingredion Inc. | | | |
| 31 | 3A | Flavor-free Maltodextrin - | 55.6 | Modified Food Starch from Tapioca - | 22.2 |
| | | Ingredion Inc. | | Ingredion Inc. | |
| 32 | 3E | Flavor-free Maltodextrin - | 55.6 | Modified Food Starch from Tapioca - | 22.2 |
| | | Ingredion Inc. | | Ingredion Inc. | |
| 33 | 3B | Flavor-free Maltodextrin - | 55.6 | Modified Food Starch from Tapioca - | 22.2 |
| | | Ingredion Inc. | | Ingredion Inc. | |
| 34 | 3C | Flavor-free Maltodextrin - | 55.6 | Modified Food Starch from Tapioca - | 22.2 |
| | | Ingredion Inc. | | Ingredion Inc. | |
| 35 | 3F | Flavor-free Maltodextrin - | 55.6 | Modified Food Starch from Tapioca - | 22.2 |
| | | Ingredion Inc. | | Ingredion Inc. | |
| 36 | 3A7D7D1 | Emulsifying Starch - | 16.7 | Modified Food Starch from Tapioca - | 16.7 |
| | | Cargill Inc. | | Ingredion Inc. | |
| 37 | 3A7D7E1 | Inulin, water soluble fiber - | 16.7 | Modified Food Starch from Tapioca - | 16.7 |
| | | It's Just | | Ingredion Inc. | |
| 38 | 3A16A | Flavor-free Maltodextrin - | 22.2 | Organic Gum Acacia - Ingredion Inc. | 22.2 |
| | | Ingredion Inc. | | | |
| 39 | 3A17A1 | Flavor-free Maltodextrin - | 22.2 | Modified Gum Acacia - Ingredion Inc. | 22.2 |
| | | Ingredion Inc. | | | |
| 40 | 3A7D2A1 | Flavor-free Maltodextrin - | 12.5 | Modified Food Starch from Tapioca - | 12.5 |
| | | Ingredion Inc. | | Ingredion Inc. | |
| 41 | 3A7F2 | Flavor-free Maltodextrin - | 22.2 | Modified Food Starch from Tapioca - | 22.2 |
| | | Ingredion Inc. | | Ingredion Inc. | |
| 42 | 3A7G1 | Flavor-free Maltodextrin - | 16.7 | Modified Food Starch from Tapioca - | 16.7 |
| | | Ingredion Inc. | | Ingredion Inc. | |
| 43 | 1H1 | Flavor-free Maltodextrin - | 11.1 | Modified Food Starch from Waxy | 22.2 |
| | | Ingredion Inc. | | Maize - Ingredion Inc. | |
| 44 | 3Z2 | Maltodextrin, 90% Dietary | 55.6 | Modified Food Starch from Tapioca - | 22.2 |
| | | Fiber - Fibersol | | Ingredion Inc. | |

### Examples 45-48

Tables S004A and S004B show an ability to control the particle sizes in stable emulsions for electrostatic spray drying. In particular, Tables S004A and S004B show the importance of processing parameters, e.g., pressure and number of passes at higher pressure in affecting average particle diameter and particle distribution. Examples 45-49 are of the same composition as Examples 12-15 discussed above and shown in Table S001. Furthermore, each in of Examples 45-49: the carrier oil in the hydrophobic phase was MCT, the usage rate of carrier oil was 20.0% w/w, the ABI in the hydrophobic phase was full spectrum distillate, the usage rate of the ABI was 20.0% w/w, the encapsulant in the hydrophilic phase was Flavor-Free Maltodextrin from Ingredion Inc., the usage rate of the encapsulant was 10.0% w/w, the emulsifier in the hydrophilic phase was Modified Food Starch from Tapioca from Ingredion Inc., the usage rate of the emulsifier was 10.0% w/w, the continuous phase in the hydrophilic phase was distilled water, the usage rate of the continuous phase was 40.0% w/w, the percent solids in the emulation was 60.0%, and the percentage CBD in the powder of the emulsion was 28.3%.

The formulations and resulting particle sizes shown in these tables relate to the disclosures herein at least at paragraphs [0031], [0062], [0063], [0065], [0066], [0081], [00102], [00106], and [00107].

TABLE S004A
Particle Size Information

| | | | | | | Span | |
|---|---|---|---|---|---|---|---|
| | Prototype | Homogenization | Median Size | Harmonic | StDev | (10×- | Mean |
| Example | ID | Pressure (psi) | (nm) | Mean (nm) | (nm) | 90×) | RCA in g |
| 45 | 3A7G1A | 3,200 psi | 75.24 | 72.78 | 19.81 | 0.557 | 2290 |
| 46 | 3A7G1B | 10,000 psi | 38.98 | 34.11 | 12.29 | 0.756 | 2290 |
| 47 | 3A7G1C | 20,000 psi | 37.75 | 35.53 | 10.24 | 0.710 | 2290 |
| 48 | 3A7G1D | 30,000 psi | 38.18 | 38.8 | 7.56 | 0.458 | 2290 |

TABLE S004B
Particle Size Quantiles (in nm)

| | Prototype | Homogenization | | | | | |
|---|---|---|---|---|---|---|---|
| Example | ID | Pressure (psi) | ≤10% | ≤16% | ≤50% | ≤84% | ≤90% |
| 45 | 3A7G1A | 3,200 psi | 55.88 | 60.16 | 75.24 | 92.1 | 97.79 |
| 46 | 3A7G1B | 10,000 psi | 23.45 | 24.19 | 38.98 | 48.49 | 52.91 |
| 47 | 3A7G1C | 20,000 psi | 23.41 | 25.08 | 37.75 | 47.34 | 50.21 |
| 48 | 3A7G1D | 30,000 psi | 32.7 | 33.25 | 38.18 | 47.4 | 50.19 |

### Examples 49-63

Tables S004C, S004D, and S004F show an ability to control the particle sizes in liquid emulsions. Table S004C shows the compositions of various hydrophobic phases that go into the example liquid emulsions, Table S004D shows the compositions of various hydrophilic phases that go into the example liquid emulsions, and Table S004F show the processing parameters and corresponding resulting particle size properties of the liquid emulsions.

The formulations and resulting particle sizes shown in these tables relate to the disclosures herein at least at paragraphs [0031], [0062], [0063], [0065], [0066], [0081], [00102], [00106], and [00107].

TABLE S004C
Hydrophobic Phase

| | | | Usage | | Usage |
|---|---|---|---|---|---|
| Exam- | Prototype | Active Biological | Rate | Carrier | Rate |
| ple | ID | Ingredient | (% w/w) | Oil | (% w/w) |
| 49 | 25G3A- | Broad Spectrum | 2.0 | MCT | 2.0 |
| | 5k | Distillate | | | |
| 50 | 25G3A- | Broad Spectrum | 2.0 | MCT | 2.0 |
| | 10k | Distillate | | | |
| 51 | 25G3A- | Broad Spectrum | 2.0 | MCT | 2.0 |
| | 20k | Distillate | | | |
| 52 | 25G3A- | Broad Spectrum | 2.0 | MCT | 2.0 |
| | 30k-1p | Distillate | | | |
| 53 | 25G3A- | Broad Spectrum | 2.0 | MCT | 2.0 |
| | 30k-2p | Distillate | | | |
| 54 | 64G-5k | Broad Spectrum | 2.0 | MCT | 2.0 |
| | | Distillate | | | |
| 55 | 64G-10k | Broad Spectrum | 2.0 | MCT | 2.0 |
| | | Distillate | | | |
| 56 | 64G-20k | Broad Spectrum | 2.0 | MCT | 2.0 |
| | | Distillate | | | |
| 57 | 64G-30k- | Broad Spectrum | 2.0 | MCT | 2.0 |
| | 1p | Distillate | | | |
| 58 | 64G-30k- | Broad Spectrum | 2.0 | MCT | 2.0 |
| | 2p | Distillate | | | |
| 59 | 25G5C1 | Broad Spectrum | 5.0 | MCT | 7.5 |
| | | Distillate | | | |
| 60 | 25G5 | CBD Isolate | 1 | MCT | 1.5 |
| 61 | 10A2C6 | Full Spectrum | 5 | MCT | 12 |
| | | Distillate | | | |
| 62 | 10A2C5A | Full Spectrum | 5 | MCT | 16 |
| | | Distillate | | | |
| 63 | 10A2C5B | Full Spectrum | 5 | MCT | 25 |
| | | Distillate | | | |

TABLE S004D
Hydrophilic Phase

| | | | Usage | | Usage | | Usage |
|---|---|---|---|---|---|---|---|
| | | | Rate | | Rate | | Rate |
| | Prototype | | (% | | (% | | (% |
| Example | ID | Emulsifier | w/w) | Emulsifier | w/w) | Continuous Phase | w/w) |
| 49 | 25G3A-5k | Sunflower Lecithin, ≥99% - | 3.0 | | | Glycerin | 93.0 |
| | | Compass Foods Pte Ltd | | | | | |
| 50 | 25G3A-10k | Sunflower Lecithin, ≥99% - | 3.0 | | | Glycerin | 93.0 |
| | | Compass Foods Pte Ltd | | | | | |
| 51 | 25G3A-20k | Sunflower Lecithin, ≥99% - | 3.0 | | | Glycerin | 93.0 |
| | | Compass Foods Pte Ltd | | | | | |
| 52 | 25G3A- | Sunflower Lecithin, ≥99% - | 3.0 | | | Glycerin | 93.0 |
| | 30k-1p | Compass Foods Pte Ltd | | | | | |
| 53 | 25G3A- | Sunflower Lecithin, ≥99% - | 3.0 | | | Glycerin | 93.0 |
| | 30k-2p | Compass Foods Pte Ltd | | | | | |
| 54 | 64G-5k | Modified Food Starch | 3.0 | | | Glycerin | 93.0 |
| | | from Waxy Maize - | | | | | |
| | | Ingredion Inc. | | | | | |
| 55 | 64G-10k | Modified Food Starch | 3.0 | | | Glycerin | 93.0 |
| | | from Waxy Maize - | | | | | |
| | | Ingredion Inc. | | | | | |
| 56 | 64G-20k | Modified Food Starch | 3.0 | | | Glycerin | 93.0 |
| | | from Waxy Maize - | | | | | |
| | | Ingredion Inc. | | | | | |
| 57 | 64G-30k-1p | Modified Food Starch | 3.0 | | | Glycerin | 93.0 |
| | | from Waxy Maize - | | | | | |
| | | Ingredion Inc. | | | | | |
| 58 | 64G-30k-2p | Modified Food Starch | 3.0 | | | Glycerin | 93.0 |
| | | from Waxy Maize - | | | | | |
| | | Ingredion Inc. | | | | | |
| 59 | 25G5C1 | Sunflower Lecithin, ≥99% - | 3.8 | Sucrose | 1.9 | Glycerin | 81.8 |
| | | Compass Foods Pte Ltd | | Monopa | | | |
| | | | | Imitate | | | |
| | | | | P90 | | | |
| 60 | 25G5 | Sunflower Lecithin, ≥99% - | 2 | Sucrose | 1 | Glycerin | 94.5 |
| | | Compass Foods Pte Ltd | | Monoester | | | |
| | | | | of | | | |
| | | | | Palmitic | | | |
| | | | | Acid - | | | |
| | | | | Compass | | | |
| | | | | Foods | | | |
| | | | | Pte Ltd | | | |
| 61 | 10A2C6 | Quillaja Saponaria | 5 | Sucrose | 2.5 | Glycerin | 75.5 |
| | | Extract - Ingredion | | Monoester | | | |
| | | Inc. | | of | | | |
| | | | | Palmitic | | | |
| | | | | Acid - | | | |
| | | | | Compass | | | |
| | | | | Foods | | | |
| | | | | Pte Ltd | | | |
| 62 | 10A2C5A | Quillaja Saponaria | 5 | Sucrose | 2.5 | Glycerin | 71.5 |
| | | Extract - Ingredion | | Monoester | | | |
| | | Inc. | | of | | | |
| | | | | Palmitic | | | |
| | | | | Acid - | | | |
| | | | | Compass | | | |
| | | | | Foods | | | |
| | | | | Pte Ltd | | | |
| 63 | 10A2C5B | Quillaja Saponaria | 5 | Sucrose | 2.5 | Glycerin | 62.5 |
| | | Extract - Ingredion | | Monoester | | | |
| | | Inc. | | of | | | |
| | | | | Palmitic | | | |
| | | | | Acid - | | | |
| | | | | Compass | | | |
| | | | | Foods | | | |
| | | | | Pte Ltd | | | |

TABLE S004E
Liquid Emulsion pressures and resulting particle sizes

| | | | Turbidity (NTU) | | |
|---|---|---|---|---|---|
| | | | Corresponding to | | |
| | Prototype | Homogenization | 10 mg CBD per | Effective | |
| Example | ID | Pressure (psi) | 355 mL water | Diameter (nm) | Polydispersity |
| 49 | 25G3A-5k | 5,000 | 226 | 271.20 | 22.2% |
| 50 | 25G3A-10k | 10,000 | 180 | 241.20 | 6.4% |
| 51 | 25G3A-20k | 20,000 | 45 | 143.98 | 12.8% |
| 52 | 25G3A-30k-1p | 30,000 1 pass | 23.8 | 113.88 | 13.8% |
| 53 | 25G3A-30k-2p | 30,000 2 passes | 16.25 | 97.98 | 13.4% |
| 54 | 64G-5k | 5000 | 200 | 322.60 | 8.8% |
| 55 | 64G-10k | 10000 | 186 | 287.50 | 9.0% |
| 56 | 64G-20k | 20000 | 85.6 | 319.40 | 28.9% |
| 57 | 64G-30k-1p | 30,000 1 pass | 94.2 | 237.50 | 13.2% |
| 58 | 64G-30k-2p | 30,000 2 passes | 132 | 233.50 | 19.2% |
| 59 | 25G5C1 | 30,000 | 17 | 197.04 | 8.1% |
| 60 | 25G5 | 30,000 | 3 | 83.67 | 17.8% |
| 61 | 10A2C6 | 30,000 | 29 | 146.33 | 10.7% |
| 62 | 10A2C5A | 30,000 | 42 | 162.72 | 8.6% |
| 63 | 10A2C5B | 30,000 | 109 | 197.7 | 13.2% |

### Examples 64-211

Tables S005A, S005B, and S005C show examples of stable emulsions using various active biological ingredients and blends thereof. These formulations are not limited to the ABIs listed herein and can be prepared with other lipophilic ABIs and blends thereof as listed above at least at paragraphs [0033], [0034], [0035], [0036], and [0037]. The results in these tables show that the formulations exhibit robustness, i.e. varying the ABI and other ingredients still achieves the desired outcomes and parameters.

TABLE S005A

| | | | Usage | | Usage | | |
|---|---|---|---|---|---|---|---|
| | | | Rate | | Rate | | Usage |
| | Prototype | | (% | | (% | Flavorant or | Rate (% |
| Example | ID | ABI | w/w) | Carrier Oil | w/w) | Bittermasker | w/w) |
| 64 | WGU-1- | CBD | 0.5 | MCT | 0.5 | | |
| | 5000 | Isolate | | | | | |
| 65 | 25G2 | CBD | 1.0 | MCT | 1.5 | | |
| | | Isolate | | | | | |
| 66 | 25G5 | CBD | 1.0 | MCT | 1.5 | | |
| | | Isolate | | | | | |
| 67 | 25G5-CO1 | CBD | 1.0 | Refined | 1.5 | | |
| | | Isolate | | Sesame Oil | | | |
| | | | | (IV-1) | | | |
| 68 | 25G5- | CBD | 1.0 | Hemp Seed | 1.5 | | |
| | CO12 | Isolate | | Oil (bna-170-r) | | | |
| 69 | 9B | CBD | 1.0 | Glyceril | 2.0 | | |
| | | Isolate | | Monooleate | | | |
| | | | | (Type 40) - | | | |
| | | | | Gattefossé | | | |
| | | | | SAS | | | |
| 70 | WSP-3 | CBD | 1.0 | MCT | 1.0 | | |
| | | Isolate | | | | | |
| 71 | 25G711 | CBD | 2.0 | MCT | 3.0 | Bitterness Blocker, | 0.7 |
| | | Isolate | | | | Natural, Type A - | |
| | | | | | | Allen Flavors Inc. | |
| 72 | 25G5F | CBD | 2.0 | Hemp Seed | 1.5 | | |
| | | Isolate | | Oil | | | |
| 73 | WQN-3A | CBD | 2.0 | MCT | 1.0 | | |
| | | Isolate | | | | | |
| 74 | 25A3 | CBD | 2.0 | MCT | 3.0 | | |
| | | Isolate | | | | | |
| 75 | 56F3H1B | CBD | 2.0 | MCT | 3.3 | Bitterness Blocker, | 0.7 |
| | | Isolate | | | | Natural, Type A - | |
| | | | | | | Allen Flavors Inc. | |
| 76 | 56F3H1B | CBD | 2.0 | MCT | 3.3 | Bitterness Blocker, | 0.7 |
| | | Isolate | | | | Natural, Type A - | |
| | | | | | | Allen Flavors Inc. | |
| 77 | 250 | CBD | 2.0 | MCT | 3.0 | | |
| | | Isolate | | | | | |
| 78 | 25G2B | CBD | 2.0 | MCT | 3.0 | Bitterness Blocker, | 0.1 |
| | | Isolate | | | | Natural, Type A - | |
| | | | | | | Allen Flavors Inc. | |
| 79 | 25G2A | CBD | 2.0 | MCT | 3.0 | | |
| | | Isolate | | | | | |
| 80 | 10A2C5A9 | CBD | 2.5 | MCT | 7.0 | Bitterness Blocker, | 0.5 |
| | | Isolate | | | | Natural, Type B - | |
| | | | | | | Allen Flavors Inc. | |
| 81 | 25N | CBD | 3.0 | MCT | 4.0 | | |
| | | Isolate | | | | | |
| 82 | 56F3C | CBD | 3.0 | MCT | 7.0 | Bitterness Blocker, | 0.1 |
| | | Isolate | | | | Natural, Type C - | |
| | | | | | | Allen Flavors Inc. | |
| 83 | 10A2C4 | CBD | 5.0 | MCT | 10.0 | | |
| | | Isolate | | | | | |
| 84 | WTAM-2- | CBD | 6.0 | MCT | 6.0 | | |
| | 5000 | Isolate | | | | | |
| 85 | 56H | CBD | 7.5 | MCT | 7.5 | | |
| | | Isolate | | | | | |
| 86 | 10A2C5A1C | CBD | 8.0 | MCT | 22.4 | Bitterness Blocker, | 0.3 |
| | | Isolate | | | | Natural, Type C - | |
| | | | | | | Allen Flavors Inc. | |
| 87 | 25A5B- | CBD | 8.1 | MCT | 12.2 | Lemon Oil Flavor - | 0.5 |
| | LE3 | Isolate | | | | Flavor Insights | |
| | | | | | | Bitterness Blocker, | 0.3 |
| | | | | | | Natural, Type A - | |
| | | | | | | Allen Flavors Inc. | |
| 88 | 25A5C-GR | CBD | 8.1 | MCT | 12.2 | Grapefruit Oil | 0.5 |
| | | Isolate | | | | Flavor - Flavor | |
| | | | | | | Insights | |
| | | | | | | Bitterness Blocker, | 0.3 |
| | | | | | | Natural, Type A - | |
| | | | | | | Allen Flavors Inc. | |
| 89 | 10A2C3 | CBD | 8.5 | MCT | 8.5 | | |
| | | Isolate | | | | | |
| 90 | 64 | CBD | 10.0 | MCT | 10.0 | | |
| | | Isolate | | | | | |
| 91 | 64A | CBD | 10.0 | MCT | 10.0 | | |
| | | Isolate | | | | | |
| 92 | | CBD | 12.5 | MCT | 12.5 | | |
| | | Isolate | | | | | |
| 93 | | CBD | 15.0 | MCT | 15.0 | | |
| | | Isolate | | | | | |
| 94 | | CBD | 17.5 | MCT | 17.5 | | |
| | | Isolate | | | | | |
| 95 | | CBD | 20.0 | MCT | 20.0 | | |
| | | Isolate | | | | | |
| 96 | 3A5A | CBD | 16.7 | MCT | 16.7 | | |
| | | Isolate | | | | | |
| 97 | 3A5B | CBD | 18.4 | MCT | 18.4 | | |
| | | Isolate | | | | | |
| 98 | 3A5B1 | CBD | 21.7 | MCT | 21.7 | | |
| | | Isolate | | | | | |
| 99 | 3A7D6A | CBD | 22.5 | MCT | 22.5 | | |
| | | Isolate | | | | | |
| 100 | 3A7D6B | CBD | 22.5 | MCT | 22.5 | | |
| | | Isolate | | | | | |
| 101 | 43 | Broad | 1.0 | Grape seed | 1.0 | | |
| | | Spectrum | | oil | | | |
| | | Distillate | | | | | |
| 102 | 25G51 | Broad | 1.0 | MCT | 1.5 | | |
| | | Spectrum | | | | | |
| | | Distillate | | | | | |
| 103 | 9T | Broad | 1.0 | MCT | 1.0 | | |
| | | Spectrum | | | | | |
| | | Distillate | | | | | |
| 104 | 25G | Broad | 1.0 | MCT | 1.0 | | |
| | | Spectrum | | | | | |
| | | Distillate | | | | | |
| 105 | 25M | Broad | 1.0 | Refined Olive | 1.0 | | |
| | | Spectrum | | Oil | | | |
| | | Distillate | | | | | |
| 106 | 20K | Broad | 1.0 | MCT | 1.0 | | |
| | | Spectrum | | | | | |
| | | Distillate | | | | | |
| 107 | 20D | Broad | 1.0 | MCT | 1.0 | | |
| | | Spectrum | | | | | |
| | | Distillate | | | | | |
| 108 | 56B | Broad | 1.0 | MCT | 1.0 | | |
| | | Spectrum | | | | | |
| | | Distillate | | | | | |
| 109 | 58 | Broad | 1.0 | MCT | 1.0 | | |
| | | Spectrum | | | | | |
| | | Distillate | | | | | |
| 110 | 34C | Broad | 1.0 | Refined Olive | 1.0 | | |
| | | Spectrum | | Oil | | | |
| | | Distillate | | | | | |
| 111 | 25G3 | Broad | 2.0 | Refined Olive | 2.0 | | |
| | | Spectrum | | Oil | | | |
| | | Distillate | | | | | |
| 112 | 10C1 | Broad | 2.0 | MCT | 6.0 | | |
| | | Spectrum | | | | | |
| | | Distillate | | | | | |
| 113 | 44B | Broad | 3.0 | MCT | 3.0 | | |
| | | Spectrum | | | | | |
| | | Distillate | | | | | |
| 114 | 7A | Broad | 3.0 | MCT | 3.0 | | |
| | | Spectrum | | | | | |
| | | Distillate | | | | | |
| 115 | 43E | Broad | 3.0 | MCT | 3.0 | | |
| | | Spectrum | | | | | |
| | | Distillate | | | | | |
| 116 | 61A | Broad | 4.0 | MCT | 6.0 | | |
| | | Spectrum | | | | | |
| | | Distillate | | | | | |
| 117 | 10A1 | Broad | 4.0 | MCT | 4.0 | | |
| | | Spectrum | | | | | |
| | | Distillate | | | | | |
| 118 | 9G | Broad | 4.0 | MCT | 3.0 | | |
| | | Spectrum | | | | | |
| | | Distillate | | | | | |
| 119 | 56F3H1A - | Broad | 4.0 | MCT | 6.7 | Bitterness Blocker, | 0.7 |
| | BSD | Spectrum | | | | Natural, Type A - | |
| | | Distillate | | | | Allen Flavors Inc. | |
| 120 | 10F5 | Broad | 5.0 | MCT | 7.5 | | |
| | | Spectrum | | | | | |
| | | Distillate | | | | | |
| 121 | 10A4 | Broad | 5.0 | MCT | 10.0 | | |
| | | Spectrum | | | | | |
| | | Distillate | | | | | |
| 122 | 64C1 | Broad | 5.0 | MCT | 15.0 | | |
| | | Spectrum | | | | | |
| | | Distillate | | | | | |
| 123 | 64D | Broad | 5.0 | MCT | 20.0 | | |
| | | Spectrum | | | | | |
| | | Distillate | | | | | |
| 124 | 10A2C5A5D1 | Broad | 8.0 | MCT | 30.0 | Bitterness Blocker, | 1.4 |
| | | Spectrum | | | | Natural, Type C - | |
| | | Distillate | | | | Allen Flavors Inc. | |
| 125 | 25G5C3B2A | Broad | 9.0 | MCT | 12.0 | | |
| | | Spectrum | | | | | |
| | | Distillate | | | | | |
| 126 | 64B | Broad | 10.0 | MCT | 10.0 | | |
| | | Spectrum | | | | | |
| | | Distillate | | | | | |
| 127 | 64C | Broad | 10.0 | MCT | 10.0 | | |
| | | Spectrum | | | | | |
| | | Distillate | | | | | |
| 128 | | Broad | 12.5 | MCT | 12.5 | | |
| | | Spectrum | | | | | |
| | | Distillate | | | | | |
| 129 | | Broad | 15.0 | MCT | 15.0 | | |
| | | Spectrum | | | | | |
| | | Distillate | | | | | |
| 130 | | Broad | 17.5 | MCT | 17.5 | | |
| | | Spectrum | | | | | |
| | | Distillate | | | | | |
| 131 | | Broad | 20.0 | MCT | 20.0 | | |
| | | Spectrum | | | | | |
| | | Distillate | | | | | |
| 132 | 3A16A | Broad | 12.5 | MCT | 12.5 | | |
| | | Spectrum | | | | | |
| | | Distillate | | | | | |
| 133 | 3A17A1 | Broad | 12.5 | MCT | 12.5 | | |
| | | Spectrum | | | | | |
| | | Distillate | | | | | |
| 134 | 1G | Broad | 15.0 | MCT | 15.0 | | |
| | | Spectrum | | | | | |
| | | Distillate | | | | | |
| 135 | 6D | Broad | 15.0 | MCT | 15.0 | | |
| | | Spectrum | | | | | |
| | | Distillate | | | | | |
| 136 | 9C | Broad | 15.0 | MCT | 15.0 | | |
| | | Spectrum | | | | | |
| | | Distillate | | | | | |
| 137 | 3A10A | Broad | 18.0 | MCT | 18.0 | | |
| | | Spectrum | | | | | |
| | | Distillate | | | | | |
| 138 | 3A7D7H | Broad | 20.0 | Hemp Seed | 19.2 | Orange Oil Flavor - | 0.8 |
| | | Spectrum | | Oil | | Flavor Insights | |
| | | Distillate | | | | | |
| 139 | 3A7D7D1 | Broad | 20.0 | MCT | 20.0 | | |
| | | Spectrum | | | | | |
| | | Distillate | | | | | |
| 140 | 3A7D7E1 | Broad | 20.0 | MCT | 20.0 | | |
| | | Spectrum | | | | | |
| | | Distillate | | | | | |
| 141 | 3A7D2A1 | Broad | 22.5 | MCT | 22.5 | | |
| | | Spectrum | | | | | |
| | | Distillate | | | | | |
| 142 | 29A | Full | 1.0 | MCT | 1.0 | | |
| | | Spectrum | | | | | |
| | | Distillate | | | | | |
| 143 | 9 | Full | 1.0 | MCT | 1.0 | | |
| | | Spectrum | | | | | |
| | | Distillate | | | | | |
| 144 | 10 | Full | 1.0 | MCT | 1.0 | | |
| | | Spectrum | | | | | |
| | | Distillate | | | | | |
| 145 | 26A | Full | 1.0 | MCT | 1.0 | | |
| | | Spectrum | | | | | |
| | | Distillate | | | | | |
| 146 | 5C | Full | 1.0 | MCT | 1.0 | | |
| | | Spectrum | | | | | |
| | | Distillate | | | | | |
| 147 | 25Q | Full | 1.0 | MCT | 1.0 | | |
| | | Spectrum | | | | | |
| | | Distillate | | | | | |
| 148 | L201112- | Full | 1.0 | MCT | 0.3 | | |
| | 1J | Spectrum | | | | | |
| | | Distillate | | | | | |
| 149 | L201112- | Full | 1.0 | MCT | 0.3 | | |
| | 1J | Spectrum | | | | | |
| | | Distillate | | | | | |
| 150 | 1H | Full | 1.0 | MCT | 1.0 | | |
| | | Spectrum | | | | | |
| | | Distillate | | | | | |
| 151 | L201112- | Full | 1.0 | MCT | 0.3 | | |
| | 1F | Spectrum | | | | | |
| | | Distillate | | | | | |
| 152 | 9 | Full | 1.0 | MCT | 1.0 | | |
| | | Spectrum | | | | | |
| | | Distillate | | | | | |
| 153 | 29B | Full | 1.0 | MCT | 1.0 | | |
| | | Spectrum | | | | | |
| | | Distillate | | | | | |
| 154 | 12 | Full | 1.0 | MCT | 1.0 | | |
| | | Spectrum | | | | | |
| | | Distillate | | | | | |
| 155 | 43A2 | Full | 2.0 | MCT | 2.0 | | |
| | | Spectrum | | | | | |
| | | Distillate | | | | | |
| 156 | 21D | Full | 2.0 | Maisine CC | 2.0 | | |
| | | Spectrum | | | | | |
| | | Distillate | | | | | |
| 157 | 21C | Full | 3.0 | MCT | 3.0 | | |
| | | Spectrum | | | | | |
| | | Distillate | | | | | |
| 158 | L201112- | Full | 3.0 | MCT | 1.0 | | |
| | 1G | Spectrum | | | | | |
| | | Distillate | | | | | |
| 159 | 11A | Full | 3.0 | MCT | 3.0 | | |
| | | Spectrum | | | | | |
| | | Distillate | | | | | |
| 160 | 25G5C2-1 | Full | 4.0 | MCT | 10.0 | | |
| | | Spectrum | | | | | |
| | | Distillate | | | | | |
| 161 | 10F11B | Full | 5.0 | MCT | 10.0 | | |
| | | Spectrum | | | | | |
| | | Distillate | | | | | |
| 162 | 10A2C6 | Full | 5.0 | MCT | 12.0 | | |
| | | Spectrum | | | | | |
| | | Distillate | | | | | |
| 163 | 10F1-CFH | Full | 7.5 | MCT | 7.5 | | |
| | | Spectrum | | | | | |
| | | Distillate | | | | | |
| | | (CFH) | | | | | |
| 164 | | Full | 10.0 | MCT | 10.0 | | |
| | | Spectrum | | | | | |
| | | Distillate | | | | | |
| 165 | | Full | 12.5 | MCT | 12.5 | | |
| | | Spectrum | | | | | |
| | | Distillate | | | | | |
| 166 | | Full | 15.0 | MCT | 15.0 | | |
| | | Spectrum | | | | | |
| | | Distillate | | | | | |
| 167 | | Full | 17.5 | MCT | 17.5 | | |
| | | Spectrum | | | | | |
| | | Distillate | | | | | |
| 168 | | Full | 20.0 | MCT | 20.0 | | |
| | | Spectrum | | | | | |
| | | Distillate | | | | | |
| 169 | 3A7F2 | Full | 12.5 | MCT | 12.5 | | |
| | | Spectrum | | | | | |
| | | Distillate | | | | | |
| 170 | 3A16B | Full | 12.5 | MCT | 12.5 | | |
| | | Spectrum | | | | | |
| | | Distillate | | | | | |
| 171 | 3A17A2 | Full | 12.5 | MCT | 12.5 | | |
| | | Spectrum | | | | | |
| | | Distillate | | | | | |
| 172 | 1H | Full | 15.0 | MCT | 15.0 | | |
| | | Spectrum | | | | | |
| | | Distillate | | | | | |
| 173 | 6E | Full | 15.0 | MCT | 15.0 | | |
| | | Spectrum | | | | | |
| | | Distillate | | | | | |
| 174 | 9D | Full | 15.0 | MCT | 15.0 | | |
| | | Spectrum | | | | | |
| | | Distillate | | | | | |
| 175 | 3A7D7G | Full | 20.0 | MCT | 20.0 | | |
| | | Spectrum | | | | | |
| | | Distillate | | | | | |
| 176 | 3A7D7D2 | Full | 20.0 | MCT | 20.0 | | |
| | | Spectrum | | | | | |
| | | Distillate | | | | | |
| 177 | 3A7D7E2 | Full | 20.0 | MCT | 20.0 | | |
| | | Spectrum | | | | | |
| | | Distillate | | | | | |
| 178 | 25G5-CBN | CBN | 1.0 | MCT | 1.5 | | |
| | | Isolate | | | | | |
| 179 | 10F-CBN | CBN | 1.5 | MCT | 1.2 | | |
| | | Isolate | | | | | |
| 180 | 25G5-CBG | CBG | 1.0 | MCT | 1.5 | | |
| | Isolate | Isolate | | | | | |
| 181 | 25G5-CBG | CBG | 1.0 | MCT | 1.5 | | |
| | Distillate | Distillate | | | | | |
| 182 | 25A5A1 | CBD | 1.4 | MCT | 7.8 | Bitterness Blocker, | 0.1 |
| | | Isolate | | | | Natural, Type A - | |
| | | CBN | 1.4 | | | Allen Flavors Inc. | |
| | | Isolate | | | | | |
| 183 | 25A5A1- | CBD | 1.4 | MCT | 7.8 | Lemon Oil Flavor - | 0.2 |
| | LE | Isolate | | | | Flavor Insights | |
| | | CBN | 1.4 | | | Bitterness Blocker, | 0.1 |
| | | Isolate | | | | Natural, Type A - | |
| | | | | | | Allen Flavors Inc. | |
| 184 | 64A2 | CBD | 3.3 | MCT | 10.0 | | |
| | | Isolate | | | | | |
| | | CBN | 1.7 | | | | |
| | | Isolate | | | | | |
| 185 | 64A3 | CBD | 3.3 | MCT | 15.0 | | |
| | | Isolate | | | | | |
| | | CBN | 1.7 | | | | |
| | | Isolate | | | | | |
| 186 | 64A4 | CBD | 3.3 | MCT | 15.0 | | |
| | | Isolate | | | | | |
| | | CBN | 1.7 | | | | |
| | | Isolate | | | | | |
| 187 | 64A1 | CBD | 6.7 | MCT | 10.0 | | |
| | | Isolate | | | | | |
| | | CBN | 3.3 | | | | |
| | | Isolate | | | | | |
| 188 | 64A5 | CBD | 3.3 | MCT | 15.0 | | |
| | | Isolate | | | | | |
| | | CBN | 1.7 | | | | |
| | | Isolate | | | | | |
| 189 | 64A6 | CBD | 3.3 | MCT | 20.0 | | |
| | | Isolate | | | | | |
| | | CBN | 1.7 | | | | |
| | | Isolate | | | | | |
| 190 | 10A2C5A5J | CBD | 5.2 | MCT | 30.0 | Bitterness Blocker, | 1.4 |
| | | Isolate | | | | Natural, Type C - | |
| | | CBN | 2.6 | | | Allen Flavors Inc. | |
| | | Isolate | | | | | |
| 191 | 56F3H2 | CBD | 2.0 | MCT | 5.0 | Bitterness Blocker, | 0.5 |
| | | Isolate | | | | Natural, Type A - | |
| | | CBN | 1.0 | | | Allen Flavors Inc. | |
| | | Isolate | | | | | |
| 192 | 56F3H2A | Broad | 2.0 | MCT | 5.0 | Bitterness Blocker, | 0.5 |
| | | Spectrum | | | | Natural, Type A - | |
| | | Distillate | | | | Allen Flavors Inc. | |
| | | CBN | 1.0 | | | | |
| | | Isolate | | | | | |
| 193 | 56F3H2B | Broad | 2.0 | Hemp Seed | 4.5 | Bitterness Blocker, | 0.5 |
| | | Spectrum | | Oil | | Natural, Type A - | |
| | | Distillate | | | | Allen Flavors Inc. | |
| | | CBN | 1.0 | MCT | 0.5 | | |
| | | Isolate | | | | | |
| 194 | 56H6A | Broad | 2.0 | MCT | 5.0 | | |
| | | Spectrum | | | | | |
| | | Distillate | | | | | |
| | | CBG | 1.0 | | | | |
| | | Distillate | | | | | |
| 195 | 64C9 | Broad | 7.0 | MCT | 10.0 | | |
| | | Spectrum | | | | | |
| | | Distillate | | | | | |
| | | CBG | 3.0 | | | | |
| | | Distillate | | | | | |
| 196 | 10A2C5A1E2 | CBD | 2.3 | MCT | 7.8 | Bitterness Blocker, | 0.1 |
| | | Isolate | | | | Natural, Type C - | |
| | | CBG | 0.2 | | | Allen Flavors Inc. | |
| | | Distillate | | | | | |
| | | CBN | 0.2 | | | | |
| | | Isolate | | | | | |
| 197 | 64D2 | Broad | 2.5 | MCT | 20.0 | | |
| | | Spectrum | | | | | |
| | | Distillate | | | | | |
| | | CBD | 2.5 | | | | |
| | | Isolate | | | | | |
| 198 | 64D3 | Broad | 5.0 | MCT | 15.0 | | |
| | | Spectrum | | | | | |
| | | Distillate | | | | | |
| | | CBD | 5.0 | | | | |
| | | Isolate | | | | | |
| 199 | 64D4 | Broad | 5.0 | MCT | 15.0 | | |
| | | Spectrum | | | | | |
| | | Distillate | | | | | |
| | | CBD | 5.0 | | | | |
| | | Isolate | | | | | |
| 200 | 64D5 | Broad | 5.0 | MCT | 15.0 | | |
| | | Spectrum | | | | | |
| | | Distillate | | | | | |
| | | CBD | 5.0 | | | | |
| | | Isolate | | | | | |
| 201 | 64D6 | Broad | 5.0 | MCT | 15.0 | | |
| | | Spectrum | | | | | |
| | | Distillate | | | | | |
| | | CBD | 5.0 | | | | |
| | | Isolate | | | | | |
| 202 | 64D4A1 | Broad | 5.0 | Glyceril | 13.0 | | |
| | | Spectrum | | Monooleate | | | |
| | | Distillate | | (Type 40) - | | | |
| | | | | Gattefossé | | | |
| | | | | SAS | | | |
| | | CBD | 5.0 | MCT | 2.0 | | |
| | | Isolate | | | | | |
| 203 | 64D4A2 | Broad | 5.0 | Refined | 14.5 | | |
| | | Spectrum | | Soybean Oil | | | |
| | | Distillate | | | | | |
| | | CBD | 5.0 | MCT | 0.5 | | |
| | | Isolate | | | | | |
| 204 | 1H1 | Full | 7.5 | MCT | 15.0 | | |
| | | Spectrum | | | | | |
| | | Distillate | | | | | |
| | | CBN | 7.5 | | | | |
| | | Isolate | | | | | |
| 205 | 3Z2 | Full | 2.5 | MCT | 5.0 | | |
| | | Spectrum | | | | | |
| | | Distillate | | | | | |
| | | CBG | 2.5 | | | | |
| | | Distillate | | | | | |
| 206 | 3A7D7G1 | Full | 6.0 | Olive Oil | 19.0 | Bitterness Blocker, | 1.0 |
| | | Spectrum | | | | Natural, Type A - | |
| | | Distillate | | | | Allen Flavors Inc. | |
| | | CBN | 7.0 | | | | |
| | | Isolate | | | | | |
| | | CBG | 7.0 | | | | |
| | | isolate | | | | | |
| 207 | 3A7D7D3A | Broad | 7.0 | MCT | 20.0 | | |
| | | Spectrum | | | | | |
| | | Distillate | | | | | |
| | | CBN | 7.0 | | | | |
| | | Isolate | | | | | |
| | | CBG | 6.0 | | | | |
| | | isolate | | | | | |
| 208 | 7B | Broad | 4.0 | MCT | 8.0 | | |
| | | Spectrum | | | | | |
| | | Distillate | | | | | |
| | | CBN | 4.0 | | | | |
| | | Isolate | | | | | |
| 209 | 9C | Broad | 7.5 | MCT | 15.0 | | |
| | | Spectrum | | | | | |
| | | Distillate | | | | | |
| | | CBG | 7.5 | | | | |
| | | Distillate | | | | | |
| 210 | 3A7D7F | Broad | 10.0 | Hemp Seed | 18.8 | Lemon Oil Flavor - | 1.2 |
| | | Spectrum | | oil | | Flavor Insights | |
| | | Distillate | | | | | |
| | | CBN | 5.0 | | | | |
| | | Isolate | | | | | |
| 211 | 3A7D7G | Broad | 4.0 | Olive Oil | 18.5 | Modified Food | 5.0 |
| | | Spectrum | | | | Starch from Waxy | |
| | | Distillate | | | | Maize - Ingredion | |
| | | | | | | Inc. | |
| | | CBG | 8.0 | | | Bitterness Blocker, | 1.5 |
| | | distillate | | | | Natural, Type C - | |
| | | | | | | Allen Flavors Inc. | |

TABLE S005B

| | Primary | Usage rate | Secondary | Usage Rate | Tertiary | Usage Rate |
|---|---|---|---|---|---|---|
| Example | Emulsifier | (% w/w) | Emulsifier | (% w/w) | Emulsifier | (% w/w) |
| 64 | Pre-hydrated | 1.5 | | | | |
| | Gum Arabic | | | | | |
| 65 | Sunflower | 2.0 | Enzyme | 0.5 | Sucrose | 0.5 |
| | Lecithin, ≥99% - | | Modified Soy | | Monoester of | |
| | Compass | | Lecithin - | | Palmitic Acid - | |
| | Foods Pte | | American | | Compass | |
| | Ltd | | Lecithin | | Foods Pte | |
| | | | Company | | Ltd | |
| 66 | Sunflower | 2.0 | Sucrose | 1.0 | | |
| | Lecithin, ≥99% - | | Monoester of | | | |
| | Compass | | Palmitic Acid - | | | |
| | Foods Pte | | Compass | | | |
| | Ltd | | Foods Pte | | | |
| | | | Ltd | | | |
| 67 | Sunflower | 2.0 | Sucrose | 1.0 | | |
| | Lecithin, ≥99% - | | Monoester of | | | |
| | Compass | | Palmitic Acid - | | | |
| | Foods Pte | | Compass | | | |
| | Ltd | | Foods Pte | | | |
| | | | Ltd | | | |
| 68 | Sunflower | 2.0 | Sucrose | 1.0 | | |
| | Lecithin, ≥99% - | | Monoester of | | | |
| | Compass | | Palmitic Acid - | | | |
| | Foods Pte | | Compass | | | |
| | Ltd | | Foods Pte | | | |
| | | | Ltd | | | |
| 69 | Quillaja | 4.0 | | | | |
| | Saponaria | | | | | |
| | Extract - | | | | | |
| | Ingredion | | | | | |
| | Inc. | | | | | |
| 70 | Quillaja | 5.0 | | | | |
| | Saponaria | | | | | |
| | Extract - | | | | | |
| | Naturex | | | | | |
| 71 | Sunflower | 4.0 | Sucrose | 2.0 | | |
| | Lecithin, ≥99% - | | Monoester of | | | |
| | Compass | | Palmitic Acid - | | | |
| | Foods Pte | | Compass | | | |
| | Ltd | | Foods Pte | | | |
| | | | Ltd | | | |
| 72 | Sunflower | 4.0 | Sucrose | 2.0 | | |
| | Lecithin, ≥99% - | | Monoester of | | | |
| | Compass | | Palmitic Acid - | | | |
| | Foods Pte | | Compass | | | |
| | Ltd | | Foods Pte | | | |
| | | | Ltd | | | |
| 73 | Quillaja | 5.0 | | | | |
| | Saponaria | | | | | |
| | Extract - | | | | | |
| | Ingredion | | | | | |
| | Inc. | | | | | |
| 74 | Sunflower | 3.0 | | | | |
| | Lecithin, ≥99% - | | | | | |
| | Compass | | | | | |
| | Foods Pte | | | | | |
| | Ltd | | | | | |
| 75 | Quillaja | 4.0 | Sucrose | 0.7 | | |
| | Saponaria | | Monoester of | | | |
| | Extract - | | Palmitic Acid - | | | |
| | Naturex | | Compass | | | |
| | | | Foods Pte | | | |
| | | | Ltd | | | |
| 76 | Quillaja | 4.0 | Sucrose | 0.7 | | |
| | Saponaria | | Monoester of | | | |
| | Extract - | | Palmitic Acid - | | | |
| | Naturex | | Compass | | | |
| | | | Foods Pte | | | |
| | | | Ltd | | | |
| 77 | Sunflower | 0.5 | Enzyme | 0.5 | Sucrose | 0.5 |
| | Lecithin, ≥99% - | | Modified Soy | | Monoester of | |
| | Compass | | Lecithin - | | Palmitic Acid - | |
| | Foods Pte | | American | | Compass | |
| | Ltd | | Lecithin | | Foods Pte | |
| | | | Company | | Ltd | |
| 78 | Sunflower | 4.0 | Enzyme | 1.0 | Sucrose | 1.0 |
| | Lecithin, ≥99% - | | Modified Soy | | Monoester of | |
| | Compass | | Lecithin - | | Palmitic Acid - | |
| | Foods Pte | | American | | Compass | |
| | Ltd | | Lecithin | | Foods Pte | |
| | | | Company | | Ltd | |
| 79 | Sunflower | 4.0 | Enzyme | 1.0 | Sucrose | 1.0 |
| | Lecithin, ≥99% - | | Modified Soy | | Monoester of | |
| | | | Lecithin - | | Palmitic Acid - | |
| | Compass | | American | | Compass | |
| | Foods Pte | | Lecithin | | Foods Pte | |
| | Ltd | | Company | | Ltd | |
| 80 | Quillaja | 2.5 | Sucrose | 1.3 | | |
| | Saponaria | | Monoester of | | | |
| | Extract - | | Palmitic Acid - | | | |
| | Ingredion | | Compass | | | |
| | Inc. | | Foods Pte | | | |
| | | | Ltd | | | |
| 81 | Sunflower | 0.5 | Enzyme | 0.5 | Sucrose | 0.5 |
| | Lecithin, ≥99% - | | Modified Soy | | Monoester of | |
| | Compass | | Lecithin - | | Palmitic Acid - | |
| | Foods Pte | | American | | Compass | |
| | Ltd | | Lecithin | | Foods Pte | |
| | | | Company | | Ltd | |
| 82 | Quillaja | 6.0 | Sucrose | 1.0 | | |
| | Saponaria | | Monoester of | | | |
| | Extract - | | Palmitic Acid - | | | |
| | Naturex | | Compass | | | |
| | | | Foods Pte | | | |
| | | | Ltd | | | |
| 83 | Quillaja | 5.0 | Sucrose | 2.5 | | |
| | Saponaria | | Monoester of | | | |
| | Extract - | | Palmitic Acid - | | | |
| | Ingredion | | Compass | | | |
| | Inc. | | Foods Pte | | | |
| | | | Ltd | | | |
| 84 | Gum Acacia - | 6.0 | | | | |
| | Ingredion | | | | | |
| | Inc. | | | | | |
| 85 | Quillaja | 2.5 | Sunflower | 0.5 | | |
| | Saponaria | | Lecithin, ≥99% - | | | |
| | Extract - | | Compass | | | |
| | Naturex | | Foods Pte | | | |
| | | | Ltd | | | |
| 86 | Quillaja | 8.0 | Sucrose | 2.5 | | |
| | Saponaria | | Monoester of | | | |
| | Extract - | | Palmitic Acid - | | | |
| | Ingredion | | Compass | | | |
| | Inc. | | Foods Pte | | | |
| | | | Ltd | | | |
| 87 | Sunflower | 4.2 | Sucrose | 1.0 | | |
| | Lecithin, ≥99% - | | Monoester of | | | |
| | Compass | | Palmitic Acid - | | | |
| | Foods Pte | | Compass | | | |
| | Ltd | | Foods Pte | | | |
| | | | Ltd | | | |
| 88 | Sunflower | 4.2 | Sucrose | 1.0 | | |
| | Lecithin, ≥99% - | | Monoester of | | | |
| | Compass | | Palmitic Acid - | | | |
| | Foods Pte | | Compass | | | |
| | Ltd | | Foods Pte | | | |
| | | | Ltd | | | |
| 89 | Quillaja | 5.0 | Sucrose | 2.5 | | |
| | Saponaria | | Monoester of | | | |
| | Extract - | | Palmitic Acid - | | | |
| | Ingredion | | Compass | | | |
| | Inc. | | Foods Pte | | | |
| | | | Ltd | | | |
| 90 | Flavor-free | 5.0 | Modified | 5.0 | | |
| | Maltodextrin - | | Food Starch | | | |
| | Ingredion | | from Tapioca - | | | |
| | Inc. | | Ingredion | | | |
| | | | Inc. | | | |
| 91 | Modified | 10.0 | | | | |
| | Food Starch | | | | | |
| | from Tapioca - | | | | | |
| | Ingredion | | | | | |
| | Inc. | | | | | |
| 92 | Modified | 10.0 | | | | |
| | Food Starch | | | | | |
| | from Waxy | | | | | |
| | Maize - | | | | | |
| | Ingredion | | | | | |
| | Inc. | | | | | |
| 93 | Modified | 10.0 | | | | |
| | Food Starch | | | | | |
| | from Waxy | | | | | |
| | Maize - | | | | | |
| | Ingredion | | | | | |
| | Inc. | | | | | |
| 94 | Modified | 10.0 | | | | |
| | Food Starch | | | | | |
| | from Waxy | | | | | |
| | Maize - | | | | | |
| | Ingredion | | | | | |
| | Inc. | | | | | |
| 95 | Modified | 10.0 | | | | |
| | Food Starch | | | | | |
| | from Waxy | | | | | |
| | Maize - | | | | | |
| | Ingredion | | | | | |
| | Inc. | | | | | |
| 96 | Modified | 11.1 | | | | |
| | Food Starch | | | | | |
| | from Tapioca - | | | | | |
| | Ingredion | | | | | |
| | Inc. | | | | | |
| 97 | Modified | 12.2 | | | | |
| | Food Starch | | | | | |
| | from Tapioca - | | | | | |
| | Ingredion | | | | | |
| | Inc. | | | | | |
| 98 | Modified | 14.4 | | | | |
| | Food Starch | | | | | |
| | from Tapioca - | | | | | |
| | Ingredion | | | | | |
| | Inc. | | | | | |
| 99 | Modified | 7.5 | | | | |
| | Food Starch | | | | | |
| | from Tapioca - | | | | | |
| | Ingredion | | | | | |
| | Inc. | | | | | |
| 100 | Modified | 5.0 | | | | |
| | Food Starch | | | | | |
| | from Tapioca - | | | | | |
| | Ingredion | | | | | |
| | Inc. | | | | | |
| 101 | Fat-free | 1.0 | | | | |
| | Soybean | | | | | |
| | Lecithin with | | | | | |
| | 45% | | | | | |
| | phosphatidyl | | | | | |
| | choline - | | | | | |
| | Lipoid | | | | | |
| 102 | Sunflower | 2.0 | Quillaja | 1.0 | | |
| | Lecithin, ≥99% - | | Saponaria | | | |
| | Compass | | Extract - | | | |
| | Foods Pte | | Ingredion | | | |
| | Ltd | | Inc. | | | |
| 103 | Quillaja | 30.0 | | | | |
| | Saponaria | | | | | |
| | Extract - | | | | | |
| | Ingredion | | | | | |
| | Inc. | | | | | |
| 104 | Sunflower | 3.0 | Enzyme | 0.5 | Sucrose | 0.5 |
| | Lecithin, ≥99% - | | Modified Soy | | Monoester of | |
| | Compass | | Lecithin - | | Palmitic Acid - | |
| | Foods Pte | | American | | Compass | |
| | Ltd | | Lecithin | | Foods Pte | |
| | | | Company | | Ltd | |
| 105 | Sunflower | 2.0 | Enzyme | 0.5 | Sucrose | 0.5 |
| | Lecithin, ≥99% - | | Modified Soy | | Monoester of | |
| | Compass | | Lecithin - | | Palmitic Acid - | |
| | Foods Pte | | American | | Compass | |
| | Ltd | | Lecithin | | Foods Pte | |
| | | | Company | | Ltd | |
| 106 | Enzyme | 1.0 | Fatfree | 1.0 | Sucrose | 0.5 |
| | Modified Soy | | Sunflower | | Monoester of | |
| | Lecithin - | | Lecithin | | Palmitic Acid - | |
| | American | | (non-GMO) | | Compass | |
| | Lecithin | | with 60% | | Foods Pte | |
| | Company | | Phosphatidyl | | Ltd | |
| | | | choline - | | | |
| | | | Lipoid | | | |
| 107 | Enzyme | 1.0 | “Soy Lecithin | 1.0 | De-oiled | 1.0 |
| | Modified Soy | | (with | | Powdered | |
| | Lecithin - | | Ethoxylated | | Soy Lecithin - | |
| | American | | Mono- | | American | |
| | Lecithin | | diglycerides | | Lecithin | |
| | Company | | and | | Company | |
| | | | Propylene | | | |
| | | | Glycol) - | | | |
| | | | Americal | | | |
| | | | Lecithin | | | |
| | | | Company” | | | |
| 108 | Quillaja | 6.0 | | | | |
| | Saponaria | | | | | |
| | Extract - | | | | | |
| | Naturex | | | | | |
| 109 | Sucrose | 1.0 | | | | |
| | Ester of | | | | | |
| | Fatty Acids, | | | | | |
| | M182CD182 - | | | | | |
| | Compass | | | | | |
| | Foods Pte | | | | | |
| | Ltd | | | | | |
| 110 | Modified | 5.0 | | | | |
| | Gum Acacia - | | | | | |
| | Ingredion | | | | | |
| | Inc. | | | | | |
| 111 | Sunflower | 1.0 | Enzyme | 0.5 | Sucrose | 0.5 |
| | Palmitic Acid - | | Modified Soy | | Monoester of | |
| | Compass | | Lecithin - | | Palmitic Acid - | |
| | Compass | | American | | Compass | |
| | Foods Pte | | Lecithin | | Foods Pte | |
| | Ltd | | Company | | Ltd | |
| 112 | Quillaja | 4.0 | Sucrose | 4.0 | | |
| | Saponaria | | Monoester of | | | |
| | Extract - | | Palmitic Acid - | | | |
| | Ingredion | | Compass | | | |
| | Inc. | | Foods Pte | | | |
| | | | Ltd | | | |
| 113 | Soybean | 4.0 | | | | |
| | Phospholipids | | | | | |
| | with 70% | | | | | |
| | Phosphatidyl | | | | | |
| | choline - | | | | | |
| | Lipoid | | | | | |
| 114 | De-oiled | 2.0 | Sucrose | 2.0 | | |
| | Powdered | | Monoester of | | | |
| | Soy Lecithin - | | Palmitic Acid - | | | |
| | American | | Compass | | | |
| | Lecithin | | Foods Pte | | | |
| | Company | | Ltd | | | |
| 115 | Fat-free | 1.0 | Sucrose | 1.0 | | |
| | Soybean | | Monoester of | | | |
| | Lecithin with | | Palmitic Acid - | | | |
| | 45% | | Compass | | | |
| | phosphatidyl | | Foods Pte | | | |
| | choline - | | Ltd | | | |
| | Lipoid | | | | | |
| 116 | Sunflower | 2.0 | Sucrose | 1.0 | | |
| | Lecithin, ≥99% - | | Monoester of | | | |
| | Compass | | Palmitic Acid - | | | |
| | Foods Pte | | Compass | | | |
| | Ltd | | Foods Pte | | | |
| | | | Ltd | | | |
| 117 | Quillaja | 4.0 | Sucrose | 2.0 | | |
| | Saponaria | | Monoester of | | | |
| | Extract - | | Palmitic Acid - | | | |
| | Ingredion | | Compass | | | |
| | Inc. | | Foods Pte | | | |
| | | | Ltd | | | |
| 118 | Quillaja | 2.0 | Enzyme | 1.0 | Sucrose | 0.5 |
| | Saponaria | | Modified Soy | | Monoester of | |
| | Extract - | | Lecithin - | | Palmitic Acid - | |
| | Ingredion | | American | | Compass | |
| | Inc. | | Lecithin | | Foods Pte | |
| | | | Company | | Ltd | |
| 119 | Quillaja | 8.0 | Sucrose | 1.3 | | |
| | Saponaria | | Monoester of | | | |
| | Extract - | | Palmitic Acid - | | | |
| | Naturex | | Compass | | | |
| | | | Foods Pte | | | |
| | | | Ltd | | | |
| 120 | Quillaja | 2.5 | Sunflower | 0.5 | Sucrose | 0.1 |
| | Saponaria | | Lecithin, ≥99% - | | Monoester of | |
| | Extract - | | Compass | | Palmitic Acid - | |
| | Ingredion | | Foods Pte | | Compass | |
| | Inc. | | Ltd | | Foods Pte | |
| | | | | | Ltd | |
| 121 | Quillaja | 4.0 | Sucrose | 4.0 | | |
| | Saponaria | | Monoester of | | | |
| | Extract - | | Palmitic Acid - | | | |
| | Ingredion | | Compass | | | |
| | Inc. | | Foods Pte | | | |
| | | | Ltd | | | |
| 122 | Flavor-free | 5.0 | Modified | 5.0 | | |
| | Maltodextrin - | | Food Starch | | | |
| | Ingredion | | from Tapioca - | | | |
| | Inc. | | Ingredion | | | |
| | | | Inc. | | | |
| 123 | | | Modified | 10.0 | | |
| | | | Food Starch | | | |
| | | | from Waxy | | | |
| | | | Maize - | | | |
| | | | Ingredion | | | |
| | | | Inc. | | | |
| 124 | Quillaja | 8.0 | Sucrose | 4.0 | | |
| | Saponaria | | Monoester of | | | |
| | Extract - | | Palmitic Acid - | | | |
| | Ingredion | | Compass | | | |
| | Inc. | | Foods Pte | | | |
| | | | Ltd | | | |
| 125 | Sunflower | 3.0 | Sucrose | 1.0 | | |
| | Lecithin, ≥99% - | | Monoester of | | | |
| | Compass | | Palmitic Acid - | | | |
| | Foods Pte | | Compass | | | |
| | Ltd | | Foods Pte | | | |
| | | | Ltd | | | |
| 126 | | | Modified | 10.0 | | |
| | | | Food Starch | | | |
| | | | from Tapioca - | | | |
| | | | Ingredion | | | |
| | | | Inc. | | | |
| 127 | Flavor-free | 5.0 | Modified | 5.0 | | |
| | Maltodextrin - | | Food Starch | | | |
| | Ingredion | | from Tapioca - | | | |
| | Inc. | | Ingredion | | | |
| | | | Inc. | | | |
| 128 | Modified | 10.0 | | | | |
| | Food Starch | | | | | |
| | from Waxy | | | | | |
| | Maize - | | | | | |
| | Ingredion | | | | | |
| | Inc. | | | | | |
| 129 | Modified | 10.0 | | | | |
| | Food Starch | | | | | |
| | from Waxy | | | | | |
| | Maize - | | | | | |
| | Ingredion | | | | | |
| | Inc. | | | | | |
| 130 | Modified | 10.0 | | | | |
| | Food Starch | | | | | |
| | from Waxy | | | | | |
| | Maize - | | | | | |
| | Ingredion | | | | | |
| | Inc. | | | | | |
| 131 | Modified | 10.0 | | | | |
| | Food Starch | | | | | |
| | from Waxy | | | | | |
| | Maize - | | | | | |
| | Ingredion | | | | | |
| | Inc. | | | | | |
| 132 | Organic | 10.0 | | | | |
| | Gum Acacia - | | | | | |
| | Ingredion | | | | | |
| | Inc. | | | | | |
| 133 | Modified | 10.0 | | | | |
| | Gum Acacia - | | | | | |
| | Ingredion | | | | | |
| | Inc. | | | | | |
| 134 | Modified | 10.0 | | | | |
| | Food Starch | | | | | |
| | from Waxy | | | | | |
| | Maize - | | | | | |
| | Ingredion | | | | | |
| | Inc. | | | | | |
| 135 | Modified | 10.0 | | | | |
| | Food Starch | | | | | |
| | Refined from | | | | | |
| | Waxy Maize - | | | | | |
| | Ingredion | | | | | |
| | Inc. | | | | | |
| 136 | Quillaja | 50.0 | | | | |
| | Saponaria | | | | | |
| | Extract - | | | | | |
| | Ingredion | | | | | |
| | Inc. | | | | | |
| 137 | Modified | 4.5 | | | | |
| | Starch from | | | | | |
| | Waxy Maize - | | | | | |
| | Ingredion | | | | | |
| | Inc. | | | | | |
| 138 | Modified | 5.0 | Modified | 2.5 | | |
| | Food Starch | | Food Starch | | | |
| | from Tapioca - | | from Waxy | | | |
| | Ingredion | | Maize - | | | |
| | Inc. | | Ingredion | | | |
| | | | Inc. | | | |
| | | | Modified | 2.5 | | |
| | | | Food Starch | | | |
| | | | Refined from | | | |
| | | | Waxy Maize | | | |
| | | | - Ingredion | | | |
| | | | Inc. | | | |
| 139 | Modified | 10.0 | | | | |
| | Food Starch | | | | | |
| | from Tapioca - | | | | | |
| | Ingredion | | | | | |
| | Inc. | | | | | |
| 140 | Modified | 10.0 | | | | |
| | Food Starch | | | | | |
| | from Tapioca - | | | | | |
| | Ingredion | | | | | |
| | Inc. | | | | | |
| 141 | Modified | 7.5 | | | | |
| | Food Starch | | | | | |
| | from Tapioca - | | | | | |
| | Ingredion | | | | | |
| | Inc. | | | | | |
| 142 | Lysophos- | 1.0 | | | | |
| | phatidylcholine | | | | | |
| | from | | | | | |
| | soybean | | | | | |
| | lecithin (non- | | | | | |
| | GMO), 80% - | | | | | |
| | Lipoid | | | | | |
| 143 | Quillaja | 1.0 | | | | |
| | Saponaria | | | | | |
| | Extract - | | | | | |
| | Ingredion | | | | | |
| | Inc. | | | | | |
| 144 | Quillaja | 0.5 | Sucrose | 0.5 | | |
| | Saponaria | | Monoester of | | | |
| | Extract - | | Palmitic Acid - | | | |
| | Ingredion | | Compass | | | |
| | Inc. | | Foods Pte | | | |
| | | | Ltd | | | |
| 145 | Sucrose | 4.0 | | | | |
| | Monolaureate | | | | | |
| | L90 | | | | | |
| 146 | De-oiled | 0.5 | Quillaja | 0.5 | Sucrose | 0.5 |
| | Powdered | | Saponaria | | Monoester of | |
| | Soy Lecithin - | | Extract - | | Palmitic Acid - | |
| | American | | Ingredion | | Compass | |
| | Lecithin | | nc. | | Foods Pte | |
| | Company | | | | Ltd | |
| 147 | Sunflower | 0.5 | | | | |
| | Lecithin, ≥99% - | | | | | |
| | Compass | | | | | |
| | Foods Pte | | | | | |
| | Ltd | | | | | |
| 148 | De-oiled | 0.2 | | | | |
| | Powdered | | | | | |
| | Soy Lecithin - | | | | | |
| | American | | | | | |
| | Lecithin | | | | | |
| | Company | | | | | |
| 149 | De-oiled | 0.2 | | | | |
| | Powdered | | | | | |
| | Soy Lecithin - | | | | | |
| | American | | | | | |
| | Lecithin | | | | | |
| | Company | | | | | |
| 150 | De-oiled | 0.3 | | | | |
| | Powdered | | | | | |
| | Soy Lecithin - | | | | | |
| | American | | | | | |
| | Lecithin | | | | | |
| | Company | | | | | |
| 151 | De-oiled | 0.7 | | | | |
| | Powdered | | | | | |
| | Soy Lecithin - | | | | | |
| | American | | | | | |
| | Lecithin | | | | | |
| | Company | | | | | |
| 152 | Quillaja | 1.0 | | | | |
| | Saponaria | | | | | |
| | Extract - | | | | | |
| | Ingredion | | | | | |
| | Inc. | | | | | |
| 153 | Lysophos- | 0.2 | | | | |
| | phatidylcholine | | | | | |
| | from | | | | | |
| | soybean | | | | | |
| | lecithin (non- | | | | | |
| | GMO), 80% - | | | | | |
| | Lipoid | | | | | |
| 154 | Phosphatidyl | 0.5 | | | | |
| | choline from | | | | | |
| | soybean, | | | | | |
| | D,L-α- | | | | | |
| | tocopherol | | | | | |
| | (n.m.t. 0.3%) - | | | | | |
| | Lipoid | | | | | |
| 155 | Fat-free | 3.0 | | | | |
| | Soybean | | | | | |
| | Lecithin with | | | | | |
| | 45% | | | | | |
| | phosphatidyl | | | | | |
| | choline - | | | | | |
| | Lipoid | | | | | |
| 156 | Lecithin with | 4.0 | | | | |
| | Ethoxylated | | | | | |
| | mono di- | | | | | |
| | glycerides - | | | | | |
| | Solae | | | | | |
| 157 | Lecithin with | 6.0 | | | | |
| | Ethoxylated | | | | | |
| | mono di- | | | | | |
| | glycerides - | | | | | |
| | Solae | | | | | |
| 158 | De-oiled | 2.0 | | | | |
| | Powdered | | | | | |
| | Soy Lecithin - | | | | | |
| | American | | | | | |
| | Lecithin | | | | | |
| | Company | | | | | |
| 159 | Sucrose | 1.5 | | | | |
| | Monoester of | | | | | |
| | Palmitic Acid - | | | | | |
| | Compass | | | | | |
| | Foods Pte | | | | | |
| | Ltd | | | | | |
| 160 | Sunflower | 2.5 | Sucrose | 1.5 | | |
| | Lecithin, ≥99% - | | Monoester of | | | |
| | Compass | | Palmitic Acid - | | | |
| | Foods Pte | | Compass | | | |
| | Ltd | | Foods Pte | | | |
| | | | Ltd | | | |
| 161 | Quillaja | 2.5 | Sunflower | 0.5 | | |
| | Saponaria | | Lecithin, ≥99% - | | | |
| | Extract - | | Compass | | | |
| | Ingredion | | Foods Pte | | | |
| | Inc. | | Ltd | | | |
| 162 | Quillaja | 5.0 | Sucrose | 2.5 | | |
| | Saponaria | | Monoester of | | | |
| | Extract - | | Palmitic Acid - | | | |
| | Ingredion | | Compass | | | |
| | Inc. | | Foods Pte | | | |
| | | | Ltd | | | |
| 163 | Quillaja | 2.5 | Sunflower | 0.5 | | |
| | Saponaria | | Lecithin, ≥99% - | | | |
| | Extract - | | Compass | | | |
| | Ingredion | | Foods Pte | | | |
| | Inc. | | Ltd | | | |
| 164 | Modified | 10.0 | | | | |
| | Food Starch | | | | | |
| | from Waxy | | | | | |
| | Maize - | | | | | |
| | Ingredion | | | | | |
| | Inc. | | | | | |
| 165 | Modified | 10.0 | | | | |
| | Food Starch | | | | | |
| | from Waxy | | | | | |
| | Maize - | | | | | |
| | Ingredion | | | | | |
| | Inc. | | | | | |
| 166 | Modified | 10.0 | | | | |
| | Food Starch | | | | | |
| | from Waxy | | | | | |
| | Maize - | | | | | |
| | Ingredion | | | | | |
| | Inc. | | | | | |
| 167 | Modified | 10.0 | | | | |
| | Food Starch | | | | | |
| | from Waxy | | | | | |
| | Maize - | | | | | |
| | Ingredion | | | | | |
| | Inc. | | | | | |
| 168 | Modified | 10.0 | | | | |
| | Food Starch | | | | | |
| | from Waxy | | | | | |
| | Maize - | | | | | |
| | Ingredion | | | | | |
| | Inc. | | | | | |
| 169 | Modified | 10.0 | | | | |
| | Food Starch | | | | | |
| | from Tapioca - | | | | | |
| | Ingredion | | | | | |
| | Inc. | | | | | |
| 170 | Organic | 10.0 | | | | |
| | Gum Acacia - | | | | | |
| | Ingredion | | | | | |
| | Inc. | | | | | |
| 171 | Modified | 10.0 | | | | |
| | Gum Acacia - | | | | | |
| | Ingredion | | | | | |
| | Inc. | | | | | |
| 172 | Modified | 10.0 | | | | |
| | Food Starch | | | | | |
| | from Waxy | | | | | |
| | Maize - | | | | | |
| | Ingredion | | | | | |
| | Inc. | | | | | |
| 173 | Modified | 10.0 | | | | |
| | Food Starch | | | | | |
| | Refined from | | | | | |
| | Waxy Maize - | | | | | |
| | Ingredion | | | | | |
| | Inc. | | | | | |
| 174 | Quillaja | 50.0 | | | | |
| | Saponaria | | | | | |
| | Extract - | | | | | |
| | Ingredion | | | | | |
| | Inc. | | | | | |
| 175 | Modified | 10.0 | | | | |
| | Food Starch | | | | | |
| | from Tapioca - | | | | | |
| | Ingredion | | | | | |
| | Inc. | | | | | |
| 176 | Modified | 10.0 | | | | |
| | Food Starch | | | | | |
| | from Tapioca - | | | | | |
| | Ingredion | | | | | |
| | Inc. | | | | | |
| 177 | Modified | 10.0 | | | | |
| | Food Starch | | | | | |
| | from Tapioca - | | | | | |
| | Ingredion | | | | | |
| | Inc. | | | | | |
| 178 | Sunflower | 2.0 | Sucrose | 1.0 | | |
| | Lecithin, ≥99% - | | Monoester of | | | |
| | Compass | | Palmitic Acid - | | | |
| | Foods Pte | | Compass | | | |
| | Ltd | | Foods Pte | | | |
| | | | Ltd | | | |
| 179 | Quillaja | 0.5 | Sunflower | 0.1 | | |
| | Saponaria | | Lecithin, ≥99% - | | | |
| | Extract - | | Compass | | | |
| | Ingredion | | Foods Pte | | | |
| | Inc. | | Ltd | | | |
| 180 | Sunflower | 2.0 | Sucrose | 1.0 | | |
| | Lecithin, ≥99% - | | Monoester of | | | |
| | Compass | | Palmitic Acid - | | | |
| | Foods Pte | | Compass | | | |
| | Ltd | | Foods Pte | | | |
| | | | Ltd | | | |
| 181 | Sunflower | 2.0 | Sucrose | 1.0 | | |
| | Lecithin, ≥99% - | | Monoester of | | | |
| | Compass | | Palmitic Acid - | | | |
| | Foods Pte | | Compass | | | |
| | Ltd | | Foods Pte | | | |
| | | | Ltd | | | |
| 182 | Sunflower | 4.2 | | | | |
| | Lecithin, ≥99% - | | | | | |
| | Compass | | | | | |
| | Foods Pte | | | | | |
| | Ltd | | | | | |
| 183 | Sunflower | 4.2 | | | | |
| | Lecithin, ≥99% - | | | | | |
| | Compass | | | | | |
| | Foods Pte | | | | | |
| | Ltd | | | | | |
| 184 | Flavor-free | 5.0 | Modified | 5.0 | | |
| | Maltodextrin - | | Food Starch | | | |
| | Ingredion | | from Tapioca - | | | |
| | Inc. | | Ingredion | | | |
| | | | Inc. | | | |
| 185 | Flavor-free | 5.0 | Modified | 5.0 | | |
| | Maltodextrin - | | Food Starch | | | |
| | Ingredion | | from Tapioca - | | | |
| | Inc. | | Ingredion | | | |
| | | | Inc. | | | |
| 186 | | | Modified | 10.0 | | |
| | | | Food Starch | | | |
| | | | from Tapioca - | | | |
| | | | Ingredion | | | |
| | | | Inc. | | | |
| 187 | Flavor-free | 5.0 | Modified | 5.0 | | |
| | Maltodextrin - | | Food Starch | | | |
| | Ingredion | | from Tapioca - | | | |
| | Inc. | | Ingredion | | | |
| | | | Inc. | | | |
| 188 | Flavor-free | 5.0 | Modified | 10.0 | | |
| | Maltodextrin - | | Food Starch | | | |
| | Ingredion | | from Tapioca - | | | |
| | Inc. | | Ingredion | | | |
| | | | Inc. | | | |
| 189 | | | Modified | 10.0 | | |
| | | | Food Starch | | | |
| | | | from Tapioca - | | | |
| | | | Ingredion | | | |
| | | | Inc. | | | |
| 190 | Quillaja | 8.0 | Sucrose | 4.0 | | |
| | Saponaria | | Monoester of | | | |
| | Extract - | | Palmitic Acid - | | | |
| | Ingredion | | Compass | | | |
| | Inc. | | Foods Pte | | | |
| | | | Ltd | | | |
| 191 | Quillaja | 6.0 | Sucrose | 1.0 | | |
| | Saponaria | | Monoester of | | | |
| | Extract - | | Palmitic Acid - | | | |
| | Naturex | | Compass | | | |
| | | | Foods Pte | | | |
| | | | Ltd | | | |
| 192 | Quillaja | 6.0 | Sucrose | 1.0 | | |
| | Saponaria | | Monoester of | | | |
| | Extract - | | Palmitic Acid - | | | |
| | Naturex | | Compass | | | |
| | | | Foods Pte | | | |
| | | | Ltd | | | |
| 193 | Quillaja | 6.0 | Sucrose | 1.0 | | |
| | Saponaria | | Monoester of | | | |
| | Extract - | | Palmitic Acid - | | | |
| | Naturex | | Compass | | | |
| | | | Foods Pte | | | |
| | | | Ltd | | | |
| 194 | Quillaja | 6.0 | Sunflower | 3.0 | | |
| | Saponaria | | Lecithin, ≥99% - | | | |
| | Extract - | | Compass | | | |
| | Naturex | | Foods Pte | | | |
| | | | Ltd | | | |
| 195 | Flavor-free | 5.0 | Modified | 5.0 | | |
| | Maltodextrin - | | Food Starch | | | |
| | Ingredion | | from Tapioca - | | | |
| | Inc. | | Ingredion | | | |
| | | | Inc. | | | |
| 196 | Quillaja | 2.8 | Sucrose | 1.4 | | |
| | Saponaria | | Monoester of | | | |
| | Extract - | | Palmitic Acid - | | | |
| | Ingredion | | Compass | | | |
| | Inc. | | Foods Pte | | | |
| | | | Ltd | | | |
| 197 | Modified | 10.0 | | | | |
| | Food Starch | | | | | |
| | from Waxy | | | | | |
| | Maize - | | | | | |
| | Ingredion | | | | | |
| | Inc. | | | | | |
| 198 | Modified | 10.0 | | | | |
| | Food Starch | | | | | |
| | from Waxy | | | | | |
| | Maize - | | | | | |
| | Ingredion | | | | | |
| | Inc. | | | | | |
| 199 | Modified | 12.5 | | | | |
| | Food Starch | | | | | |
| | from Waxy | | | | | |
| | Maize - | | | | | |
| | Ingredion | | | | | |
| | Inc. | | | | | |
| 200 | Modified | 7.5 | | | | |
| | Food Starch | | | | | |
| | from Waxy | | | | | |
| | Maize - | | | | | |
| | Ingredion | | | | | |
| | Inc. | | | | | |
| 201 | Modified | 10.0 | Flavor-free | 5.0 | | |
| | Food Starch | | Maltodextrin - | | | |
| | from Waxy | | Ingredion | | | |
| | Maize - | | Inc. | | | |
| | Ingredion | | | | | |
| | Inc. | | | | | |
| 202 | Modified | 15.0 | | | | |
| | Food Starch | | | | | |
| | from Waxy | | | | | |
| | Maize - | | | | | |
| | Ingredion | | | | | |
| | Inc. | | | | | |
| 203 | Modified | 15.0 | | | | |
| | Food Starch | | | | | |
| | from Waxy | | | | | |
| | Maize - | | | | | |
| | Ingredion | | | | | |
| | Inc. | | | | | |
| 204 | Modified | 10.0 | | | | |
| | Food Starch | | | | | |
| | from Waxy | | | | | |
| | Maize - | | | | | |
| | Ingredion | | | | | |
| | Inc. | | | | | |
| 205 | Modified | 10.0 | | | | |
| | Food Starch | | | | | |
| | from Tapioca - | | | | | |
| | Ingredion | | | | | |
| | Inc. | | | | | |
| 206 | Modified | 10.0 | | | | |
| | Food Starch | | | | | |
| | from Tapioca - | | | | | |
| | Ingredion | | | | | |
| | Inc. | | | | | |
| 207 | Modified | 10.0 | | | | |
| | Food Starch | | | | | |
| | from Tapioca - | | | | | |
| | Ingredion | | | | | |
| | Inc. | | | | | |
| 208 | Modified | 24.0 | | | | |
| | Food Starch | | | | | |
| | from Waxy | | | | | |
| | Maize - | | | | | |
| | Ingredion | | | | | |
| | Inc. | | | | | |
| 209 | Quillaja | 50.0 | | | | |
| | Saponaria | | | | | |
| | Extract - | | | | | |
| | Ingredion | | | | | |
| | Inc. | | | | | |
| 210 | Modified | 10.0 | | | | |
| | Food Starch | | | | | |
| | from Tapioca - | | | | | |
| | Ingredion | | | | | |
| | Inc. | | | | | |
| 211 | Modified | 5.0 | | | | |
| | Food Starch | | | | | |
| | from Tapioca - | | | | | |
| | Ingredion | | | | | |
| | Inc. | | | | | |

TABLE S005C

| | | Usage | | Usage | | Usage |
|---|---|---|---|---|---|---|
| | | rate | | rate | Continuous | rate |
| Example | Encapsulant | (% w/w) | Co-Solvent | (% w/w) | Phase | (% w/w) |
| 64 | | | | | Deionized Water | 97.5 |
| 65 | | | | | Glycerin | 94.5 |
| 66 | | | | | Glycerin | 94.5 |
| 67 | | | | | Glycerin | 94.5 |
| 68 | | | | | Glycerin | 94.5 |
| 69 | | | | | Glycerin | 93.0 |
| 70 | | | | | Deionized Water | 93.0 |
| 71 | | | | | Glycerin | 88.3 |
| 72 | | | | | Glycerin | 90.5 |
| 73 | | | | | Deionized Water | 92.0 |
| 74 | | | | | Glycerin | 92.0 |
| 75 | | | | | Glycerin | 89.3 |
| 76 | | | | | Glycerin | 89.3 |
| 77 | | | | | Glycerin | 93.5 |
| 78 | | | | | Glycerin | 88.9 |
| 79 | | | | | Glycerin | 89.0 |
| 80 | | | | | Glycerin | 86.3 |
| 81 | | | | | Glycerin | 91.5 |
| 82 | | | | | Glycerin | 82.9 |
| 83 | | | | | Glycerin | 77.5 |
| 84 | | | | | Deionized Water | 82.0 |
| 85 | | | | | Glycerin | 82.0 |
| 86 | | | | | Glycerin | 58.8 |
| 87 | | | | | Glycerin | 73.7 |
| 88 | | | | | Glycerin | 73.7 |
| 89 | | | | | Glycerin | 75.5 |
| 90 | | | Distilled Water | 15.0 | Glycerin | 55.0 |
| 91 | | | Distilled Water | 15.0 | Glycerin | 55.0 |
| 92 | | | Distilled Water | 15.0 | Glycerin | 50.0 |
| 93 | | | Distilled Water | 15.0 | Glycerin | 45.0 |
| 94 | | | Distilled Water | 15.0 | Glycerin | 40.0 |
| 95 | | | Distilled Water | 15.0 | Glycerin | 35.0 |
| 96 | Flavor-free | 5.5 | | | Distilled Water | 50.0 |
| | Maltodextrin - | | | | | |
| | Ingredion Inc. | | | | | |
| 97 | Flavor-free | 6.0 | | | Distilled Water | 45.0 |
| | Maltodextrin - | | | | | |
| | Ingredion Inc. | | | | | |
| 98 | Flavor-free | 7.2 | | | Distilled Water | 35.0 |
| | Maltodextrin - | | | | | |
| | Ingredion Inc. | | | | | |
| 99 | Flavor-free | 7.5 | | | Distilled Water | 40.0 |
| | Maltodextrin - | | | | | |
| | Ingredion Inc. | | | | | |
| 100 | Flavor-free | 10.0 | | | Distilled Water | 40.0 |
| | Maltodextrin - | | | | | |
| | Ingredion Inc. | | | | | |
| 101 | | | | | Glycerin | 97.0 |
| 102 | | | | | Glycerin | 94.5 |
| 103 | | | | | Glycerin | 68.0 |
| 104 | | | | | Glycerin | 94.0 |
| 105 | | | | | Glycerin | 95.0 |
| 106 | | | | | Glycerin | 95.5 |
| 107 | | | | | Glycerin | 96.0 |
| 108 | | | | | Glycerin | 92.0 |
| 109 | | | | | Glycerin | 97.0 |
| 110 | | | | | Distilled Water | 93.0 |
| 111 | | | | | Glycerin | 94.0 |
| 112 | | | | | Glycerin | 84.0 |
| 113 | | | | | Glycerin | 90.0 |
| 114 | | | | | Glycerin | 90.0 |
| 115 | | | | | Glycerin | 92.0 |
| 116 | | | Distilled Water | 6.5 | Glycerin | 80.5 |
| 117 | | | | | Glycerin | 86.0 |
| 118 | | | | | Glycerin | 89.5 |
| 119 | | | | | Glycerin | 79.3 |
| 120 | | | | | Glycerin | 84.4 |
| 121 | | | | | Glycerin | 77.0 |
| 122 | | | Distilled Water | 15.0 | Glycerin | 55.0 |
| 123 | | | Distilled Water | 15.0 | Glycerin | 50.0 |
| 124 | | | | | Glycerin | 48.6 |
| 125 | | | | | Glycerin | 75.0 |
| 126 | | | Distilled Water | 15.0 | Glycerin | 55.0 |
| 127 | | | Distilled Water | 15.0 | Glycerin | 55.0 |
| 128 | | | Distilled Water | 15.0 | Glycerin | 50.0 |
| 129 | | | Distilled Water | 15.0 | Glycerin | 45.0 |
| 130 | | | Distilled Water | 15.0 | Glycerin | 40.0 |
| 131 | | | Distilled Water | 15.0 | Glycerin | 35.0 |
| 132 | Flavor-free | 10.0 | | | Distilled Water | 55.0 |
| | Maltodextrin - | | | | | |
| | Ingredion Inc. | | | | | |
| 133 | Flavor-free | 10.0 | | | Distilled Water | 55.0 |
| | Maltodextrin - | | | | | |
| | Ingredion Inc. | | | | | |
| 134 | Flavor-free | 5.0 | | | Distilled Water | 55.0 |
| | Maltodextrin - | | | | | |
| | Ingredion Inc. | | | | | |
| 135 | Flavor-free | 5.0 | | | Distilled Water | 55.0 |
| | Maltodextrin - | | | | | |
| | Ingredion Inc. | | | | | |
| 136 | Inulin, water | 5.0 | | | Distilled Water | 15.0 |
| | soluble fiber - | | | | | |
| | It's Just | | | | | |
| 137 | Flavor-free | 4.5 | | | Distilled Water | 55.0 |
| | Maltodextrin - | | | | | |
| | Ingredion Inc. | | | | | |
| 138 | Flavor-free | 10.0 | | | Distilled Water | 40.0 |
| | Maltodextrin - | | | | | |
| | Ingredion Inc. | | | | | |
| 139 | Emulsifying | 10.0 | | | Distilled Water | 40.0 |
| | Starch - | | | | | |
| | Cargill Inc. | | | | | |
| 140 | Inulin, water | 10.0 | | | Distilled Water | 40.0 |
| | soluble fiber - | | | | | |
| | It's Just | | | | | |
| 141 | Flavor-free | 7.5 | | | Distilled Water | 40.0 |
| | Maltodextrin - | | | | | |
| | Ingredion Inc. | | | | | |
| 142 | | | | | Glycerin | 97.0 |
| 143 | | | | | Glycerin | 97.0 |
| 144 | | | | | Glycerin | 97.0 |
| 145 | | | | | Glycerin | 94.0 |
| 146 | | | | | Glycerin | 96.5 |
| 147 | | | | | Glycerin | 97.5 |
| 148 | | | | | Glycerin | 98.5 |
| 149 | | | | | Glycerin | 98.5 |
| 150 | | | | | Glycerin | 97.7 |
| 151 | | | | | Glycerin | 98.0 |
| 152 | | | | | Glycerin | 97.0 |
| 153 | | | | | Glycerin | 97.8 |
| 154 | | | | | Glycerin | 97.5 |
| 155 | | | | | Glycerin | 93.0 |
| 156 | | | | | Glycerin | 92.0 |
| 157 | | | | | Glycerin | 88.0 |
| 158 | | | | | Glycerin | 94.0 |
| 159 | | | | | Glycerin | 92.5 |
| 160 | | | | | Glycerin | 82.0 |
| 161 | | | | | Glycerin | 82.0 |
| 162 | | | | | Glycerin | 75.5 |
| 163 | | | | | Glycerin | 82.0 |
| 164 | | | Distilled Water | 15.0 | Glycerin | 55.0 |
| 165 | | | Distilled Water | 15.0 | Glycerin | 50.0 |
| 166 | | | Distilled Water | 15.0 | Glycerin | 45.0 |
| 167 | | | Distilled Water | 15.0 | Glycerin | 40.0 |
| 168 | | | Distilled Water | 15.0 | Glycerin | 35.0 |
| 169 | Flavor-free | 10.0 | | | Distilled Water | 55.0 |
| | Maltodextrin - | | | | | |
| | Ingredion Inc. | | | | | |
| 170 | Flavor-free | 10.0 | | | Distilled Water | 55.0 |
| | Maltodextrin - | | | | | |
| | Ingredion Inc. | | | | | |
| 171 | Flavor-free | 10.0 | | | Distilled Water | 55.0 |
| | Maltodextrin - | | | | | |
| | Ingredion Inc. | | | | | |
| 172 | Flavor-free | 5.0 | | | Distilled Water | 55.0 |
| | Maltodextrin - | | | | | |
| | Ingredion Inc. | | | | | |
| 173 | Flavor-free | 5.0 | | | Distilled Water | 55.0 |
| | Maltodextrin - | | | | | |
| | Ingredion Inc. | | | | | |
| 174 | Inulin, water | 5.0 | | | Distilled Water | 15.0 |
| | soluble fiber - | | | | | |
| | It's Just | | | | | |
| 175 | Flavor-free | 10.0 | | | Distilled Water | 40.0 |
| | Maltodextrin - | | | | | |
| | Ingredion Inc. | | | | | |
| 176 | Emulsifying | 10.0 | | | Distilled Water | 40.0 |
| | Starch - | | | | | |
| | Cargill Inc. | | | | | |
| 177 | Inulin, water | 10.0 | | | Distilled Water | 40.0 |
| | soluble fiber - | | | | | |
| | It's Just | | | | | |
| 178 | | | | | Glycerin | 94.5 |
| 179 | | | | | Glycerin | 96.7 |
| 180 | | | | | Glycerin | 94.5 |
| 181 | | | | | Glycerin | 94.5 |
| 182 | | | | | Glycerin | 85.1 |
| 183 | | | | | Glycerin | 86.3 |
| 184 | | | Distilled Water | 15.0 | Glycerin | 60.0 |
| 185 | | | Distilled Water | 15.0 | Glycerin | 55.0 |
| 186 | | | Distilled Water | 15.0 | Glycerin | 55.0 |
| 187 | | | Distilled Water | 15.0 | Glycerin | 55.0 |
| 188 | | | Distilled Water | 15.0 | Glycerin | 50.0 |
| 189 | | | Distilled Water | 15.0 | Glycerin | 50.0 |
| 190 | | | | | Glycerin | 48.8 |
| 191 | | | | | Glycerin | 84.5 |
| 192 | | | | | Glycerin | 84.5 |
| 193 | | | | | Glycerin | 84.5 |
| 194 | | | | | Glycerin | 83.0 |
| 195 | | | Distilled Water | 15.0 | Glycerin | 55.0 |
| 196 | | | | | Glycerin | 85.1 |
| 197 | | | Distilled Water | 15.0 | Glycerin | 50.0 |
| 198 | | | Distilled Water | 15.0 | Glycerin | 50.0 |
| 199 | | | Distilled Water | 15.0 | Glycerin | 47.5 |
| 200 | | | Distilled Water | 15.0 | Glycerin | 52.5 |
| 201 | | | Distilled Water | 15.0 | Glycerin | 45.0 |
| 202 | | | Distilled Water | 15.0 | Glycerin | 45.0 |
| 203 | | | Distilled Water | 15.0 | Glycerin | 45.0 |
| 204 | Flavor-free | 5.0 | | | Distilled Water | 55.0 |
| | Maltodextrin - | | | | | |
| | Ingredion Inc. | | | | | |
| 205 | Maltodextrin, | 25.0 | | | Distilled Water | 55.0 |
| | 90% Dietary | | | | | |
| | Fiber - | | | | | |
| | Fibersol | | | | | |
| 206 | Flavor-free | 10.0 | | | Distilled Water | 40.0 |
| | Maltodextrin - | | | | | |
| | Ingredion Inc. | | | | | |
| 207 | Emulsifying | 10.0 | | | Distilled Water | 40.0 |
| | Starch - | | | | | |
| | Cargill Inc. | | | | | |
| 208 | | | | | Distilled Water | 60.0 |
| 209 | Inulin, water | 5.0 | | | Distilled Water | 15.0 |
| | soluble fiber - | | | | | |
| | It's Just | | | | | |
| 210 | Flavor-free | 10.0 | | | Distilled Water | 45.0 |
| | Maltodextrin - | | | | | |
| | Ingredion Inc. | | | | | |
| 211 | Flavor-free | 10.0 | | | Distilled Water | 48.0 |
| | Maltodextrin - | | | | | |
| | Ingredion Inc. | | | | | |

### Examples 212-263

Tables S006A and S006B below show examples of using different carrier oils and carrier oil mixtures. These show that systems of the disclosure are robust, allowing for a variety of different carrier oils. These formulations are not limited to the carrier oils listed herein and can be prepared with other carrier oils and blends thereof as listed above at paragraph [0040]. These examples also relate to disclosures discussed above at least at paragraphs [0038], [0039], [0040], [0041], and [0042].

TABLE S006A
the Hydrophobic Phase compositions, showing various carrier oils.

| | | | Usage | | Usage | Flavorant or | Usage |
|---|---|---|---|---|---|---|---|
| Example | Prototype ID | ABI | Rate | Carrier Oil | Rate | Bittermasker | Rate |
| 212 | 25G5-CO1 | CBD Isolate | 1.0 | Refined | 1.5 | | |
| | | | | Sesame | | | |
| | | | | Oil (IV-1) | | | |
| 213 | 25G5-CO2 | CBD Isolate | 1.0 | Refined | 1.5 | | |
| | | | | Olive Oil | | | |
| | | | | (IV) | | | |
| 214 | 20E5 | Broad | 1.0 | Refined | 1.0 | | |
| | | Spectrum | | Olive Oil | | | |
| | | Distillate | | | | | |
| 215 | 20E6 | Broad | 1.0 | Refined | 1.0 | | |
| | | Spectrum | | Olive Oil | | | |
| | | Distillate | | | | | |
| 216 | 43C | Broad | 1.0 | Refined | 1.0 | | |
| | | Spectrum | | Olive Oil | | | |
| | | Distillate | | | | | |
| 217 | 25M | Broad | 1.0 | Refined | 1.0 | | |
| | | Spectrum | | Olive Oil | | | |
| | | Distillate | | | | | |
| 218 | 34C | Broad | 1.0 | Refined | 1.0 | | |
| | | Spectrum | | Olive Oil | | | |
| | | Distillate | | | | | |
| 219 | 35D | Broad | 1.0 | Refined | 1.0 | | |
| | | Spectrum | | Olive Oil | | | |
| | | Distillate | | | | | |
| 220 | 11B | Broad | 3.0 | Refined | 3.0 | | |
| | | Spectrum | | Olive Oil | | | |
| | | Distillate | | | | | |
| 221 | 25G3 | Broad | 2.0 | Refined | 2.0 | | |
| | | Spectrum | | Olive Oil | | | |
| | | Distillate | | | | | |
| 222 | 44D | Broad | 1.0 | Refined | 1.0 | | |
| | | Spectrum | | Olive Oil | | | |
| | | Distillate | | | | | |
| 223 | 3A7D7G1 | Full Spectrum | 6.0 | Olive Oil | 19.0 | Bitterness | 1.0 |
| | | Distillate | | | | Blocker, | |
| | | CBN Isolate | 7.0 | | | Natural, Type | |
| | | CBG isolate | 7.0 | | | A - Allen | |
| | | | | | | Flavors Inc. | |
| 224 | 3A7D7G | Broad | 4.0 | Refined | 11.5 | Bitterness | 1.5 |
| | | Spectrum | | Olive Oil | | Blocker, | |
| | | Distillate | | | | Natural, Type | |
| | | CBG distillate | 7.0 | | | C - Allen | |
| | | | | | | Flavors Inc. | |
| 225 | 25G5-CO3 | CBD Isolate | 1.0 | Refined | 1.5 | | |
| | | | | Soybean | | | |
| | | | | Oil (IV) | | | |
| 226 | 25G5-CO4 | CBD Isolate | 1.0 | Peceol | 1.5 | | |
| | | | | (3088) | | | |
| 227 | 9B | CBD Isolate | 1.0 | Glyceril | 2.0 | | |
| | | | | Monooleate | | | |
| | | | | (Type 40) - | | | |
| | | | | Gattefossé | | | |
| | | | | SAS | | | |
| 228 | 21E | Full Spectrum | 2.0 | Glyceril | 2.0 | | |
| | | Distillate | | Monooleate | | | |
| | | | | (Type 40) - | | | |
| | | | | Gattefossé | | | |
| | | | | SAS | | | |
| 229 | 25G5-CO11 | CBD Isolate | 1.0 | Grape | 1.5 | | |
| | | | | Seed Oil | | | |
| | | | | (I-003-bna- | | | |
| | | | | 10) | | | |
| 230 | 43 | Broad | 1.0 | Grape | 1.0 | | |
| | | Spectrum | | seed oil | | | |
| | | Distillate | | | | | |
| 231 | 21F | Full Spectrum | 2.0 | Grape | 2.0 | | |
| | | Distillate | | seed oil | | | |
| 232 | 25G5-CO13 | CBD Isolate | 1.0 | Triacetin | 1.5 | | |
| 233 | 25G5-CO14 | CBD Isolate | 1.0 | Canola Oil | 1.5 | | |
| 234 | 20H | Broad | 1.0 | Canola Oil | 1.0 | | |
| | | Spectrum | | | | | |
| | | Distillate | | | | | |
| 235 | 25G5F | CBD Isolate | 2.0 | Hemp | 1.5 | | |
| | | | | Seed Oil | | | |
| 236 | 25G5-CO12 | CBD Isolate | 1.0 | Hemp | 1.5 | | |
| | | | | Seed Oil | | | |
| | | | | (bna-170-r) | | | |
| 237 | 21G | Full Spectrum | 2.0 | Hemp | 2.0 | | |
| | | Distillate | | seed oil | | | |
| 238 | 25G5C2-4 | Full Spectrum | 4.0 | Hemp | 10.0 | | |
| | | Distillate | | Seed Oil | | | |
| 239 | 3A7D7F | Broad | 5.0 | Hemp | 18.8 | Lemon Oil | 1.2 |
| | | Spectrum | | Seed oil | | Flavor - | |
| | | Distillate | | | | Flavor | |
| | | CBN Isolate | 5.0 | | | Insights | |
| 240 | 21D | Full Spectrum | 2.0 | Maisine | 2.0 | | |
| | | Distillate | | CC | | | |
| 241 | 59 | Broad | 1.0 | MCT | 1.0 | | |
| | | Spectrum | | | | | |
| | | Distillate | | | | | |
| 242 | 33A | Broad | 1.0 | MCT | 1.0 | | |
| | | Spectrum | | | | | |
| | | Distillate | | | | | |
| 243 | 9A | Full Spectrum | 1.0 | MCT | 1.0 | | |
| | | Distillate | | | | | |
| 244 | 29A | Full Spectrum | 1.0 | MCT | 1.0 | | |
| | | Distillate | | | | | |
| 245 | 25G5-CBG | CBG Isolate | 1.0 | MCT | 1.5 | | |
| | Isolate | | | | | | |
| 246 | WTAM-1C- | CBD Isolate | 3.0 | MCT | 3.0 | | |
| | 17000 | | | | | | |
| 247 | 56F3H2 | CBD Isolate | 2.0 | MCT | 5.0 | Bitterness | 0.5 |
| | | CBN Isolate | 1.0 | | | Blocker, | |
| | | | | | | Natural, Type | |
| | | | | | | A - Allen | |
| | | | | | | Flavors Inc. | |
| 248 | 56H | CBD Isolate | 7.5 | MCT | 7.5 | | |
| 249 | 10A2C5A1E2 | CBD Isolate | 2.3 | MCT | 7.8 | Bitterness | 0.1 |
| | | CBG Distillate | 0.2 | | | Blocker, | |
| | | CBN Isolate | 0.2 | | | Natural, Type | |
| | | | | | | C - Allen | |
| | | | | | | Flavors Inc. | |
| 250 | 64A3 | CBD Isolate | 3.3 | MCT | 15.0 | | |
| | | CBN Isolate | 1.7 | | | | |
| 251 | 10A2C5A | Full Spectrum | 5.0 | MCT | 16.0 | | |
| | | Distillate | | | | | |
| 252 | 64C3 | Broad | 5.0 | MCT | 20.0 | | |
| | | Spectrum | | | | | |
| | | Distillate | | | | | |
| 253 | 10A2C5A5C | CBD Isolate | 8.0 | MCT | 22.4 | Bitterness | 1.4 |
| | | | | | | Blocker, | |
| | | | | | | Natural, Type | |
| | | | | | | C - Allen | |
| | | | | | | Flavors Inc. | |
| 254 | 10A2C5A1A | CBD Isolate | 8.0 | MCT | 22.4 | Bitterness | 0.3 |
| | | | | | | Blocker, | |
| | | | | | | Natural, Type | |
| | | | | | | C - Allen | |
| | | | | | | Flavors Inc. | |
| 255 | 10A2C5A1C | CBD Isolate | 8.0 | MCT | 22.4 | Bitterness | 0.3 |
| | | | | | | Blocker, | |
| | | | | | | Natural, Type | |
| | | | | | | C - Allen | |
| | | | | | | Flavors Inc. | |
| 256 | 10A2C5A5D1 | Broad | 8.0 | MCT | 30.0 | Bitterness | 1.4 |
| | | Spectrum | | | | Blocker, | |
| | | Distillate | | | | Natural, Type | |
| | | | | | | C - Allen | |
| | | | | | | Flavors Inc. | |
| 257 | 10A2C5A5D1 | Broad | 8.0 | MCT | 30.0 | Bitterness | 1.4 |
| | | Spectrum | | | | Blocker, | |
| | | Distillate | | | | Natural, Type | |
| | | | | | | C - Allen | |
| | | | | | | Flavors Inc. | |
| 258 | 10A2C5A5D | CBD Isolate | 8.0 | MCT | 30.0 | Bitterness | 1.4 |
| | | | | | | Blocker, | |
| | | | | | | Natural, Type | |
| | | | | | | C - Allen | |
| | | | | | | Flavors Inc. | |
| 259 | 10A2C5A1B | CBD Isolate | 8.0 | MCT | 30.0 | Bitterness | 0.3 |
| | | | | | | Blocker, | |
| | | | | | | Natural, Type | |
| | | | | | | C - Allen | |
| | | | | | | Flavors Inc. | |
| 260 | 64D4A1 | Broad | 5.0 | Glyceril | 13.0 | | |
| | | Spectrum | | Monooleate | | | |
| | | Distillate | | (Type 40) - | | | |
| | | | | Gattefossé | | | |
| | | | | SAS | | | |
| | | CBD Isolate | 5.0 | MCT | 2.0 | | |
| 261 | 64D4A2 | Broad | 5.0 | Refined | 14.5 | | |
| | | Spectrum | | Soybean | | | |
| | | Distillate | | Oil | | | |
| | | CBD Isolate | 5.0 | MCT | 0.5 | | |
| 262 | 56F3H2B | Broad | 2.0 | Hemp | 4.5 | Bitterness | 0.5 |
| | | Spectrum | | Seed Oil | | Blocker, | |
| | | Distillate | | | | Natural, Type | |
| | | CBN Isolate | 1.0 | MCT | 0.5 | A - Allen | |
| | | | | | | Flavors Inc. | |
| 263 | 25G51E | Broad | 1.0 | Grape | 1.4 | | |
| | | Spectrum | | Seed Oil | | | |
| | | Distillate | | MCT | 0.1 | | |

TABLE S006B
compositions of the corresponding Hydrophilic phase for examples 212-263.

| | | Us- | | | | Us- | | Us- | | Us- | Contin- | Us- |
|---|---|---|---|---|---|---|---|---|---|---|---|---|
| Exam- | | age | 2nd | Us- | Tertiary | age | Encap- | age | Co- | age | uous | age |
| ple | Emulsifier | rate | Emulsifier | age | Emulsifier | rate | sulant | rate | Solvent | Rate | Phase | Rate |
| 212 | Sunflower | 2.0 | Sucrose | 1.0 | | | | | | | Glycerin | 94.5 |
| | Lecithin, ≥99% - | | Monoester of | | | | | | | | | |
| | Compass | | Palmitic | | | | | | | | | |
| | Foods | | Acid - | | | | | | | | | |
| | Pte Ltd | | Compass | | | | | | | | | |
| | | | Foods | | | | | | | | | |
| | | | Pte Ltd | | | | | | | | | |
| 213 | Sunflower | 2.0 | Sucrose | 1.0 | | | | | | | Glycerin | 94.5 |
| | Lecithin, ≥99% - | | Monoester of | | | | | | | | | |
| | Compass | | Palmitic | | | | | | | | | |
| | Foods | | Acid - | | | | | | | | | |
| | Pte Ltd | | Compass | | | | | | | | | |
| | | | Foods | | | | | | | | | |
| | | | Pte Ltd | | | | | | | | | |
| 214 | Enzyme | 1.0 | Fatfree | 1.0 | | | | | | | Glycerin | 96.0 |
| | Modified | | Sunflower | | | | | | | | | |
| | Soy | | Lecithin | | | | | | | | | |
| | Lecithin - | | (non-GMO) | | | | | | | | | |
| | American | | with 60% | | | | | | | | | |
| | Lecithin | | Phosphatidyl- | | | | | | | | | |
| | Company | | choline - | | | | | | | | | |
| | | | Lipoid | | | | | | | | | |
| 215 | Enzyme | 1.0 | Fatfree | 1.0 | | | | | | | Glycerin | 96.0 |
| | Modified | | Sunflower | | | | | | | | | |
| | Soy | | Lecithin | | | | | | | | | |
| | Lecithin - | | (non-GMO) | | | | | | | | | |
| | American | | with 60% | | | | | | | | | |
| | Lecithin | | Phosphatidyl- | | | | | | | | | |
| | Company | | choline - | | | | | | | | | |
| | | | Lipoid | | | | | | | | | |
| 216 | Fat-free | 1.0 | Fatfree | 1.0 | | | | | | | Glycerin | 96.0 |
| | Soybean | | Sunflower | | | | | | | | | |
| | Lecithin | | Lecithin | | | | | | | | | |
| | with 45% | | (non-GMO) | | | | | | | | | |
| | phosphatidyl- | | with 60% | | | | | | | | | |
| | choline - | | Phosphatidyl- | | | | | | | | | |
| | Lipoid | | choline - | | | | | | | | | |
| | | | Lipoid | | | | | | | | | |
| 217 | Sunflower | 2.0 | Enzyme | 0.5 | Sucrose | 0.5 | | | | | Glycerin | 95.0 |
| | Lecithin, ≥99% - | | Modified | | Monoester | | | | | | | |
| | Compass | | Soy | | of | | | | | | | |
| | Foods | | Lecithin - | | Palmitic | | | | | | | |
| | Pte Ltd | | American | | Acid - | | | | | | | |
| | | | Lecithin | | Compass | | | | | | | |
| | | | Company | | Foods | | | | | | | |
| | | | | | Pte Ltd | | | | | | | |
| 218 | Modified | 5.0 | | | | | | | | | Distilled | 93.0 |
| | Gum | | | | | | | | | | Water | |
| | Acacia - | | | | | | | | | | | |
| | Ingredion | | | | | | | | | | | |
| | Inc. | | | | | | | | | | | |
| 219 | Modified | 5.0 | | | | | | | | | Distilled | 93.0 |
| | Gum | | | | | | | | | | Water | |
| | Acacia - | | | | | | | | | | | |
| | Ingredion | | | | | | | | | | | |
| | Inc. | | | | | | | | | | | |
| 220 | Sucrose | 1.5 | | | | | | | | | Glycerin | 92.5 |
| | Monoester | | | | | | | | | | | |
| | of | | | | | | | | | | | |
| | Palmitic | | | | | | | | | | | |
| | Acid - | | | | | | | | | | | |
| | Compass | | | | | | | | | | | |
| | Foods | | | | | | | | | | | |
| | Pte Ltd | | | | | | | | | | | |
| 221 | Sunflower | 1.0 | Enzyme | 0.5 | Sucrose | 0.5 | | | | | Glycerin | 94.0 |
| | Lecithin, ≥99% - | | Modified | | Monoester | | | | | | | |
| | Compass | | Soy | | of | | | | | | | |
| | Foods | | Lecithin - | | Palmitic | | | | | | | |
| | Pte Ltd | | American | | Acid - | | | | | | | |
| | | | Lecithin | | Compass | | | | | | | |
| | | | Company | | Foods | | | | | | | |
| | | | | | Pte Ltd | | | | | | | |
| 222 | Soybean | 1.0 | Sucrose | 1.0 | | | | | | | Glycerin | 96.0 |
| | Phospholipids | | Monoester | | | | | | | | | |
| | with 70% | | of | | | | | | | | | |
| | Phosphatidyl- | | Palmitic | | | | | | | | | |
| | choline - | | Acid - | | | | | | | | | |
| | Lipoid | | Compass | | | | | | | | | |
| | | | Foods | | | | | | | | | |
| | | | Pte Ltd | | | | | | | | | |
| 223 | Modified | 10.0 | | | | | Flavor- | 10.0 | | | Distilled | 40.0 |
| | Food | | | | | | free | | | | Water | |
| | Starch | | | | | | Malto- | | | | | |
| | from | | | | | | dextrin - | | | | | |
| | Tapioca - | | | | | | Ingredion | | | | | |
| | Ingredion | | | | | | Inc. | | | | | |
| | Inc. | | | | | | | | | | | |
| 224 | Modified | 5.0 | Modified | 5.0 | | | Flavor- | 10.0 | | | Distilled | 56.0 |
| | Food | | Food | | | | free | | | | Water | |
| | Starch | | Starch | | | | Malto- | | | | | |
| | from | | from | | | | dextrin - | | | | | |
| | Tapioca - | | Waxy | | | | Ingredion | | | | | |
| | Ingredion | | Maize - | | | | Inc. | | | | | |
| | Inc. | | Ingredion | | | | | | | | | |
| | | | Inc. | | | | | | | | | |
| 225 | Sunflower | 2.0 | Sucrose | 1.0 | | | | | | | Glycerin | 94.5 |
| | Lecithin, ≥99% - | | Monoester | | | | | | | | | |
| | Compass | | of | | | | | | | | | |
| | Foods | | Palmitic | | | | | | | | | |
| | Pte Ltd | | Acid - | | | | | | | | | |
| | | | Compass | | | | | | | | | |
| | | | Foods | | | | | | | | | |
| | | | Pte Ltd | | | | | | | | | |
| 226 | Sunflower | 2.0 | Sucrose | 1.0 | | | | | | | Glycerin | 94.5 |
| | Lecithin, ≥99% - | | Monoester | | | | | | | | | |
| | Compass | | of | | | | | | | | | |
| | Foods | | Palmitic | | | | | | | | | |
| | Pte Ltd | | Acid - | | | | | | | | | |
| | | | Compass | | | | | | | | | |
| | | | Foods | | | | | | | | | |
| | | | Pte Ltd | | | | | | | | | |
| 227 | Quillaja | 4.0 | | | | | | | | | Glycerin | 93.0 |
| | Saponaria | | | | | | | | | | | |
| | Extract - | | | | | | | | | | | |
| | Ingredion | | | | | | | | | | | |
| | Inc. | | | | | | | | | | | |
| 228 | Lecithin | 4.0 | | | | | | | | | Glycerin | 92.0 |
| | with | | | | | | | | | | | |
| | Ethoxylated | | | | | | | | | | | |
| | mono di- | | | | | | | | | | | |
| | glycerides - | | | | | | | | | | | |
| | Solae | | | | | | | | | | | |
| 229 | Sunflower | 2.0 | Sucrose | 1.0 | | | | | | | Glycerin | 94.5 |
| | Lecithin, ≥99% - | | Monoester | | | | | | | | | |
| | Compass | | of | | | | | | | | | |
| | Foods | | Palmitic | | | | | | | | | |
| | Pte Ltd | | Acid - | | | | | | | | | |
| | | | Compass | | | | | | | | | |
| | | | Foods | | | | | | | | | |
| | | | Pte Ltd | | | | | | | | | |
| 230 | Fat-free | 1.0 | | | | | | | | | Glycerin | 97.0 |
| | Soybean | | | | | | | | | | | |
| | Lecithin | | | | | | | | | | | |
| | with 45% | | | | | | | | | | | |
| | phosphatidyl- | | | | | | | | | | | |
| | choline - | | | | | | | | | | | |
| | Lipoid | | | | | | | | | | | |
| 231 | Lecithin | 4.0 | | | | | | | | | Glycerin | 92.0 |
| | with | | | | | | | | | | | |
| | Ethoxylated | | | | | | | | | | | |
| | mono di- | | | | | | | | | | | |
| | glycerides - | | | | | | | | | | | |
| | Solae | | | | | | | | | | | |
| 232 | Sunflower | 2.0 | Sucrose | 1.0 | | | | | | | Glycerin | 94.5 |
| | Lecithin, ≥99% - | | Monoester | | | | | | | | | |
| | Compass | | of | | | | | | | | | |
| | Foods | | Palmitic | | | | | | | | | |
| | Pte Ltd | | Acid - | | | | | | | | | |
| | | | Compass | | | | | | | | | |
| | | | Foods | | | | | | | | | |
| | | | Pte Ltd | | | | | | | | | |
| 233 | Sunflower | 2.0 | Sucrose | 1.0 | | | | | | | Glycerin | 94.5 |
| | Lecithin, ≥99% - | | Monoester | | | | | | | | | |
| | Compass | | of | | | | | | | | | |
| | Foods | | Palmitic | | | | | | | | | |
| | Pte Ltd | | Acid - | | | | | | | | | |
| | | | Compass | | | | | | | | | |
| | | | Foods | | | | | | | | | |
| | | | Pte Ltd | | | | | | | | | |
| 234 | Enzyme | 1.0 | Fatfree | 1.0 | | | | | | | Glycerin | 96.0 |
| | Modified | | Sunflower | | | | | | | | | |
| | Soy | | Lecithin | | | | | | | | | |
| | Lecithin - | | (non-GMO) | | | | | | | | | |
| | American | | with 60% | | | | | | | | | |
| | Lecithin | | Phosphatidyl- | | | | | | | | | |
| | Company | | choline - | | | | | | | | | |
| | | | Lipoid | | | | | | | | | |
| 235 | Sunflower | 4.0 | Sucrose | 2.0 | | | | | | | Glycerin | 90.5 |
| | Lecithin, ≥99% - | | Monoester | | | | | | | | | |
| | Compass | | of | | | | | | | | | |
| | Foods | | Palmitic | | | | | | | | | |
| | Pte Ltd | | Acid - | | | | | | | | | |
| | | | Compass | | | | | | | | | |
| | | | Foods | | | | | | | | | |
| | | | Pte Ltd | | | | | | | | | |
| 236 | Sunflower | 2.0 | Sucrose | 1.0 | | | | | | | Glycerin | 94.5 |
| | Lecithin, ≥99% - | | Monoester | | | | | | | | | |
| | Compass | | of | | | | | | | | | |
| | Foods | | Palmitic | | | | | | | | | |
| | Pte Ltd | | Acid - | | | | | | | | | |
| | | | Compass | | | | | | | | | |
| | | | Foods | | | | | | | | | |
| | | | Pte Ltd | | | | | | | | | |
| 237 | Lecithin | 4.0 | | | | | | | | | Glycerin | 92.0 |
| | with | | | | | | | | | | | |
| | Ethoxylated | | | | | | | | | | | |
| | mono di- | | | | | | | | | | | |
| | glycerides - | | | | | | | | | | | |
| | Solae | | | | | | | | | | | |
| 238 | Sunflower | 2.5 | Sucrose | 1.5 | | | | | | | Glycerin | 82.0 |
| | Lecithin, ≥99% - | | Monoester | | | | | | | | | |
| | Compass | | of | | | | | | | | | |
| | Foods | | Palmitic | | | | | | | | | |
| | Pte Ltd | | Acid - | | | | | | | | | |
| | | | Compass | | | | | | | | | |
| | | | Foods | | | | | | | | | |
| | | | Pte Ltd | | | | | | | | | |
| 239 | Modified | 10.0 | | | | | Flavor- | 10.0 | | | Distilled | 50.0 |
| | Food | | | | | | free | | | | Water | |
| | Starch | | | | | | Malto- | | | | | |
| | from | | | | | | dextrin - | | | | | |
| | Tapioca - | | | | | | Ingredion | | | | | |
| | Ingredion | | | | | | Inc. | | | | | |
| | Inc. | | | | | | | | | | | |
| 240 | Lecithin | 4.0 | | | | | | | | | Glycerin | 92.0 |
| | with | | | | | | | | | | | |
| | Ethoxylated | | | | | | | | | | | |
| | mono di- | | | | | | | | | | | |
| | glycerides - | | | | | | | | | | | |
| | Solae | | | | | | | | | | | |
| 241 | Sucrose | 1.0 | | | | | | | | | Glycerin | 97.0 |
| | Ester of | | | | | | | | | | | |
| | Fatty | | | | | | | | | | | |
| | Acids, | | | | | | | | | | | |
| | M175D | | | | | | | | | | | |
| | C175 - | | | | | | | | | | | |
| | Compass | | | | | | | | | | | |
| | Foods | | | | | | | | | | | |
| | Pte Ltd | | | | | | | | | | | |
| 242 | Pre- | 1.0 | | | | | | | | | Propylene | 97.0 |
| | hydrated | | | | | | | | | | Glycol | |
| | Gum | | | | | | | | | | | |
| | Arabic | | | | | | | | | | | |
| 243 | Quillaja | 1.0 | | | | | | | Distilled | 10.0 | 42 DE | 87.0 |
| | Saponaria | | | | | | | | Water | | Corn | |
| | Extract - | | | | | | | | | | Syrup | |
| | Ingredion | | | | | | | | | | | |
| | Inc. | | | | | | | | | | | |
| 244 | Lysophosphatidyl- | 1.0 | | | | | | | | | Glycerin | 97.0 |
| | choline from | | | | | | | | | | | |
| | soybean | | | | | | | | | | | |
| | lecithin | | | | | | | | | | | |
| | (non-GMO), | | | | | | | | | | | |
| | 80% - | | | | | | | | | | | |
| | Lipoid | | | | | | | | | | | |
| 245 | Sunflower | 2.0 | Sucrose | 1.0 | | | | | | | Glycerin | 94.5 |
| | Lecithin, ≥99% - | | Monoester | | | | | | | | | |
| | Compass | | of | | | | | | | | | |
| | Foods | | Palmitic | | | | | | | | | |
| | Pte Ltd | | Acid - | | | | | | | | | |
| | | | Compass | | | | | | | | | |
| | | | Foods | | | | | | | | | |
| | | | Pte Ltd | | | | | | | | | |
| 246 | Gum | 6.0 | Quillaja | 1.0 | | | | | | | Glycerin | 87.0 |
| | Acacia - | | Saponaria | | | | | | | | | |
| | Ingredion | | Extract - | | | | | | | | | |
| | Inc. | | Naturex | | | | | | | | | |
| 247 | Quillaja | 6.0 | Sucrose | 1.0 | | | | | | | Glycerin | 84.5 |
| | Saponaria | | Monoester | | | | | | | | | |
| | Extract - | | of | | | | | | | | | |
| | Naturex | | Palmitic | | | | | | | | | |
| | | | Acid - | | | | | | | | | |
| | | | Compass | | | | | | | | | |
| | | | Foods | | | | | | | | | |
| | | | Pte Ltd | | | | | | | | | |
| 248 | Quillaja | 2.5 | Sunflower | 0.5 | | | | | | | Glycerin | 82.0 |
| | Saponaria | | Lecithin, ≥99% - | | | | | | | | | |
| | Extract - | | Compass | | | | | | | | | |
| | Naturex | | Foods | | | | | | | | | |
| | | | Pte Ltd | | | | | | | | | |
| 249 | Quillaja | 2.8 | Sucrose | 1.4 | | | | | | | Glycerin | 85.1 |
| | Saponaria | | Monoester | | | | | | | | | |
| | Extract - | | of | | | | | | | | | |
| | Ingredion | | Palmitic | | | | | | | | | |
| | Inc. | | Acid - | | | | | | | | | |
| | | | Compass | | | | | | | | | |
| | | | Foods | | | | | | | | | |
| | | | Pte Ltd | | | | | | | | | |
| 250 | Flavor- | 5.0 | Modified | 5.0 | | | | | Distilled | 15.0 | Glycerin | 55.0 |
| | free | | Food | | | | | | Water | | | |
| | Malto- | | Starch | | | | | | | | | |
| | dextrin - | | from | | | | | | | | | |
| | Ingredion | | Tapioca - | | | | | | | | | |
| | Inc. | | Ingredion | | | | | | | | | |
| | | | Inc. | | | | | | | | | |
| 251 | Quillaja | 5.0 | Sucrose | 2.5 | | | | | | | Glycerin | 71.5 |
| | Saponaria | | Monoester | | | | | | | | | |
| | Extract - | | of | | | | | | | | | |
| | Ingredion | | Palmitic | | | | | | | | | |
| | Inc. | | Acid - | | | | | | | | | |
| | | | Compass | | | | | | | | | |
| | | | Foods | | | | | | | | | |
| | | | Pte Ltd | | | | | | | | | |
| 252 | Flavor- | 5.0 | Modified | 10.0 | | | | | Distilled | 15.0 | Glycerin | 45.0 |
| | free | | Food | | | | | | Water | | | |
| | Malto- | | Starch | | | | | | | | | |
| | dextrin - | | from | | | | | | | | | |
| | Ingredion | | Tapioca - | | | | | | | | | |
| | Inc. | | Ingredion | | | | | | | | | |
| | | | Inc. | | | | | | | | | |
| 253 | Quillaja | 8.0 | Sucrose | 4.0 | | | | | | | Glycerin | 56.2 |
| | Saponaria | | Monoester | | | | | | | | | |
| | Extract - | | of | | | | | | | | | |
| | Ingredion | | Palmitic | | | | | | | | | |
| | Inc. | | Acid - | | | | | | | | | |
| | | | Compass | | | | | | | | | |
| | | | Foods | | | | | | | | | |
| | | | Pte Ltd | | | | | | | | | |
| 254 | Quillaja | 8.0 | Sucrose | 4.0 | | | | | | | Glycerin | 57.3 |
| | Saponaria | | Monoester | | | | | | | | | |
| | Extract - | | of | | | | | | | | | |
| | Ingredion | | Palmitic | | | | | | | | | |
| | Inc. | | Acid - | | | | | | | | | |
| | | | Compass | | | | | | | | | |
| | | | Foods | | | | | | | | | |
| | | | Pte Ltd | | | | | | | | | |
| 255 | Quillaja | 8.0 | Sucrose | 2.5 | | | | | | | Glycerin | 58.8 |
| | Saponaria | | Monoester | | | | | | | | | |
| | Extract - | | of | | | | | | | | | |
| | Ingredion | | Palmitic | | | | | | | | | |
| | Inc. | | Acid - | | | | | | | | | |
| | | | Compass | | | | | | | | | |
| | | | Foods | | | | | | | | | |
| | | | Pte Ltd | | | | | | | | | |
| 256 | Quillaja | 8.0 | Sucrose | 4.0 | | | | | | | Glycerin | 48.6 |
| | Saponaria | | Monoester | | | | | | | | | |
| | Extract - | | of | | | | | | | | | |
| | Ingredion | | Palmitic | | | | | | | | | |
| | Inc. | | Acid - | | | | | | | | | |
| | | | Compass | | | | | | | | | |
| | | | Foods | | | | | | | | | |
| | | | Pte Ltd | | | | | | | | | |
| 257 | Quillaja | 8.0 | Sucrose | 4.0 | | | | | | | Glycerin | 48.6 |
| | Saponaria | | Monoester | | | | | | | | | |
| | Extract - | | of | | | | | | | | | |
| | Ingredion | | Palmitic | | | | | | | | | |
| | Inc. | | Acid - | | | | | | | | | |
| | | | Compass | | | | | | | | | |
| | | | Foods | | | | | | | | | |
| | | | Pte Ltd | | | | | | | | | |
| 258 | Quillaja | 8.0 | Sucrose | 4.0 | | | | | | | Glycerin | 48.6 |
| | Saponaria | | Monoester | | | | | | | | | |
| | Extract - | | of | | | | | | | | | |
| | Ingredion | | Palmitic | | | | | | | | | |
| | Inc. | | Acid - | | | | | | | | | |
| | | | Compass | | | | | | | | | |
| | | | Foods | | | | | | | | | |
| | | | Pte Ltd | | | | | | | | | |
| 259 | Quillaja | 8.0 | Sucrose | 4.0 | | | | | | | Glycerin | 49.7 |
| | Saponaria | | Monoester | | | | | | | | | |
| | Extract - | | of | | | | | | | | | |
| | Ingredion | | Palmitic | | | | | | | | | |
| | Inc. | | Acid - | | | | | | | | | |
| | | | Compass | | | | | | | | | |
| | | | Foods | | | | | | | | | |
| | | | Pte Ltd | | | | | | | | | |
| 260 | | | Modified | 15.0 | | | | | Distilled | 15.0 | Glycerin | 45.0 |
| | | | Food | | | | | | Water | | | |
| | | | Starch | | | | | | | | | |
| | | | from | | | | | | | | | |
| | | | Waxy | | | | | | | | | |
| | | | Maize - | | | | | | | | | |
| | | | Ingredion | | | | | | | | | |
| | | | Inc. | | | | | | | | | |
| 261 | | | Modified | 15.0 | | | | | Distilled | 15.0 | Glycerin | 45.0 |
| | | | Food | | | | | | Water | | | |
| | | | Starch | | | | | | | | | |
| | | | from | | | | | | | | | |
| | | | Waxy | | | | | | | | | |
| | | | Maize - | | | | | | | | | |
| | | | Ingredion | | | | | | | | | |
| | | | Inc. | | | | | | | | | |
| 262 | Quillaja | 6.0 | Sucrose | 1.0 | | | | | | | Glycerin | 84.5 |
| | Saponaria | | Monoester | | | | | | | | | |
| | Extract - | | of | | | | | | | | | |
| | Naturex | | Palmitic | | | | | | | | | |
| | | | Acid - | | | | | | | | | |
| | | | Compass | | | | | | | | | |
| | | | Foods | | | | | | | | | |
| | | | Pte Ltd | | | | | | | | | |
| 263 | Sunflower | 2.0 | Quillaja | 1.0 | | | | | | | Glycerin | 94.5 |
| | Lecithin, ≥99% - | | Saponaria | | | | | | | | | |
| | Compass | | Extract - | | | | | | | | | |
| | Foods | | Ingredion | | | | | | | | | |
| | Pte Ltd | | Inc. | | | | | | | | | |

### Examples 264-436

Tables S007A and S007B show example formulations with one, two, or three emulsifiers. These examples also illustrate the scope of ABIs and demonstrate that water-soluble ABIs can be used within the context of this disclosure. These examples also show different continuous phases. Other continuous phases as listed above at paragraph [0047] and blends thereof may also be used, as well as other emulsifiers listed above at paragraphs [0049], [0051], [0053], [0056] and blends thereof, and other co-solvents as listed above at paragraph [0059] and blends thereof can all be used in these emulsions.

TABLE S007A
the hydrophobic phase compositions used in the emulsions

| | | | | | | Flavorant | |
|---|---|---|---|---|---|---|---|
| | | | Usage | | Usage | or | Usage |
| Example | Prototype ID | ABI | Rate | Carrier Oil | Rate | Bittermasker | Rate |
| 264 | 25A3 | CBD | 2.0 | MCT | 3.0 | | |
| | | Isolate | | | | | |
| 265 | 25A5A-OR | CBD | 2.8 | MCT | 7.8 | Orange Oil | 0.2 |
| | | Isolate | | | | Flavor - | |
| | | | | | | Flavor | |
| | | | | | | Insights | |
| | | | | | | Bitterness | 0.1 |
| | | | | | | Blocker, | |
| | | | | | | Natural, | |
| | | | | | | Type A - | |
| | | | | | | Allen | |
| | | | | | | Flavors | |
| | | | | | | Inc. | |
| 266 | 9B | CBD | 1.0 | Glyceril | 2.0 | | |
| | | Isolate | | Monooleate | | | |
| | | | | (Type 40) - | | | |
| | | | | Gattefossé | | | |
| | | | | SAS | | | |
| 267 | 9D3 | CBD | 2.0 | MCT | 2.0 | | |
| | | Isolate | | | | | |
| 268 | 9D4 | CBD | 2.0 | MCT | 2.0 | | |
| | | Isolate | | | | | |
| 269 | 56F1A | CBD | 3.0 | MCT | 2.0 | Glyceryl | 1.0 |
| | | Isolate | | | | dibehenate - | |
| | | | | | | Gattefossé | |
| | | | | | | SAS | |
| 270 | 56E | CBD | 1.5 | MCT | 1.5 | | |
| | | Isolate | | | | | |
| 271 | 64F | CBD | 10.0 | MCT | 15.0 | | |
| | | Isolate | | | | | |
| 272 | 64F1 | CBD | 10.0 | MCT | 15.0 | | |
| | | Isolate | | | | | |
| 273 | 64F2 | CBD | 10.0 | MCT | 15.0 | | |
| | | Isolate | | | | | |
| 274 | 64A | CBD | 10.0 | MCT | 10.0 | | |
| | | Isolate | | | | | |
| 275 | WTAM-1A-17000 | CBD | 3.0 | MCT | 3.0 | | |
| | | Isolate | | | | | |
| 276 | WTAM-1B-17000 | CBD | 3.0 | MCT | 5.0 | | |
| | | Isolate | | | | | |
| 277 | WTAM-1D-17000 | CBD | 3.0 | MCT | 3.0 | | |
| | | Isolate | | | | | |
| 278 | 25A5A1 | CBD | 1.4 | MCT | 7.8 | Bitterness | 0.1 |
| | | Isolate | | | | Blocker, | |
| | | | | | | Natural, | |
| | | | | | | Type A - | |
| | | CBN | 1.4 | | | Allen | |
| | | Isolate | | | | Flavors | |
| | | | | | | Inc. | |
| 279 | 25A5A1-LE | CBD | 1.4 | MCT | 7.8 | Lemon Oil | 0.2 |
| | | Isolate | | | | Flavor - | |
| | | | | | | Flavor | |
| | | | | | | Insights | |
| | | CBN | 1.4 | | | Bitterness | 0.1 |
| | | Isolate | | | | Blocker, | |
| | | | | | | Natural, | |
| | | | | | | Type A - | |
| | | | | | | Allen | |
| | | | | | | Flavors | |
| | | | | | | Inc. | |
| 280 | 64A4 | CBD | 3.3 | MCT | 15.0 | | |
| | | Isolate | | | | | |
| | | CBN | 1.7 | | | | |
| | | Isolate | | | | | |
| 281 | 64A6 | CBD | 3.3 | MCT | 20.0 | | |
| | | Isolate | | | | | |
| | | CBN | 1.7 | | | | |
| | | Isolate | | | | | |
| 282 | 64D2 | Broad | 2.5 | MCT | 20.0 | | |
| | | Spectrum | | | | | |
| | | Distillate | | | | | |
| | | CBD | 2.5 | | | | |
| | | Isolate | | | | | |
| 283 | 64D3 | Broad | 5.0 | MCT | 15.0 | | |
| | | Spectrum | | | | | |
| | | Distillate | | | | | |
| | | CBD | 5.0 | | | | |
| | | Isolate | | | | | |
| 284 | 64D4 | Broad | 5.0 | MCT | 15.0 | | |
| | | Spectrum | | | | | |
| | | Distillate | | | | | |
| | | CBD | 5.0 | | | | |
| | | Isolate | | | | | |
| 285 | 64D5 | Broad | 5.0 | MCT | 15.0 | | |
| | | Spectrum | | | | | |
| | | Distillate | | | | | |
| | | CBD | 5.0 | | | | |
| | | Isolate | | | | | |
| 286 | 64D4A1 | Broad | 5.0 | Glyceril | 13.0 | | |
| | | Spectrum | | Monooleate | | | |
| | | Distillate | | (Type 40) - | | | |
| | | | | Gattefossé | | | |
| | | | | SAS | | | |
| | | CBD | 5.0 | MCT | 2.0 | | |
| | | Isolate | | | | | |
| 287 | 64D4A2 | Broad | 5.0 | Refined | 14.5 | | |
| | | Spectrum | | Soybean | | | |
| | | Distillate | | Oil | | | |
| | | CBD | 5.0 | MCT | 0.5 | | |
| | | Isolate | | | | | |
| 288 | 44B | Broad | 3.0 | MCT | 3.0 | | |
| | | Spectrum | | | | | |
| | | Distillate | | | | | |
| 289 | 43 | Broad | 1.0 | Grape | 1.0 | | |
| | | Spectrum | | seed oil | | | |
| | | Distillate | | | | | |
| 290 | 20P | Broad | 1.0 | MCT | 1.0 | Lime | 0.5 |
| | | Spectrum | | | | Flavor | |
| | | Distillate | | | | | |
| 291 | 20Q | Broad | 1.0 | MCT | 1.0 | Lemon | 0.5 |
| | | Spectrum | | | | Flavor - FI | |
| | | Distillate | | | | | |
| 292 | 121 | Broad | 1.0 | MCT | 1.0 | | |
| | | Spectrum | | | | | |
| | | Distillate | | | | | |
| 293 | 9T | Broad | 1.0 | MCT | 1.0 | | |
| | | Spectrum | | | | | |
| | | Distillate | | | | | |
| 294 | 9S | Broad | 1.0 | MCT | 1.0 | | |
| | | Spectrum | | | | | |
| | | Distillate | | | | | |
| 295 | 56 | Broad | 1.0 | MCT | 1.0 | | |
| | | Spectrum | | | | | |
| | | Distillate | | | | | |
| 296 | 56A | Broad | 1.0 | MCT | 1.0 | | |
| | | Spectrum | | | | | |
| | | Distillate | | | | | |
| 297 | 56B | Broad | 1.0 | MCT | 1.0 | | |
| | | Spectrum | | | | | |
| | | Distillate | | | | | |
| 298 | 56C | Broad | 1.0 | MCT | 1.0 | | |
| | | Spectrum | | | | | |
| | | Distillate | | | | | |
| 299 | 57 | Broad | 1.0 | MCT | 1.0 | | |
| | | Spectrum | | | | | |
| | | Distillate | | | | | |
| 300 | 58 | Broad | 1.0 | MCT | 1.0 | | |
| | | Spectrum | | | | | |
| | | Distillate | | | | | |
| 301 | 59 | Broad | 1.0 | MCT | 1.0 | | |
| | | Spectrum | | | | | |
| | | Distillate | | | | | |
| 302 | 46 | Broad | 1.0 | MCT | 1.0 | | |
| | | Spectrum | | | | | |
| | | Distillate | | | | | |
| 303 | 34A | Broad | 1.0 | MCT | 1.0 | | |
| | | Spectrum | | | | | |
| | | Distillate | | | | | |
| 304 | 33A | Broad | 1.0 | MCT | 1.0 | | |
| | | Spectrum | | | | | |
| | | Distillate | | | | | |
| 305 | 64B | Broad | 10.0 | MCT | 10.0 | | |
| | | Spectrum | | | | | |
| | | Distillate | | | | | |
| 306 | 64E | Broad | 5.0 | MCT | 20.0 | | |
| | | Spectrum | | | | | |
| | | Distillate | | | | | |
| 307 | 20S | Broad | 1.0 | MCT | 1.0 | | |
| | | Spectrum | | | | | |
| | | Distillate | | | | | |
| 308 | 31 | Full | 1.0 | MCT | 1.0 | | |
| | | Spectrum | | | | | |
| | | Distillate | | | | | |
| 309 | 32 | Full | 1.0 | MCT | 1.0 | | |
| | | Spectrum | | | | | |
| | | Distillate | | | | | |
| 310 | 1W | Full | 1.0 | MCT | 1.0 | | |
| | | Spectrum | | | | | |
| | | Distillate | | | | | |
| 311 | 29F | Full | 1.0 | MCT | 1.0 | | |
| | | Spectrum | | | | | |
| | | Distillate | | | | | |
| 312 | 1Y | Full | 1.0 | MCT | 1.0 | | |
| | | Spectrum | | | | | |
| | | Distillate | | | | | |
| 313 | 1Z | Full | 1.0 | MCT | 1.0 | | |
| | | Spectrum | | | | | |
| | | Distillate | | | | | |
| 314 | 29E | Full | 3.0 | MCT | 3.0 | | |
| | | Spectrum | | | | | |
| | | Distillate | | | | | |
| 315 | 43A2 | Full | 2.0 | MCT | 2.0 | | |
| | | Spectrum | | | | | |
| | | Distillate | | | | | |
| 316 | 21D | Full | 2.0 | Maisine | 2.0 | | |
| | | Spectrum | | CC | | | |
| | | Distillate | | | | | |
| 317 | 21E | Full | 2.0 | Glyceril | 2.0 | | |
| | | Spectrum | | Monooleate | | | |
| | | Distillate | | (Type 40) - | | | |
| | | | | Gattefossé | | | |
| | | | | SAS | | | |
| 318 | 21C | Full | 3.0 | MCT | 3.0 | | |
| | | Spectrum | | | | | |
| | | Distillate | | | | | |
| 319 | 29A | Full | 1.0 | MCT | 1.0 | | |
| | | Spectrum | | | | | |
| | | Distillate | | | | | |
| 320 | 18A | Full | 1.0 | MCT | 1.0 | | |
| | | Spectrum | | | | | |
| | | Distillate | | | | | |
| 321 | 20A | Full | 1.0 | MCT | 1.0 | | |
| | | Spectrum | | | | | |
| | | Distillate | | | | | |
| 322 | L201112-1G | Full | 3.0 | MCT | 1.0 | | |
| | | Spectrum | | | | | |
| | | Distillate | | | | | |
| 323 | 12A | Full | 1.0 | MCT | 1.0 | | |
| | | Spectrum | | | | | |
| | | Distillate | | | | | |
| 324 | 40 | Full | 1.0 | MCT | 1.0 | | |
| | | Spectrum | | | | | |
| | | Distillate | | | | | |
| 325 | 9D | Full | 1.0 | MCT | 1.0 | | |
| | | Spectrum | | | | | |
| | | Distillate | | | | | |
| 326 | 9 | Full | 1.0 | MCT | 1.0 | | |
| | | Spectrum | | | | | |
| | | Distillate | | | | | |
| 327 | 9A | Full | 1.0 | MCT | 1.0 | | |
| | | Spectrum | | | | | |
| | | Distillate | | | | | |
| 328 | 11A | Full | 3.0 | MCT | 3.0 | | |
| | | Spectrum | | | | | |
| | | Distillate | | | | | |
| 329 | 26A | Full | 1.0 | MCT | 1.0 | | |
| | | Spectrum | | | | | |
| | | Distillate | | | | | |
| 330 | 26 | Full | 1.0 | MCT | 1.0 | | |
| | | Spectrum | | | | | |
| | | Distillate | | | | | |
| 331 | 11 | Full | 1.0 | MCT | 1.0 | | |
| | | Spectrum | | | | | |
| | | Distillate | | | | | |
| 332 | 12 | Full | 1.0 | MCT | 1.0 | | |
| | | Spectrum | | | | | |
| | | Distillate | | | | | |
| 333 | 35 | Full | 1.0 | MCT | 1.0 | | |
| | | Spectrum | | | | | |
| | | Distillate | | | | | |
| 334 | 35A | Broad | 1.0 | MCT | 1.0 | | |
| | | Spectrum | | | | | |
| | | Distillate | | | | | |
| 335 | 19 | Full | 1.0 | MCT | 1.0 | | |
| | | Spectrum | | | | | |
| | | Distillate | | | | | |
| 336 | 19A | Full | 1.0 | MCT | 1.0 | | |
| | | Spectrum | | | | | |
| | | Distillate | | | | | |
| 337 | 64D | Broad | 5.0 | MCT | 20.0 | | |
| | | Spectrum | | | | | |
| | | Distillate | | | | | |
| 338 | 25G5 | CBD | 1.0 | MCT | 1.5 | | |
| | | Isolate | | | | | |
| 339 | 25G711 | CBD | 2.0 | MCT | 3.0 | Bitterness | 0.7 |
| | | Isolate | | | | Blocker, | |
| | | | | | | Natural, | |
| | | | | | | Type A - | |
| | | | | | | Allen | |
| | | | | | | Flavors | |
| | | | | | | Inc. | |
| 340 | 25A5C | CBD | 8.1 | MCT | 12.2 | Bitterness | 0.3 |
| | | Isolate | | | | Blocker, | |
| | | | | | | Natural, | |
| | | | | | | Type A - | |
| | | | | | | Allen | |
| | | | | | | Flavors | |
| | | | | | | Inc. | |
| 341 | 25A5B-LE3 | CBD | 8.1 | MCT | 12.2 | Lemon Oil | 0.5 |
| | | Isolate | | | | Flavor - | |
| | | | | | | Flavor | |
| | | | | | | Insights | |
| | | | | | | Bitterness | 0.3 |
| | | | | | | Blocker, | |
| | | | | | | Natural, | |
| | | | | | | Type A - | |
| | | | | | | Allen | |
| | | | | | | Flavors | |
| | | | | | | Inc. | |
| 342 | 25A5C-GR | CBD | 8.1 | MCT | 12.2 | Grapefruit | 0.5 |
| | | Isolate | | | | Oil Flavor - | |
| | | | | | | Flavor | |
| | | | | | | Insights | |
| | | | | | | Bitterness | 0.3 |
| | | | | | | Blocker, | |
| | | | | | | Natural, | |
| | | | | | | Type A - | |
| | | | | | | Allen | |
| | | | | | | Flavors | |
| | | | | | | Inc. | |
| 343 | 25A5C-OR | CBD | 8.1 | MCT | 12.2 | Orange Oil | 0.5 |
| | | Isolate | | | | Flavor - | |
| | | | | | | Flavor | |
| | | | | | | Insights | |
| | | | | | | Bitterness | 0.3 |
| | | | | | | Blocker, | |
| | | | | | | Natural, | |
| | | | | | | Type A - | |
| | | | | | | Allen | |
| | | | | | | Flavors | |
| | | | | | | Inc. | |
| 344 | 25G5F | CBD | 2.0 | Hemp | 1.5 | | |
| | | Isolate | | Seed Oil | | | |
| 345 | 25G5C6A3 | CBD | 4.0 | MCT | 8.0 | | |
| | | Isolate | | | | | |
| 346 | 25G5-CO1 | CBD | 1.0 | Refined | 1.5 | | |
| | | Isolate | | Sesame | | | |
| | | | | Oil (IV-1) | | | |
| 347 | 25G5-CO2 | CBD | 1.0 | Refined | 1.5 | | |
| | | Isolate | | Olive Oil | | | |
| | | | | (IV) | | | |
| 348 | 25G5-CO3 | CBD | 1.0 | Refined | 1.5 | | |
| | | Isolate | | Soybean | | | |
| | | | | Oil (IV) | | | |
| 349 | 25G5-CO4 | CBD | 1.0 | Peceol | 1.5 | | |
| | | Isolate | | (3088) | | | |
| 350 | 25G5-CO11 | CBD | 1.0 | Grape | 1.5 | | |
| | | Isolate | | Seed Oil | | | |
| | | | | (I-003-bna- | | | |
| | | | | 10) | | | |
| 351 | 25G5-CO12 | CBD | 1.0 | Hemp | 1.5 | | |
| | | Isolate | | Seed Oil | | | |
| | | | | (bna-170-r) | | | |
| 352 | 25G5-CO13 | CBD | 1.0 | Triacetin | 1.5 | | |
| | | Isolate | | | | | |
| 353 | 25G5-CO14 | CBD | 1.0 | Canola Oil | 1.5 | | |
| | | Isolate | | | | | |
| 354 | 25G5-CP4 | CBD | 1.0 | MCT | 1.5 | | |
| | | Isolate | | | | | |
| 355 | 10A2C3 | CBD | 8.5 | MCT | 8.5 | | |
| | | Isolate | | | | | |
| 356 | 10A2C5B1 | CBD | 2.5 | MCT | 7.0 | Bitterness | 0.1 |
| | | Isolate | | | | Blocker, | |
| | | | | | | Type A - | |
| | | | | | | Givaudan | |
| 357 | 10A2C4 | CBD | 5.0 | MCT | 10.0 | | |
| | | Isolate | | | | | |
| 358 | 10A2C5A1C | CBD | 8.0 | MCT | 22.4 | Bitterness | 0.3 |
| | | Isolate | | | | Blocker, | |
| | | | | | | Natural, | |
| | | | | | | Type C - | |
| | | | | | | Allen | |
| | | | | | | Flavors | |
| | | | | | | Inc. | |
| 359 | 10A2C5C1 | CBD | 2.5 | MCT | 7.0 | Bitterness | 0.1 |
| | | Isolate | | | | Blocker, | |
| | | | | | | Type B - | |
| | | | | | | Givaudan | |
| 360 | 10A2C5A9 | CBD | 2.5 | MCT | 7.0 | Bitterness | 0.5 |
| | | Isolate | | | | Blocker, | |
| | | | | | | Natural, | |
| | | | | | | Type B - | |
| | | | | | | Allen | |
| | | | | | | Flavors | |
| | | | | | | Inc. | |
| 361 | 10A2C5A5A | CBD | 2.5 | MCT | 7.0 | Tangerine | 0.2 |
| | | Isolate | | | | flavor | |
| 362 | 10A2C5E1 | CBD | 5.0 | MCT | 25.0 | | |
| | | Isolate | | | | | |
| 363 | 10F17A | CBD | 4.0 | MCT | 4.0 | AF103282 | 1.0 |
| | | Isolate | | | | | |
| 364 | 10A2C5A5F | CBD | 8.0 | MCT | 22.4 | Bitterness | 1.4 |
| | | Isolate | | | | Blocker, | |
| | | | | | | Natural, | |
| | | | | | | Type C - | |
| | | | | | | Allen | |
| | | | | | | Flavors | |
| | | | | | | Inc. | |
| 365 | 56H | CBD | 7.5 | MCT | 7.5 | | |
| | | Isolate | | | | | |
| 366 | 56H1 | CBD | 3.0 | MCT | 5.0 | | |
| | | Isolate | | | | | |
| 367 | 56F3C | CBD | 3.0 | MCT | 7.0 | Bitterness | 0.1 |
| | | Isolate | | | | Blocker, | |
| | | | | | | Natural, | |
| | | | | | | Type C - | |
| | | | | | | Allen | |
| | | | | | | Flavors | |
| | | | | | | Inc. | |
| 368 | 56F4B | CBD | 5.0 | MCT | 5.0 | Bitterness | 0.9 |
| | | Isolate | | | | Blocker, | |
| | | | | | | Natural, | |
| | | | | | | Type C - | |
| | | | | | | Allen | |
| | | | | | | Flavors | |
| | | | | | | Inc. | |
| 369 | 56F3H1B | CBD | 2.0 | MCT | 3.3 | Bitterness | 0.7 |
| | | Isolate | | | | Blocker, | |
| | | | | | | Natural, | |
| | | | | | | Type A - | |
| | | | | | | Allen | |
| | | | | | | Flavors | |
| | | | | | | Inc. | |
| 370 | 56F4C1 | CBD | 5.0 | MCT | 8.3 | Bitterness | 1.3 |
| | | Isolate | | | | Blocker, | |
| | | | | | | Natural, | |
| | | | | | | Type C - | |
| | | | | | | Allen | |
| | | | | | | Flavors | |
| | | | | | | Inc. | |
| 371 | 64 | CBD | 10.0 | MCT | 10.0 | | |
| | | Isolate | | | | | |
| 372 | WTAM-1C-17000 | CBD | 3.0 | MCT | 3.0 | | |
| | | Isolate | | | | | |
| 373 | 25G5-CBN | CBN | 1.0 | MCT | 1.5 | | |
| | | Isolate | | | | | |
| 374 | 10F-CBN | CBN | 1.5 | MCT | 1.2 | | |
| | | Isolate | | | | | |
| 375 | 25G5-CBG | CBG | 1.0 | MCT | 1.5 | | |
| | Isolate | Isolate | | | | | |
| 376 | 25G5-CBG | CBG | 1.0 | MCT | 1.5 | | |
| | Distillate | Distillate | | | | | |
| 377 | 64A2 | CBD | 3.3 | MCT | 10.0 | | |
| | | Isolate | | | | | |
| | | CBN | 1.7 | | | | |
| | | Isolate | | | | | |
| 378 | 64A3 | CBD | 3.3 | MCT | 15.0 | | |
| | | Isolate | | | | | |
| | | CBN | 1.7 | | | | |
| | | Isolate | | | | | |
| 379 | 64A1 | CBD | 6.7 | MCT | 10.0 | | |
| | | Isolate | | | | | |
| | | CBN | 3.3 | | | | |
| | | Isolate | | | | | |
| 380 | 64A5 | CBD | 3.3 | MCT | 15.0 | | |
| | | Isolate | | | | | |
| | | CBN | 1.7 | | | | |
| | | Isolate | | | | | |
| 381 | 10A2C5A5J | CBD | 5.2 | MCT | 30.0 | Bitterness | 1.4 |
| | | Isolate | | | | Blocker, | |
| | | CBN | 2.6 | | | Natural, | |
| | | Isolate | | | | Type C - | |
| | | | | | | Allen | |
| | | | | | | Flavors | |
| | | | | | | Inc. | |
| 382 | 56F3H2 | CBD | 2.0 | MCT | 5.0 | Bitterness | 0.5 |
| | | Isolate | | | | Blocker, | |
| | | CBN | 1.0 | | | Natural, | |
| | | Isolate | | | | Type A - | |
| | | | | | | Allen | |
| | | | | | | Flavors | |
| | | | | | | Inc. | |
| 383 | 56F3H2A | Broad | 2.0 | MCT | 5.0 | Bitterness | 0.5 |
| | | Spectrum | | | | Blocker, | |
| | | Distillate | | | | Natural, | |
| | | CBN | 1.0 | | | Type A - | |
| | | Isolate | | | | Allen | |
| | | | | | | Flavors | |
| | | | | | | Inc. | |
| 384 | 56F3H2B | Broad | 2.0 | Hemp | 4.5 | Bitterness | 0.5 |
| | | Spectrum | | Seed Oil | | Blocker, | |
| | | Distillate | | | | Natural, | |
| | | CBN | 1.0 | | | Type A - | |
| | | Isolate | | MCT | 0.5 | Allen | |
| | | | | | | Flavors | |
| | | | | | | Inc. | |
| 385 | 56H6A | Broad | 2.0 | MCT | 5.0 | | |
| | | Spectrum | | | | | |
| | | Distillate | | | | | |
| | | CBG | 1.0 | | | | |
| | | Distillate | | | | | |
| 386 | 64C9 | Broad | 7.0 | MCT | 10.0 | | |
| | | Spectrum | | | | | |
| | | Distillate | | | | | |
| | | CBG | 3.0 | | | | |
| | | Distillate | | | | | |
| 387 | 10A2C5A1E2 | CBD | 2.3 | MCT | 7.8 | Bitterness | 0.1 |
| | | Isolate | | | | Blocker, | |
| | | CBG | 0.2 | | | Natural, | |
| | | Distillate | | | | Type C - | |
| | | CBN | 0.2 | | | Allen | |
| | | Isolate | | | | Flavors | |
| | | | | | | Inc. | |
| 388 | 64D6 | Broad | 5.0 | MCT | 15.0 | | |
| | | Spectrum | | | | | |
| | | Distillate | | | | | |
| | | CBD | 5.0 | | | | |
| | | Isolate | | | | | |
| 389 | 20E4 | Broad | 1.0 | MCT | 1.0 | Orange Oil | 0.4 |
| | | Spectrum | | | | Flavor - | |
| | | Distillate | | | | Flavor | |
| | | | | | | Insights | |
| 390 | 20E6 | Broad | 1.0 | Refined | 1.0 | | |
| | | Spectrum | | Olive Oil | | | |
| | | Distillate | | | | | |
| 391 | 43C | Broad | 1.0 | Refined | 1.0 | | |
| | | Spectrum | | Olive Oil | | | |
| | | Distillate | | | | | |
| 392 | 20H | Broad | 1.0 | Canola Oil | 1.0 | | |
| | | Spectrum | | | | | |
| | | Distillate | | | | | |
| 393 | 17B | Broad | 1.0 | MCT | 2.0 | | |
| | | Spectrum | | | | | |
| | | Distillate | | | | | |
| 394 | 7A | Broad | 3.0 | MCT | 3.0 | | |
| | | Spectrum | | | | | |
| | | Distillate | | | | | |
| 395 | 61A | Broad | 4.0 | MCT | 6.0 | | |
| | | Spectrum | | | | | |
| | | Distillate | | | | | |
| 396 | 25G5C3D | Broad | 4.0 | MCT | 6.0 | | |
| | | Spectrum | | | | | |
| | | Distillate | | | | | |
| 397 | 25G5C6A2 | Broad | 4.0 | MCT | 8.0 | | |
| | | Spectrum | | | | | |
| | | Distillate | | | | | |
| 398 | 61 | Broad | 4.0 | MCT | 6.0 | | |
| | | Spectrum | | | | | |
| | | Distillate | | | | | |
| 399 | 43E | Broad | 3.0 | MCT | 3.0 | | |
| | | Spectrum | | | | | |
| | | Distillate | | | | | |
| 400 | 18B | Broad | 1.0 | MCT | 1.0 | | |
| | | Spectrum | | | | | |
| | | Distillate | | | | | |
| 401 | 25G5C2A1-2 | Broad | 4.0 | MCT | 10.0 | | |
| | | Spectrum | | | | | |
| | | Distillate | | | | | |
| 402 | 25G5C3B2A | Broad | 9.0 | MCT | 12.0 | | |
| | | Spectrum | | | | | |
| | | Distillate | | | | | |
| 403 | 25G51 | Broad | 1.0 | MCT | 1.5 | | |
| | | Spectrum | | | | | |
| | | Distillate | | | | | |
| 404 | 10A1 | Broad | 4.0 | MCT | 4.0 | | |
| | | Spectrum | | | | | |
| | | Distillate | | | | | |
| 405 | 10A4 | Broad | 5.0 | MCT | 10.0 | | |
| | | Spectrum | | | | | |
| | | Distillate | | | | | |
| 406 | 10C1 | Broad | 2.0 | MCT | 6.0 | | |
| | | Spectrum | | | | | |
| | | Distillate | | | | | |
| 407 | 10A2C5A5D1 | Broad | 8.0 | MCT | 30.0 | Bitterness | 1.4 |
| | | Spectrum | | | | Blocker, | |
| | | Distillate | | | | Natural, | |
| | | | | | | Type C - | |
| | | | | | | Allen | |
| | | | | | | Flavors | |
| | | | | | | Inc. | |
| 408 | 10A2C5D1 | Broad | 5.0 | MCT | 25.0 | | |
| | | Spectrum | | | | | |
| | | Distillate | | | | | |
| 409 | 64C | Broad | 10.0 | MCT | 10.0 | | |
| | | Spectrum | | | | | |
| | | Distillate | | | | | |
| 410 | 64C1 | Broad | 5.0 | MCT | 15.0 | | |
| | | Spectrum | | | | | |
| | | Distillate | | | | | |
| 411 | 64C2 | Broad | 5.0 | MCT | 15.0 | | |
| | | Spectrum | | | | | |
| | | Distillate | | | | | |
| 412 | 64C3 | Broad | 5.0 | MCT | 20.0 | | |
| | | Spectrum | | | | | |
| | | Distillate | | | | | |
| 413 | 64C4 | Broad | 5.0 | MCT | 20.0 | | |
| | | Spectrum | | | | | |
| | | Distillate | | | | | |
| 414 | 64C5 | Broad | 5.0 | MCT | 20.0 | | |
| | | Spectrum | | | | | |
| | | Distillate | | | | | |
| 415 | 64C6 | Broad | 5.0 | MCT | 20.0 | | |
| | | Spectrum | | | | | |
| | | Distillate | | | | | |
| 416 | 64C7 | Broad | 5.0 | MCT | 20.0 | | |
| | | Spectrum | | | | | |
| | | Distillate | | | | | |
| 417 | 56F3H1A | Broad | 4.0 | MCT | 6.7 | Bitterness | 0.7 |
| | | Spectrum | | | | Blocker, | |
| | | Distillate | | | | Natural, | |
| | | | | | | Type A - | |
| | | | | | | Allen | |
| | | | | | | Flavors | |
| | | | | | | Inc. | |
| 418 | 25G5C2-1 | Full | 4.0 | MCT | 10.0 | | |
| | | Spectrum | | | | | |
| | | Distillate | | | | | |
| 419 | 10F1-CFH | Full | 7.5 | MCT | 7.5 | | |
| | | Spectrum | | | | | |
| | | Distillate | | | | | |
| | | (CFH) | | | | | |
| 420 | 10F11B | Full | 5.0 | MCT | 10.0 | | |
| | | Spectrum | | | | | |
| | | Distillate | | | | | |
| 421 | 10A2C6 | Ful | 5.0 | MCT | 12.0 | | |
| | | Spectrum | | | | | |
| | | Distillate | | | | | |
| 422 | 10A2C5A | Full | 5.0 | MCT | 16.0 | | |
| | | Spectrum | | | | | |
| | | Distillate | | | | | |
| 423 | 10 | Full | 1.0 | MCT | 1.0 | | |
| | | Spectrum | | | | | |
| | | Distillate | | | | | |
| 424 | 25S | Full | 1.0 | MCT | 1.0 | | |
| | | Spectrum | | | | | |
| | | Distillate | | | | | |
| 425 | 1X | Full | 0.8 | MCT | 0.8 | | |
| | | Spectrum | | | | | |
| | | Distillate | | | | | |
| 426 | 19B | Broad | 1.0 | MCT | 1.0 | | |
| | | Spectrum | | | | | |
| | | Distillate | | | | | |
| 427 | 20C | Broad | 1.0 | MCT | 1.0 | | |
| | | Spectrum | | | | | |
| | | Distillate | | | | | |
| 428 | 25N | CBD | 3.0 | MCT | 4.0 | | |
| | | Isolate | | | | | |
| 429 | 25G2B | CBD | 2.0 | MCT | 3.0 | Bitterness | 0.1 |
| | | Isolate | | | | Blocker, | |
| | | | | | | Natural, | |
| | | | | | | Type A - | |
| | | | | | | Allen | |
| | | | | | | Flavors | |
| | | | | | | Inc. | |
| 430 | 25G2A | CBD | 2.0 | MCT | 3.0 | | |
| | | Isolate | | | | | |
| 431 | 25M | Broad | 1.0 | Refined | 1.0 | | |
| | | Spectrum | | Olive Oil | | | |
| | | Distillate | | | | | |
| 432 | 9G | Broad | 4.0 | MCT | 3.0 | | |
| | | Spectrum | | | | | |
| | | Distillate | | | | | |
| 433 | 20D | Broad | 1.0 | MCT | 1.0 | | |
| | | Spectrum | | | | | |
| | | Distillate | | | | | |
| 434 | 25G3 | Broad | 2.0 | Refined | 2.0 | | |
| | | Spectrum | | Olive Oil | | | |
| | | Distillate | | | | | |
| 435 | 25M | Broad | 1.0 | Refined | 1.0 | | |
| | | Spectrum | | Olive Oil | | | |
| | | Distillate | | | | | |
| 436 | 50 | Full | 1.0 | MCT | 1.0 | | |
| | | Spectrum | | | | | |
| | | Distillate | | | | | |

TABLE S007B
the hydrophilic phase compositions used in the emulsions corresponding to examples 264-436 above.

| | Primary | Usage | Secondary | Usage | Tertiary | Usage | | Usage | Co- | Usage | Continuous | Usage | Water |
|---|---|---|---|---|---|---|---|---|---|---|---|---|---|
| Example | Emulsifier | Rate | Emulsifier | Rate | Emulsifier | Rate | Encapsulant | Rate | Solvent | Rate | Phase | Rate | Activity |
| 264 | Sunflower | 3.0 | | | | | | | | | Glycerin | 92.0 | 0.083 |
| | Lecithin, ≥99% - | | | | | | | | | | | | |
| | Compass Foods | | | | | | | | | | | | |
| | Pte Ltd | | | | | | | | | | | | |
| 265 | Sunflower | 4.2 | | | | | | | | | Glycerin | 84.9 | 0.079 |
| | Lecithin, ≥99% - | | | | | | | | | | | | |
| | Compass Foods | | | | | | | | | | | | |
| | Pte Ltd | | | | | | | | | | | | |
| 266 | Quillaja Saponaria | 4.0 | | | | | | | | | Glycerin | 93.0 | |
| | Extract - Ingredion | | | | | | | | | | | | |
| | Inc. | | | | | | | | | | | | |
| 267 | Quillaja Saponaria | 20.0 | | | | | | | | | Glycerin | 76.0 | |
| | Extract - Ingredion | | | | | | | | | | | | |
| | Inc. | | | | | | | | | | | | |
| 268 | Quillaja Saponaria | 16.0 | | | | | | | | | Glycerin | 80.0 | |
| | Extract - Ingredion | | | | | | | | | | | | |
| | Inc. | | | | | | | | | | | | |
| 269 | Quillaja Saponaria | 3.0 | | | | | | | | | Glycerin | 91.0 | |
| | Extract - Naturex | | | | | | | | | | | | |
| 270 | Quillaja Saponaria | 9.1 | | | | | | | | | Glycerin | 87.9 | |
| | Extract - Naturex | | | | | | | | | | | | |
| 271 | Modified Food | 10.0 | | | | | | | Distilled | 15.0 | Glycerin | 50.0 | |
| | Starch from | | | | | | | | Water | | | | |
| | Waxy Maize - | | | | | | | | | | | | |
| | Ingredion Inc. | | | | | | | | | | | | |
| 272 | Modified Food | 12.5 | | | | | | | Distilled | 15.0 | Glycerin | 47.5 | 0.585 |
| | Starch from | | | | | | | | Water | | | | |
| | Waxy Maize - | | | | | | | | | | | | |
| | Ingredion Inc. | | | | | | | | | | | | |
| 273 | Modified Food | 7.5 | | | | | | | Distilled | 15.0 | Glycerin | 52.5 | |
| | Starch from | | | | | | | | Water | | | | |
| | Waxy Maize - | | | | | | | | | | | | |
| | Ingredion Inc. | | | | | | | | | | | | |
| 274 | Modified Food | 10.0 | | | | | | | Distilled | 15.0 | Glycerin | 55.0 | |
| | Starch from | | | | | | | | Water | | | | |
| | Tapioca - | | | | | | | | | | | | |
| | Ingredion Inc. | | | | | | | | | | | | |
| 275 | Gum Acacia - | 6.0 | | | | | | | | | Glycerin | 88.0 | |
| | Ingredion Inc. | | | | | | | | | | | | |
| 276 | Gum Acacia - | 12.0 | | | | | | | | | Glycerin | 80.0 | |
| | Ingredion Inc. | | | | | | | | | | | | |
| 277 | Gum Acacia - | 6.0 | | | | | | | Distilled | 13.2 | Glycerin | 74.8 | |
| | Ingredion Inc. | | | | | | | | Water | | | | |
| 278 | Sunflower | 4.2 | | | | | | | | | Glycerin | 86.5 | |
| | Lecithin, ≥99% - | | | | | | | | | | | | |
| | Compass Foods | | | | | | | | | | | | |
| | Pte Ltd | | | | | | | | | | | | |
| 279 | Sunflower | 4.2 | | | | | | | | | Glycerin | 86.3 | |
| | Lecithin, ≥99% - | | | | | | | | | | | | |
| | Compass Foods | | | | | | | | | | | | |
| | Pte Ltd | | | | | | | | | | | | |
| 280 | Modified Food | 10.0 | | | | | | | Distilled | 15.0 | Glycerin | 55.0 | |
| | Starch from | | | | | | | | Water | | | | |
| | Tapioca - | | | | | | | | | | | | |
| | Ingredion Inc. | | | | | | | | | | | | |
| 281 | Modified Food | 10.0 | | | | | | | Distilled | 15.0 | Glycerin | 50.0 | |
| | Starch from | | | | | | | | Water | | | | |
| | Tapioca - | | | | | | | | | | | | |
| | Ingredion Inc. | | | | | | | | | | | | |
| 282 | Modified Food | 10.0 | | | | | | | Distilled | 15.0 | Glycerin | 50.0 | |
| | Starch from | | | | | | | | Water | | | | |
| | Waxy Maize - | | | | | | | | | | | | |
| | Ingredion Inc. | | | | | | | | | | | | |
| 283 | Modified Food | 10.0 | | | | | | | Distilled | 15.0 | Glycerin | 50.0 | |
| | Starch from | | | | | | | | Water | | | | |
| | Waxy Maize - | | | | | | | | | | | | |
| | Ingredion Inc. | | | | | | | | | | | | |
| 284 | Modified Food | 12.5 | | | | | | | Distilled | 15.0 | Glycerin | 47.5 | |
| | Starch from | | | | | | | | Water | | | | |
| | Waxy Maize - | | | | | | | | | | | | |
| | Ingredion Inc. | | | | | | | | | | | | |
| 285 | Modified Food | 7.5 | | | | | | | Distilled | 15.0 | Glycerin | 52.5 | |
| | Starch from | | | | | | | | Water | | | | |
| | Waxy Maize - | | | | | | | | | | | | |
| | Ingredion Inc. | | | | | | | | | | | | |
| 286 | Modified Food | 15.0 | | | | | | | Distilled | 15.0 | Glycerin | 45.0 | |
| | Starch from | | | | | | | | Water | | | | |
| | Waxy Maize - | | | | | | | | | | | | |
| | Ingredion Inc. | | | | | | | | | | | | |
| 287 | Modified Food | 15.0 | | | | | | | Distilled | 15.0 | Glycerin | 45.0 | |
| | Starch from | | | | | | | | Water | | | | |
| | Waxy Maize - | | | | | | | | | | | | |
| | Ingredion Inc. | | | | | | | | | | | | |
| 288 | Soybean | 4.0 | | | | | | | | | Glycerin | 90.0 | |
| | Phospholipids | | | | | | | | | | | | |
| | with 70% | | | | | | | | | | | | |
| | Phosphatidylcholine - | | | | | | | | | | | | |
| | Lipoid | | | | | | | | | | | | |
| 289 | Fat-free | 1.0 | | | | | | | | | Glycerin | 97.0 | 0.086 |
| | Soybean | | | | | | | | | | | | |
| | Lecithin | | | | | | | | | | | | |
| | with 45% | | | | | | | | | | | | |
| | Phosphatidylcholine - | | | | | | | | | | | | |
| | Lipoid | | | | | | | | | | | | |
| 290 | Enzyme Modified | 2.0 | | | | | | | | | Glycerin | 95.5 | |
| | Soy Lecithin - | | | | | | | | | | | | |
| | American Lecithin | | | | | | | | | | | | |
| | Company | | | | | | | | | | | | |
| 291 | Enzyme Modified | 2.0 | | | | | | | | | Glycerin | 95.5 | |
| | Soy Lecithin - | | | | | | | | | | | | |
| | American Lecithin | | | | | | | | | | | | |
| | Company | | | | | | | | | | | | |
| 292 | Phosphatidylcholine | 6.0 | | | | | | | | | Glycerin | 92.0 | |
| | from soybean, | | | | | | | | | | | | |
| | D,L-α-tocopherol | | | | | | | | | | | | |
| | (n.m.t. 0.3%) - | | | | | | | | | | | | |
| | Lipoid | | | | | | | | | | | | |
| 293 | Quillaja Saponaria | 30.0 | | | | | | | | | Glycerin | 68.0 | 0.577 |
| | Extract - Ingredion | | | | | | | | | | | | |
| | Inc. | | | | | | | | | | | | |
| 294 | Quillaja Saponaria | 19.0 | | | | | | | | | Glycerin | 79.0 | |
| | Extract - Ingredion | | | | | | | | | | | | |
| | Inc. | | | | | | | | | | | | |
| 295 | Quillaja Saponaria | 1.0 | | | | | | | | | Glycerin | 97.0 | |
| | Extract - Naturex | | | | | | | | | | | | |
| 296 | Quillaja Saponaria | 3.8 | | | | | | | | | Glycerin | 94.2 | |
| | Extract - Naturex | | | | | | | | | | | | |
| 297 | Quillaja Saponaria | 6.0 | | | | | | | | | Glycerin | 92.0 | |
| | Extract - Naturex | | | | | | | | | | | | |
| 298 | Quillaja Saponaria | 5.0 | | | | | | | | | Glycerin | 93.0 | |
| | Extract - Naturex | | | | | | | | | | | | |
| 299 | Sucrose Ester of | 1.0 | | | | | | | | | Glycerin | 97.0 | |
| | Fatty Acids, | | | | | | | | | | | | |
| | M185CD185 - | | | | | | | | | | | | |
| | Compass Foods | | | | | | | | | | | | |
| | Pte Ltd | | | | | | | | | | | | |
| 300 | Sucrose Ester of | 1.0 | | | | | | | | | Glycerin | 97.0 | |
| | Fatty Acids, | | | | | | | | | | | | |
| | M182CD182 - | | | | | | | | | | | | |
| | Compass Foods | | | | | | | | | | | | |
| | Pte Ltd | | | | | | | | | | | | |
| 301 | Sucrose Ester of | 1.0 | | | | | | | | | Glycerin | 97.0 | |
| | Fatty Acids, | | | | | | | | | | | | |
| | M175DC175 - | | | | | | | | | | | | |
| | Compass Foods | | | | | | | | | | | | |
| | Pte Ltd | | | | | | | | | | | | |
| 302 | Sucrose Acetate | 1.0 | | | | | | | | | Glycerin | 97.0 | |
| | Isobutryate | | | | | | | | | | | | |
| 303 | Modified Gum | 2.0 | | | | | | | | | Propylene | 96.0 | |
| | Acacia - | | | | | | | | | | Glycol | | |
| | Ingredion Inc. | | | | | | | | | | | | |
| 304 | Pre-hydrated | 1.0 | | | | | | | | | Propylene | 97.0 | |
| | Gum Arabic | | | | | | | | | | Glycol | | |
| 305 | Modified Food | 10.0 | | | | | | | Distilled | 15.0 | Glycerin | 55.0 | |
| | Starch from | | | | | | | | Water | | | | |
| | Tapioca - | | | | | | | | | | | | |
| | Ingredion Inc. | | | | | | | | | | | | |
| 306 | Gum Acacia - | 10.0 | | | | | | | Distilled | 15.0 | Glycerin | 50.0 | |
| | Ingredion Inc. | | | | | | | | Water | | | | |
| 307 | Enzyme Modified | 2.0 | | | | | | | Ethanol | 0.5 | Glycerin | 95.5 | |
| | Soy Lecithin - | | | | | | | | | | | | |
| | American Lecithin | | | | | | | | | | | | |
| | Company | | | | | | | | | | | | |
| 308 | Apple Pectin - | 2.0 | | | | | | | | | Propylene | 96.0 | |
| | Ingredion Inc. | | | | | | | | | | Glycol | | |
| 309 | Pectin - | 2.0 | | | | | | | | | Propylene | 96.0 | |
| | Ingredion Inc. | | | | | | | | | | Glycol | | |
| 310 | De-oiled | 0.5 | | | | | | | Ethanol | 0.5 | Glycerin | 97.0 | |
| | Powdered Soy | | | | | | | | | | | | |
| | Lecithin - | | | | | | | | | | | | |
| | American Lecithin | | | | | | | | | | | | |
| | Company | | | | | | | | | | | | |
| 311 | Lysophosphatidylcholine | 0.5 | | | | | | | Ethanol | 10.0 | Glycerin | 87.5 | |
| | from soybean | | | | | | | | | | | | |
| | lecithin (non-GMO), | | | | | | | | | | | | |
| | 80% - Lipoid | | | | | | | | | | | | |
| 312 | De-oiled | 1.0 | | | | | | | Glycerin | 10.0 | Propylene | 87.0 | |
| | Powdered Soy | | | | | | | | | | Glycol | | |
| | Lecithin - | | | | | | | | | | | | |
| | American Lecithin | | | | | | | | | | | | |
| | Company | | | | | | | | | | | | |
| 313 | De-oiled | 1.0 | | | | | | | Glycerin | 47.0 | Propylene | 50.0 | |
| | Powdered Soy | | | | | | | | | | Glycol | | |
| | Lecithin - | | | | | | | | | | | | |
| | American Lecithin | | | | | | | | | | | | |
| | Company | | | | | | | | | | | | |
| 314 | Lysophosphatidylcholine | 3.0 | | | | | | | | | Glycerin | 91.0 | |
| | from soybean | | | | | | | | | | | | |
| | lecithin (non-GMO), | | | | | | | | | | | | |
| | 80% - Lipoid | | | | | | | | | | | | |
| 315 | Fat-free Soybean | 3.0 | | | | | | | | | Glycerin | 93.0 | |
| | Lecithin with 45% | | | | | | | | | | | | |
| | phosphatidylcholine - | | | | | | | | | | | | |
| | Lipoid | | | | | | | | | | | | |
| 316 | Lecithin with | 4.0 | | | | | | | | | Glycerin | 92.0 | |
| | Ethoxylated | | | | | | | | | | | | |
| | mono di- | | | | | | | | | | | | |
| | glycerides - Solae | | | | | | | | | | | | |
| 317 | Lecithin with | 4.0 | | | | | | | | | Glycerin | 92.0 | |
| | Ethoxylated | | | | | | | | | | | | |
| | mono di- | | | | | | | | | | | | |
| | glycerides - Solae | | | | | | | | | | | | |
| 318 | Lecithin with | 6.0 | | | | | | | | | Glycerin | 88.0 | |
| | Ethoxylated | | | | | | | | | | | | |
| | mono di- | | | | | | | | | | | | |
| | glycerides - Solae | | | | | | | | | | | | |
| 319 | Lysophosphatidylcholine | 1.0 | | | | | | | | | Glycerin | 97.0 | |
| | from soybean | | | | | | | | | | | | |
| | lecithin (non-GMO), | | | | | | | | | | | | |
| | 80% - Lipoid | | | | | | | | | | | | |
| 320 | Acetylated | 4.0 | | | | | | | | | Glycerin | 94.0 | |
| | Lecithins - | | | | | | | | | | | | |
| | Cargill Inc. | | | | | | | | | | | | |
| 321 | Enzyme Modified | 3.0 | | | | | | | | | Glycerin | 95.0 | |
| | Soy Lecithin - | | | | | | | | | | | | |
| | American Lecithin | | | | | | | | | | | | |
| | Company | | | | | | | | | | | | |
| 322 | De-oiled Powdered | 2.0 | | | | | | | | | Glycerin | 94.0 | |
| | Soy Lecithin - | | | | | | | | | | | | |
| | American Lecithin | | | | | | | | | | | | |
| | Company | | | | | | | | | | | | |
| 323 | Phosphatidylcholine | 2.0 | | | | | | | | | Glycerin | 96.0 | |
| | from soybean, | | | | | | | | | | | | |
| | D,L-α-tocopherol | | | | | | | | | | | | |
| | (n.m.t. 0.3%) - | | | | | | | | | | | | |
| | Lipoid | | | | | | | | | | | | |
| 324 | Fatfree Sunflower | 1.0 | | | | | | | | | Glycerin | 97.0 | |
| | Lecithin (non-GMO) | | | | | | | | | | | | |
| | with 60% | | | | | | | | | | | | |
| | Phosphatidylcholine - | | | | | | | | | | | | |
| | Lipoid | | | | | | | | | | | | |
| 325 | Quillaja Saponaria | 12.0 | | | | | | | | | Glycerin | 86.0 | |
| | Extract - | | | | | | | | | | | | |
| | Ingredion Inc. | | | | | | | | | | | | |
| 326 | Quillaja Saponaria | 1.0 | | | | | | | | | Glycerin | 97.0 | |
| | Extract - | | | | | | | | | | | | |
| | Ingredion Inc. | | | | | | | | | | | | |
| 327 | Quillaja Saponaria | 1.0 | | | | | | | Distilled | 10.0 | 42 DE | 87.0 | |
| | Extract - | | | | | | | | Water | | Corn | | |
| | Ingredion Inc. | | | | | | | | | | Syrup | | |
| 328 | Sucrose Monoester | 1.5 | | | | | | | | | Glycerin | 92.5 | |
| | of Palmitic Acid - | | | | | | | | | | | | |
| | Compass Foods | | | | | | | | | | | | |
| | Pte Ltd | | | | | | | | | | | | |
| 329 | Sucrose Monolaureate | 4.0 | | | | | | | | | Glycerin | 94.0 | |
| | L90 | | | | | | | | | | | | |
| 330 | Sucrose Monolaureate | 1.0 | | | | | | | | | Glycerin | 97.0 | |
| | L90 | | | | | | | | | | | | |
| 331 | JBR Rhamnolipid | 1.0 | | | | | | | | | Glycerin | 97.0 | |
| 332 | R90 Rhamnolipid | 0.5 | | | | | | | | | Glycerin | 97.5 | |
| 333 | BioToLife | 0.5 | | | | | | | | | Glycerin | 97.5 | |
| | Sophorolipid | | | | | | | | | | | | |
| 334 | BioToLife | 4.0 | | | | | | | | | Glycerin | 94.0 | |
| | Sophorolipid | | | | | | | | | | | | |
| 335 | “Soy Lecithin | 1.0 | | | | | | | | | Glycerin | 97.0 | |
| | (with Ethoxylated | | | | | | | | | | | | |
| | Mono-diglycerides | | | | | | | | | | | | |
| | and Propylene | | | | | | | | | | | | |
| | Glycol) - Americal | | | | | | | | | | | | |
| | Lecithin Company” | | | | | | | | | | | | |
| 336 | “Soy Lecithin | 3.0 | | | | | | | | | Glycerin | 95.0 | |
| | (with Ethoxylated | | | | | | | | | | | | |
| | Mono-diglycerides | | | | | | | | | | | | |
| | and Propylene | | | | | | | | | | | | |
| | Glycol) - Americal | | | | | | | | | | | | |
| | Lecithin Company” | | | | | | | | | | | | |
| 337 | Modified Food | 10.0 | | | | | | | Distilled | 15.0 | Glycerin | 50.0 | |
| | Starch from | | | | | | | | Water | | | | |
| | Waxy Maize - | | | | | | | | | | | | |
| | Ingredion Inc. | | | | | | | | | | | | |
| 338 | Sunflower | 2.0 | Sucrose | 1.0 | | | | | | | Glycerin | 95.5 | |
| | Lecithin, ≥99% - | | Monoester of | | | | | | | | | | |
| | Compass Foods | | Palmitic Acid - | | | | | | | | | | |
| | Pte Ltd | | Compass Foods | | | | | | | | | | |
| | | | Pte Ltd | | | | | | | | | | |
| 339 | Sunflower | 4.0 | Sucrose | 2.0 | | | | | | | Glycerin | 88.3 | |
| | Lecithin, ≥99% - | | Monoester | | | | | | | | | | |
| | Compass Foods | | of Palmitic | | | | | | | | | | |
| | Pte Ltd | | Acid - | | | | | | | | | | |
| | | | Compass | | | | | | | | | | |
| | | | Foods Pte | | | | | | | | | | |
| | | | Ltd | | | | | | | | | | |
| 340 | Sunflower | 4.2 | Sucrose | 1.0 | | | | | | | Glycerin | 74.2 | |
| | Lecithin, ≥99% - | | Monoester | | | | | | | | | | |
| | Compass Foods | | of | | | | | | | | | | |
| | Pte Ltd | | Palmitic | | | | | | | | | | |
| | | | Acid - | | | | | | | | | | |
| | | | Compass | | | | | | | | | | |
| | | | Foods Pte | | | | | | | | | | |
| | | | Ltd | | | | | | | | | | |
| 341 | Sunflower | 4.2 | Sucrose | 1.0 | | | | | | | Glycerin | 73.7 | |
| | Lecithin, ≥99% - | | Monoester | | | | | | | | | | |
| | Compass Foods | | of | | | | | | | | | | |
| | Pte Ltd | | Palmitic | | | | | | | | | | |
| | | | Acid - | | | | | | | | | | |
| | | | Compass | | | | | | | | | | |
| | | | Foods Pte | | | | | | | | | | |
| | | | Ltd | | | | | | | | | | |
| 342 | Sunflower | 4.2 | Sucrose | 1.0 | | | | | | | Glycerin | 73.7 | |
| | Lecithin, ≥99% - | | Monoester | | | | | | | | | | |
| | Compass Foods | | of | | | | | | | | | | |
| | Pte Ltd | | Palmitic | | | | | | | | | | |
| | | | Acid - | | | | | | | | | | |
| | | | Compass | | | | | | | | | | |
| | | | Foods Pte | | | | | | | | | | |
| | | | Ltd | | | | | | | | | | |
| 343 | Sunflower | 4.2 | Sucrose | 1.0 | | | | | | | Glycerin | 73.7 | |
| | Lecithin, ≥99% - | | Monoester | | | | | | | | | | |
| | Compass Foods | | of | | | | | | | | | | |
| | Pte Ltd | | Palmitic | | | | | | | | | | |
| | | | Acid - | | | | | | | | | | |
| | | | Compass | | | | | | | | | | |
| | | | Foods Pte | | | | | | | | | | |
| | | | Ltd | | | | | | | | | | |
| 344 | Sunflower | 4.0 | Sucrose | 2.0 | | | | | | | Glycerin | 90.5 | |
| | Lecithin, ≥99% - | | Monoester | | | | | | | | | | |
| | Compass Foods | | of | | | | | | | | | | |
| | Pte Ltd | | Palmitic | | | | | | | | | | |
| | | | Acid - | | | | | | | | | | |
| | | | Compass | | | | | | | | | | |
| | | | Foods Pte | | | | | | | | | | |
| | | | Ltd | | | | | | | | | | |
| 345 | Sunflower | 2.0 | Sucrose | 1.5 | | | | | Distilled | 12.7 | Glycerin | 71.8 | |
| | Lecithin, ≥99% - | | Monoester | | | | | | Water | | | | |
| | Compass Foods | | of | | | | | | | | | | |
| | Pte Ltd | | Palmitic | | | | | | | | | | |
| | | | Acid - | | | | | | | | | | |
| | | | Compass | | | | | | | | | | |
| | | | Foods Pte | | | | | | | | | | |
| | | | Ltd | | | | | | | | | | |
| 346 | Sunflower | 2.0 | Sucrose | 1.0 | | | | | | | Glycerin | 94.5 | |
| | Lecithin, ≥99% - | | Monoester | | | | | | | | | | |
| | Compass Foods | | of | | | | | | | | | | |
| | Pte Ltd | | Palmitic | | | | | | | | | | |
| | | | Acid - | | | | | | | | | | |
| | | | Compass | | | | | | | | | | |
| | | | Foods Pte | | | | | | | | | | |
| | | | Ltd | | | | | | | | | | |
| 347 | Sunflower | 2.0 | Sucrose | 1.0 | | | | | | | Glycerin | 94.5 | |
| | Lecithin, ≥99% - | | Monoester | | | | | | | | | | |
| | Compass Foods | | of | | | | | | | | | | |
| | Pte Ltd | | Palmitic | | | | | | | | | | |
| | | | Acid - | | | | | | | | | | |
| | | | Compass | | | | | | | | | | |
| | | | Foods Pte | | | | | | | | | | |
| | | | Ltd | | | | | | | | | | |
| 348 | Sunflower | 2.0 | Sucrose | 1.0 | | | | | | | Glycerin | 94.5 | |
| | Lecithin, ≥99% - | | Monoester | | | | | | | | | | |
| | Compass Foods | | of | | | | | | | | | | |
| | Pte Ltd | | Palmitic | | | | | | | | | | |
| | | | Acid - | | | | | | | | | | |
| | | | Compass | | | | | | | | | | |
| | | | Foods Pte | | | | | | | | | | |
| | | | Ltd | | | | | | | | | | |
| 349 | Sunflower | 2.0 | Sucrose | 1.0 | | | | | | | Glycerin | 94.5 | |
| | Lecithin, ≥99% - | | Monoester | | | | | | | | | | |
| | Compass Foods | | of | | | | | | | | | | |
| | Pte Ltd | | Palmitic | | | | | | | | | | |
| | | | Acid | | | | | | | | | | |
| | | | Compass | | | | | | | | | | |
| | | | Foods Pte | | | | | | | | | | |
| | | | Ltd | | | | | | | | | | |
| 350 | Sunflower | 2.0 | Sucrose | 1.0 | | | | | | | Glycerin | 94.5 | |
| | Lecithin, ≥99% - | | Monoester | | | | | | | | | | |
| | Compass Foods | | of | | | | | | | | | | |
| | Pte Ltd | | Palmitic | | | | | | | | | | |
| | | | Acid - | | | | | | | | | | |
| | | | Compass | | | | | | | | | | |
| | | | Foods Pte | | | | | | | | | | |
| | | | Ltd | | | | | | | | | | |
| 351 | Sunflower | 2.0 | Sucrose | 1.0 | | | | | | | Glycerin | 94.5 | |
| | Lecithin, ≥99% - | | Monoester | | | | | | | | | | |
| | Compass Foods | | of | | | | | | | | | | |
| | Pte Ltd | | Palmitic | | | | | | | | | | |
| | | | Acid - | | | | | | | | | | |
| | | | Compass | | | | | | | | | | |
| | | | Foods Pte | | | | | | | | | | |
| | | | Ltd | | | | | | | | | | |
| 352 | Sunflower | 2.0 | Sucrose | 1.0 | | | | | | | Glycerin | 94.5 | |
| | Lecithin, ≥99% - | | Monoester | | | | | | | | | | |
| | Compass Foods | | of Palmitic | | | | | | | | | | |
| | Pte Ltd | | Acid - | | | | | | | | | | |
| | | | Compass | | | | | | | | | | |
| | | | Foods Pte | | | | | | | | | | |
| | | | Ltd | | | | | | | | | | |
| 353 | Sunflower | 2.0 | Sucrose | 1.0 | | | | | | | Glycerin | 94.5 | |
| | Lecithin, ≥99% - | | Monoester | | | | | | | | | | |
| | Compass Foods | | of | | | | | | | | | | |
| | Pte Ltd | | Palmitic | | | | | | | | | | |
| | | | Acid - | | | | | | | | | | |
| | | | Compass | | | | | | | | | | |
| | | | Foods Pte | | | | | | | | | | |
| | | | Ltd | | | | | | | | | | |
| 354 | Sunflower | 2.0 | Sucrose | 1.0 | | | | | | | Propylene | 94.5 | |
| | Lecithin, ≥99% - | | Monoester | | | | | | | | Glycol | | |
| | Compass Foods | | of | | | | | | | | | | |
| | Pte Ltd | | Palmitic | | | | | | | | | | |
| | | | Acid - | | | | | | | | | | |
| | | | Compass | | | | | | | | | | |
| | | | Foods Pte | | | | | | | | | | |
| | | | Ltd | | | | | | | | | | |
| 355 | Quillaja Saponaria | 5.0 | Sucrose | 2.5 | | | | | | | Glycerin | 75.5 | |
| | Extract - | | Monoester | | | | | | | | | | |
| | Ingredion Inc. | | of | | | | | | | | | | |
| | | | Palmitic | | | | | | | | | | |
| | | | Acid - | | | | | | | | | | |
| | | | Compass | | | | | | | | | | |
| | | | Foods Pte | | | | | | | | | | |
| | | | Ltd | | | | | | | | | | |
| 356 | Quillaja Saponaria | 2.5 | Sucrose | 1.3 | | | | | | | Glycerin | 86.7 | |
| | Extract - | | Monoester | | | | | | | | | | |
| | Ingredion Inc. | | of | | | | | | | | | | |
| | | | Palmitic | | | | | | | | | | |
| | | | Acid - | | | | | | | | | | |
| | | | Compass | | | | | | | | | | |
| | | | Foods Pte | | | | | | | | | | |
| | | | Ltd | | | | | | | | | | |
| 357 | Quillaja Saponaria | 5.0 | Sucrose | 2.5 | | | | | | | Glycerin | 77.5 | |
| | Extract - | | Monoester | | | | | | | | | | |
| | Ingredion Inc. | | of Palmitic | | | | | | | | | | |
| | | | Acid - | | | | | | | | | | |
| | | | Compass | | | | | | | | | | |
| | | | Foods Pte | | | | | | | | | | |
| | | | Ltd | | | | | | | | | | |
| 358 | Quillaja Saponaria | 8.0 | Sucrose | 2.5 | | | | | | | Glycerin | 58.8 | |
| | Extract - | | Monoester | | | | | | | | | | |
| | Ingredion Inc. | | of | | | | | | | | | | |
| | | | Palmitic | | | | | | | | | | |
| | | | Acid - | | | | | | | | | | |
| | | | Compass | | | | | | | | | | |
| | | | Foods Pte | | | | | | | | | | |
| | | | Ltd | | | | | | | | | | |
| 359 | Quillaja Saponaria | 2.5 | Sucrose | 1.3 | | | | | | | Glycerin | 86.7 | |
| | Extract - | | Monoester | | | | | | | | | | |
| | Ingredion Inc. | | of | | | | | | | | | | |
| | | | Palmitic | | | | | | | | | | |
| | | | Acid - | | | | | | | | | | |
| | | | Compass | | | | | | | | | | |
| | | | Foods Pte | | | | | | | | | | |
| | | | Ltd | | | | | | | | | | |
| 360 | Quillaja Saponaria | 2.5 | Sucrose | 1.3 | | | | | | | Glycerin | 86.3 | |
| | Extract - | | Monoester | | | | | | | | | | |
| | Ingredion Inc. | | of | | | | | | | | | | |
| | | | Palmitic | | | | | | | | | | |
| | | | Acid - | | | | | | | | | | |
| | | | Compass | | | | | | | | | | |
| | | | Foods Pte | | | | | | | | | | |
| | | | Ltd | | | | | | | | | | |
| 361 | Quillaja Saponaria | 2.5 | Sucrose | 1.3 | | | | | | | Glycerin | 86.6 | |
| | Extract - | | Monoester | | | | | | | | | | |
| | Ingredion Inc. | | of | | | | | | | | | | |
| | | | Palmitic | | | | | | | | | | |
| | | | Acid - | | | | | | | | | | |
| | | | Compass | | | | | | | | | | |
| | | | Foods Pte | | | | | | | | | | |
| | | | Ltd | | | | | | | | | | |
| 362 | Quillaja Saponaria | 5.0 | Sucrose | 2.5 | | | | | Distilled | 5.7 | Glycerin | 56.8 | 0.414 |
| | Extract - | | Monoester | | | | | | Water | | | | |
| | Ingredion Inc. | | of | | | | | | | | | | |
| | | | Palmitic | | | | | | | | | | |
| | | | Acid - | | | | | | | | | | |
| | | | Compass | | | | | | | | | | |
| | | | Foods Pte | | | | | | | | | | |
| | | | Ltd | | | | | | | | | | |
| 363 | Quillaja Saponaria | 4.0 | Sunflower | 4.0 | | | | | | | Glycerin | 83.0 | |
| | Extract - | | Lecithin, ≥99% - | | | | | | | | | | |
| | Ingredion Inc. | | Compass | | | | | | | | | | |
| | | | Foods Pte | | | | | | | | | | |
| | | | Ltd | | | | | | | | | | |
| 364 | Quillaja Saponaria | 8.0 | Sunflower | 4.0 | | | | | | | Glycerin | 56.2 | |
| | Extract - | | Lecithin, ≥ | | | | | | | | | | |
| | Ingredion Inc. | | 99% - | | | | | | | | | | |
| | | | Compass | | | | | | | | | | |
| | | | Foods Pte | | | | | | | | | | |
| | | | Ltd | | | | | | | | | | |
| 365 | Quillaja Saponaria | 2.5 | Sunflower | 0.5 | | | | | | | Glycerin | 82.0 | |
| | Extract - Naturex | | Lecithin, ≥99% - | | | | | | | | | | |
| | | | Compass | | | | | | | | | | |
| | | | Foods Pte | | | | | | | | | | |
| | | | Ltd | | | | | | | | | | |
| 366 | Quillaja Saponaria | 6.0 | Sunflower | 1.0 | | | | | | | Glycerin | 85.0 | |
| | Extract - Naturex | | Lecithin, ≥99% - | | | | | | | | | | |
| | | | Compass | | | | | | | | | | |
| | | | Foods Pte | | | | | | | | | | |
| | | | Ltd | | | | | | | | | | |
| 367 | Quillaja Saponaria | 6.0 | Sucrose | 1.0 | | | | | | | Glycerin | 82.9 | |
| | Extract - Naturex | | Monoester | | | | | | | | | | |
| | | | of | | | | | | | | | | |
| | | | Palmitic | | | | | | | | | | |
| | | | Acid - | | | | | | | | | | |
| | | | Compass | | | | | | | | | | |
| | | | Foods Pte | | | | | | | | | | |
| | | | Ltd | | | | | | | | | | |
| 368 | Quillaja Saponaria | 10.0 | Sucrose | 1.7 | | | | | | | Glycerin | 77.4 | |
| | Extract - Naturex | | Monoester | | | | | | | | | | |
| | | | of | | | | | | | | | | |
| | | | Palmitic | | | | | | | | | | |
| | | | Acid - | | | | | | | | | | |
| | | | Compass | | | | | | | | | | |
| | | | Foods Pte | | | | | | | | | | |
| | | | Ltd | | | | | | | | | | |
| 369 | Quillaja Saponaria | 4.0 | Sucrose | 0.7 | | | | | | | Glycerin | 89.3 | |
| | Extract - Naturex | | Monoester | | | | | | | | | | |
| | | | of | | | | | | | | | | |
| | | | Palmitic | | | | | | | | | | |
| | | | Acid - | | | | | | | | | | |
| | | | Compass | | | | | | | | | | |
| | | | Foods Pte | | | | | | | | | | |
| | | | Ltd | | | | | | | | | | |
| 370 | Quillaja Saponaria | 10.0 | Sucrose | 1.7 | | | | | | | Glycerin | 73.8 | |
| | Extract - Naturex | | Monoester | | | | | | | | | | |
| | | | of | | | | | | | | | | |
| | | | Palmitic | | | | | | | | | | |
| | | | Acid - | | | | | | | | | | |
| | | | Compass | | | | | | | | | | |
| | | | Foods Pte | | | | | | | | | | |
| | | | Ltd | | | | | | | | | | |
| 371 | Modified Food | 5.0 | Flavor- | 5.0 | | | | | Distilled | 15.0 | Glycerin | 55.0 | |
| | Starch from | | free Maltodextrin - | | | | | | Water | | | | |
| | Tapioca - | | Ingredion | | | | | | | | | | |
| | Ingredion Inc. | | Inc. | | | | | | | | | | |
| 372 | Gum Acacia - | 6.0 | Quillaja | 1.0 | | | | | | | Glycerin | 87.0 | |
| | Ingredion Inc. | | Saponaria | | | | | | | | | | |
| | | | Extract - | | | | | | | | | | |
| | | | Naturex | | | | | | | | | | |
| 373 | Sunflower | 2.0 | Sucrose | 1.0 | | | | | | | Glycerin | 94.5 | |
| | Lecithin, ≥99% - | | Monoester | | | | | | | | | | |
| | Compass Foods | | of | | | | | | | | | | |
| | Pte Ltd | | Palmitic | | | | | | | | | | |
| | | | Acid - | | | | | | | | | | |
| | | | Compass | | | | | | | | | | |
| | | | Foods Pte | | | | | | | | | | |
| | | | Ltd | | | | | | | | | | |
| 374 | Quillaja Saponaria | 0.5 | Sunflower | 1.0 | | | | | | | Glycerin | 96.7 | |
| | Extract - | | Lecithin, ≥99% - | | | | | | | | | | |
| | Ingredion Inc. | | Compass | | | | | | | | | | |
| | | | Foods Pte | | | | | | | | | | |
| | | | Ltd | | | | | | | | | | |
| 375 | Sunflower | 2.0 | Sucrose | 1.0 | | | | | | | Glycerin | 94.5 | |
| | Lecithin, ≥99% - | | Monoester | | | | | | | | | | |
| | Compass Foods | | of | | | | | | | | | | |
| | Pte Ltd | | Palmitic | | | | | | | | | | |
| | | | Acid - | | | | | | | | | | |
| | | | Compass | | | | | | | | | | |
| | | | Foods Pte | | | | | | | | | | |
| | | | Ltd | | | | | | | | | | |
| 376 | Sunflower | 2.0 | Sucrose | 1.0 | | | | | | | Glycerin | 94.5 | |
| | Lecithin, ≥99% - | | Monoester | | | | | | | | | | |
| | Compass Foods | | of | | | | | | | | | | |
| | Pte Ltd | | Palmitic | | | | | | | | | | |
| | | | Acid - | | | | | | | | | | |
| | | | Compass | | | | | | | | | | |
| | | | Foods Pte | | | | | | | | | | |
| | | | Ltd | | | | | | | | | | |
| 377 | Modified Food | 5.0 | Flavor- | 5.0 | | | | | Distilled | 15.0 | Glycerin | 60.0 | |
| | Starch from | | free | | | | | | Water | | | | |
| | Tapioca - | | Maltodextrin - | | | | | | | | | | |
| | Ingredion Inc. | | Ingredion | | | | | | | | | | |
| | | | Inc. | | | | | | | | | | |
| 378 | Modified Food | 5.0 | Flavor- | 5.0 | | | | | Distilled | 15.0 | Glycerin | 55.0 | |
| | Starch from | | free | | | | | | Water | | | | |
| | Tapioca - | | Maltodextrin - | | | | | | | | | | |
| | Ingredion Inc. | | Ingredion | | | | | | | | | | |
| | | | Inc. | | | | | | | | | | |
| 379 | Modified Food | 5.0 | Flavor- | 5.0 | | | | | Distilled | 15.0 | Glycerin | 55.0 | |
| | Starch from | | free | | | | | | Water | | | | |
| | Tapioca - | | Maltodextrin - | | | | | | | | | | |
| | Ingredion Inc. | | Ingredion | | | | | | | | | | |
| | | | Inc. | | | | | | | | | | |
| 380 | Modified Food | 10.0 | Flavor- | 5.0 | | | | | Distilled | 15.0 | Glycerin | 50.0 | |
| | Starch from | | free | | | | | | Water | | | | |
| | Tapioca - | | Maltodext | | | | | | | | | | |
| | Ingredion Inc. | | rin - | | | | | | | | | | |
| | | | Ingredion | | | | | | | | | | |
| | | | Inc. | | | | | | | | | | |
| 381 | Quillaja Saponaria | 8.0 | Sucrose | 4.0 | | | | | | | Glycerin | 48.8 | |
| | Extract - | | Monoester | | | | | | | | | | |
| | Ingredion Inc. | | of | | | | | | | | | | |
| | | | Palmitic | | | | | | | | | | |
| | | | Acid - | | | | | | | | | | |
| | | | Compass | | | | | | | | | | |
| | | | Foods Pte | | | | | | | | | | |
| | | | Ltd | | | | | | | | | | |
| 382 | Quillaja Saponaria | 6.0 | Sucrose | 1.0 | | | | | | | Glycerin | 84.5 | |
| | Extract - Naturex | | Monoester | | | | | | | | | | |
| | | | of | | | | | | | | | | |
| | | | Palmitic | | | | | | | | | | |
| | | | Acid - | | | | | | | | | | |
| | | | Compass | | | | | | | | | | |
| | | | Foods Pte | | | | | | | | | | |
| | | | Ltd | | | | | | | | | | |
| 383 | Quillaja Saponaria | 6.0 | Sucrose | 1.0 | | | | | | | Glycerin | 84.5 | |
| | Extract - Naturex | | Monoester | | | | | | | | | | |
| | | | of | | | | | | | | | | |
| | | | Palmitic | | | | | | | | | | |
| | | | Acid - | | | | | | | | | | |
| | | | Compass | | | | | | | | | | |
| | | | Foods Pte | | | | | | | | | | |
| | | | Ltd | | | | | | | | | | |
| 384 | Quillaja Saponaria | 6.0 | Sucrose | 1.0 | | | | | | | Glycerin | 84.5 | |
| | Extract - Naturex | | Monoester | | | | | | | | | | |
| | | | of | | | | | | | | | | |
| | | | Palmitic | | | | | | | | | | |
| | | | Acid - | | | | | | | | | | |
| | | | Compass | | | | | | | | | | |
| | | | Foods Pte | | | | | | | | | | |
| | | | Ltd | | | | | | | | | | |
| 385 | Quillaja Saponaria | 6.0 | Sunflower | 3.0 | | | | | | | Glycerin | 83.0 | |
| | Extract - Naturex | | Lecithin, ≥99% - | | | | | | | | | | |
| | | | Compass | | | | | | | | | | |
| | | | Foods Pte | | | | | | | | | | |
| | | | Ltd | | | | | | | | | | |
| 386 | Modified Food | 5.0 | Flavor- | 5.0 | | | | | Distilled | 15.0 | Glycerin | 55.0 | |
| | Starch from | | free | | | | | | Water | | | | |
| | Tapioca - | | Maltodextrin - | | | | | | | | | | |
| | Ingredion Inc. | | Ingredion | | | | | | | | | | |
| | | | Inc. | | | | | | | | | | |
| 387 | Quillaja Saponaria | 2.8 | Sucrose | 1.4 | | | | | | | Glycerin | 85.1 | |
| | Extract - | | Monoester | | | | | | | | | | |
| | Ingredion Inc. | | of | | | | | | | | | | |
| | | | Palmitic | | | | | | | | | | |
| | | | Acid - | | | | | | | | | | |
| | | | Compass | | | | | | | | | | |
| | | | Foods Pte | | | | | | | | | | |
| | | | Ltd | | | | | | | | | | |
| 388 | Modified Food | 10.0 | Flavor- | 1.0 | | | | | Distilled | 15.0 | Glycerin | 45.0 | |
| | Starch from | | free | | | | | | Water | | | | |
| | Waxy Maize - | | Maltodextrin - | | | | | | | | | | |
| | Ingredion Inc. | | Ingredion | | | | | | | | | | |
| | | | Inc. | | | | | | | | | | |
| 389 | Enzyme Modified | 1.0 | Fatfree | 1.0 | | | | | | | Glycerin | 95.6 | |
| | Soy Lecithin - | | Sunflower | | | | | | | | | | |
| | American Lecithin | | Lecithin | | | | | | | | | | |
| | Company | | (non- | | | | | | | | | | |
| | | | GMO) | | | | | | | | | | |
| | | | with 60% | | | | | | | | | | |
| | | | Phosphati- | | | | | | | | | | |
| | | | dylcholine - | | | | | | | | | | |
| | | | Lipoid | | | | | | | | | | |
| 390 | Enzyme Modified | 1.0 | Fatfree | 1.0 | | | | | | | Glycerin | 96.0 | |
| | Soy Lecithin - | | Sunflower | | | | | | | | | | |
| | American Lecithin | | Lecithin | | | | | | | | | | |
| | Company | | (non- | | | | | | | | | | |
| | | | GMO) | | | | | | | | | | |
| | | | with 60% | | | | | | | | | | |
| | | | Phosphati- | | | | | | | | | | |
| | | | dylcholine - | | | | | | | | | | |
| | | | Lipoid | | | | | | | | | | |
| 391 | Fat-free Soybean | 1.0 | Fatfree | 1.0 | | | | | | | Glycerin | 96.0 | |
| | Lecithin with 45% | | Sunflower | | | | | | | | | | |
| | Phosphatidylcholine - | | Lecithin | | | | | | | | | | |
| | Lipoid | | (non- | | | | | | | | | | |
| | | | GMO) | | | | | | | | | | |
| | | | with 60% | | | | | | | | | | |
| | | | Phosphati- | | | | | | | | | | |
| | | | dylcholine - | | | | | | | | | | |
| | | | Lipoid | | | | | | | | | | |
| 392 | Enzyme Modified | 1.0 | Fatfree | 1.0 | | | | | | | Glycerin | 96.0 | |
| | Soy Lecithin - | | Sunflower | | | | | | | | | | |
| | American Lecithin | | Lecithin | | | | | | | | | | |
| | Company | | (non- GMO) | | | | | | | | | | |
| | | | with 60% | | | | | | | | | | |
| | | | Phosphati- | | | | | | | | | | |
| | | | dylcholine - | | | | | | | | | | |
| | | | Lipoid | | | | | | | | | | |
| 393 | Hydroxylated | 3.0 | Fatfree | 1.0 | | | | | | | Glycerin | 93.0 | |
| | liquid soy | | Sunflower | | | | | | | | | | |
| | lecithin - | | Lecithin | | | | | | | | | | |
| | Cargill Inc. | | (non- | | | | | | | | | | |
| | | | GMO) | | | | | | | | | | |
| | | | with 60% | | | | | | | | | | |
| | | | Phosphati- | | | | | | | | | | |
| | | | dylcholine - | | | | | | | | | | |
| | | | Lipoid | | | | | | | | | | |
| 394 | De-oiled | 2.0 | Sucrose | 2.0 | | | | | | | Glycerin | 90.0 | |
| | Powdered | | Monoester | | | | | | | | | | |
| | Soy Lecithin - | | of Palmitic | | | | | | | | | | |
| | American Lecithin | | Acid - | | | | | | | | | | |
| | Company | | Compass | | | | | | | | | | |
| | | | Foods Pte | | | | | | | | | | |
| | | | Ltd | | | | | | | | | | |
| 395 | Sunflower | 2.0 | Sucrose | 1.0 | | | | | Distilled | 6.5 | Glycerin | 80.5 | |
| | Lecithin, ≥99% - | | Monoester | | | | | | Water | | | | |
| | Compass Foods | | of Palmitic | | | | | | | | | | |
| | Pte Ltd | | Acid - | | | | | | | | | | |
| | | | Compass | | | | | | | | | | |
| | | | Foods Pte | | | | | | | | | | |
| | | | Ltd | | | | | | | | | | |
| 396 | Sunflower | 3.0 | Sucrose | 1.0 | | | | | Distilled | 12.9 | Glycerin | 73.1 | |
| | Lecithin, ≥99% - | | Monoester | | | | | | Water | | | | |
| | Compass Foods | | of | | | | | | | | | | |
| | Pte Ltd | | Palmitic | | | | | | | | | | |
| | | | Acid - | | | | | | | | | | |
| | | | Compass | | | | | | | | | | |
| | | | Foods Pte | | | | | | | | | | |
| | | | Ltd | | | | | | | | | | |
| 397 | Sunflower | 2.0 | Sucrose | 1.5 | | | | | Distilled | 12.7 | Glycerin | 71.8 | |
| | Lecithin, ≥99% - | | Monoester | | | | | | Water | | | | |
| | Compass Foods | | of | | | | | | | | | | |
| | Pte Ltd | | Palmitic | | | | | | | | | | |
| | | | Acid - | | | | | | | | | | |
| | | | Compass | | | | | | | | | | |
| | | | Foods Pte | | | | | | | | | | |
| | | | Ltd | | | | | | | | | | |
| 398 | Sunflower | 2.0 | Sucrose | 1.0 | | | | | Distilled | 13.1 | Glycerin | 74.0 | |
| | Lecithin, ≥99% - | | Monoester | | | | | | Water | | | | |
| | Compass Foods | | of | | | | | | | | | | |
| | Pte Ltd | | Palmitic | | | | | | | | | | |
| | | | Acid - | | | | | | | | | | |
| | | | Compass | | | | | | | | | | |
| | | | Foods Pte | | | | | | | | | | |
| | | | Ltd | | | | | | | | | | |
| 399 | Fat-free | 1.0 | Sucrose | 1.0 | | | | | | | Glycerin | 92.0 | |
| | Soybean | | Monoester | | | | | | | | | | |
| | Lecithin | | of | | | | | | | | | | |
| | with 45% | | Palmitic | | | | | | | | | | |
| | Phosphatidylcholine - | | Acid - | | | | | | | | | | |
| | Lipoid | | Compass | | | | | | | | | | |
| | | | Foods Pte | | | | | | | | | | |
| | | | Ltd | | | | | | | | | | |
| 400 | Acetylated | 3.0 | Sucrose | 0.5 | | | | | | | Glycerin | 94.5 | |
| | Lecithins - | | Monoester | | | | | | | | | | |
| | Cargill Inc. | | of Palmitic | | | | | | | | | | |
| | | | Acid - | | | | | | | | | | |
| | | | Compass | | | | | | | | | | |
| | | | Foods Pte | | | | | | | | | | |
| | | | Ltd | | | | | | | | | | |
| 401 | Sunflower | 2.5 | Sucrose | 1.5 | | | | | Distilled | 12.3 | Glycerin | 69.7 | |
| | Lecithin, ≥99% - | | Monoester | | | | | | Water | | | | |
| | Compass Foods | | of | | | | | | | | | | |
| | Pte Ltd | | Palmitic | | | | | | | | | | |
| | | | Acid - | | | | | | | | | | |
| | | | Compass | | | | | | | | | | |
| | | | Foods Pte | | | | | | | | | | |
| | | | Ltd | | | | | | | | | | |
| 402 | Sunflower | 3.0 | Sucrose | 1.0 | | | | | | | Glycerin | 75.0 | |
| | Lecithin, ≥99% - | | Monoester | | | | | | | | | | |
| | Compass Foods | | of | | | | | | | | | | |
| | Pte Ltd | | Palmitic | | | | | | | | | | |
| | | | Acid - | | | | | | | | | | |
| | | | Compass | | | | | | | | | | |
| | | | Foods Pte | | | | | | | | | | |
| | | | Ltd | | | | | | | | | | |
| 403 | Sunflower | 2.0 | Quillaja | 1.0 | | | | | | | Glycerin | 94.5 | |
| | Lecithin, ≥99% - | | Saponaria | | | | | | | | | | |
| | Compass Foods | | Extract - | | | | | | | | | | |
| | Pte Ltd | | Ingredion | | | | | | | | | | |
| | | | Inc. | | | | | | | | | | |
| 404 | Quillaja Saponaria | 4.0 | Sucrose | 2.0 | | | | | | | Glycerin | 86.0 | |
| | Extract - | | Monoester | | | | | | | | | | |
| | Ingredion Inc. | | of | | | | | | | | | | |
| | | | Palmitic | | | | | | | | | | |
| | | | Acid - | | | | | | | | | | |
| | | | Compass | | | | | | | | | | |
| | | | Foods Pte | | | | | | | | | | |
| | | | Ltd | | | | | | | | | | |
| 405 | Quillaja Saponaria | 4.0 | Sucrose | 4.0 | | | | | | | Glycerin | 77.0 | |
| | Extract - | | Monoester | | | | | | | | | | |
| | Ingredion Inc. | | of Palmitic | | | | | | | | | | |
| | | | Acid - | | | | | | | | | | |
| | | | Compass | | | | | | | | | | |
| | | | Foods Pte | | | | | | | | | | |
| | | | Ltd | | | | | | | | | | |
| 406 | Quillaja Saponaria | 4.0 | Sucrose | 4.0 | | | | | | | Glycerin | 84.0 | |
| | Extract - | | Monoester | | | | | | | | | | |
| | Ingredion Inc. | | of | | | | | | | | | | |
| | | | Palmitic | | | | | | | | | | |
| | | | Acid - | | | | | | | | | | |
| | | | Compass | | | | | | | | | | |
| | | | Foods Pte | | | | | | | | | | |
| | | | Ltd | | | | | | | | | | |
| 407 | Quillaja Saponaria | 8.0 | Sucrose | 4.0 | | | | | | | Glycerin | 48.6 | 0.359 |
| | Extract - | | Monoester | | | | | | | | | | |
| | Ingredion Inc. | | of | | | | | | | | | | |
| | | | Palmitic | | | | | | | | | | |
| | | | Acid - | | | | | | | | | | |
| | | | Compass | | | | | | | | | | |
| | | | Foods Pte | | | | | | | | | | |
| | | | Ltd | | | | | | | | | | |
| 408 | Quillaja Saponaria | 5.0 | Sucrose | 2.5 | | | | | Distilled | 5.7 | Glycerin | 56.8 | |
| | Extract - | | Monoester | | | | | | Water | | | | |
| | Ingredion Inc. | | of | | | | | | | | | | |
| | | | Palmitic | | | | | | | | | | |
| | | | Acid - | | | | | | | | | | |
| | | | Compass | | | | | | | | | | |
| | | | Foods Pte | | | | | | | | | | |
| | | | Ltd | | | | | | | | | | |
| 409 | Flavor-free | 5.0 | Modified | 5.0 | | | | | Distilled | 15.0 | Glycerin | 55.0 | |
| | Maltodextrin - | | Food | | | | | | Water | | | | |
| | Ingredion Inc. | | Starch | | | | | | | | | | |
| | | | from | | | | | | | | | | |
| | | | Tapioca - | | | | | | | | | | |
| | | | Ingredion | | | | | | | | | | |
| | | | Inc. | | | | | | | | | | |
| 410 | Flavor-free | 5.0 | Modified | 5.0 | | | | | Distilled | 15.0 | Glycerin | 55.0 | |
| | Maltodextrin - | | Food | | | | | | Water | | | | |
| | Ingredion Inc. | | Starch from | | | | | | | | | | |
| | | | Tapioca - | | | | | | | | | | |
| | | | Ingredion | | | | | | | | | | |
| | | | Inc. | | | | | | | | | | |
| 411 | Flavor-free | 5.0 | Modified | 10.0 | | | | | Distilled | 15.0 | Glycerin | 50.0 | |
| | Maltodextrin - | | Food | | | | | | Water | | | | |
| | Ingredion Inc. | | Starch | | | | | | | | | | |
| | | | from | | | | | | | | | | |
| | | | Tapioca - | | | | | | | | | | |
| | | | Ingredion | | | | | | | | | | |
| | | | Inc. | | | | | | | | | | |
| 412 | Flavor-free | 5.0 | Modified | 10.0 | | | | | Distilled | 15.0 | Glycerin | 45.0 | |
| | Maltodextrin - | | Food | | | | | | Water | | | | |
| | Ingredion Inc. | | Starch | | | | | | | | | | |
| | | | from | | | | | | | | | | |
| | | | Tapioca - | | | | | | | | | | |
| | | | Ingredion | | | | | | | | | | |
| | | | Inc. | | | | | | | | | | |
| 413 | Flavor-free | 5.0 | Modified | 15.0 | | | | | Distilled | 1 | Glycerin | 40.0 | |
| | Maltodextrin - | | Food | | | | | | Water | 5 | | | |
| | Ingredion Inc. | | Starch | | | | | | | 0 | | | |
| | | | from | | | | | | | | | | |
| | | | Tapioca - | | | | | | | | | | |
| | | | Ingredion | | | | | | | | | | |
| | | | Inc. | | | | | | | | | | |
| 414 | Flavor-free | 10.0 | Modified | 10.0 | | | | | Distilled | 15.0 | Glycerin | 40.0 | |
| | Maltodextrin - | | Food | | | | | | Water | | | | |
| | Ingredion Inc. | | Starch | | | | | | | | | | |
| | | | from | | | | | | | | | | |
| | | | Tapioca - | | | | | | | | | | |
| | | | Ingredion | | | | | | | | | | |
| | | | Inc. | | | | | | | | | | |
| 415 | Flavor-free | 5.0 | Modified | 7.5 | | | | | Distilled | 15.0 | Glycerin | 47.5 | |
| | Maltodextrin - | | Food | | | | | | Water | | | | |
| | Ingredion Inc. | | Starch | | | | | | | | | | |
| | | | from | | | | | | | | | | |
| | | | Tapioca - | | | | | | | | | | |
| | | | Ingredion | | | | | | | | | | |
| | | | Inc. | | | | | | | | | | |
| 416 | Flavor-free | 7.5 | Modified | 7.5 | | | | | Distilled | 15.0 | Glycerin | 45.0 | |
| | Maltodextrin - | | Food | | | | | | Water | | | | |
| | Ingredion Inc. | | Starch from | | | | | | | | | | |
| | | | Tapioca - | | | | | | | | | | |
| | | | Ingredion | | | | | | | | | | |
| | | | Inc. | | | | | | | | | | |
| 417 | Quillaja Saponaria | 8.0 | Sucrose | 1.3 | | | | | | | Glycerin | 79.3 | 0.264 |
| | Extract - Naturex | | Monoester | | | | | | | | | | |
| | | | of | | | | | | | | | | |
| | | | Palmitic | | | | | | | | | | |
| | | | Acid - | | | | | | | | | | |
| | | | Compass | | | | | | | | | | |
| | | | Foods Pte | | | | | | | | | | |
| | | | Ltd | | | | | | | | | | |
| 418 | Sunflower | 2.5 | Sucrose | 1.5 | | | | | | | Glycerin | 79.3 | 0.264 |
| | Lecithin, ≥99% - | | Monoester | | | | | | | | | | |
| | Compass Foods | | of | | | | | | | | | | |
| | Pte Ltd | | Palmitic | | | | | | | | | | |
| | | | Acid - | | | | | | | | | | |
| | | | Compass | | | | | | | | | | |
| | | | Foods Pte | | | | | | | | | | |
| | | | Ltd | | | | | | | | | | |
| 419 | Quillaja Saponaria | 2.5 | Sunflower | 0.5 | | | | | | | Glycerin | 82.0 | |
| | Extract - | | Lecithin, ≥99% - | | | | | | | | | | |
| | Ingredion Inc. | | Compass | | | | | | | | | | |
| | | | Foods Pte | | | | | | | | | | |
| | | | Ltd | | | | | | | | | | |
| 420 | Quillaja Saponaria | 2.5 | Sunflower | 0.5 | | | | | | | Glycerin | 82.0 | |
| | Extract - | | Lecithin, ≥99% - | | | | | | | | | | |
| | Ingredion Inc. | | Compass | | | | | | | | | | |
| | | | Foods Pte | | | | | | | | | | |
| | | | Ltd | | | | | | | | | | |
| 421 | Quillaja Saponaria | 5.0 | Sucrose | 2.5 | | | | | | | Glycerin | 75.5 | |
| | Extract - | | Monoester | | | | | | | | | | |
| | Ingredion Inc. | | of | | | | | | | | | | |
| | | | Palmitic | | | | | | | | | | |
| | | | Acid - | | | | | | | | | | |
| | | | Compass | | | | | | | | | | |
| | | | Foods Pte | | | | | | | | | | |
| | | | Ltd | | | | | | | | | | |
| 422 | Quillaja Saponaria | 5.0 | Sucrose | 2.5 | | | | | | | Glycerin | 71.5 | |
| | Extract - | | Monoester of | | | | | | | | | | |
| | Ingredion Inc. | | Palmitic | | | | | | | | | | |
| | | | Acid - | | | | | | | | | | |
| | | | Compass | | | | | | | | | | |
| | | | Foods Pte | | | | | | | | | | |
| | | | Ltd | | | | | | | | | | |
| 423 | Quillaja Saponaria | 0.5 | Sucrose | 0.5 | | | | | | | Glycerin | 97.0 | |
| | Extract - | | Monoester | | | | | | | | | | |
| | Ingredion Inc. | | of | | | | | | | | | | |
| | | | Palmitic | | | | | | | | | | |
| | | | Acid - | | | | | | | | | | |
| | | | Compass | | | | | | | | | | |
| | | | Foods Pte | | | | | | | | | | |
| | | | Ltd | | | | | | | | | | |
| 424 | Sunflower | 1.0 | Sucrose | 0.5 | | | | | Distilled | 20.0 | Glycerin | 76.5 | |
| | Lecithin, ≥99% - | | Monoester | | | | | | Water | | | | |
| | Compass Foods | | of | | | | | | | | | | |
| | Pte Ltd | | Palmitic | | | | | | | | | | |
| | | | Acid - | | | | | | | | | | |
| | | | Compass | | | | | | | | | | |
| | | | Foods Pte | | | | | | | | | | |
| | | | Ltd | | | | | | | | | | |
| 425 | De-oiled | 0.8 | | | | | | | Distilled | 25.0 | 42 DE | 72.6 | 0.891 |
| | Powdered Soy | | | | | | | | Water | | Corn | | |
| | Lecithin - | | | | | | | | | | Syrup | | |
| | American | | | | | | | | | | | | |
| | Lecithin | | | | | | | | | | | | |
| | Company | | | | | | | | | | | | |
| 426 | “Soy Lecithin | 3.0 | Enzyme | 1.0 | | | | | | | Glycerin | 94.0 | |
| | (with Ethoxylated | | Modified | | | | | | | | | | |
| | Mono-diglycerides | | Soy | | | | | | | | | | |
| | and Propylene | | Lecithin - | | | | | | | | | | |
| | Glycol) - Americal | | American | | | | | | | | | | |
| | Lecithin Company” | | Lecithin | | | | | | | | | | |
| | | | Company | | | | | | | | | | |
| 427 | Enzyme Modified | 3.0 | “Soy | 1.0 | | | | | | | Glycerin | 94.0 | |
| | Soy Lecithin - | | Lecithin | | | | | | | | | | |
| | American Lecithin | | (with | | | | | | | | | | |
| | Company | | Ethoxylated | | | | | | | | | | |
| | | | Mono- | | | | | | | | | | |
| | | | diglycerides | | | | | | | | | | |
| | | | and | | | | | | | | | | |
| | | | Propylene | | | | | | | | | | |
| | | | Glycol) - | | | | | | | | | | |
| | | | Americal | | | | | | | | | | |
| | | | Lecithin | | | | | | | | | | |
| | | | Company” | | | | | | | | | | |
| 428 | Sunflower | 0.5 | Enzyme | 0.5 | Sucrose | 0.5 | | | | | Glycerin | 91.5 | |
| | Lecithin, ≥99% - | | Modified | | Monoester | | | | | | | | |
| | Compass Foods | | Soy | | of | | | | | | | | |
| | Pte Ltd | | Lecithin - | | Palmitic | | | | | | | | |
| | | | American | | Acid - | | | | | | | | |
| | | | Lecithin | | Compass | | | | | | | | |
| | | | Company | | Foods | | | | | | | | |
| | | | | | Pte Ltd | | | | | | | | |
| 429 | Sunflower | 4.0 | Enzyme | 1.0 | Sucrose | 1 | | | | | Glycerin | 88.9 | |
| | Lecithin, ≥99% - | | Modified | | Monoester | | | | | | | | |
| | Compass Foods | | Soy | | of | | | | | | | | |
| | Pte Ltd | | Lecithin - | | Palmitic | | | | | | | | |
| | | | American | | Acid - | | | | | | | | |
| | | | Lecithin | | Compass | | | | | | | | |
| | | | Company | | Foods | | | | | | | | |
| | | | | | Pte Ltd | | | | | | | | |
| 430 | Sunflower | 4.0 | Enzyme | 1.0 | Sucrose | 1 | | | | | Glycerin | 89.0 | |
| | Lecithin, ≥99% - | | Modified | | Monoester | | | | | | | | |
| | Compass Foods | | Soy | | of | | | | | | | | |
| | Pte Ltd | | Lecithin - | | Palmitic | | | | | | | | |
| | | | American | | Acid - | | | | | | | | |
| | | | Lecithin | | Compass | | | | | | | | |
| | | | Company | | Foods | | | | | | | | |
| | | | | | Pte Ltd | | | | | | | | |
| 431 | Sunflower | 2.0 | Enzyme | 0.5 | Sucrose | 0.5 | | | | | Glycerin | 95.0 | |
| | Lecithin, ≥99% - | | Modified | | Monoester | | | | | | | | |
| | Compass Foods | | Soy | | of Palmitic | | | | | | | | |
| | Pte Ltd | | Lecithin - | | Acid - | | | | | | | | |
| | | | American | | Compass | | | | | | | | |
| | | | Lecithin | | Foods | | | | | | | | |
| | | | Company | | Pte Ltd | | | | | | | | |
| 432 | Quillaja Saponaria | 2.0 | Enzyme | 1.0 | Sucrose | 0.5 | | | | | Glycerin | 89.5 | |
| | Extract - | | Modified | | Monoester | | | | | | | | |
| | Ingredion Inc. | | Soy | | of | | | | | | | | |
| | | | Lecithin - | | Palmitic | | | | | | | | |
| | | | American | | Acid - | | | | | | | | |
| | | | Lecithin | | Compass | | | | | | | | |
| | | | Company | | Foods | | | | | | | | |
| | | | | | Pte Ltd | | | | | | | | |
| 433 | Enzyme Modified | 1.0 | “Soy | 1.0 | De-oiled | 1 | | | | | Glycerin | 95.0 | |
| | Soy Lecithin - | | Lecithin | | Powdered | | | | | | | | |
| | American Lecithin | | (with | | Soy | | | | | | | | |
| | Company | | Ethoxylated | | Lecithin - | | | | | | | | |
| | | | Mono- | | American | | | | | | | | |
| | | | diglycerides | | Lecithin | | | | | | | | |
| | | | and | | Company | | | | | | | | |
| | | | Propylene | | | | | | | | | | |
| | | | Glycol) - | | | | | | | | | | |
| | | | Americal | | | | | | | | | | |
| | | | Lecithin | | | | | | | | | | |
| | | | Company” | | | | | | | | | | |
| 434 | Sunflower | 1.0 | Enzyme | 0.5 | Sucrose | 0.5 | | | | | Glycerin | 94.0 | |
| | Lecithin, ≥99% - | | Modified | | Monoester | | | | | | | | |
| | Compass Foods | | Soy | | of | | | | | | | | |
| | Pte Ltd | | Lecithin - | | Palmitic | | | | | | | | |
| | | | American | | Acid - | | | | | | | | |
| | | | Lecithin | | Compass | | | | | | | | |
| | | | Company | | Foods | | | | | | | | |
| | | | | | Pte Ltd | | | | | | | | |
| 435 | Sunflower | 2.0 | Enzyme | 0.5 | Sucrose | 0.5 | | | | | Glycerin | 95.0 | |
| | Lecithin, ≥99% - | | Modified | | Monoester | | | | | | | | |
| | Compass Foods | | Soy | | of Palmitic | | | | | | | | |
| | Pte Ltd | | Lecithin - | | Acid - | | | | | | | | |
| | | | American | | Compass | | | | | | | | |
| | | | Lecithin | | Foods | | | | | | | | |
| | | | Company | | Pte Ltd | | | | | | | | |
| 436 | De-oiled | 0.5 | Quillaja | 0.5 | Sucrose | 0.5 | | | | | Glycerin | 96.5 | |
| | Powdered Soy | | Saponaria | | Monoester | | | | | | | | |
| | Lecithin - | | Extract - | | of Palmitic | | | | | | | | |
| | American Lecithin | | Ingredion | | Acid - | | | | | | | | |
| | Company | | Inc. | | Compass | | | | | | | | |
| | | | | | Foods | | | | | | | | |
| | | | | | Pte Ltd | | | | | | | | |

### Comparative Examples 437-446

Next, tables S007C and S007D shows comparative examples that didn't work, because they either didn't homogenize successfully or phase-separated, i.e. didn't stay homogenized/suspended.

TABLE S007C
Comparative Examples, the hydrophobic phase

| Comparative | Prototype | | Usage | Carrier | Usage | Flavorant or | Usage |
|---|---|---|---|---|---|---|---|
| Example | ID | ABI | Rate | Oil | Rate | Bittermasker | Rate |
| 437 | 39 | Full Spectrum Distillate | 1.0 | MCT | 1.0 | | |
| 438 | 39A | Broad Spectrum | 1.0 | MCT | 1.0 | | |
| | | Distillate | | | | | |
| 439 | 41A | Broad Spectrum | 5.0 | MCT | 5.0 | | |
| | | Distillate | | | | | |
| 440 | 34 | Full Spectrum Distillate | 1.0 | MCT | 1.0 | | |
| 441 | 31 | Full Spectrum Distillate | 1.0 | MCT | 1.0 | | |
| 442 | 47 | Broad Spectrum | 1.0 | MCT | 1.0 | | |
| | | Distillate | | | | | |
| 443 | WGU- | CBD Isolate | 0.5 | MCT | 0.5 | | |
| | 1A- | | | | | | |
| | 30000 | | | | | | |
| 444 | 29F | Full Spectrum Distillate | 1.0 | MCT | 1.0 | | |
| 445 | 25A5B- | CBD Isolate | 8.1 | MCT | 26.3 | Lemon Oil | 0.49 |
| | LE | | | | | Flavor - | |
| | | | | | | Flavor | |
| | | | | | | Insights | |
| | | | | | | Bitterness | 0.32 |
| | | | | | | Blocker, | |
| | | | | | | Natural, | |
| | | | | | | Type A - | |
| | | | | | | Allen | |
| | | | | | | Flavors Inc. | |
| 446 | 25A5B- | CBD Isolate | 8.1 | MCT | 26.3 | Lemon Oil | 0.49 |
| | LE2 | | | | | Flavor - | |
| | | | | | | Flavor | |
| | | | | | | Insights | |
| | | | | | | Bitterness | 0.32 |
| | | | | | | Blocker, | |
| | | | | | | Natural, | |
| | | | | | | Type A - | |
| | | | | | | Allen | |
| | | | | | | Flavors Inc. | |

TABLE S007D
Comparative Examples, the hydrophilic phase used in the emulsion of

| Comparative | | Usage | | Usage | Continuous | Usage | |
|---|---|---|---|---|---|---|---|
| Example | Primary Emulsifier | Rate | Co-Solvent | Rate | Phase | Rate | Note on failure |
| 437 | Hydrogenated | 1.0 | | | Glycerin | 97.0 | Homogenization |
| | phosphatidylcholine | | | | | | unsuccessful - |
| | from genetically non- | | | | | | High viscosity |
| | modified sunflower | | | | | | |
| | lecithin. | | | | | | |
| 438 | Hydrogenated | 1.0 | | | Propylene | 97.0 | Homogenization |
| | phosphatidylcholine | | | | Glycol | | unsuccessful - |
| | from genetically non- | | | | | | Clump formation |
| | modified sunflower | | | | | | post-processing |
| | lecithin. | | | | | | |
| 439 | Sunflower | 10.0 | | | Glycerin | 80.0 | Homogenization |
| | phosphatidylcholine | | | | | | unsuccessful - |
| | (non-GMO) with 90% | | | | | | High viscosity |
| | phosphatidylcholine | | | | | | |
| 440 | Modified Gum Acacia - | 2.0 | | | Glycerin | 96.0 | Homogenization |
| | Ingredion Inc. | | | | | | unsuccessful - |
| | | | | | | | High viscosity |
| 441 | Apple Pectin - | 2.0 | | | Propylene | 96.0 | Homogenization |
| | Ingredion Inc. | | | | Glycol | | unsuccessful - |
| | | | | | | | Immediate |
| | | | | | | | separation post- |
| | | | | | | | processing |
| 442 | Grass-fed Bovine | 1.0 | | | Glycerin | 97.0 | Poor stability |
| | Collagen Peptides - | | | | | | |
| | PeptPure | | | | | | |
| 443 | Pre-hydrated Gum | 1.5 | | | Glycerin | 97.5 | Poor stability |
| | Arabic | | | | | | |
| 444 | Lysophosphatidylcholine | 0.5 | Ethanol | 10.0 | Glycerin | 87.5 | Poor stability |
| | from soybean | | | | | | |
| | lecithin (non-GMO), | | | | | | |
| | 80% | | | | | | |
| 445 | Sunflower Lecithin, ≥99% - | 12.2 | | | Glycerin | 52.62 | Homogenization |
| | Compass | | | | | | unsuccessful - |
| | Foods Pte Ltd | | | | | | High viscosity |
| 446 | Sunflower Lecithin, ≥99% - | 8.1 | | | Glycerin | 56.69 | Homogenization |
| | Compass | | | | | | unsuccessful - |
| | Foods Pte Ltd | | | | | | High viscosity |

### Examples 447-463

Table S007E shows example formulations in which using water as the continuous phase led to water activity values higher than 0.9.

TABLE S007E
Water Activity values

| | Prototype | | Usage | Carrier | Usage | Primary | Usage | Secondary | Usage | Continuous | Usage | Water |
|---|---|---|---|---|---|---|---|---|---|---|---|---|
| Example | ID | ABI | Rate | Oil | Rate | Emulsifier | Rate | Emulsifier | Rate | Phase | Rate | Activity |
| 447 | WQN-1 | CBD | 1.0 | MCT | 1.0 | Quillaja | 1.0 | | | Distilled | 97.0 | 0.969 |
| | | Isolate | | | | Saponaria | | | | Water | | |
| | | | | | | Extract - | | | | | | |
| | | | | | | Ingredion | | | | | | |
| | | | | | | Inc. | | | | | | |
| 448 | WQN-2 | CBD | 1.0 | MCT | 1.0 | Quillaja | 3.0 | | | Distilled | 95.0 | |
| | | Isolate | | | | Saponaria | | | | Water | | |
| | | | | | | Extract - | | | | | | |
| | | | | | | Ingredion | | | | | | |
| | | | | | | Inc. | | | | | | |
| 449 | WQN-3 | CBD | 1.0 | MCT | 1.0 | Quillaja | 5.0 | | | Distilled | 93.0 | |
| | | Isolate | | | | Saponaria | | | | Water | | |
| | | | | | | Extract - | | | | | | |
| | | | | | | Ingredion | | | | | | |
| | | | | | | Inc. | | | | | | |
| 450 | WQN- | CBD | 2.0 | MCT | 1.0 | Quillaja | 5.0 | | | Distilled | 92.0 | 0.952 |
| | 3A | Isolate | | | | Saponaria | | | | Water | | |
| | | | | | | Extract - | | | | | | |
| | | | | | | Ingredion | | | | | | |
| | | | | | | Inc. | | | | | | |
| 451 | WSP-1 | CBD | 1.0 | MCT | 1.0 | Quillaja | 1.0 | | | Distilled | 97.0 | |
| | | Isolate | | | | Saponaria | | | | Water | | |
| | | | | | | Extract - | | | | | | |
| | | | | | | Naturex | | | | | | |
| 452 | WSP- | CBD | 2.0 | MCT | 1.0 | Quillaja | 1.0 | | | Distilled | 96.0 | |
| | 1A | Isolate | | | | Saponaria | | | | Water | | |
| | | | | | | Extract - | | | | | | |
| | | | | | | Naturex | | | | | | |
| 453 | WSP-2 | CBD | 1.0 | MCT | 1.0 | Quillaja | 3.0 | | | Distilled | 95.0 | |
| | | Isolate | | | | Saponaria | | | | Water | | |
| | | | | | | Extract - | | | | | | |
| | | | | | | Naturex | | | | | | |
| 454 | WSP-3 | CBD | 1.0 | MCT | 1.0 | Quillaja | 5.0 | | | Distilled | 93.0 | |
| | | Isolate | | | | Saponaria | | | | Water | | |
| | | | | | | Extract - | | | | | | |
| | | | | | | Naturex | | | | | | |
| 455 | WTA- | CBD | 3.0 | MCT | 3.0 | Modified | 4.5 | | | Distilled | 89.5 | |
| | 1-5000 | Isolate | | | | Gum | | | | Water | | |
| | | | | | | Acacia - | | | | | | |
| | | | | | | Ingredion | | | | | | |
| | | | | | | Inc. | | | | | | |
| 456 | WTA- | CBD | 3.0 | MCT | 3.0 | Modified | 6.0 | | | Distilled | 88.0 | |
| | 2-5000 | Isolate | | | | Gum | | | | Water | | |
| | | | | | | Acacia - | | | | | | |
| | | | | | | Ingredion | | | | | | |
| | | | | | | Inc. | | | | | | |
| 457 | WTAM-1- | CBD | 3.0 | MCT | 3.0 | Gum | 6.0 | | | Distilled | 88.0 | |
| | 5000 | Isolate | | | | Acacia - | | | | Water | | |
| | | | | | | Ingredion | | | | | | |
| | | | | | | Inc. | | | | | | |
| 458 | WTAM-1- | CBD | 3.0 | MCT | 3.0 | Gum | 6.0 | | | Distilled | 88.0 | |
| | 10000 | Isolate | | | | Acacia - | | | | Water | | |
| | | | | | | Ingredion | | | | | | |
| | | | | | | Inc. | | | | | | |
| 459 | WTAM-2- | CBD | 6.0 | MCT | 6.0 | Gum | 6.0 | | | Distilled | 82.0 | 0.959 |
| | 5000 | Isolate | | | | Acacia - | | | | Water | | |
| | | | | | | Ingredion | | | | | | |
| | | | | | | Inc. | | | | | | |
| 460 | WGU- | CBD | 0.5 | MCT | 0.5 | Pre- | 1.5 | | | Distilled | 97.5 | |
| | 1-5000 | Isolate | | | | hydrated | | | | Water | | |
| | | | | | | Gum | | | | | | |
| | | | | | | Arabic | | | | | | |
| 461 | 34C | Broad | 1.0 | Refined | 1.0 | Modified | 5.0 | | | Distilled | 93.0 | |
| | | Spectrum | | Olive | | Gum | | | | Water | | |
| | | Distillate | | Oil | | Acacia - | | | | | | |
| | | | | | | Ingredion | | | | | | |
| | | | | | | Inc. | | | | | | |
| 462 | 35D | Broad | 1.0 | Refined | 1.0 | Modified | 5.0 | | | Distilled | 93.0 | |
| | | Spectrum | | Olive | | Gum | | | | Water | | |
| | | Distillate | | Oil | | Acacia - | | | | | | |
| | | | | | | Ingredion | | | | | | |
| | | | | | | Inc. | | | | | | |
| 463 | 25G5- | CBD | 1.0 | MCT | 1.5 | Sunflower | 2.0 | Sucrose | 1.0 | Distilled | 94.5 | |
| | CP1 | Isolate | | | | Lecithin, ≥99% - | | Monoester | | Water | | |
| | | | | | | Compass | | of | | | | |
| | | | | | | Foods Pte | | Palmitic | | | | |
| | | | | | | Ltd | | Acid - | | | | |
| | | | | | | | | Compass | | | | |
| | | | | | | | | Foods | | | | |
| | | | | | | | | Pte Ltd | | | | |

### Examples 464-474

Table S007F shows examples of when a water-soluble active biological ingredient is included in the hydrophilic phase. These examples relate to the disclosure above at least at paragraphs [0060] and [0061] where active biological ingredients are discussed.

TABLE S007F
Water Soluble ABI in the hydrophilic phase

| | | | Usage | Continuous | Usage | Homogenization |
|---|---|---|---|---|---|---|
| Example | Prototype ID | ABI | Rate | Phase | Rate | Method |
| 464 | KSM66V-230921- | KSM-66 | 24 | Water | 76 | High Speed (500 |
| | 1A | Ashwagandha (Non- | | | | rpm) |
| | | Vegan) | | | | |
| 465 | KSM66V-230921- | KSM-66 | 24 | Glycerin | 76 | High Speed (500 |
| | 1B | Ashwagandha (Non- | | | | rpm) |
| | | Vegan) | | | | |
| 466 | KSM66V-230921- | KSM-66 | 24 | Glycerin | 76 | High Shear |
| | 1C | Ashwagandha (Non- | | | | (13,500 rpm) |
| | | Vegan) | | | | |
| 467 | KSM66NV- | KSM-66 | 24 | Glycerin | 76 | High Pressure |
| | 011021-1A | Ashwagandha | | | | (30,000 psi) |
| | | (Vegan) | | | | |
| 468 | KSM66NV- | KSM-66 | 12 | Glycerin | 88 | High Pressure |
| | 011021-1B | Ashwagandha | | | | (30,000 psi) |
| | | (Vegan) | | | | |
| 469 | KSM66NV- | KSM-66 | 9 | Glycerin | 91 | High Pressure |
| | 011021-1C | Ashwagandha | | | | (30,000 psi) |
| | | (Vegan) | | | | |
| 470 | SensA-230921- | Sensoril | 16 | Water | 84 | High Speed (500 |
| | 1A | Ashwagandha | | | | rpm) |
| 471 | SensA-230921- | Sensoril | 16 | Glycerin | 84 | High Speed (500 |
| | 1B | Ashwagandha | | | | rpm) |
| 472 | SensA-230921- | Sensoril | 16 | Glycerin | 84 | High Shear |
| | 1C | Ashwagandha | | | | (13,500 rpm) |
| 473 | SensA-230921- | Sensoril | 16 | Glycerin | 84 | High Pressure |
| | 1D | Ashwagandha | | | | (30,000 psi) |
| 474 | SensA-230921- | Sensoril | 8 | Glycerin | 92 | High Pressure |
| | 1E | Ashwagandha | | | | (30,000 psi) |

### Examples 475-556

Tables S008A, S008B, and S008C show different usage rates and ingredients for stable water-dispersible emulsions for electrostatic spray drying- and the particulates resulting from these example specific emulsions. The examples in these tables also demonstrate the ability to include at least 22.5% of the ABI in the emulsion for spray drying. Additionally, the resulting particulate formulation has higher usage rates, up to 40% ABI in the powder formed from spray drying. Note that all these formulations can be prepared with blends of emulsifiers, ABIs, and carrier oils listed in this disclosure above, as well as continuous phases listed at paragraph [0074] above and the encapsulants listed at paragraph [0080] above, and blends thereof.

TABLE S008A
compositions of the hydrophobic phase

| Exam- | | | Usage | Carrier | Usage |
|---|---|---|---|---|---|
| ple | Prototype ID | ABI | Rate | Oil | Rate |
| 475 | P210412-1A | CBD Isolate | 8.0 | MCT | 8.0 |
| 476 | 1C | CBD Isolate | 10.0 | MCT | 10.0 |
| 477 | 1D | CBD Isolate | 15.0 | MCT | 15.0 |
| 478 | P210330-2B | CBD Isolate | 5.0 | MCT | 5.0 |
| 479 | P210409-2C2 | CBD Isolate | 5.0 | MCT | 5.0 |
| 480 | P210412-3 | CBD Isolate | 5.0 | MCT | 5.0 |
| 481 | 3A | CBD Isolate | 5.0 | MCT | 5.0 |
| 482 | 3A1 | CBD Isolate | 10.0 | MCT | 10.0 |
| 483 | 3A2 | CBD Isolate | 5.0 | MCT | 5.0 |
| 484 | 3A3 | CBD Isolate | 10.0 | MCT | 10.0 |
| 485 | 3A4 | CBD Isolate | 10.0 | MCT | 10.0 |
| 486 | 3A5 | CBD Isolate | 15.0 | MCT | 15.0 |
| 487 | 3D | CBD Isolate | 5.0 | MCT | 5.0 |
| 488 | 3A5A | CBD Isolate | 16.7 | MCT | 16.7 |
| 489 | 3A5B | CBD Isolate | 18.4 | MCT | 18.4 |
| 490 | 3A5B1 | CBD Isolate | 21.7 | MCT | 21.7 |
| 491 | 3A5G | CBD Isolate | 17.0 | MCT | 17.0 |
| 492 | 3A5G1 | CBD Isolate | 17.0 | MCT | 13.0 |
| 493 | 3A7 | CBD Isolate | 12.5 | MCT | 12.5 |
| 494 | 3A7B | CBD Isolate | 15.0 | MCT | 15.0 |
| 495 | 3A7C | CBD Isolate | 17.5 | MCT | 17.5 |
| 496 | 3A7D | CBD Isolate | 20.0 | MCT | 20.0 |
| 497 | 3A7D1 | CBD Isolate | 21.5 | MCT | 21.5 |
| 498 | 3A7D2 | CBD Isolate | 22.5 | MCT | 22.5 |
| 499 | 3A7D6A | CBD Isolate | 22.5 | MCT | 22.5 |
| 500 | 3A7D6B | CBD Isolate | 22.5 | MCT | 22.5 |
| 501 | 3A7D3 | CBD Isolate | 20.0 | MCT | 20.0 |
| 502 | 3A7D4 | CBD Isolate | 20.0 | MCT | 20.0 |
| 503 | 3A7D7 | CBD Isolate | 20.0 | MCT | 20.0 |
| 504 | 3A7D7C | CBD Isolate | 20.0 | MCT | 20.0 |
| 505 | 3A7D7D | CBD Isolate | 20.0 | MCT | 20.0 |
| 506 | 3A7D7E | CBD Isolate | 20.0 | MCT | 20.0 |
| 507 | 3A9 | CBD Isolate | 15.0 | MCT | 15.0 |
| 508 | 3A10 | CBD Isolate | 18.0 | MCT | 18.0 |
| 509 | 3A14-2.2kg | CBD Isolate | 7.2 | MCT | 7.2 |
| 510 | 3A15 | CBD Isolate | 12.5 | MCT | 12.5 |
| 511 | 3A15A | CBD Isolate | 15.0 | MCT | 15.0 |
| 512 | 3A16 | CBD Isolate | 12.5 | MCT | 12.5 |
| 513 | 3A17 | CBD Isolate | 12.5 | MCT | 12.5 |
| 514 | 3A17A | CBD Isolate | 12.5 | MCT | 12.5 |
| 515 | 6 | CBD Isolate | 15.0 | MCT | 15.0 |
| 516 | 7 | CBD Isolate | 8.0 | MCT | 8.0 |
| 517 | 9 | CBD Isolate | 15.0 | MCT | 15.0 |
| 518 | 3A7D2A1 | Broad Spectrum | 22.5 | MCT | 22.5 |
| | | Distillate | | | |
| 519 | 1G | Broad Spectrum | 15.0 | MCT | 15.0 |
| | | Distillate | | | |
| 520 | 3Z | Broad Spectrum | 5.0 | MCT | 5.0 |
| | | Distillate | | | |
| 521 | 3A7D7H | Broad Spectrum | 20.0 | Hemp | 19.2 |
| | | Distillate | | Seed | |
| | | | | Oil | |
| 522 | 3A7D7D1 | Broad Spectrum | 20.0 | MCT | 20.0 |
| | | Distillate | | | |
| 523 | 3A7D7E1 | Broad Spectrum | 20.0 | MCT | 20.0 |
| | | Distillate | | | |
| 524 | 3A10A | Broad Spectrum | 18.0 | MCT | 18.0 |
| | | Distillate | | | |
| 525 | 3A16A | Broad Spectrum | 12.5 | MCT | 12.5 |
| | | Distillate | | | |
| 526 | 3A17A1 | Broad Spectrum | 12.5 | MCT | 12.5 |
| | | Distillate | | | |
| 527 | 6D | Broad Spectrum | 15.0 | MCT | 15.0 |
| | | Distillate | | | |
| 528 | 7B | Broad Spectrum | 8.0 | MCT | 8.0 |
| | | Distillate | | | |
| 529 | 7D | Broad Spectrum | 8.0 | MCT | 8.0 |
| | | Distillate | | | |
| 530 | 9C | Broad Spectrum | 15.0 | MCT | 15.0 |
| | | Distillate | | | |
| 531 | 3A7D-CFH | Full Spectrum | 20.0 | MCT | 20.0 |
| | | Distillate - CFH | | | |
| 532 | 3A7F2 | Full Spectrum | 12.5 | MCT | 12.5 |
| | | Distillate | | | |
| 533 | 3A7G1 | Full Spectrum | 20.0 | MCT | 20.0 |
| | | Distillate | | | |
| 534 | 1H | Full Spectrum | 15.0 | MCT | 15.0 |
| | | Distillate | | | |
| 535 | 3Z1 | Full Spectrum | 5.0 | MCT | 5.0 |
| | | Distillate | | | |
| 536 | 3A7D7G | Full Spectrum | 20.0 | MCT | 20.0 |
| | | Distillate | | | |
| 537 | 3A7D7D2 | Full Spectrum | 20.0 | MCT | 20.0 |
| | | Distillate | | | |
| 538 | 3A7D7E2 | Full Spectrum | 20.0 | MCT | 20.0 |
| | | Distillate | | | |
| 539 | 3A10B | Full Spectrum | 18.0 | MCT | 18.0 |
| | | Distillate | | | |
| 540 | 3A16B | Full Spectrum | 12.5 | MCT | 12.5 |
| | | Distillate | | | |
| 541 | 3A17A2 | Full Spectrum | 12.5 | MCT | 12.5 |
| | | Distillate | | | |
| 542 | 3A17A3 | Full Spectrum | 5.0 | MCT | 5.0 |
| | | Distillate | | | |
| 543 | 3A17A4 | Full Spectrum | 5.0 | MCT | 5.0 |
| | | Distillate | | | |
| 544 | 6E | Full Spectrum | 15.0 | MCT | 15.0 |
| | | Distillate | | | |
| 545 | 7C | Full Spectrum | 8.0 | MCT | 8.0 |
| | | Distillate | | | |
| 546 | 9D | Full Spectrum | 15.0 | MCT | 15.0 |
| | | Distillate | | | |
| 547 | 1H1 | Full Spectrum | 7.5 | MCT | 15.0 |
| | | Distillate | | | |
| | | CBN Isolate | 7.5 | | |
| 548 | 3Z2 | Full Spectrum | 2.5 | MCT | 5.0 |
| | | Distillate | | | |
| | | CBG Distillate | 2.5 | | |
| 549 | 3A7D7G1 | Full Spectrum | 6.0 | Olive | 19.0 |
| | | Distillate | | Oil | |
| | | CBN Isolate | 7.0 | | |
| | | CBG isolate | 7.0 | | |
| 550 | 3A7D7G2 | Full Spectrum | 2.5 | Olive | 5.0 |
| | | Distillate | | Oil | |
| | | CBN Isolate | 2.5 | | |
| | | CBG isolate | 2.5 | | |
| 551 | 3A7D7G2 | Full Spectrum | 2.5 | Olive | 5.0 |
| | | Distillate | | Oil | |
| | | CBN Isolate | 2.5 | | |
| | | CBG isolate | 2.5 | | |
| 552 | 3A7D7D3A | Broad Spectrum | 7.0 | MCT | 20.0 |
| | | Distillate | | | |
| | | CBN Isolate | 7.0 | | |
| | | CBG isolate | 6.0 | | |
| 553 | 7B | Broad Spectrum | 4.0 | MCT | 8.0 |
| | | Distillate | | | |
| | | CBN Isolate | 4.0 | | |
| 554 | 9C | Broad Spectrum | 7.5 | MCT | 15.0 |
| | | Distillate | | | |
| | | CBG Distillate | 7.5 | | |
| 555 | 3A7D7F | Broad Spectrum | 5.0 | Hemp | 18.8 |
| | | Distillate | | Seed oil | |
| | | CBN Isolate | 5.0 | | |
| 556 | 3A7D7G | Broad Spectrum | 4.0 | Olive | 18.5 |
| | | Distillate | | Oil | |
| | | CBG distillate | 8.0 | | |

TABLE S008B
compositions of the corresponding hydrophilic phase, and resulting solid weight percentages of the resulting emulsions

| | | | | | | | | | | | Continuous | |
|---|---|---|---|---|---|---|---|---|---|---|---|---|
| | | | | | | | | | | | Phase of | Resulting |
| | | | | | | | | | | | Water | Emulsion |
| | | Usage | Primary | Usage | Secondary | Usage | Tertiary | Usage | Additional | Usage | Usage | % |
| Example | Encapsulant | Rate | Emulsifier | Rate | Emulsifier | Rate | Emulsifier | Rate | Components | Rate | Rate | Solid |
| 475 | | | Modified | 24.0 | | | | | | | 60.0 | 40.0 |
| | | | Food | | | | | | | | | |
| | | | Starch | | | | | | | | | |
| | | | from | | | | | | | | | |
| | | | Waxy | | | | | | | | | |
| | | | Maize - | | | | | | | | | |
| | | | Ingredion | | | | | | | | | |
| | | | Inc. | | | | | | | | | |
| 476 | | | Modified | 20.0 | | | | | | | 60.0 | 40.0 |
| | | | Food | | | | | | | | | |
| | | | Starch | | | | | | | | | |
| | | | from | | | | | | | | | |
| | | | Waxy | | | | | | | | | |
| | | | Maize - | | | | | | | | | |
| | | | Ingredion | | | | | | | | | |
| | | | Inc. | | | | | | | | | |
| 477 | Flavor- | 5.0 | Modified | 10.0 | | | | | | | 55.0 | 45.0 |
| | free | | Food | | | | | | | | | |
| | Maltodextrin - | | Starch | | | | | | | | | |
| | Ingredion | | from | | | | | | | | | |
| | Inc. | | Waxy | | | | | | | | | |
| | | | Maize - | | | | | | | | | |
| | | | Ingredion | | | | | | | | | |
| | | | Inc. | | | | | | | | | |
| 478 | Maltodextrin, | 25.0 | Modified | 10.0 | | | | | | | 55.0 | 45.0 |
| | Tapioca | | Corn | | | | | | | | | |
| | Based - | | Starch - | | | | | | | | | |
| | Ingredion | | Ingredion | | | | | | | | | |
| | Inc. | | Inc. | | | | | | | | | |
| 479 | Maltodextrin, | 25.0 | Modified | 10.0 | | | | | | | 55.0 | 45.0 |
| | 90% | | Corn | | | | | | | | | |
| | Dietary | | Starch - | | | | | | | | | |
| | Fiber - | | Ingredion | | | | | | | | | |
| | Fibersol | | Inc. | | | | | | | | | |
| 480 | Maltodextrin, | 25.0 | Modified | 10.0 | | | | | | | 55.0 | 45.0 |
| | 90% | | Food | | | | | | | | | |
| | Dietary | | Starch | | | | | | | | | |
| | Fiber - | | from | | | | | | | | | |
| | Fibersol | | Tapioca - | | | | | | | | | |
| | | | Ingredion | | | | | | | | | |
| | | | Inc. | | | | | | | | | |
| 481 | Flavor- | 25.0 | Modified | 10.0 | | | | | | | 55.0 | 45.0 |
| | free | | Food | | | | | | | | | |
| | Maltodextrin - | | Starch | | | | | | | | | |
| | Ingredion | | from | | | | | | | | | |
| | Inc. | | Tapioca - | | | | | | | | | |
| | | | Ingredion | | | | | | | | | |
| | | | Inc. | | | | | | | | | |
| 482 | Flavor- | 12.5 | Modified | 5.0 | | | | | | | 62.5 | 37.5 |
| | free | | Food | | | | | | | | | |
| | Maltodextrin - | | Starch | | | | | | | | | |
| | Ingredion | | from | | | | | | | | | |
| | Inc. | | Tapioca - | | | | | | | | | |
| | | | Ingredion | | | | | | | | | |
| | | | Inc. | | | | | | | | | |
| 483 | Flavor- | 12.5 | Modified | 5.0 | | | | | | | 72.5 | 27.5 |
| | free | | Food | | | | | | | | | |
| | Maltodextrin - | | Starch | | | | | | | | | |
| | Ingredion | | from | | | | | | | | | |
| | Inc. | | Tapioca - | | | | | | | | | |
| | | | Ingredion | | | | | | | | | |
| | | | Inc. | | | | | | | | | |
| 484 | Flavor- | 15.0 | Modified | 10.0 | | | | | | | 55.0 | 45.0 |
| | free | | Food | | | | | | | | | |
| | Maltodextrin - | | Starch | | | | | | | | | |
| | Ingredion | | from | | | | | | | | | |
| | Inc. | | Tapioca - | | | | | | | | | |
| | | | Ingredion | | | | | | | | | |
| | | | Inc. | | | | | | | | | |
| 485 | Flavor- | 20.0 | Modified | 5.0 | | | | | | | 55.0 | 45.0 |
| | free | | Food | | | | | | | | | |
| | Maltodextrin - | | Starch | | | | | | | | | |
| | Ingredion | | from | | | | | | | | | |
| | Inc. | | Tapioca - | | | | | | | | | |
| | | | Ingredion | | | | | | | | | |
| | | | Inc. | | | | | | | | | |
| 486 | Flavor- | 5.0 | Modified | 10.0 | | | | | | | 55.0 | 45.0 |
| | free | | Food | | | | | | | | | |
| | Maltodextrin - | | Starch | | | | | | | | | |
| | Ingredion | | from | | | | | | | | | |
| | Inc. | | Tapioca - | | | | | | | | | |
| | | | Ingredion | | | | | | | | | |
| | | | Inc. | | | | | | | | | |
| 487 | Flavor- | 25.0 | Modified | 10.0 | | | | | | | 55.0 | 45.0 |
| | free | | Food | | | | | | | | | |
| | Maltodextrin - | | Starch | | | | | | | | | |
| | Ingredion | | from | | | | | | | | | |
| | Inc. | | Tapioca - | | | | | | | | | |
| | | | Ingredion | | | | | | | | | |
| | | | Inc. | | | | | | | | | |
| 488 | Flavor- | 5.5 | Modified | 11.1 | | | | | | | 50.0 | 50.0 |
| | free | | Food | | | | | | | | | |
| | Maltodextrin - | | Starch | | | | | | | | | |
| | Ingredion | | from | | | | | | | | | |
| | Inc. | | Tapioca - | | | | | | | | | |
| | | | Ingredion | | | | | | | | | |
| | | | Inc. | | | | | | | | | |
| 489 | Flavor- | 6.0 | Modified | 12.2 | | | | | | | 45.0 | 55.0 |
| | free | | Food | | | | | | | | | |
| | Maltodextrin - | | Starch | | | | | | | | | |
| | Ingredion | | from | | | | | | | | | |
| | Inc. | | Tapioca - | | | | | | | | | |
| | | | Ingredion | | | | | | | | | |
| | | | Inc. | | | | | | | | | |
| 490 | Flavor- | 7.2 | Modified | 14.4 | | | | | | | 35.0 | 65.0 |
| | free | | Food | | | | | | | | | |
| | Maltodextrin - | | Starch | | | | | | | | | |
| | Ingredion | | from | | | | | | | | | |
| | Inc. | | Tapioca - | | | | | | | | | |
| | | | Ingredion | | | | | | | | | |
| | | | Inc. | | | | | | | | | |
| 491 | Flavor- | 5.0 | Modified | 6.0 | | | | | | | 55.0 | 45.0 |
| | free | | Food | | | | | | | | | |
| | Maltodextrin - | | Starch | | | | | | | | | |
| | Ingredion | | from | | | | | | | | | |
| | Inc. | | Tapioca - | | | | | | | | | |
| | | | Ingredion | | | | | | | | | |
| | | | Inc. | | | | | | | | | |
| 492 | Flavor- | 5.0 | Modified | 10.0 | | | | | | | 55.0 | 45.0 |
| | free | | Food | | | | | | | | | |
| | Maltodextrin - | | Starch | | | | | | | | | |
| | Ingredion | | from | | | | | | | | | |
| | Inc. | | Tapioca - | | | | | | | | | |
| | | | Ingredion | | | | | | | | | |
| | | | Inc. | | | | | | | | | |
| 493 | Flavor- | 10.0 | Modified | 10.0 | | | | | | | 55.0 | 45.0 |
| | free | | Food | | | | | | | | | |
| | Maltodextrin - | | Starch | | | | | | | | | |
| | Ingredion | | from | | | | | | | | | |
| | Inc. | | Tapioca - | | | | | | | | | |
| | | | Ingredion | | | | | | | | | |
| | | | Inc. | | | | | | | | | |
| 494 | Flavor- | 10.0 | Modified | 10.0 | | | | | | | 50.0 | 50.0 |
| | free | | Food | | | | | | | | | |
| | Maltodextrin - | | Starch | | | | | | | | | |
| | Ingredion | | from | | | | | | | | | |
| | Inc. | | Tapioca - | | | | | | | | | |
| | | | Ingredion | | | | | | | | | |
| | | | Inc. | | | | | | | | | |
| 495 | Flavor- | 10.0 | Modified | 10.0 | | | | | | | 45.0 | 55.0 |
| | free | | Food | | | | | | | | | |
| | Maltodextrin - | | Starch | | | | | | | | | |
| | Ingredion | | from | | | | | | | | | |
| | Inc. | | Tapioca - | | | | | | | | | |
| | | | Ingredion | | | | | | | | | |
| | | | Inc. | | | | | | | | | |
| 496 | Flavor- | 10.0 | Modified | 10.0 | | | | | | | 40.0 | 60.0 |
| | free | | Food | | | | | | | | | |
| | Maltodextrin - | | Starch | | | | | | | | | |
| | Ingredion | | from | | | | | | | | | |
| | Inc. | | Tapioca - | | | | | | | | | |
| | | | Ingredion | | | | | | | | | |
| | | | Inc. | | | | | | | | | |
| 497 | Flavor- | 8.5 | Modified | 8.5 | | | | | | | 40.0 | 60.0 |
| | free | | Food | | | | | | | | | |
| | Maltodextrin - | | Starch | | | | | | | | | |
| | Ingredion | | from | | | | | | | | | |
| | Inc. | | Tapioca - | | | | | | | | | |
| | | | Ingredion | | | | | | | | | |
| | | | Inc. | | | | | | | | | |
| 498 | Flavor- | 7.5 | Modified | 7.5 | | | | | | | 40.0 | 60.0 |
| | free | | Food | | | | | | | | | |
| | Maltodextrin - | | Starch | | | | | | | | | |
| | Ingredion | | from | | | | | | | | | |
| | Inc. | | Tapioca - | | | | | | | | | |
| | | | Ingredion | | | | | | | | | |
| | | | Inc. | | | | | | | | | |
| 499 | Flavor- | 7.5 | Modified | 7.5 | | | | | | | 40.0 | 60.0 |
| | free | | Food | | | | | | | | | |
| | Maltodextrin - | | Starch | | | | | | | | | |
| | Ingredion | | from | | | | | | | | | |
| | Inc. | | Tapioca - | | | | | | | | | |
| | | | Ingredion | | | | | | | | | |
| | | | Inc. | | | | | | | | | |
| 500 | Flavor- | 10.0 | Modified | 5.0 | | | | | | | 40.0 | 60.0 |
| | free | | Food | | | | | | | | | |
| | Maltodextrin - | | Starch | | | | | | | | | |
| | Ingredion | | from | | | | | | | | | |
| | Inc. | | Tapioca - | | | | | | | | | |
| | | | Ingredion | | | | | | | | | |
| | | | Inc. | | | | | | | | | |
| 501 | Flavor- | 7.2 | Modified | 10.0 | | | | | Calcium | 2.8 | 40.0 | 60.0 |
| | free | | Food | | | | | | Disodium | | | |
| | Maltodextrin - | | Starch | | | | | | EDTA | | | |
| | Ingredion | | from | | | | | | | | | |
| | Inc. | | Tapioca - | | | | | | | | | |
| | | | Ingredion | | | | | | | | | |
| | | | Inc. | | | | | | | | | |
| 502 | Flavor- | 10.0 | Modified | 10.0 | | | | | Citric | 0.03 | 40.0 | 60.0 |
| | free | | Food | | | | | | Acid | | | |
| | Maltodextrin - | | Starch | | | | | | | | | |
| | Ingredion | | from | | | | | | | | | |
| | Inc. | | Tapioca - | | | | | | | | | |
| | | | Ingredion | | | | | | | | | |
| | | | Inc. | | | | | | | | | |
| 503 | Flavor- | 10.0 | Modified | 10.0 | | | | | | | 40.0 | 60.0 |
| | free | | Food | | | | | | | | | |
| | Maltodextrin - | | Starch | | | | | | | | | |
| | Ingredion | | from | | | | | | | | | |
| | Inc. | | Tapioca - | | | | | | | | | |
| | | | Ingredion | | | | | | | | | |
| | | | Inc. | | | | | | | | | |
| 504 | Maltodextrin, | 10.0 | Modified | 10.0 | | | | | | | 40.0 | 60.0 |
| | 90% | | Food | | | | | | | | | |
| | Dietary | | Starch | | | | | | | | | |
| | Fiber - | | from | | | | | | | | | |
| | Fibersol | | Tapioca - | | | | | | | | | |
| | | | Ingredion | | | | | | | | | |
| | | | Inc. | | | | | | | | | |
| 505 | Emulsifying | 10.0 | Modified | 10.0 | | | | | | | 40.0 | 60.0 |
| | Starch - | | Food | | | | | | | | | |
| | Cargill | | Starch | | | | | | | | | |
| | Inc. | | from | | | | | | | | | |
| | | | Tapioca - | | | | | | | | | |
| | | | Ingredion | | | | | | | | | |
| | | | Inc. | | | | | | | | | |
| 506 | Inulin, | 10.0 | Modified | 10.0 | | | | | | | 40.0 | 60.0 |
| | water | | Food | | | | | | | | | |
| | soluble | | Starch | | | | | | | | | |
| | fiber - It's | | from | | | | | | | | | |
| | Just | | Tapioca - | | | | | | | | | |
| | | | Ingredion | | | | | | | | | |
| | | | Inc. | | | | | | | | | |
| 507 | Flavor- | 5.0 | Modified | 10.0 | | | | | | | 55.0 | 45.0 |
| | free | | Starch | | | | | | | | | |
| | Maltodextrin - | | from | | | | | | | | | |
| | Ingredion | | Waxy | | | | | | | | | |
| | Inc. | | Maize - | | | | | | | | | |
| | | | Ingredion | | | | | | | | | |
| | | | Inc. | | | | | | | | | |
| 508 | Flavor- | 4.5 | Modified | 4.5 | | | | | | | 55.0 | 45.0 |
| | free | | Starch | | | | | | | | | |
| | Maltodextrin - | | from | | | | | | | | | |
| | Ingredion | | Waxy | | | | | | | | | |
| | Inc. | | Maize - | | | | | | | | | |
| | | | Ingredion | | | | | | | | | |
| | | | Inc. | | | | | | | | | |
| 509 | Flavor- | 36.1 | Modified | 14.5 | | | | | | | 35.0 | 65.0 |
| | free | | Food | | | | | | | | | |
| | Maltodextrin - | | Starch | | | | | | | | | |
| | Ingredion | | from | | | | | | | | | |
| | Inc. | | Tapioca - | | | | | | | | | |
| | | | Ingredion | | | | | | | | | |
| | | | Inc. | | | | | | | | | |
| 510 | | | Organic | 20.0 | | | | | | | 55.0 | 45.0 |
| | | | Gum | | | | | | | | | |
| | | | Acacia- | | | | | | | | | |
| | | | Ingredion | | | | | | | | | |
| | | | Inc. | | | | | | | | | |
| 511 | | | Organic | 20.0 | | | | | | | 50.0 | 50.0 |
| | | | Gum | | | | | | | | | |
| | | | Acacia- | | | | | | | | | |
| | | | Ingredion | | | | | | | | | |
| | | | Inc. | | | | | | | | | |
| 512 | Flavor- | 10.0 | Organic | 10.0 | | | | | | | 55.0 | 45.0 |
| | free | | Gum | | | | | | | | | |
| | Maltodextrin - | | Acacia - | | | | | | | | | |
| | Ingredion | | Ingredion | | | | | | | | | |
| | Inc. | | Inc. | | | | | | | | | |
| 513 | | | Modified | 20.0 | | | | | | | 55.0 | 45.0 |
| | | | Gum | | | | | | | | | |
| | | | Acacia - | | | | | | | | | |
| | | | Ingredion | | | | | | | | | |
| | | | Inc. | | | | | | | | | |
| 514 | Flavor- | 10.0 | Modified | 10.0 | | | | | | | 55.0 | 45.0 |
| | free | | Gum | | | | | | | | | |
| | Maltodextrin - | | Acacia - | | | | | | | | | |
| | Ingredion | | Ingredion | | | | | | | | | |
| | Inc. | | Inc. | | | | | | | | | |
| 515 | Flavor- | 5.0 | Modified | 10.0 | | | | | | | 55.0 | 45.0 |
| | free | | Food | | | | | | | | | |
| | Maltodextrin - | | Starch | | | | | | | | | |
| | Ingredion | | Refined | | | | | | | | | |
| | Inc. | | from | | | | | | | | | |
| | | | Waxy | | | | | | | | | |
| | | | Maize - | | | | | | | | | |
| | | | Ingredion | | | | | | | | | |
| | | | Inc. | | | | | | | | | |
| 516 | | | Modified | 24.0 | | | | | | | 60.0 | 40.0 |
| | | | Food | | | | | | | | | |
| | | | Starch | | | | | | | | | |
| | | | from | | | | | | | | | |
| | | | Waxy | | | | | | | | | |
| | | | Maize - | | | | | | | | | |
| | | | Ingredion | | | | | | | | | |
| | | | Inc. | | | | | | | | | |
| 517 | Inulin, | 5.0 | Quillaja | 50.0 | | | | | | | 15.0 | 45.0 |
| | water | | Saponaria | | | | | | | | | |
| | soluble | | Extract - | | | | | | | | | |
| | fiber - It's | | Ingredion | | | | | | | | | |
| | Just | | Inc. | | | | | | | | | |
| 518 | Flavor- | 7.5 | Modified | 7.5 | | | | | | | 40.0 | 60.0 |
| | free | | Food | | | | | | | | | |
| | Maltodextrin - | | Starch | | | | | | | | | |
| | Ingredion | | from | | | | | | | | | |
| | Inc. | | Tapioca - | | | | | | | | | |
| | | | Ingredion | | | | | | | | | |
| | | | Inc. | | | | | | | | | |
| 519 | Flavor- | 5.0 | Modified | 10.0 | | | | | | | 55.0 | 45.0 |
| | free | | Food | | | | | | | | | |
| | Maltodextrin - | | Starch | | | | | | | | | |
| | Ingredion | | from | | | | | | | | | |
| | Inc. | | Waxy | | | | | | | | | |
| | | | Maize - | | | | | | | | | |
| | | | Ingredion | | | | | | | | | |
| | | | Inc. | | | | | | | | | |
| 520 | Maltodextrin, | 25.0 | Modified | 10.0 | | | | | | | 55.0 | 45.0 |
| | 90% | | Food | | | | | | | | | |
| | Dietary | | Starch | | | | | | | | | |
| | Fiber - | | from | | | | | | | | | |
| | Fibersol | | Tapioca - | | | | | | | | | |
| | | | Ingredion | | | | | | | | | |
| | | | Inc. | | | | | | | | | |
| 52 | Flavor- | 10.0 | Modified | 5.0 | Modified | 2.5 | Modified | 2.5 | Orange | 0.8 | 40.0 | 60.0 |
| | free | | Food | | Food | | Food | | Oil | | | |
| | Maltodextrin - | | Starch | | Starch | | Starch | | Flavor - | | | |
| | Ingredion | | from | | from | | Refined | | Flavor | | | |
| | Inc. | | Tapioca - | | Waxy | | from | | Insights | | | |
| | | | Ingredion | | Maize - | | Waxy | | | | | |
| | | | Inc. | | Ingredion | | Maize - | | | | | |
| | | | | | Inc. | | Ingredion | | | | | |
| | | | | | | | Inc. | | | | | |
| 522 | Emulsifying | 10.0 | Modified | 10.0 | | | | | | | 40.0 | 60.0 |
| | Starch - | | Food | | | | | | | | | |
| | Cargill | | Starch | | | | | | | | | |
| | Inc. | | from | | | | | | | | | |
| | | | Tapioca - | | | | | | | | | |
| | | | Ingredion | | | | | | | | | |
| | | | Inc. | | | | | | | | | |
| 523 | Inulin, | 10.0 | Modified | 10.0 | | | | | | | 40.0 | 60.0 |
| | water | | Food | | | | | | | | | |
| | soluble | | Starch | | | | | | | | | |
| | fiber - It's | | from | | | | | | | | | |
| | Just | | Tapioca - | | | | | | | | | |
| | | | Ingredion | | | | | | | | | |
| | | | Inc. | | | | | | | | | |
| 524 | Flavor- | 4.5 | Modified | 4.5 | | | | | | | 55.0 | 45.0 |
| | free | | Starch | | | | | | | | | |
| | Maltodextrin - | | from | | | | | | | | | |
| | Ingredion | | Waxy | | | | | | | | | |
| | Inc. | | Maize - | | | | | | | | | |
| | | | Ingredion | | | | | | | | | |
| | | | Inc. | | | | | | | | | |
| 525 | Flavor- | 10.0 | Organic | 10.0 | | | | | | | 55.0 | 45.0 |
| | free | | Gum | | | | | | | | | |
| | Maltodextrin - | | Acacia - | | | | | | | | | |
| | Ingredion | | Ingredion | | | | | | | | | |
| | Inc. | | Inc. | | | | | | | | | |
| 526 | Flavor- | 10.0 | Modified | 10.0 | | | | | | | 55.0 | 45.0 |
| | free | | Gum | | | | | | | | | |
| | Maltodextrin - | | Acacia - | | | | | | | | | |
| | Ingredion | | Ingredion | | | | | | | | | |
| | Inc. | | Inc. | | | | | | | | | |
| 527 | Flavor- | 5.0 | Modified | 10.0 | | | | | | | 55.0 | 45.0 |
| | free | | Food | | | | | | | | | |
| | Maltodextrin - | | Starch | | | | | | | | | |
| | Ingredion | | Refined | | | | | | | | | |
| | Inc. | | from | | | | | | | | | |
| | | | Waxy | | | | | | | | | |
| | | | Maize - | | | | | | | | | |
| | | | Ingredion | | | | | | | | | |
| | | | Inc. | | | | | | | | | |
| 528 | | | Modified | 24.0 | | | | | | | 60.0 | 40.0 |
| | | | Food | | | | | | | | | |
| | | | Starch | | | | | | | | | |
| | | | from | | | | | | | | | |
| | | | Waxy | | | | | | | | | |
| | | | Maize - | | | | | | | | | |
| | | | Ingredion | | | | | | | | | |
| | | | Inc. | | | | | | | | | |
| 529 | | | Modified | 5.0 | Modified | 18.0 | Modified | 11.0 | | | 50.0 | 50.0 |
| | | | Food | | Gum | | Starch | | | | | |
| | | | Starch | | Acacia - | | from | | | | | |
| | | | from | | Ingredion | | Waxy | | | | | |
| | | | Waxy | | Inc. | | Maize - | | | | | |
| | | | Maize - | | | | Ingredion | | | | | |
| | | | Ingredion | | | | Inc. | | | | | |
| | | | Inc. | | | | | | | | | |
| 530 | Inulin, | 5.0 | Quillaja | 50.0 | | | | | | | 15.0 | 45.0 |
| | water | | Saponaria | | | | | | | | | |
| | soluble | | Extract - | | | | | | | | | |
| | fiber - It's | | Ingredion | | | | | | | | | |
| | Just | | Inc. | | | | | | | | | |
| 531 | Flavor- | 10.0 | Modified | 10.0 | | | | | | | 40.0 | 60.0 |
| | free | | Food | | | | | | | | | |
| | Maltodextrin - | | Starch | | | | | | | | | |
| | Ingredion | | from | | | | | | | | | |
| | Inc. | | Tapioca - | | | | | | | | | |
| | | | Ingredion | | | | | | | | | |
| | | | Inc. | | | | | | | | | |
| 532 | Flavor- | 10.0 | Modified | 10.0 | | | | | | | 55.0 | 45.0 |
| | free | | Food | | | | | | | | | |
| | Maltodextrin - | | Starch | | | | | | | | | |
| | Ingredion | | from | | | | | | | | | |
| | Inc. | | Tapioca - | | | | | | | | | |
| | | | Ingredion | | | | | | | | | |
| | | | Inc. | | | | | | | | | |
| 533 | Flavor- | 10.0 | Modified | 10.0 | | | | | | | 40.0 | 60.0 |
| | free | | Food | | | | | | | | | |
| | Maltodextrin - | | Starch | | | | | | | | | |
| | Ingredion | | from | | | | | | | | | |
| | Inc. | | Tapioca - | | | | | | | | | |
| | | | Ingredion | | | | | | | | | |
| | | | Inc. | | | | | | | | | |
| 534 | Flavor- | 5.0 | Modified | 10.0 | | | | | | | 55.0 | 45.0 |
| | free | | Food | | | | | | | | | |
| | Maltodextrin - | | Starch | | | | | | | | | |
| | Ingredion | | from | | | | | | | | | |
| | Inc. | | Waxy | | | | | | | | | |
| | | | Maize - | | | | | | | | | |
| | | | Ingredion | | | | | | | | | |
| | | | Inc. | | | | | | | | | |
| 535 | Maltodextrin, | 25.0 | Modified | 10.0 | | | | | | | 55.0 | 45.0 |
| | 90% | | Food | | | | | | | | | |
| | Dietary | | Starch | | | | | | | | | |
| | Fiber - | | from | | | | | | | | | |
| | Fibersol | | Tapioca - | | | | | | | | | |
| | | | Ingredion | | | | | | | | | |
| | | | Inc. | | | | | | | | | |
| 536 | Flavor- | 10.0 | Modified | 10.0 | | | | | | | 40.0 | 60.0 |
| | free | | Food | | | | | | | | | |
| | Maltodextrin - | | Starch | | | | | | | | | |
| | Ingredion | | from | | | | | | | | | |
| | Inc. | | Tapioca - | | | | | | | | | |
| | | | Ingredion | | | | | | | | | |
| | | | Inc. | | | | | | | | | |
| 537 | Emulsifying | 10.0 | Modified | 10.0 | | | | | | | 40.0 | 60.0 |
| | Starch - | | Food | | | | | | | | | |
| | Cargill | | Starch | | | | | | | | | |
| | Inc. | | from | | | | | | | | | |
| | | | Tapioca - | | | | | | | | | |
| | | | Ingredion | | | | | | | | | |
| | | | Inc. | | | | | | | | | |
| 538 | Inulin, | 10.0 | Modified | 10.0 | | | | | | | 40.0 | 60.0 |
| | water | | Food | | | | | | | | | |
| | soluble | | Starch | | | | | | | | | |
| | fiber - It's | | from | | | | | | | | | |
| | Just | | Tapioca - | | | | | | | | | |
| | | | Ingredion | | | | | | | | | |
| | | | Inc. | | | | | | | | | |
| 539 | Flavor- | 4.5 | Modified | 4.5 | | | | | | | 55.0 | 45.0 |
| | free | | Starch | | | | | | | | | |
| | Maltodextrin - | | from | | | | | | | | | |
| | Ingredion | | Waxy | | | | | | | | | |
| | Inc. | | Maize - | | | | | | | | | |
| | | | Ingredion | | | | | | | | | |
| | | | Inc. | | | | | | | | | |
| 540 | Flavor- | 10.0 | Organic | 10.0 | | | | | | | 55.0 | 45.0 |
| | free | | Gum | | | | | | | | | |
| | Maltodextrin - | | Acacia - | | | | | | | | | |
| | Ingredion | | Ingredion | | | | | | | | | |
| | Inc. | | Inc. | | | | | | | | | |
| 541 | Flavor- | 10.0 | Modified | 10.0 | | | | | | | 55.0 | 45.0 |
| | free | | Gum | | | | | | | | | |
| | Maltodextrin - | | Acacia - | | | | | | | | | |
| | Ingredion | | Ingredion | | | | | | | | | |
| | Inc. | | Inc. | | | | | | | | | |
| 542 | | | Modified | 5.0 | Modified | 50.0 | | | | | 35.0 | 65.0 |
| | | | Gum | | Food | | | | | | | |
| | | | Acacia - | | Starch | | | | | | | |
| | | | Ingredion | | Refined | | | | | | | |
| | | | Inc. | | from | | | | | | | |
| | | | | | Waxy | | | | | | | |
| | | | | | Maize - | | | | | | | |
| | | | | | Ingredion | | | | | | | |
| | | | | | Inc. | | | | | | | |
| 543 | | | Modified | 2.5 | Modified | 2.5 | Modified | 50.0 | | | 35.0 | 65.0 |
| | | | Gum | | Food | | Food | | | | | |
| | | | Acacia - | | Starch | | Starch, | | | | | |
| | | | Ingredion | | Refined | | Tapioca, | | | | | |
| | | | Inc. | | from | | 06670301 | | | | | |
| | | | | | Waxy | | | | | | | |
| | | | | | Maize - | | | | | | | |
| | | | | | Ingredion | | | | | | | |
| | | | | | Inc. | | | | | | | |
| 544 | Flavor- | 5.0 | Modified | 10.0 | | | | | | | 55.0 | 45.0 |
| | free | | Food | | | | | | | | | |
| | Maltodextrin - | | Starch | | | | | | | | | |
| | Ingredion | | Refined | | | | | | | | | |
| | Inc. | | from | | | | | | | | | |
| | | | Waxy | | | | | | | | | |
| | | | Maize - | | | | | | | | | |
| | | | Ingredion | | | | | | | | | |
| | | | Inc. | | | | | | | | | |
| 545 | | | Modified | 24.0 | | | | | | | 60.0 | 40.0 |
| | | | Food | | | | | | | | | |
| | | | Starch | | | | | | | | | |
| | | | from | | | | | | | | | |
| | | | Waxy | | | | | | | | | |
| | | | Maize - | | | | | | | | | |
| | | | Ingredion | | | | | | | | | |
| | | | Inc. | | | | | | | | | |
| 546 | Inulin, | 5.0 | Quillaja | 50.0 | | | | | | | 15.0 | 45.0 |
| | water | | Saponaria | | | | | | | | | |
| | soluble | | Extract - | | | | | | | | | |
| | fiber - It's | | Ingredion | | | | | | | | | |
| | Just | | Inc. | | | | | | | | | |
| 547 | Flavor- | 5.0 | Modified | 10.0 | | | | | | | 55.0 | 45.0 |
| | free | | Food | | | | | | | | | |
| | Maltodextrin - | | Starch | | | | | | | | | |
| | Ingredion | | from | | | | | | | | | |
| | Inc. | | Waxy | | | | | | | | | |
| | | | Maize - | | | | | | | | | |
| | | | Ingredion | | | | | | | | | |
| | | | Inc. | | | | | | | | | |
| 548 | Maltodextrin, | 25.0 | Modified | 10.0 | | | | | | | 55.0 | 45.0 |
| | 90% | | Food | | | | | | | | | |
| | Dietary | | Starch | | | | | | | | | |
| | Fiber - | | from | | | | | | | | | |
| | Fibersol | | Tapioca - | | | | | | | | | |
| | | | Ingredion | | | | | | | | | |
| | | | Inc. | | | | | | | | | |
| 549 | Flavor- | 10.0 | Modified | 10.0 | | | | | Bitterness | 1.0 | 40.0 | 60.0 |
| | free | | Food | | | | | | Blocker, | | | |
| | Maltodextrin - | | Starch | | | | | | Natural, | | | |
| | Ingredion | | from | | | | | | Type A - | | | |
| | Inc. | | Tapioca - | | | | | | Allen | | | |
| | | | Ingredion | | | | | | Flavors | | | |
| | | | Inc. | | | | | | Inc. | | | |
| 550 | | | Modified | 2.5 | Modified | 50.0 | Modified | 2.5 | | | 32.5 | 67.5 |
| | | | Food | | Gum | | Food | | | | | |
| | | | Starch | | Acacia - | | Starch | | | | | |
| | | | from | | Ingredion | | from | | | | | |
| | | | Tapioca - | | Inc. | | Waxy | | | | | |
| | | | Ingredion | | | | Maize - | | | | | |
| | | | Inc. | | | | Ingredion | | | | | |
| | | | | | | | Inc. | | | | | |
| 551 | | | Modified | 2.5 | Modified | 2.5 | Modified | 50.0 | | | 32.5 | 67.5 |
| | | | Food | | Gum | | Food | | | | | |
| | | | Starch | | Acacia - | | Starch | | | | | |
| | | | from | | Ingredion | | from | | | | | |
| | | | Tapioca - | | Inc. | | Waxy | | | | | |
| | | | Ingredion | | | | Maize - | | | | | |
| | | | Inc. | | | | Ingredion | | | | | |
| | | | | | | | Inc. | | | | | |
| 552 | Emulsifying | 10.0 | Modified | 10.0 | | | | | | | 40.0 | 60.0 |
| | Starch - | | Food | | | | | | | | | |
| | Cargill | | Starch | | | | | | | | | |
| | Inc. | | from | | | | | | | | | |
| | | | Tapioca - | | | | | | | | | |
| | | | Ingredion | | | | | | | | | |
| | | | Inc. | | | | | | | | | |
| 553 | | | Modified | 24.0 | | | | | | | 60.0 | 40.0 |
| | | | Food | | | | | | | | | |
| | | | Starch | | | | | | | | | |
| | | | from | | | | | | | | | |
| | | | Waxy | | | | | | | | | |
| | | | Maize - | | | | | | | | | |
| | | | Ingredion | | | | | | | | | |
| | | | Inc. | | | | | | | | | |
| 554 | Inulin, | 5.0 | Quillaja | 50.0 | | | | | | | 15.0 | 45.0 |
| | water | | Sapon | | | | | | | | | |
| | soluble | | aria | | | | | | | | | |
| | fiber - It's | | Extract - | | | | | | | | | |
| | Just | | Ingredion | | | | | | | | | |
| | | | Inc. | | | | | | | | | |
| 555 | Flavor- | 10.0 | Modified | 10.0 | | | | | Lemon | 1.2 | 50.0 | 50.0 |
| | free | | Food | | | | | | Oil | | | |
| | Maltodextrin - | | Starch | | | | | | Flavor - | | | |
| | Ingredion | | from | | | | | | Flavor | | | |
| | Inc. | | Tapioca - | | | | | | Insights | | | |
| | | | Ingredion | | | | | | | | | |
| | | | Inc. | | | | | | | | | |
| 556 | Flavor- | 10.0 | Modified | 5.0 | Modified | 5.0 | | | Bitterness | 1.5 | 48.0 | 52.0 |
| | free | | Food | | Food | | | | Blocker, | | | |
| | Maltodextrin - | | Starch | | Starch | | | | Natural, | | | |
| | Ingredion | | from | | from | | | | Type C - | | | |
| | Inc. | | Tapioca - | | Waxy | | | | Allen | | | |
| | | | Ingredion | | Maize - | | | | Flavors | | | |
| | | | Inc. | | Ingredion | | | | Inc. | | | |
| | | | | | Inc. | | | | | | | |

Table S008C shows spray dried particulate formulations resulting from electrostatic spray drying of the water dispersible emulsions having formulations of examples 475-556 as detailed above in tables S008A and S008B. Each particulate formulation is produced vis spray drying of the water-dispersible emulsion.

TABLE S008C

| | | Powder | | Powder | Powder | Powder | Powder | |
|---|---|---|---|---|---|---|---|---|
| | Powder | Carrier | Powder | Primary | Secondary | Tertiary | Additional | |
| | ABI | Oil | Encapsulate | Emulsifier | Emulsifier | Emulsifier | Components | |
| | Usage | Usage | Usage | Usage | Usage | Usage | Usage | Water |
| Example | Rate | Rate | Rate | Rate | Rate | Rate | Rate | Activity |
| 475 | 20.0 | 20.0 | | 60.0 | | | | 0.192 |
| 476 | 25.0 | 25.0 | | 50.0 | | | | |
| 477 | 33.3 | 33.3 | 11.1 | 22.2 | | | | |
| 478 | 11.1 | 11.1 | 55.6 | 22.2 | | | | 0.048 |
| 479 | 11.1 | 11.1 | 55.6 | 22.2 | | | | 0.185 |
| 480 | 11.1 | 11.1 | 55.6 | 22.2 | | | | 0.053 |
| 481 | 11.1 | 11.1 | 55.6 | 22.2 | | | | 0.034 |
| 482 | 26.7 | 26.7 | 33.3 | 13.3 | | | | 0.144 |
| 483 | 18.2 | 18.2 | 45.5 | 18.2 | | | | 0.108 |
| 484 | 22.2 | 22.2 | 33.3 | 22.2 | | | | 0.074 |
| 485 | 22.2 | 22.2 | 44.4 | 11.1 | | | | 0.119 |
| 486 | 33.3 | 33.3 | 11.1 | 22.2 | | | | 0.141 |
| 487 | 11.1 | 11.1 | 55.6 | 22.2 | | | | 0.066 |
| 488 | 33.4 | 33.4 | 11.0 | 22.2 | | | | 0.148 |
| 489 | 33.4 | 33.4 | 10.9 | 22.2 | | | | 0.137 |
| 490 | 33.4 | 33.4 | 11.1 | 22.2 | | | | 0.123 |
| 491 | 37.8 | 37.8 | 11.1 | 13.3 | | | | |
| 492 | 37.8 | 28.9 | 11.1 | 22.2 | | | | |
| 493 | 27.8 | 27.8 | 22.2 | 22.2 | | | | 0.248 |
| 494 | 30.0 | 30.0 | 20.0 | 20.0 | | | | 0.191 |
| 495 | 31.8 | 31.8 | 18.2 | 18.2 | | | | 0.143 |
| 496 | 33.3 | 33.3 | 16.7 | 16.7 | | | | 0.186 |
| 497 | 35.8 | 35.8 | 14.2 | 14.2 | | | | 0.148 |
| 498 | 37.5 | 37.5 | 12.5 | 12.5 | | | | 0.176 |
| 499 | 37.5 | 37.5 | 12.5 | 12.5 | | | | 0.102 |
| 500 | 37.5 | 37.5 | 16.7 | 8.3 | | | | 0.110 |
| 501 | 33.3 | 33.3 | 12.0 | 16.7 | | | 4.7 | 0.206 |
| 502 | 33.3 | 33.3 | 16.6 | 16.7 | | | 0.05 | 0.149 |
| 503 | 33.3 | 33.3 | 16.7 | 16.7 | | | | 0.147 |
| 504 | 33.3 | 33.3 | 16.7 | 16.7 | | | | |
| 505 | 33.3 | 33.3 | 16.7 | 16.7 | | | | |
| 506 | 33.3 | 33.3 | 16.7 | 16.7 | | | | |
| 507 | 33.3 | 33.3 | 11.1 | 22.2 | | | | 0.249 |
| 508 | 40.0 | 40.0 | 10.0 | 10.0 | | | | 0.190 |
| 509 | 11.1 | 11.1 | 55.5 | 22.3 | | | | |
| 510 | 27.8 | 27.8 | | 44.4 | | | | 0.132 |
| 511 | 30.0 | 30.0 | | 40.0 | | | | 0.051 |
| 512 | 27.8 | 27.8 | 22.2 | 22.2 | | | | 0.067 |
| 513 | 27.8 | 27.8 | | 44.4 | | | | 0.066 |
| 514 | 27.8 | 27.8 | 22.2 | 22.2 | | | | 0.155 |
| 515 | 33.3 | 33.3 | 11.1 | 22.2 | | | | 0.210 |
| 516 | 20.0 | 20.0 | | 60.0 | | | | 0.201 |
| 517 | 33.3 | 33.3 | 11.1 | 22.2 | | | | 0.234 |
| 518 | 37.5 | 37.5 | 12.5 | 12.5 | | | | 0.127 |
| 519 | 33.3 | 33.3 | 11.1 | 22.2 | | | | |
| 520 | 11.1 | 11.1 | 55.6 | 22.2 | | | | |
| 521 | 33.3 | 32.0 | 16.7 | 8.3 | 4.2 | 4.2 | 1.3 | |
| 522 | 33.3 | 33.3 | 16.7 | 16.7 | | | | |
| 523 | 33.3 | 33.3 | 16.7 | 16.7 | | | | |
| 524 | 40.0 | 40.0 | 10.0 | 10.0 | | | | |
| 525 | 27.8 | 27.8 | 22.2 | 22.2 | | | | |
| 526 | 27.8 | 27.8 | 22.2 | 22.2 | | | | |
| 527 | 33.3 | 33.3 | 11.1 | 22.2 | | | | |
| 528 | 20.0 | 20.0 | | 60.0 | | | | |
| 529 | 16.0 | 16.0 | | 10.0 | 36.0 | 22.0 | | |
| 530 | 33.3 | 33.3 | 11.1 | 22.2 | | | | |
| 531 | 33.3 | 33.3 | 16.7 | 16.7 | | | | 0.108 |
| 532 | 27.8 | 27.8 | 22.2 | 22.2 | | | | 0.292 |
| 533 | 33.3 | 33.3 | 16.7 | 16.7 | | | | 0.095 |
| 534 | 33.3 | 33.3 | 11.1 | 22.2 | | | | |
| 535 | 11.1 | 11.1 | 55.6 | 22.2 | | | | |
| 536 | 33.3 | 33.3 | 16.7 | 16.7 | | | | |
| 537 | 33.3 | 33.3 | 16.7 | 16.7 | | | | |
| 538 | 33.3 | 33.3 | 16.7 | 16.7 | | | | |
| 539 | 40.0 | 40.0 | 10.0 | 10.0 | | | | |
| 540 | 27.8 | 27.8 | 22.2 | 22.2 | | | | |
| 541 | 27.8 | 27.8 | 22.2 | 22.2 | | | | |
| 542 | 7.7 | 7.7 | | 7.7 | 76.9 | | | |
| 543 | 7.7 | 7.7 | | 3.8 | 3.8 | 76.9 | | |
| 544 | 33.3 | 33.3 | 11.1 | 22.2 | | | | |
| 545 | 20.0 | 20.0 | | 60.0 | | | | |
| 546 | 33.3 | 33.3 | 11.1 | 22.2 | | | | |
| 547 | 16.7 | 33.3 | 11.1 | 22.2 | | | | |
| | 16.7 | | | | | | | |
| 548 | 5.6 | 11.1 | 55.6 | 22.2 | | | | |
| | 5.6 | | | | | | | |
| 549 | 10.0 | 31.7 | 16.7 | 16.7 | | | 1.7 | |
| | 11.7 | | | | | | | |
| | 11.7 | | | | | | | |
| 550 | 3.7 | 7.4 | | 3.7 | 74.1 | 3.7 | | |
| | 3.7 | | | | | | | |
| | 3.7 | | | | | | | |
| 551 | 3.7 | 7.4 | | 3.7 | 3.7 | 74.1 | | |
| | 3.7 | | | | | | | |
| | 3.7 | | | | | | | |
| 552 | 11.7 | 33.3 | 16.7 | 16.7 | | | | |
| | 11.7 | | | | | | | |
| | 10.0 | | | | | | | |
| 553 | 10.0 | 20.0 | | 60.0 | | | | |
| | 10.0 | | | | | | | |
| 554 | 16.7 | 33.3 | 11.1 | 22.2 | | | | |
| | 16.7 | | | | | | | |
| 555 | 10.0 | 37.6 | 20.0 | 20.0 | | | 2.4 | |
| | 10.0 | | | | | | | |
| 556 | 7.7 | 35.6 | 19.2 | 9.6 | 9.6 | | 2.9 | |
| | 15.4 | | | | | | | |

### Comparative Examples 557-600

Table S008C shows comparative examples that do not work, in that there was separation in the emulsion or unsuccessful spray drying. In each of these compositions, the ABI was CBD Isolate, the continuous phase in the hydrophilic phase was water at 55.0% usage rate, and there was a 45% solid percent of the resulting emulsion.

TABLE S008C
Comparative Examples

| | | | | | | | | | | Particulate | | | |
|---|---|---|---|---|---|---|---|---|---|---|---|---|---|
| | | | | | | | | | Particulate | Carrier | Particulate | | |
| Com- | Proto- | ABI | | | | | | | ABI | Oil | Emulsifier | | Notes |
| parative | type | Usage | Carrier | Usage | | Usage | Primary | Usage | Usage | Usage | Usage | Water | on |
| Example | ID | Rate | Oil | Rate | Encapsulant | Rate | Emulsifier | Rate | Rate | Rate | Rate | Activity | failure |
| 557 | 3A8 | 15.0 | MCT | 15.0 | Flavor- | 15.0 | | | | | | | Rapid |
| | | | | | free | | | | | | | | separation |
| | | | | | Maltodextrin- | | | | | | | | of the |
| | | | | | Ingredion | | | | | | | | emulsion |
| | | | | | Inc. | | | | | | | | observed |
| | | | | | | | | | | | | | prior |
| | | | | | | | | | | | | | to |
| | | | | | | | | | | | | | spray |
| | | | | | | | | | | | | | drying |
| 558 | 3A13 | 15.0 | MCT | 15.0 | | | Modified | 15.0 | 33.3 | 33.3 | 33.3 | 0.818 | Large |
| | | | | | | | Food | | | | | | deposits |
| | | | | | | | Starch | | | | | | of |
| | | | | | | | from | | | | | | powder |
| | | | | | | | Tapioca- | | | | | | formed |
| | | | | | | | Ingredion | | | | | | in |
| | | | | | | | Inc. | | | | | | the |
| | | | | | | | | | | | | | spray |
| | | | | | | | | | | | | | dryer |
| | | | | | | | | | | | | | with |
| | | | | | | | | | | | | | clay- |
| | | | | | | | | | | | | | like |
| | | | | | | | | | | | | | consistency |
| 559 | 3A11 | 18.0 | MCT | 18.0 | | | Modified | 9.0 | | | | | Rapid |
| | | | | | | | Starch | | | | | | separation |
| | | | | | | | from | | | | | | of |
| | | | | | | | Waxy | | | | | | the |
| | | | | | | | Maize- | | | | | | emulsion |
| | | | | | | | Ingredion | | | | | | observed |
| | | | | | | | Inc. | | | | | | prior |
| | | | | | | | | | | | | | to |
| | | | | | | | | | | | | | spray |
| | | | | | | | | | | | | | drying |
| 600 | 3A5 | 30.0 | | | Flavor- | 5.0 | Modified | 10.0 | | | | | CBD |
| | C | | | | free | | Food | | | | | | crystallization |
| | | | | | Maltodextrin- | | Starch | | | | | | observed |
| | | | | | Ingredion | | from | | | | | | in |
| | | | | | Inc. | | Tapioca- | | | | | | the |
| | | | | | | | Ingredion | | | | | | emulsion |
| | | | | | | | Inc. | | | | | | prior |
| | | | | | | | | | | | | | to |
| | | | | | | | | | | | | | spray |
| | | | | | | | | | | | | | drying |

### Example 601-610

Tables S009A, S009B, and S009C show the stability of the formulation (and also including examples that have lower stability). These examples show low separation rates for most examples and juxtaposing formulations showing high separation (e.g. gum arabic). These examples show the importance of the selection of the emulsifier. Relevant portions of the disclosure above include paragraphs [00104] and [00106].

TABLE S009A
compositions of the hydrophobic phase

| | Prototype | | Usage | | Usage | Flavorant or | Usage |
|---|---|---|---|---|---|---|---|
| Example | ID | ABI | Rate | Carrier Oil | Rate | Bittermasker | Rate |
| 601 | 19A | Full | 1.0 | MCT | 1.0 | | |
| | | Spectrum | | | | | |
| | | Distillate | | | | | |
| 602 | 18D | Broad | 1 | MCT | 1 | | |
| | | Spectrum | | | | | |
| | | Distillate | | | | | |
| 603 | 10F16 | CBD | 4 | MCT | 4 | Bitterness Blocker, | 0.72 |
| | | Isolate | | | | Natural, Type C- | |
| | | | | | | Allen Flavors Inc. | |
| 604 | 56F4B | CBD | 5.0 | MCT | 5.0 | Bitterness Blocker, | 0.9 |
| | | Isolate | | | | Natural, Type C- | |
| | | | | | | Allen Flavors Inc. | |
| 605 | 21G | Ful | 2.0 | Hemp | 2.0 | | |
| | | Spectrum | | seed oil | | | |
| | | Distillate | | | | | |
| 606 | 56F4 | CBD | 5 | MCT | 5 | AF103282 | 0.9 |
| | | Isolate | | | | | |
| 607 | 18A | Ful | 1.0 | MCT | 1.0 | | |
| | | Spectrum | | | | | |
| | | Distillate | | | | | |
| 608 | 60 | CBD | 1.0 | MCT | 1.0 | | |
| | | Isolate | | | | | |
| 609 | 47 | Broad | 1.0 | MCT | 1.0 | | |
| | | Spectrum | | | | | |
| | | Distillate | | | | | |
| 610 | WGU-1A- | CBD | 0.5 | MCT | 0.5 | | |
| | 30000 | Isolate | | | | | |

TABLE S009B
compositions of the hydrophilic phase

| | | Usage | Secondary | Usage | Continuous | Usage |
|---|---|---|---|---|---|---|
| Example | Primary Emulsifier | Rate | Emulsifier | Rate | Phase | Rate |
| 60 | “Soy Lecithin (with | 3.0 | | | Glycerin | 95.0 |
| | Ethoxylated Mono- | | | | | |
| | diglycerides and | | | | | |
| | Propylene Glycol)- | | | | | |
| | Americal Lecithin | | | | | |
| | Company” | | | | | |
| 602 | Acetylated Lecithins- | 3 | Fatfree Sunflower | 1 | Glycerin | 94 |
| | Cargill Inc. | | Lecithin (non-GMO) | | | |
| | | | with 60% | | | |
| | | | Phosphatidylcholine- | | | |
| | | | Lipoid | | | |
| 603 | Quillaja Saponaria | 4 | Sunflower Lecithin, | 4 | Glycerin | 83.28 |
| | Extract-Ingredion Inc. | | ≥99%-Compass | | | |
| | | | Foods Pte Ltd | | | |
| 604 | Quillaja Saponaria | 10.0 | Sucrose Monoester | 1.7 | Glycerin | 77.4 |
| | Extract-Naturex | | of Palmitic Acid- | | | |
| | | | Compass Foods Pte | | | |
| | | | Ltd | | | |
| 605 | Lecithin with | 4.0 | | | Glycerin | 92.0 |
| | Ethoxylated mono di- | | | | | |
| | glycerides-Solae | | | | | |
| 606 | Quillaja Saponaria | 10 | Sucrose Monoester | 1.7 | Glycerin | 77.4 |
| | Extract-Naturex | | of Palmitic Acid- | | | |
| | | | Compass Foods Pte | | | |
| | | | Ltd | | | |
| 607 | Acetylated Lecithins- | 4.0 | | | Glycerin | 94.0 |
| | Cargill Inc. | | | | | |
| 608 | Modified Food Starch | 1.0 | | | Glycerin | 97.0 |
| | from Tapioca- | | | | | |
| | Ingredion Inc. | | | | | |
| 609 | Grass-fed Bovine | 1.0 | | | Glycerin | 97.0 |
| | Collagen Peptides- | | | | | |
| | PeptPure | | | | | |
| 610 | Pre-hydrated Gum | 1.5 | | | Glycerin | 97.5 |
| | Arabic | | | | | |

Table S0090C shows the utility of measuring factors such as the stability index and separation rate in evaluating the stability of example emulsions under accelerated conditions. Stable and unstable emulsions are showcased in this table as examples.

TABLE S009C
stability parameters and calculations

| | | Separation | Mean | Separation | Separation |
|---|---|---|---|---|---|
| | Stability | Rate | RCA | Rate (%/min | Rate (%/year |
| Example | Index | (%/min) | (g) | at 1 g) | at 1 g) |
| 601 | 0.007 | 0.0007 | 2214 | 0.00000032 | 0.17 |
| 602 | 0.016 | 0.0008 | 2223 | 0.00000036 | 0.19 |
| 603 | 0.02 | 0.0013 | 2196 | 0.00000059 | 0.31 |
| 604 | 0.016 | 0.0014 | 2159 | 0.00000065 | 0.34 |
| 605 | 0.006 | 0.0015 | 2220 | 0.00000068 | 0.36 |
| 606 | 0.008 | 0.0033 | 2168 | 0.00000152 | 0.80 |
| 607 | 0.003 | 0.0023 | 1241 | 0.00000185 | 0.97 |
| 608 | 0.888 | 0.3192 | 2165 | 0.00014744 | 77.49 |
| 609 | 0.816 | 1.36 | 851.2 | 0.00159774 | 100.00 |
| 610 | 0.907 | 1.379 | 2178 | 0.00063315 | 100 |

### Examples 611-616

Tables S010A, S010B, and S010B show stability (% w/w) of formulations of the invention over time.

TABLE S010A
example spray dried particulate formulations

| | | | Usage | | Usage | | Usage | | Usage | | |
|---|---|---|---|---|---|---|---|---|---|---|---|
| | | Active | Rate | | Rate | | Rate | | Rate | | |
| | Prototype | Biological | (% | Carrier | (% | | (% | Primary | (% | | Water |
| Example | ID | Ingredient | w/w) | Oil | w/w) | Encapsulant | w/w) | Emulsifier | w/w) | Total | Activity |
| 611 | 3A7D | CBD | 33.3 | MCT | 33.3 | Flavor- | 16.7 | Modified | 16.7 | 100.0 | 0.2 |
| | | Isolate | | | | free | | Food | | | |
| | | | | | | Maltodextrin- | | Starch | | | |
| | | | | | | Ingredion | | from | | | |
| | | | | | | Inc. | | Tapioca- | | | |
| | | | | | | | | Ingredion | | | |
| | | | | | | | | Inc. | | | |
| 612 | 3A7D7 | CBD | 33.3 | MCT | 33.3 | Flavor- | 16.7 | Modified | 16.7 | 100.0 | 0.1 |
| | | Isolate | | | | free | | Food | | | |
| | | | | | | Maltodextrin- | | Starch | | | |
| | | | | | | Ingredion | | from | | | |
| | | | | | | Inc. | | Tapioca- | | | |
| | | | | | | | | Ingredion | | | |
| | | | | | | | | Inc. | | | |

TABLE S010B
stability results of the example spray dried particulates

| | | Theoretical | CBD | CBD |
|---|---|---|---|---|
| | | CBD | Concentration | Concentration |
| | Prototype | Concentration | (% w/w) at 11 | (% w/w) at 13 |
| Example | ID | (% w/w) | Weeks | Weeks |
| 611 | 3A7D | 32.7 | | 31.79 |
| 612 | 3A7D7 | 32.7 | 31.60 | |

TABLE S010C
example liquid emulsions, stability results showing
CBD Concentration (% w/w) at Timepoint in weeks

| | | Theoretical | | | | | | | | | | | |
|---|---|---|---|---|---|---|---|---|---|---|---|---|---|
| | | CBD | | | | | | | | | | | |
| | | Concentration | | | | | | | | | | | |
| | Prototype | (% | | 2 | 7 | 11 | 17 | 18 | 23 | 29 | 34 | 36 | 46 |
| Example | ID | w/w) | T0 | Weeks | Weeks | Weeks | Weeks | Weeks | Weeks | Weeks | Weeks | Weeks | Weeks |
| 613 | 10F | 0 | | 1.30 | 1.22 | 1.21 | 1.20 | | | | | | |
| 614 | 56D | 0.98 | | 1.03 | 0.98 | 0.93 | 0.99 | | | | | | |
| 615 | 25G5 | 0.98 | 0.98 | | | | | 1.08 | 1.07 | 1.01 | | 1.04 | |
| 616 | 25G2 | 0.98 | | 0.97 | | | | | | | 1.01 | | 1.04 |

### Examples 617-622

Tables S011A-Z show results for wettability, where a shorter time is better. Specifically, these examples show how adjusting the voltage changes wettability—namely a higher voltage results in better wettable. Notably, pulsed voltage achieves the wettability best. These examples relate to the disclose above at least at paragraph [00111].

TABLE S011A
particulate formulations

| | | | Usage | | Usage | | Usage | | Usage |
|---|---|---|---|---|---|---|---|---|---|
| | | Active | Rate | | Rate | | Rate | | Rate |
| | Prototype | Biological | (% | Carrier | (% | | (% | Primary | (% |
| Example | ID | Ingredient | w/w) | Oil | w/w) | Encapsulant | w/w) | Emulsifier | w/w) |
| 617 | 3A7A- | CBD | 27.8 | MCT | 27.8 | Flavor-free | 22.2 | Modified | 22.2 |
| | VOL1 | Isolate | | | | Maltodextrin- | | Food | |
| | | | | | | Ingredion | | Starch | |
| | | | | | | Inc. | | from | |
| | | | | | | | | Tapioca- | |
| | | | | | | | | Ingredion | |
| | | | | | | | | Inc. | |
| 618 | 3A7A- | CBD | 27.8 | MCT | 27.8 | Flavor-free | 22.2 | Modified | 22.2 |
| | VOL2 | Isolate | | | | Maltodextrin- | | Food | |
| | | | | | | Ingredion | | Starch | |
| | | | | | | Inc. | | from | |
| | | | | | | | | Tapioca- | |
| | | | | | | | | Ingredion | |
| | | | | | | | | Inc. | |
| 619 | 3A7A- | CBD | 27.8 | MCT | 27.8 | Flavor-free | 22.2 | Modified | 22.2 |
| | VOL3 | Isolate | | | | Maltodextrin- | | Food | |
| | | | | | | Ingredion | | Starch | |
| | | | | | | Inc. | | from | |
| | | | | | | | | Tapioca- | |
| | | | | | | | | Ingredion | |
| | | | | | | | | Inc. | |
| 620 | 3A7A- | CBD | 27.8 | MCT | 27.8 | Flavor-free | 22.2 | Modified | 22.2 |
| | VOL4 | Isolate | | | | Maltodextrin- | | Food | |
| | | | | | | Ingredion | | Starch | |
| | | | | | | Inc. | | from | |
| | | | | | | | | Tapioca- | |
| | | | | | | | | Ingredion | |
| | | | | | | | | Inc. | |
| 621 | 3A7A- | CBD | 27.8 | MCT | 27.8 | Flavor-free | 22.2 | Modified | 22.2 |
| | VOL5 | Isolate | | | | Maltodextrin- | | Food | |
| | | | | | | Ingredion | | Starch | |
| | | | | | | Inc. | | from | |
| | | | | | | | | Tapioca- | |
| | | | | | | | | Ingredion | |
| | | | | | | | | Inc. | |
| 622 | 3A7A- | CBD | 27.8 | MCT | 27.8 | Flavor-free | 22.2 | Modified | 22.2 |
| | VOL6 | Isolate | | | | Maltodextrin- | | Food | |
| | | | | | | Ingredion | | Starch | |
| | | | | | | Inc. | | from | |
| | | | | | | | | Tapioca- | |
| | | | | | | | | Ingredion | |
| | | | | | | | | Inc. | |

TABLE S011B
corresponding wettability values

| | Homogenization | Gas | Drying | Atomizing | | | | |
|---|---|---|---|---|---|---|---|---|
| | Pressure | Flow | Gas | Gas | | Atomizing | Chamber | |
| | for the Feed | (Nm3/ | Heat | Pressure | Voltage | Gas | Vacuum | Wettability |
| Example | Emulsion (psi) | hr) | (C.) | (kPa) | (kV) | Heat (C.) | (kPa) | (sec) |
| 617 | 10000 | 25 | 140 | 200 | 25 | 100 | 0.1 | 38 |
| 618 | 10000 | 25 | 140 | 200 | 20 | 100 | 0.1 | 95 |
| 619 | 10000 | 25 | 140 | 200 | 15 | 100 | 0.1 | 322 |
| 620 | 10000 | 25 | 140 | 200 | 10 | 100 | 0.1 | 230 |
| 621 | 10000 | 25 | 140 | 200 | 5 | 100 | 0.1 | 313 |
| 622 | 10000 | 25 | 140 | 200 | Pulsed | 100 | 0.1 | 21 |
| | | | | | Voltage | | | |
| | | | | | (25 | | | |
| | | | | | kv, 1 s; | | | |
| | | | | | 2.5 kv, | | | |
| | | | | | 1 s) | | | |

While various embodiments of the disclosure have been described, the description is intended to be exemplary, rather than limiting and it will be apparent to those of ordinary skill in the art that many more embodiments and implementations are possible that are within the scope of the disclosure. Accordingly, the disclosure is not to be restricted except in light of the attached claims and their equivalents. Also, various modifications and changes may be made within the scope of the attached claims.

We claim:

1. A stable emulsion comprising:
a hydrophobic phase and a hydrophilic phase;
wherein the hydrophobic phase comprises an active biological ingredient and a carrier oil; and
wherein the hydrophilic phase comprises a continuous phase and an emulsifier;
wherein the emulsion has a separation rate between 0.1 percent per year and 10 percent per year.

2. The stable emulsion of claim 1, wherein the hydrophobic phase in the emulsion has a median particle size of between 1 nm and 500 nm.

3. The stable emulsion of claim 1, wherein the continuous phase is between 30 weight percent and 98.5 weight percent of the stable emulsion, based on a total weight of the stable emulsion.

4. The stable emulsion of claim 1, wherein a total weight percent of active biological ingredient in the stable emulsion is between 0.001 weight percent and 30 weight percent, based on a total weight of the stable emulsion; and
wherein the active biological ingredient is present at a concentration of between 90 percent and 100 percent of a theoretical concentration of the active biological ingredient in the stable emulsion for greater than 100 weeks.

5. The stable emulsion of claim 1, wherein a total weight percent of carrier oil in the stable emulsion is between 0.5 weight percent and 40.0 weight percent.

6. The stable emulsion of claim 1, wherein a total weight percent of emulsifier in the stable emulsion is between 0.001 weight percent and 30 weight percent.

7. The stable emulsion of claim 1, wherein a measurement of water activity of the stable emulsion is from 0.05 to 0.9.

8. A stable emulsion for electrostatic spray drying comprising:
a hydrophobic phase and a hydrophilic phase,
wherein the hydrophobic phase comprises an active biological ingredient and a carrier oil;
wherein the hydrophilic phase comprises a continuous phase and an emulsifier; and
wherein the emulsion has a separation rate between 0.1 percent per year and 75 percent per year.

9. The stable emulsion of claim 8, wherein the hydrophobic phase in the emulsion has a median particle size of between 5 nm and 1000 nm.

10. The stable emulsion of claim 8, wherein a weight percent of carrier oil in the stable emulsion is between 0.001 weight percent and 60 weight percent, based on a total weight of the stable emulsion.

11. The stable emulsion of claim 8, wherein the continuous phase is between 30 weight percent and 80 weight percent of the stable emulsion, based on a total weight of the stable emulsion.

13. The stable emulsion of claim 8, wherein a weight percent of active biological ingredient in the stable emulsion is between 0.001 weight percent and 60 weight percent, based on a total weight of the emulsion.

14. The stable emulsion of claim 8, wherein
a weight percent of emulsifier in the stable emulsion is between 0.1 weight percent and 90 weight percent, based on a total weight of the emulsion; and
wherein a weight percent of encapsulant is between 0.01 weight percent and 40.0 weight percent, based on the total weight of the emulsion.

15. The stable emulsion of claim 8, further having a solids weight percent between 20 weight percent and 70 weight percent, based on the total weight of the emulsion.

16. A stable, water-dispersible particulate comprising:
an active biological ingredient; and
a shell encapsulating the active biological ingredient, and
the shell comprising an encapsulant.

17. The stable, water-dispersible particulate of claim 16, wherein the stable, water-dispersible particulate has a median particle size between 1 μm and 500 μm.

18. The stable, water-dispersible particulate of claim 16, wherein a measurement of water activity is from 0.01 to 0.9.

19. The stable, water-dispersible particulate of claim 16, wherein the active biological ingredient is present at a concentration of between 90 percent and 100 percent of a theoretical concentration of the active biological ingredient in the stable emulsion for greater than 100 weeks.

20. The stable water-dispersible particulate of claim 16, wherein a wettability measurement of the particulate is between 0.1 seconds and 350 seconds.
